SEQUENCE LISTING (88 sequences)

SEQ ID NO: 1 - protein, 38 aa, Artificial Sequence
  Description of Artificial Sequence Synthetic polypeptide
GSMCMPCFTTDHQMARRCDDCCGGRGRGRCYGPQCLCR

SEQ ID NO: 2 - protein, 39 aa, Artificial Sequence
  Description of Artificial Sequence Synthetic polypeptide
GSEVIRCSGSKQCYGPCKQQTGCTNSKCMNKVCKCYGCG

SEQ ID NO: 3 - protein, 37 aa, Artificial Sequence
  Description of Artificial Sequence Synthetic polypeptide
GSSEKDCIKHLQRCRENKDCCSKKCSRRGTNPEKRCR

SEQ ID NO: 4 - protein, 36 aa, Artificial Sequence
  Description of Artificial Sequence Synthetic polypeptide
GSSCAKPRENCNRMNILCCRGECVCPTFGDCFCYGD

SEQ ID NO: 5 - protein, 39 aa, Artificial Sequence
  Description of Artificial Sequence Synthetic polypeptide
GSGVPINVKCRGSRDCLDPCKKAGMRFGKCINSKCHCTP

SEQ ID NO: 6 - protein, 39 aa, Artificial Sequence
  Description of Artificial Sequence Synthetic polypeptide
GSVRIPVSCKHSGQCLKPCKDAGMRFGKCMNGKCDCTPK

SEQ ID NO: 7 - protein, 39 aa, Artificial Sequence
  Description of Artificial Sequence Synthetic polypeptide
GSDCVRFWGKCSQTSDCCPHLACKSKWPRNICVWDGSVG

SEQ ID NO: 8 - protein, 38 aa, Artificial Sequence
  Description of Artificial Sequence Synthetic polypeptide
GSMCMPCFTTRPDMAQQCRACCKGRGKCFGPQCLCGYD

SEQ ID NO: 9 - protein, 35 aa, Artificial Sequence
  Description of Artificial Sequence Synthetic polypeptide
GSGCFGYKCDYYKGCCSGYVCSPTWKWCVRPGPGR

SEQ ID NO: 10 - protein, 40 aa, Artificial Sequence
  Description of Artificial Sequence Synthetic polypeptide
GSQVQTNVKCQGGSCASVCRREIGVAAGKCINGKCVCYRN

SEQ ID NO: 11 - protein, 35 aa, Artificial Sequence
  Description of Artificial Sequence Synthetic polypeptide
GSGDCLPHLKRCKENNDCCSKKCKRRGANPEKRCR

SEQ ID NO: 12 - protein, 37 aa, Artificial Sequence
  Description of Artificial Sequence Synthetic polypeptide
GSNFKVEGACSKPCRKYCIDKGARNGKCINGRCHCYY

SEQ ID NO: 13 - protein, 40 aa, Artificial Sequence
  Description of Artificial Sequence Synthetic polypeptide
GSQKILSNRCNNSSECIPHCIRIFGTRAAKCINRKCYCYP

SEQ ID NO: 14 - protein, 44 aa, Artificial Sequence
  Description of Artificial Sequence Synthetic polypeptide
GSDRDSCIDKSRCSKYGYYQECQDCCKKAGHNGGTCMFFKCKCA

SEQ ID NO: 15 - protein, 34 aa, Artificial Sequence
  Description of Artificial Sequence Synthetic polypeptide
GSAVCNLKRCQLSCRSLGLLGKCIGDKCECVKHG

SEQ ID NO: 16 - protein, 39 aa, Artificial Sequence
  Description of Artificial Sequence Synthetic polypeptide
GSQFCGTNGKPCVNGQCCGALRCVVTYHYADGVCLKMNP

SEQ ID NO: 17 - protein, 38 aa, Artificial Sequence
  Description of Artificial Sequence Synthetic polypeptide
GSRPTDIKCSASYQCFPVCKSRFGKTNGRCVNGLCDCF

SEQ ID NO: 18 - protein, 37 aa, Artificial Sequence
  Description of Artificial Sequence Synthetic polypeptide
GSNCAGYMRECKEKLCCSGYVCSSRWKWCVLPAPWRR

SEQ ID NO: 19 - protein, 39 aa, Artificial Sequence
  Description of Artificial Sequence Synthetic polypeptide
GSQFTDVKCTGSKQCWPVCKQMFGKPNGKCMNGKCRCYS

SEQ ID NO: 20 - protein, 40 aa, Artificial Sequence
  Description of Artificial Sequence Synthetic polypeptide
GSQIDTNVKCSGSSKCVKICIDRYNTRGAKCINGRCTCYP

SEQ ID NO: 21 - protein, 40 aa, Artificial Sequence
  Description of Artificial Sequence Synthetic polypeptide
GSAEIIRCSGTRECYAPCQKLTGCLNAKCMNKACKCYGCV

SEQ ID NO: 22 - protein, 38 aa, Artificial Sequence
  Description of Artificial Sequence Synthetic polypeptide
GSSDYCSNDFCFFSCRRDRCARGDCENGKCVCKNCHLN

SEQ ID NO: 23 - protein, 41 aa, Artificial Sequence
  Description of Artificial Sequence Synthetic polypeptide
GSCIGEGVPCDENDPRCCFGLVCLKPTLHGIWYKSYYCYKK

SEQ ID NO: 24 - protein, 33 aa, Artificial Sequence
  Description of Artificial Sequence Synthetic polypeptide
GSSCAKPGEMCMRIKCCDGQCGCNRGTGRCFCK

SEQ ID NO: 25 - protein, 37 aa, Artificial Sequence
  Description of Artificial Sequence Synthetic polypeptide
GSACLAEYQKCEGSTVPCCPGLSCSAGRFRKTKLCTK

SEQ ID NO: 26 - protein, 37 aa, Artificial Sequence
  Description of Artificial Sequence Synthetic polypeptide
GSVVIGQRCYRSPDCYSACKKLVGKATGKCTNGRCDC

SEQ ID NO: 27 - protein, 34 aa, Artificial Sequence
  Description of Artificial Sequence Synthetic polypeptide
GSACQFWSCNSSCISRGYRQGYCWGIQYKYCQCQ

SEQ ID NO: 28 - protein, 38 aa, Artificial Sequence
  Description of Artificial Sequence Synthetic polypeptide
GSRCPPCFTTNPNMEADCRKCCGGRGYCASYQCICPGG

SEQ ID NO: 29 - protein, 39 aa, Artificial Sequence
  Description of Artificial Sequence Synthetic polypeptide
GSVFINVKCRGSPECLPKCKEAIGKSAGKCMNGKCKCYP

SEQ ID NO: 30 - protein, 40 aa, Artificial Sequence
  Description of Artificial Sequence Synthetic polypeptide
GSQVSTNKKCSNTSQCYKTCEKVVGVAAGKCMNGKCICYP

SEQ ID NO: 31 - protein, 38 aa, Artificial Sequence
  Description of Artificial Sequence Synthetic polypeptide
GSECLEIFKACNPSNDQCCKSSKLVCSRKTRWCKYQIG

SEQ ID NO: 32 - protein, 56 aa, Artificial Sequence
  Description of Artificial Sequence Synthetic polypeptide
GSQDKCKKVYENYPVSKCQLANQCNYDCKLDKHARSGECFYDEKRNLQCICDYCEY

SEQ ID NO: 33 - protein, 39 aa, Artificial Sequence
  Description of Artificial Sequence Synthetic polypeptide
GSGVPINVRCRGSRDCLDPCRRAGMRFGRCINSRCHCTP

SEQ ID NO: 34 - protein, 46 aa, Artificial Sequence
  Description of Artificial Sequence Synthetic polypeptide
GGYSRCQLQGFNCVVRSYGLPTIPCCRGLTCRSYFPGSTYGRCQRY

SEQ ID NO: 35 - protein, 38 aa, Artificial Sequence
  Description of Artificial Sequence Synthetic polypeptide
GSMCMPCFTTDHQMARKCDDCCGGKGRGKCYGPQCLCR

SEQ ID NO: 36 - protein, 59 aa, Artificial Sequence
  Description of Artificial Sequence Synthetic polypeptide
GSAQEPVKGPVSTKPGSCPIILIRCAMLNPPNRCLKDTDCPGIKKCCEGSCGMACFVPQ

SEQ ID NO: 37 - protein, 37 aa, Artificial Sequence
  Description of Artificial Sequence Synthetic polypeptide
GSMCMPCFTTDPNMAKKCRDCCGGNGKCFGPQCLCNR

SEQ ID NO: 38 - protein, 43 aa, Artificial Sequence
  Description of Artificial Sequence Synthetic polypeptide
GSGIGDPVTCLKSGAICHPVFCPRRYKQIGTCGLPGTKCCKKP

SEQ ID NO: 39 - protein, 40 aa, Artificial Sequence
  Description of Artificial Sequence Synthetic polypeptide
GSGVPTDVKCRGSPQCIQPCKDAGMRFGKCMNGKCHCTPK

SEQ ID NO: 40 - protein, 38 aa, Artificial Sequence; identical to SEQ ID NO: 28
  Description of Artificial Sequence Synthetic polypeptide

SEQ ID NO: 41 - protein, 36 aa, Artificial Sequence
  Description of Artificial Sequence Synthetic polypeptide
MCMPCFTTDHQMARRCDDCCGGRGRGRCYGPQCLCR

SEQ ID NO: 42 - protein, 37 aa, Artificial Sequence
  Description of Artificial Sequence Synthetic polypeptide
EVIRCSGSKQCYGPCKQQTGCTNSKCMNKVCKCYGCG

SEQ ID NO: 43 - protein, 35 aa, Artificial Sequence
  Description of Artificial Sequence Synthetic polypeptide
SEKDCIKHLQRCRENKDCCSKKCSRRGTNPEKRCR

SEQ ID NO: 44 - protein, 34 aa, Artificial Sequence
  Description of Artificial Sequence Synthetic polypeptide
SCAKPRENCNRMNILCCRGECVCPTFGDCFCYGD

SEQ ID NO: 45 - protein, 37 aa, Artificial Sequence
  Description of Artificial Sequence Synthetic polypeptide
GVPINVKCRGSRDCLDPCKKAGMRFGKCINSKCHCTP

SEQ ID NO: 46 - protein, 37 aa, Artificial Sequence
  Description of Artificial Sequence Synthetic polypeptide
VRIPVSCKHSGQCLKPCKDAGMRFGKCMNGKCDCTPK

SEQ ID NO: 47 - protein, 37 aa, Artificial Sequence
  Description of Artificial Sequence Synthetic polypeptide
DCVRFWGKCSQTSDCCPHLACKSKWPRNICVWDGSVG

SEQ ID NO: 48 - protein, 36 aa, Artificial Sequence
  Description of Artificial Sequence Synthetic polypeptide
MCMPCFTTRPDMAQQCRACCKGRGKCFGPQCLCGYD

SEQ ID NO: 49 - protein, 33 aa, Artificial Sequence
  Description of Artificial Sequence Synthetic polypeptide
GCFGYKCDYYKGCCSGYVCSPTWKWCVRPGPGR

SEQ ID NO: 50 - protein, 38 aa, Artificial Sequence
  Description of Artificial Sequence Synthetic polypeptide
QVQTNVKCQGGSCASVCRREIGVAAGKCINGKCVCYRN

SEQ ID NO: 51 - protein, 33 aa, Artificial Sequence
  Description of Artificial Sequence Synthetic polypeptide
GDCLPHLKRCKENNDCCSKKCKRRGANPEKRCR

SEQ ID NO: 52 - protein, 35 aa, Artificial Sequence
  Description of Artificial Sequence Synthetic polypeptide
NFKVEGACSKPCRKYCIDKGARNGKCINGRCHCYY

SEQ ID NO: 53 - protein, 38 aa, Artificial Sequence
  Description of Artificial Sequence Synthetic polypeptide
QKILSNRCNNSSECIPHCIRIFGTRAAKCINRKCYCYP

SEQ ID NO: 54 - protein, 42 aa, Artificial Sequence
  Description of Artificial Sequence Synthetic polypeptide
DRDSCIDKSRCSKYGYYQECQDCCKKAGHNGGTCMFFKCKCA

SEQ ID NO: 55 - protein, 32 aa, Artificial Sequence
  Description of Artificial Sequence Synthetic polypeptide
AVCNLKRCQLSCRSLGLLGKCIGDKCECVKHG

SEQ ID NO: 56 - protein, 37 aa, Artificial Sequence
  Description of Artificial Sequence Synthetic polypeptide
QFCGTNGKPCVNGQCCGALRCVVTYHYADGVCLKMNP

SEQ ID NO: 57 - protein, 36 aa, Artificial Sequence
  Description of Artificial Sequence Synthetic polypeptide
RPTDIKCSASYQCFPVCKSRFGKTNGRCVNGLCDCF

SEQ ID NO: 58 - protein, 35 aa, Artificial Sequence
  Description of Artificial Sequence Synthetic polypeptide
NCAGYMRECKEKLCCSGYVCSSRWKWCVLPAPWRR

SEQ ID NO: 59 - protein, 37 aa, Artificial Sequence
  Description of Artificial Sequence Synthetic polypeptide
QFTDVKCTGSKQCWPVCKQMFGKPNGKCMNGKCRCYS

SEQ ID NO: 60 - protein, 38 aa, Artificial Sequence
  Description of Artificial Sequence Synthetic polypeptide
QIDTNVKCSGSSKCVKICIDRYNTRGAKCINGRCTCYP

SEQ ID NO: 61 - protein, 38 aa, Artificial Sequence
  Description of Artificial Sequence Synthetic polypeptide
AEIIRCSGTRECYAPCQKLTGCLNAKCMNKACKCYGCV

SEQ ID NO: 62 - protein, 36 aa, Artificial Sequence
  Description of Artificial Sequence Synthetic polypeptide
SDYCSNDFCFFSCRRDRCARGDCENGKCVCKNCHLN

SEQ ID NO: 63 - protein, 39 aa, Artificial Sequence
  Description of Artificial Sequence Synthetic polypeptide
CIGEGVPCDENDPRCCFGLVCLKPTLHGIWYKSYYCYKK

SEQ ID NO: 64 - protein, 31 aa, Artificial Sequence
  Description of Artificial Sequence Synthetic polypeptide
SCAKPGEMCMRIKCCDGQCGCNRGTGRCFCK

SEQ ID NO: 65 - protein, 35 aa, Artificial Sequence
  Description of Artificial Sequence Synthetic polypeptide
ACLAEYQKCEGSTVPCCPGLSCSAGRFRKTKLCTK

SEQ ID NO: 66 - protein, 35 aa, Artificial Sequence
  Description of Artificial Sequence Synthetic polypeptide
VVIGQRCYRSPDCYSACKKLVGKATGKCTNGRCDC

SEQ ID NO: 67 - protein, 32 aa, Artificial Sequence
  Description of Artificial Sequence Synthetic polypeptide
ACQFWSCNSSCISRGYRQGYCWGIQYKYCQCQ

SEQ ID NO: 68 - protein, 36 aa, Artificial Sequence
  Description of Artificial Sequence Synthetic polypeptide
RCPPCFTTNPNMEADCRKCCGGRGYCASYQCICPGG

SEQ ID NO: 69 - protein, 37 aa, Artificial Sequence
  Description of Artificial Sequence Synthetic polypeptide
VFINVKCRGSPECLPKCKEAIGKSAGKCMNGKCKCYP

SEQ ID NO: 70 - protein, 38 aa, Artificial Sequence
  Description of Artificial Sequence Synthetic polypeptide
QVSTNKKCSNTSQCYKTCEKVVGVAAGKCMNGKCICYP

SEQ ID NO: 71 - protein, 36 aa, Artificial Sequence
  Description of Artificial Sequence Synthetic polypeptide
ECLEIFKACNPSNDQCCKSSKLVCSRKTRWCKYQIG

SEQ ID NO: 72 - protein, 54 aa, Artificial Sequence
  Description of Artificial Sequence Synthetic polypeptide
QDKCKKVYENYPVSKCQLANQCNYDCKLDKHARSGECFYDEKRNLQCICDYCEY

SEQ ID NO: 73 - protein, 37 aa, Artificial Sequence
  Description of Artificial Sequence Synthetic polypeptide
GVPINVRCRGSRDCLDPCRRAGMRFGRCINSRCHCTP

SEQ ID NO: 74 - protein, 44 aa, Artificial Sequence
  Description of Artificial Sequence Synthetic polypeptide
YSRCQLQGFNCVVRSYGLPTIPCCRGLTCRSYFPGSTYGRCQRY

SEQ ID NO: 75 - protein, 36 aa, Artificial Sequence
  Description of Artificial Sequence Synthetic polypeptide
MCMPCFTTDHQMARKCDDCCGGKGRGKCYGPQCLCR

SEQ ID NO: 76 - protein, 57 aa, Artificial Sequence
  Description of Artificial Sequence Synthetic polypeptide
AQEPVKGPVSTKPGSCPIILIRCAMLNPPNRCLKDTDCPGIKKCCEGSCGMACFVPQ

SEQ ID NO: 77 - protein, 35 aa, Artificial Sequence
  Description of Artificial Sequence Synthetic polypeptide
MCMPCFTTDPNMAKKCRDCCGGNGKCFGPQCLCNR

SEQ ID NO: 78 - protein, 41 aa, Artificial Sequence
  Description of Artificial Sequence Synthetic polypeptide
GIGDPVTCLKSGAICHPVFCPRRYKQIGTCGLPGTKCCKKP

SEQ ID NO: 79 - protein, 38 aa, Artificial Sequence
  Description of Artificial Sequence Synthetic polypeptide
GVPTDVKCRGSPQCIQPCKDAGMRFGKCMNGKCHCTPK

SEQ ID NO: 80 - protein, 36 aa, Artificial Sequence; identical to SEQ ID NO: 68
  Description of Artificial Sequence Synthetic polypeptide

SEQ ID NO: 81 - protein, 81 aa, Artificial Sequence
  Description of Artificial Sequence Synthetic polypeptide
  MOD_RES (2)..(16): Any amino acid
  MISC_FEATURE (2)..(16): This region may encompass 1-15 residues or may be absent in its entirety
  MOD_RES (18)..(32): Any amino acid
  MISC_FEATURE (18)..(32): This region may encompass 1-15 residues or may be absent in its entirety
  MOD_RES (34)..(48): Any amino acid
  MISC_FEATURE (34)..(48): This region may encompass 1-15 residues or may be absent in its entirety
  MOD_RES (50)..(64): Any amino acid
  MISC_FEATURE (50)..(64): This region may encompass 1-15 residues or may be absent in its entirety
  MOD_RES (66)..(80): Any amino acid
  MISC_FEATURE (66)..(80): This region may encompass 1-15 residues or may be absent in its entirety
  See specification as filed for detailed description of substitutions and preferred embodiments
CXXXXXXXXXXXXXXXCXXXXXXXXXXXXXXXCXXXXXXXXXXXXXXXCXXXXXXXXXXXXXXXCXXXXXXXXXXXXXXXC

SEQ ID NO: 82 - protein, 28 aa, Artificial Sequence
  Description of Artificial Sequence Synthetic peptide
  MISC_FEATURE (1)..(28): This sequence may encompass 1-7 “Gly Gly Gly Ser” repeating units
GGGSGGGSGGGSGGGSGGGSGGGSGGGS

SEQ ID NO: 83 - protein, 40 aa, Artificial Sequence
  Description of Artificial Sequence Synthetic polypeptide
  MOD_RES (1)..(13): Any amino acid or absent
  MOD_RES (15)..(17): Any amino acid or absent
  MOD_RES (19)..(28): Any amino acid or absent
  MOD_RES (30)..(30): Any amino acid or absent
  MOD_RES (32)..(33): Any amino acid or absent
  MOD_RES (35)..(35): Any amino acid or absent
  MOD_RES (37)..(40): Any amino acid or absent
  See specification as filed for detailed description of substitutions and preferred embodiments
XXXXXXXXXXXXXCXXXCXXXXXXXXXXCXNXXCXCXXXX

SEQ ID NO: 84 - protein, 40 aa, Artificial Sequence
  Description of Artificial Sequence Synthetic polypeptide
  MOD_RES (1)..(1): Any amino acid or absent
  MOD_RES (3)..(5): Any amino acid or absent
  MOD_RES (9)..(9): Any amino acid or absent
  MOD_RES (12)..(13): Any amino acid or absent
  MOD_RES (16)..(16): Any amino acid or absent
  MOD_RES (20)..(22): Any amino acid or absent
  MOD_RES (24)..(24): Any amino acid or absent
  MOD_RES (26)..(26): Any amino acid or absent
  MOD_RES (35)..(35): Any amino acid or absent
  MOD_RES (37)..(37): Any amino acid or absent
  MOD_RES (39)..(40): Any amino acid or absent
  See specification as filed for detailed description of substitutions and preferred embodiments
XVXXXVKCXGSXXCLXPCKXXXGXRXGKCMNGKCXCXPXX

SEQ ID NO: 85 - protein, 4 aa, Artificial Sequence
  Description of Artificial Sequence Synthetic peptide
GAGA

SEQ ID NO: 86 - protein, 37 aa, Mesobuthus martensii
VGINVKCKHSGQCLKPCKDAGMRFGKCINGKCDCTPK

SEQ ID NO: 87 - protein, 37 aa, Mesobuthus martensii
VGINVKCKHSRQCLKPCKDAGMRFGKCTNGKCHCTPK

SEQ ID NO: 88 - protein, 38 aa, Leiurus quinquestriatus
GVPINVSCTGSPQCIKPCKDAGMRFGKCMNRKCHCTPK

(19) United States
(12) Patent Application Publication
(10) Pub. No.: US 2020/0262877 A1
(43) Pub. Date: Aug. 20, 2020

(54) ION CHANNEL-BINDING PEPTIDES AND METHODS OF USE THEREOF

(71) Applicants: BLAZE BIOSCIENCE, INC., Seattle, WA (US); FRED HUTCHINSON CANCER RESEARCH CENTER, Seattle, WA (US)

(72) Inventors: Christopher Mehlin, Seattle, WA (US); Natalie Winblade Nairn, Seattle, WA (US); Scott Ronald Presnell, Seattle, WA (US)

(21) Appl. No.: 16/650,350

(22) PCT Filed: Oct. 8, 2018
(86) PCT No.: PCT/US2018/054889

§ 371 Date: Mar. 24, 2020

Related U.S. Application Data
(60) Provisional application No. 62/570,071, filed on Oct. 9, 2017.

(51) Int. Cl. C07K 14/435; A61P 25/08; A61P 3/04; A61P 37/00; A61P 35/00; A61P 19/02; A61P 25/28; A61P 1/00; A61P 13/12; A61P 25/02

(52) U.S. Cl. CPC C07K 14/43513; A61P 25/08; A61P 3/04; A61P 37/00; A61P 35/00; A61K 38/00; A61P 25/28; A61P 1/00; A61P 13/12; A61P 25/02; A61P 19/02

(57) ABSTRACT

Disclosed herein are compositions and methods of use comprising peptides that bind to ion channels. Such peptides can function as active agents that target ion channels to inhibit or activate ion channels in a target tissue or cell type. In some embodiments, such peptides can be conjugated to another active agent or a detectable label.

### CROSS REFERENCE

This application claims the benefit of U.S. Provisional Patent Application No. 62/570,071, filed Oct. 9, 2017, the entire disclosure of which is incorporated by reference.

### SEQUENCE LISTING

The instant application contains a Sequence Listing which has been submitted electronically in ASCII format and is hereby incorporated by reference in its entirety. Said ASCII copy, created on Sep. 18, 2018, is named 45639-714_601_SL.txt and is 45,258 bytes in size.

### BACKGROUND

Ion channels are implicated in a wide range of physiological functions, including electrical signal transduction, chemical signaling, trans-epithelial transport, muscle contraction, hormone secretion, and regulation of ion concentrations, pH, and cell volume. Ion channel dysfunction has been associated with diseases in various tissues, such as pancreas, bone, kidney, central nervous system (“CNS”), heart, gastrointestinal system, and retina. Much of the ion channel family of proteins remains unexplored as drug targets. Thus, new methods and compositions that target ion channels or proteins thereof for the treatment and/or amelioration or prevention of ion-channel-related diseases and/or conditions are needed.

### SUMMARY

The present disclosure contemplates a composition comprising a composition comprising:

a peptide that modulates an ion channel activity, the peptide comprising an amino acid sequence according to:
X⁰X¹X²X³X⁴X⁵X⁶X⁷X⁸X⁹X¹⁰X¹¹X¹²CX¹⁴X¹⁵X¹⁶CX¹⁸X¹⁹X²⁰X²¹X²²X²³X²⁴X²⁵X²⁶X²⁷CX²⁹NX³¹X³²CX³⁴CX³⁶X³⁷X³⁸X³⁹ (SEQ ID NO: 83), wherein any one of X⁰ to X³⁹ is independently any amino acid or null. In some embodiments, the peptide comprises an amino acid sequence according to:
X⁰VX²X³X⁴VKCX⁸GSX¹¹X¹²CLX¹⁵PCKX¹⁹X²⁰X²¹GX²³RX²⁵GKCMNGKCX³⁴CX³⁶PX³⁸X³⁹ (SEQ ID NO: 84), wherein any one of X⁰ to X³⁹ is independently any amino acid or null, and wherein the peptide further comprises one or more of: X⁰ is G, Q, or null; X¹ is R, Q, V, I, or null;
X² is P, F, G, R, V, N, D, A, or null; X³ is T, I, F, or E; X⁴ is D, N, P, G, K, V, or I; X⁵ is I, V, or Q; X⁶ is K, R, S, or E; X⁷ is C or G; X⁸ is S, T, R, K, Y or A; X⁹ is A, G, H, R or C; X¹⁰ is S or T; X¹¹ is Y, K, R, G, P, or S; X¹² is Q, D, E, P, or K; X¹⁴ is F, W, L, I, Y, R, or V; X¹⁵ is P, D, K, Q, S, G, or A; X¹⁶ is V, P, K, A, Y, or I; X¹⁸ is K, R, I, or Q; X¹⁹ is S, Q, K, R, D, or E; X²⁰ is R, M, A, L, K, or Q; X²¹ is F, I, V, Y, T, or null; X²² is G or N; X²³ is K, M, A, T, or C; X²⁴ is T, P, R, S, A, or L; X²⁵ is N, F, A, T, or G; X²⁶ is G, A, or S; X²⁷ is R or K; X²⁹ is V, M, I, T, S or L; X³¹ is G, S, R, or K; X³² is L, K, R, V, or A; X³⁴ is D, R, H, K, or T; X³⁶ is F, Y, T, or null; X³⁷ is S, P, Y, G, or null; X³⁸ is K, C, null; and X³⁹ is G, V, or null. In some embodiments, any one of X⁰ to X³⁹ is independently any amino acid or null, and wherein the peptide further comprises one or more of: X⁵ is I or V; X⁶ is K or R; X⁷ is C; X¹⁰ is S; X²² is G; X²⁶ is G; X²⁷ is K; X²⁹ is V, M, I, or T; X³² is K or R; and X³⁴ is H, K, or D.

In some aspects, the peptide comprises an amino acid sequence according to: X⁰ VX²X³X⁴VKCX⁸GSX¹¹X¹²CLX¹⁵PCKX¹⁹X²⁰X²¹GX²³RX²⁵GKCMNGKCX³⁴CX³⁶PX³⁸X³⁹ (SEQ ID NO: 84), wherein any one of X⁰ to X³⁹ is independently any amino acid or null. In some embodiments, X⁰ is G, Q, or null. In some embodiments, X¹ is R, Q, V, I, or null. In some embodiments, X² is P, F, G, R, V, N, D, A, or null. In some embodiments, X³ is T, I, F, or E. In some embodiments, X⁴ is D, N, P, G, K, V, or I. In some embodiments, X⁵ is I, V, or Q. In some embodiments, X⁶ is K, R, S, or E. In some embodiments, X⁷ is C or G. In some embodiments, X⁸ is S, T, R, K, Y or A. In some embodiments, X⁹ is A, G, H, R or C. In some embodiments, X¹⁰ is S or T. In some embodiments, X¹¹ is Y, K, R, G, P, or S. In some embodiments, X¹² is Q, D, E, P, or K. In some embodiments, X¹⁴ is F, W, L, I, Y, R, or V. In some embodiments, X¹⁵ is P, D, K, Q, S, G, or A. In some embodiments, X¹⁶ is V, P, K, A, Y, or I. In some embodiments, X¹⁸ is K, R, I, or Q. In some embodiments, X¹⁹ is S, Q, K, R, D, or E. In some embodiments, X²⁰ is R, M, A, L, K, or Q. In some embodiments, X²¹ is F, I, V, Y, T, or null. In some embodiments, X²² is G or N. In some embodiments, X²³ is K, M, A, T, or C. In some embodiments, X²⁴ is T, P, R, S, A, or L. In some embodiments, X²⁵ is N, F, A, T, or G. In some embodiments, X²⁶ is G, A, or S. In some embodiments, X²⁷ is R or K. In some embodiments, X²⁹ is V, M, I, or T. In some embodiments, X³¹ is G, S, R, or K. In some embodiments, X³² is L, K, R, V, or A. In some embodiments, X³⁴ is D, R, H, K, or T. In some embodiments, X³⁶ is F, Y, T, or null. In some embodiments, X³⁷ is S, P, Y, G, or null. In some embodiments, X³⁸ is K, C, null. In some embodiments, X³⁹ is G, V, or null.

In some aspects, the peptide comprises an anti-parallel beta sheet domain that interacts with the ion channel. In some aspects, the peptide comprises 2 or more, 3 or more, 4 or more, 5 or more, 6 or more, 7 or more, 8 or more, 9 or more, or 10 or more solvent-exposed basic residues that interact with the ion channel. In some aspects, the peptide comprises a R or K that blocks an entryway of the ion channel upon binding. In some aspects, the peptide has at least 80%, 85%, 90%, 95%, 96%, 97%, 98%, 99%, or 100% sequence identity to any one of SEQ ID NO: 1-SEQ ID NO: 80 or a functional fragment thereof In some aspects, the peptide is non-naturally occurring. In some aspects, the peptide comprises GS amino acid residues at the N-terminus.

In some aspects, the peptide is a knotted peptide. In some aspects, the peptide comprises 6 or more cysteine residues. In some aspects, the peptide comprises three or more disulfide bridges formed between cysteine residues, wherein one of the disulfide bridges passes through a loop formed by two other disulfide bridges. In some aspects, the peptide comprises a plurality of disulfide bridges. In some aspects, the peptide is a cystine-dense peptide (CDP). In some aspects, the CDP comprises independent folding domains, wherein the independent folding domains comprise a high density of at least six cysteines. In some aspects, the CDP is a non-knotted CDP. In some aspects, the peptide comprises a topology of a Cysu-Cysv disulfide bond, a Cysw-Cysx disulfide bond, and a Cysy-Cysz disulfide bond, wherein the Cysw-Cysx disulfide bond passes through a macrocycle comprising the Cysu-Cysv disulfide bond and the Cysy-Cysz disulfide bond. In some aspects, the Cysw-Cysx cysteine-cysteine bond is a knotting cysteine. In some aspects, the peptide is a hitchin, and wherein the hitchin comprises a topology wherein the Cysu-Cysy disulfide bond is between cysteine 1 and cysteine 4, the Cysw-Cysx disulfide bond is between cysteine 2 and cysteine 5, and wherein the Cysy-Cysz disulfide bond is between cysteine 3 and cysteine 6. In some aspects, at least one amino acid residue of the peptide is in an L configuration or, wherein at least one amino acid residue of the knotted peptide is in a D configuration.

In other aspects, the peptide is at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 amino acid residues long. In some aspects, any one or more K residues of the peptide are replaced by an R residue or wherein any one or more R residues are replaced by for a K residue. In some aspects, the knotted peptide has a charge distribution comprising an acidic region and a basic region. In some aspects, the knotted peptide comprises 6 or more basic residues and 2 or fewer acidic residues. In some aspects, the knotted peptide comprises a 4-19 amino acid residue fragment containing at least 2 cysteine residues, and at least 2 positively charged amino acid residues. In some aspects, the knotted peptide comprises a 20-70 amino acid residue fragment containing at least 2 cysteine residues, no more than 2 basic residues and at least 2 positively charged amino acid residues. In some aspects, the knotted peptide comprises at least 3 positively charged amino acid residues. In some aspects, the positively charged amino acid residues are selected from K, R, or a combination thereof. In some aspects, the knotted peptide is selected from a potassium channel agonist, a potassium channel antagonist, a sodium channel agonist, a sodium channel antagonist, a calcium channel agonist, a calcium channel antagonist, a hadrucalcin, a theraphotoxin, a huwentoxin, a kaliotoxin, a cobatoxin, a lectin, a GABA agonist, a GABA antagonist, a NR2A/NR2B agonist, a NR2A/NR2B antagonist, a nicotinic receptor agonist, a nicotinic receptor antagonist, a TRP agonist, or a TRP antagonist. In some aspects, at least one residue of the knotted peptide comprises a chemical modification. In some aspects, the chemical modification is blocking the N-terminus of the knotted peptide. In some aspects, the chemical modification is methylation, acetylation, or acylation. In some aspects, the chemical modification is: methylation of one or more lysine residues or analogue thereof; methylation of the N-terminus; or methylation of one or more lysine residue or analogue thereof and methylation of the N-terminus.

In some aspects, the knotted peptide is linked to an acyl adduct. In some aspects, the knotted peptide is linked to an active agent. In some aspects, the active agent is fused with the knotted peptide at an N-terminus or a C-terminus of the knotted peptide. In some aspects, the knotted peptide is linked to the active agent via a cleavable linker or a non-cleavable linker. In some aspects, the active agent is a detectable agent. In some aspects, the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator.

In other aspects, a method of modulating an ion channel activity comprises administering to a subject any one of the compositions disclosed herein. In some aspects, the method of treating an ion channel-associated disorder in a subject in need thereof comprises administering to the subject in need thereof any one of the compositions described herein. In some aspects, the ion channel-associated disorder is selected from the group consisting of Bartter's syndrome, Andersen's syndrome, congenital hyperinsulinism, dilated cardiomyopathy, episodic ataxia type 1 or type 2, long QT syndrome, short QT syndrome, benign neonatal febrile convulsions, nonsyndromic deafness, polycystic kidney disease, familial episodic pain syndrome, focal segmental glomerulosclerosis, Retinitis pigmentosa, Severe myoclonic epilepsy, cerebellar ataxia, erythromelalgia, paroxysmal extreme pain disorder, congenital indifference to pain, benign familial neonatal seizures, Timothy syndrome, GI motility disorders, constipation, irritable bowel syndrome, Crohn's disease, diarrhea, inflammatory bowel disease, GI pain, rheumatoid arthritis, anklysosis spondylitis, multiple sclerosis, autoimmune disease, psoriasis, Hashimoto's thyroiditis, Sjorgen's syndrome, autoimmune glomerulonephritis, lupus, type-1 diabetes, pain, neuropathic pain, seizures, epilepsy, hypertension, renal hypertension, cancer, Parkinson's disease, neuromuscular disorders, cystic fibrosis, and dry eye. In some aspects, the peptide binds to the ion channel. In some aspects, the ion channel is a voltage-gated channel, a ligand-gated channel, or a ligand-activated channel, an inward rectifier channel, or a mechanosensitive ion channel. In some aspects, the ion channel is a potassium channel, sodium channel, calcium channel, TRP channel, GABA receptor, NMDA receptor, ionotrophic glutamate receptor channel, acetylcholine receptor, nicotinic receptor, 5-HT3 receptor, or chloride channel. In some aspects, the ion channel is selected from: 5-HT3a, alpha-4 beta-2 nicotinic receptor, alpha-3-beta-4 nicotinic receptor, Cav2.1, Cav2.2, GABA, hERG, Kir2.1, Kv1.1, Kv1.2, Kv1.3, Kv2.1, Nav1.5 (TP1), Nav1.5 (TP2), Nav1.7 (TP1), Nav1.7 (TP2), NR2A, NR2B, TRPA1, and TRPV1. In some aspects, the ion channel is selected from: 5HT3a, alpha-4 beta-2 nicotinic receptor, alpha-3-beta-4 nicotinic receptor, Cav2.2, Kv1.2, Kv2.1, NR2A, NR2B, and TRPV1. In some aspects, the peptide inhibits or activates the ion channel. In some aspects, the ion channel comprises a gain of function mutation or a loss of function mutation. In some aspects, the ion channel is overexpressed or under-expressed in a target tissue or cell type. In some aspects, the method comprises ion channel activation or deactivation is associated with a disease state. In some aspects, the peptide binds to the ion channel to induce a conformational change in the ion channel. In some aspects, the peptide binds to the ion channel to block ion movement through the channel. In some aspects, the peptide binds to the ion channel to enhance ion movement through the channel. In some aspects, the peptide binds to the ion channel to block the ion channel from ligand interaction. In some aspects, peptide affinity to an ion channel is in the range of 0.01 nM to 1000 nM. In some aspects, the peptide inhibits ion channel activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% as measured by a ScreenPatch or ChanTest assay. In some aspects, the peptide increases ion channel activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% as measured by a ScreenPatch or ChanTest assay. In some aspects, peptide binding to the ion channel results in less off-target or toxicity effects as compared to a reference compound: mibefradil dihydrochloride for calcium ion channel; E-4031 for hERG; 4-aminopyridine for potassium ion channel; Verapamil for Kv2.1; lidocaine for sodium in channel; memantine for NR2A; capsazepine for TRP channel; picrotoxin for GABA channel; ondansetron for 5HT3a channel; mecamylamine for α3β4 or α4β2; or BaCl2 for Kir2.1. In some aspects, the ion channel-related disease is a neurological disorder, cancer, autoimmune disease, GI motility disorder, irritable bowel syndrome, inflammatory bowel disease, constipation, dyspepsia, acquired neuromyotonia, renal disorder, ocular disorder, retinal disease, epilepsy, migraine, ataxia, polycystic kidney disease, seizure, long or short QT syndrome, paralysis, pain, neuropathic pain, severe pain, need for local anesthesia, migraine, cystic fibrosis, Bartter syndrome, endocrine disorder, rheumatoid arthritis, type 1 diabetes mellitus, multiple sclerosis, psoriasis, lupus, asthma, obesity, insulin resistance, hypertension, stroke, Alzheimer's disease, arrhythmia, neurodegenerative disease, or bone disease. In some aspects, cancer comprises breast cancer, cervical cancer, hepatocellular carcinoma, prostate cancer, colon cancer, squamous cell lung cancer, endometrial cancer, mammary gland cancer; adenocarcinoma, leukemia, chronic lymphocytic leukemia (CLL), acute myeloid leukemia (AML), glioma, glioblastoma, and neuroblastoma, or metastases. In some aspects, the peptide targets a tissue or cell type comprising cardiac cells; renal cells; retinal cells; cancerous cells; gastrointestinal cells; epithelial cells; neurons, such as motor neuron, Purkinje cells, GABAergic neurons, excitatory neurons, sensory neurons, and interneurons; cartilage cells; immune cells, such as T and B lymphocytes; smooth muscle cells, and skeletal muscle cells. In some aspects, administering comprises oral administration, rectal suppository, inhalation, intranasal administration, topical administration, intravenous administration, subcutaneous administration, intra-articular administration, intramuscular administration, intraperitoneal administration, intra-synovial administration, vaginal administration, rectal administration, pulmonary administration, ocular administration, buccal administration, sublingual administration, intrathecal administration, or any combination thereof. In some aspects, the subject is a human or a non-human animal.

Also contemplated herein are methods of modulating a Kv 1.1 ion channel comprising administering to a subject a peptide having at least 80%, 85%, 90%, 95%, 97%, or 99% sequence identity to any one of SEQ ID NO: 6, 19, 39, 46, 59, and 79. In other aspects, a method of modulating a Kv 1.2 ion channel comprises administering to a subject a peptide having at least 80%, 85%, 90%, 95%, 97%, or 99% sequence identity to any one of SEQ ID NO: 1-4, 11-14, 17, 22, 31, 40, 41-44, 51-54, 57, 62, 71, and 80. In other aspects, a method of modulating a Kv 1.3 ion channel comprises administering to a subject a peptide having at least 80%, 85%, 90%, 95%, 97%, or 99% sequence identity to any one of SEQ ID NO: 2, 17, 20, 33, 40, 42, 57, 60, and 62. In other aspects, a method of modulating a Nav 1.5 ion channel comprises administering to a subject a peptide having at least 80%, 85%, 90%, 95%, 97%, or 99% sequence identity to SEQ ID NO: 7 and 47. In other aspects, a method of modulating a Nav1.7 ion channel comprises administering to a subject a peptide having at least 80%, 85%, 90%, 95%, 97%, or 99% sequence identity to any one of SEQ ID NO: 7, 16, 18, 47, 56, and 58. In other aspects, a method of modulating a NR2A ion channel comprises administering to a subject a peptide having at least 80%, 85%, 90%, 95%, 97%, or 99% sequence identity to SEQ ID NO: 40 and 80. In other aspects, a method of modulating a hERG ion channel comprises administering to a subject a peptide having at least 80%, 85%, 90%, 95%, 97%, or 99% sequence identity to SEQ ID NO: 7 and 47.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BRIEF DESCRIPTION OF THE FIGURES

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates sequence alignments of potassium ion channel-binding peptides. FIG. 1A illustrates sequence alignment of Kv1.1, Kv1.2, and Kv1.3 interacting peptides, including SEQ ID NO: 42, 45, 46, 52, 57, 59, 60, 61, 66, 69, 73, and 79. BmKTX (SEQ ID NO: 86) is a potassium blocker toxin isolated from the venom of scorpion Buthus martensi. ADWX-1 (SEQ ID NO: 87) is an analog of BmKTX. Agitoxin (AgiTX) (SEQ ID NO: 88) is a toxin found in the venom of scorpion Leiurus quinquestriatus hebraeus, and binds to Shaker potassium channel. The black boxes indicate amino acid residues with >50% conservation. The gray boxes indicate amino acid residues that are similar, while white boxes indicate residues that are not conserved.

FIG. 1B illustrates sequence alignment of three Kv1.2 binding peptide candidates: SEQ ID NO: 54, 58, and 80.

FIG. 2 illustrates ion channel inhibitory activity of select peptides disclosed herein. Channel inhibition is illustrated in gray scale, ranging from white (0% inhibition) to black (100% inhibition).

FIG. 3 illustrates a stimulus voltage pattern used for measuring inhibition of Nav1.x channels expressed in mammalian cells.

FIG. 4 illustrates a stimulus voltage pattern used for measuring inhibition of Kv1.x channels expressed in mammalian cells.

FIG. 5 illustrates the Kv1.3 dose-response curves for each peptide and 4-AP control.

FIG. 6 illustrates the Kv1.5 dose-response curves for each peptide and 4-AP control.

FIG. 7 illustrates the Nav1.3 dose-response curves for each peptide and tetrodotoxin (TTX) control.

FIG. 8 illustrates the Nav1.4 dose-response curves for each peptide and TTX control.

FIG. 9 illustrates the Nav1.5 dose-response curves for each peptide and TTX control.

FIG. 10 illustrates the Nav1.7 dose-response curves for each peptide and TTX control.

### DETAILED DESCRIPTION

Disclosed herein are compositions and methods of use thereof comprising peptides that selectively target ion channels. The present disclosure describes methods of modulating an ion channel comprising administering a peptide to a subject in need thereof, and wherein the peptide comprises at least 80%, 85%, 90%, 95%, 97%, or 99% sequence identity to any one of SEQ ID NO: 1-SEQ ID NO: 80 or a functional fragment thereof. It is understood that any percentage sequence identity represented by an integer between 80% and 99% is also included (e.g., wherein the peptide comprises at least 91%, 92%, 93%, 94%, 95%, 96%, 97%, 98% or 99% sequence identity to any one of SEQ ID NO: 1-SEQ ID NO: 80). In some cases, peptides described herein can target or bind to an ion channel to result in a therapeutic effect, such as blocking or activating ion channels in a particular cell type or tissue associated with a disease or disorder. In some cases, a peptide described herein is used to treat an ion channel-related disease.

In some cases, the peptides disclosed herein function as active agents that bind to ion channels to inhibit or activate an ion channel in a target cell type or tissues. In some cases, peptides described herein serve as a carrier, scaffold, or a platform for attaching or conjugating to a payload or an active agent, such as an imaging molecule, a detectable label, a marker, toxins, immunomodulatory agents, steroids, and a therapeutic agent, an ion channel blocker, ion channel agonist, ion channel antagonist, an antibody, a small molecule, a homing agent, or another moiety for use in medical research and/or clinical applications. In some embodiments, peptides disclosed herein target ion channels to result in increase in activity, e.g., prolonged channel opening or facilitate movement of ions, or decrease in activity, e.g., blocking and/or inhibiting ion channel function. In some embodiments, peptides disclosed herein target ion channels to alter the kinetics of ion channel opening and/or closing. In some embodiments, peptides disclosed herein function as a blockage or result in steric hindrance that prevents flow of ions through the channel and/or binding of factors that activate the channel. In some embodiments, peptides described herein function as a homing agent that selectively targets an ion channel of interest as compared to non-target or off-target ion channels. In some embodiments, peptides disclosed herein result in a conformational change in an ion channel. In some embodiments, peptides disclosed herein target an ion channel to increase, decrease, alter, or modulate the activity and/or function of the ion channel. In some embodiments, peptides disclosed interact or interfere with an ion channel to prevent activation by the ion channel's ligand or activating factor. In some embodiments, peptides described herein interfere with depolarization and/or repolarization of membrane potential. In some embodiments, these functions and/or effects of peptides are produced by another molecule or moiety conjugated to the peptides disclosed herein.

Ion channels contemplated herein include voltage-gated channels, extracellular ligand-activated channels, and intracellular ligand-gated ion channels. Voltage-gated channels include, but are not limited to, sodium ion channels, potassium ion channels, and calcium ion channels. Ion channels are transmembrane proteins that create a gated pore across the membrane to selectively allow specific ions to flow across the membrane. Ion channels typically comprise protein domains that form a water-filled pore in a cell membrane. Such protein domains typically comprise four or five helices that form a barrel-like structure. Ion channels also comprise a gating mechanism that controls the opening and closure of the pore through conformational changes in the channel protein. Many ion channel-targeting drugs have low selectivity, thus resulting in various adverse side-effects due to off-target effects. Such off-target effects can prevent long-term or chronic use the compounds. As such, there is a need for therapeutic agents that target ion channels with greater specificity.

Anti-cancer drugs with known off-target effects on ion channels can be screened to identify selective ion channel inhibitors, especially for targeting ion channels that play a role in cancer development. Voltage-gated ion channels, such as sodium (Naᵥ), calcium (Caᵥ), potassium (Kᵥ, K_{ca}), chloride, and non-selective ion channels, are potential targets for various gastrointestinal (GI) motility disorders. Venoms and toxins of various snakes, scorpions, spiders, insects and marine animals include mixtures of neurotoxic peptides that have evolved for capturing prey or as defense mechanisms against predators. Such venoms and toxins often target Kᵥ, Caᵥ, and Naᵥ ion channels and some ligand-gated ion channels with high potency and selectivity. Peptides derived from various toxins are useful as a targeting platform for delivery various payloads to a target ion channel. Such peptides are compact, small peptides, stabilized by disulfide bonds and/or post-translational modifications.

Ion channel dysfunction is linked to a wide range of diseases, disorders, and/or conditions, including, but not limited to cancer; diseases, disorders, and/or conditions of kidney, CNS, cardiovascular system, peripheral nervous system, eye, retina, and gastrointestinal (GI) system; pain; and autoimmune diseases, e.g., rheumatoid arthritis, or to treat pain, affect metabolic control, thermosensation and thermoregulation.

### Peptides

The present disclosure provides peptides that can comprise or can be derived from knotted peptides. As used herein, the term “knotted peptide” can include knottins, hitchins, and cysteine-dense peptides (CDPs).

Knotted peptides, usually ranging from about 11 to about 81 amino acids in length that are often folded into a compact structure. Knottins are typically assembled into a complex tertiary structure that is characterized by a number of intramolecular disulfide crosslinks and may contain beta strands, alpha helices, and other secondary structures. The presence of the disulfide bonds can give some knottins remarkable environmental stability, allowing them to withstand extremes of temperature and pH and to resist the proteolytic enzymes, such as those of the blood stream and the digestive system. The rigidity of knottins also allows them to bind to targets without paying the “entropic penalty” that a floppy peptide accrues upon binding a target. For example, binding is adversely affected by the loss of entropy that occurs when a peptide binds a target to form a complex. Therefore, “entropic penalty” is the adverse effect on binding, and the greater the entropic loss that occurs upon this binding, the greater the “entropic penalty.” Furthermore, unbound molecules that are flexible lose more entropy when forming a complex than molecules that are rigidly structured, because of the loss of flexibility when bound up in a complex. However, rigidity in the unbound molecule also generally increases specificity by limiting the number of complexes that molecule can form. The knotted peptides can bind targets with antibody-like affinity. A wider examination of the sequence structure and sequence identity or homology of knottins reveals that they have arisen by convergent evolution in all kinds of animals and plants. In animals, they can be found in venoms, for example, the venoms of spiders and scorpions and have been implicated in the modulation of ion channels. The knottin proteins of plants can inhibit the proteolytic enzymes of animals or have antimicrobial activity, suggesting that knottins can function in the native defense of plants.

The peptides of the present disclosure can comprise cysteine amino acid residues. In some cases, the peptide has at least 6 cysteine amino acid residues. In some cases, the peptide has at least 4 cysteine amino acid residues. In some cases, the peptide has at least 8 cysteine amino acid residues. In other cases, the peptide has at least 10 cysteine amino acid residues, at least 12 cysteine amino acid residues, at least 14 cysteine amino acid residues, or at least 16 cysteine amino acid residues.

A knotted peptide can comprise disulfide bridges. A knotted peptide can be a peptide wherein 5% or more of the residues can be cysteine amino acid residues forming intramolecular disulfide bonds or cysteines. A knotted peptide can be a peptide that comprises at least 3 intramolecular disulfide bonds. A disulfide-linked peptide can be a drug scaffold. In some embodiments, the disulfide bridges form a knot. A disulfide bridge can be formed between cysteine amino acid residues, for example, between cysteine amino acid residues 1 and 4, 2 and 5, and 3 and 6. In some cases, one disulfide bridge passes through a loop formed by the other two disulfide bridges, for example, to form the knot. In other cases, the disulfide bridges can be formed between any two cysteine residues.

The present disclosure can also comprise peptides that are not canonical knottins. Some of these peptides can be hitchins, as described herein, or can have other disulfide covalent bonding topologies as compared to canonical knottins. Proteins can be differentiated from simpler peptides by size. In some embodiments, peptides can comprise less than about 50 residues long. In some embodiments, peptides do not fold into defined three-dimensional structures, as they lack enough cooperative interactions to form a stable structure, which can be accomplished through a well-packed hydrophobic core. Some exceptions can include peptides that alternately organize around cores of multiple, tightly-packed disulfide covalent bonds, which can confer extreme thermal, chemical, and proteolytic stability as set forth in Werle et al. (J Drug Target, 14(3): 137-46 (2006)), Gelly et al. (Nucleic Acids Res, 32 (Database issue): D156-9 (2004)), Reinwarth et al. (Molecules, 17(11): 12533-52 (2012)), Kolmar et al. (Curr Pharm Des, 17(38): 4329-36 (2011)), Kolmar et al. (Curr Opin Pharmacol, 9(5):608-14 (2009)), Klintzing et al. (Curr Opin Chem Biol, 34: 143-150 (2016)), and Gould et al. (Curr Pharm Des, 17(38): 4294-307 (2011)). Importantly, not all peptides with multiple disulfide covalent bonds, or cysteine-dense peptides, may have high levels of these stabilities. The archetypes of such peptides can include “inhibitor cystine knotted peptides,” also called knottins (described in the present disclosure), and the closely-related “cyclic cystine knotted peptides”, known as cyclotides, which both can have cores of at least three cystines. Examples can include venom toxins from cone snails, spiders, and scorpions; protease inhibitors from plants; and antimicrobial defensins. Knottins and cyclotides can be topologically pseudoknotted, with one cystine crossing through the macrocycle formed by the other two cystines and the interconnecting backbone. Proteins can also incorporate cystine-knotted subdomains, for example, growth factor cystine knots (GFCKs) as set forth in Vitt et al. (Mol Endocrinol, 15(5): 681-94 (2001) and Iyer et al. (FEBS J, 278(22): 4304-22 (2011)). However, the GFCK cystine-knotted element does not dominate the fold of the protein, which can include a conventional hydrophobic core, distinct from knottins and cyclotides. In some embodiments, the minimal common elements defining this class of molecules can be short sequences, constituting independent folding domains, with a high density of at least three cystines. This categorization can be referred to as “cystine-dense peptides” (CDPs), drawing a distinction with larger proteins with cystine-knotted elements, like GFCKs.

A CDP can have a knotted topology and can be defined as comprising a CDP-defining motif: sequences that can comprise six or more cysteine amino acid residues (or at least three cystines), may not be recognizable as a cytoplasmic protein or domain, a zinc finger protein, or a GFCK, can comprise a constrained distribution of cysteine amino acid residues, can be Cys-X_{[0-15]}-Cys-X_{[0-15}]-Cys-X_{[0-15]}-Cys-X_{[0-15]}-Cys-X_{[0-15]}-Cys (SEQ ID NO: 81) wherein X can be any amino acid residue, and can be from 13 to 81 residues long between the motif-bonding cysteine amino acid residues. For example, a candidate CDP can be embedded in a sequence with a recognizable leader peptide such as SignalP (Bendtsen, J. D., J Mol Biol, 340: 783-795 (2004)), can be annotated as a secreted or integral membrane protein, or can be experimentally shown to contain specific cystines, which can be used to confirm the formation of cystines in the peptide to classify the peptide as a CDP. CDPs can be embedded in larger proteins, or in tandem arrays, and can comprise an independent folding unit. The additional criterion of a minimal “cysteine density,” in which the minimal “cysteine density” can be a sequence with a cysteine amino acid residue content of at least 12%, can separate CDPs with dominant cystine cores from small proteins with emergent hydrophobic cores. This threshold CDP-defining cysteine density can be approximately 10-fold higher than the average observed for all proteins (Moura, A., PloS One, 8Ie77319 (2013); The UniProtC., Nucleic Acids Res, 45: D158-D169 (2017)). As of April 2017, there were 771 experimentally-determined structures in the PDB that can conform to this sequence-based definition.

The first level of CDP classification can be determined by disulfide bonds. Numbering the cysteines in the three-cystine core/knotting element sequentially from 1 to 6 can yield 15 theoretically possible disulfide bond classes, with most GFCKs and archetypical knottin knotted CDPs falling into the Cys1-Cys4, Cys2-Cys5, Cys3-Cys6 disulfide bond class (1-4, 2-5, 3-6), which can be referred to as the “canonical” disulfide bond class. Four other disulfide bond classes can be observed in deposited knotted CDP structures (with variable representation). Additionally, nine other disulfide bond classes can be observed in other non-knotted CDPs with three cystines. The Cys1-Cys6, Cys2-Cys3, Cys4-Cys5 disulfide bond class (1-6, 2-3, 4-5) may not observed in any natural CDPs, though can be found in wholly synthetic, designed CDPs (e.g., 5JI14.pdb (Bhardwaj, G., Nature, 538: 329-335 (2016)). Non-knotted CDPs with more than three cystines cannot be assigned to comparable disulfide bond classes, as the focus subset of three cystines cannot be defined and numbered in the same way in the absence of a knotting element, but can be lumped together in a separate CDP type, which can be referred to as type “z”.

The second level of CDP classification can be based on cystine topology and can be defined as which cystine can pseudoknot the fold, focusing on the three core cystines comprising the knotting element, and ignoring additional, accessory cystines. In any disulfide bond class, denoted as Cysu-Cysv, Cysw-Cysx, Cysy-Cysz (u-v, w-x, y-z) to indicate the core disulfide bond, there can be three theoretical topologies, each with a different knotting cystine, represented as u-v, w-x, [y-z], in which the knotting cystine can be indicated by square brackets. Knowledge of the CDP disulfide bond class and its corresponding knotting topology can indicate the structure-based knotted CDP type. Non-knotted CDPs with three cystines can be denoted solely by Cysu-Cysv, Cysw-Cysx, Cysy-Cysz disulfide bond class (u-v, w-x, y-z), and non-knotted CDPs with more than three cysteines can be denoted as “z”. Using this nomenclature, archetypical knottins can be classified as type Cys1-Cys4, Cys2-Cys5, [Cys3-Cys6] knotted CDPs (1-4, 2-5, [3-6]), which can be distinct from the [Cys1-Cys4], Cys2-Cy5, Cys3-Cys6 topology ([1-4], 2-5, 3-6) that can be observed in GFCKs despite a common disulfide bond. The second most commonly observed topology type in this knotted CDP disulfide bond class can be Cys1-Cys4, [Cys2-Cys5], Cys3-Cys6 (1-4, [2-5], 3-6). Third topology type can be a GFCK-like, topology in this disulfide bond class: [Cys1-Cys4], Cys2-Cys5, Cys3-Cys6 ([1-4], 2-5, 3-6). Following the knottin nomenclature, the type 1-4, [2-5], 3-6 knotted CDPs can be referred to as “hitchins”, type [1-4], 2-5, 3-6 GFCKs can be referred to as “shanks” (a shank can be a type of knot used to shorten a length of rope), and rare type [1-4], 2-5, 3-6 knotted CDPs can be referred to as “shankins”. Though far fewer described knotted CDP structures can have non-canonical disulfide bond classes, examples of nine additional knotted CDP types have been reported. The distribution of 771 CDPs among the different disulfide bond classes and types was predominately in the knottins, z-class, and hitchins. This proposed scheme can provide an unambiguous method for structural classification and comparison of CDPs independent of source organism, sequence homology, or functional annotation. Advantages can include avoiding broadly-applied annotations, like “defensin”, which can denote cysteine-rich, cationic, antimicrobial host defense peptides, but which can also encompass a wide range of structurally-dissimilar knotted and non-knotted CDP types, including many hitchins and knottins.

The peptides of the present disclosure can include, but are not limited to, knottins, hitchins, or other CDPs, as well as peptides that are not knotted. While the density of the cysteines and the optional presence of a knot can provide resistance to denaturation, reduction, proteases, and other structural degradations, peptides with knots or high cystine density can have varying resistance to such degradations, and some peptides can be much more stable and resistant. The peptides of the present disclosure can be more resistant to one or more chemical or physical degradation pathways.

In some embodiments, the tertiary structure and electrostatics of a peptide of the disclosure can impact stability. Structural analysis or analysis of charge distribution can be a strategy to predict residues important in biological. For example, several peptides of this disclosure that are stable can be grouped into a structural class defined above as “hitchins,” and can share the properties of disulfide linkages between Cys1-Cys4, Cys2-Cys5, and Cys3-Cys6. The folding topologies of peptides knotted through three disulfide linkages (Cys1-Cys4, Cys2-Cys5, and Cys3-Cys6), can be broken down into structural families based on the three-dimensional arrangement of the disulfides. Knottins can have the C3-C6 disulfide linkage passing through the macrocycle formed by the Cys1-Cys4 and Cys2-Cys5 disulfide linkages. Hitchins can have the Cys2-Cys5 disulfide linkage passing through the macrocycle formed by the Cys1-Cys4 and Cys3-Cys6 disulfide linkages. Other structural families can have the Cys1-Cys4 disulfide linkage passing through the macrocycle formed by the Cys2-Cys5 and Cys3-Cys6 disulfide linkages. Variants of “hitchin” class peptides with preserved disulfide linkages at these cysteine residues, primary sequence identity, and/or structural homology can be a method of identifying or predicting other potential knottin peptide candidates that can have high biological stability.

The present disclosure further includes peptide scaffolds that, e.g., can be used as a starting point for generating additional peptides. In some embodiments, these scaffolds can be derived from a variety of knotted peptides. In certain embodiments, knotted peptides can be assembled into a complex tertiary structure that is characterized by a number of intramolecular disulfide crosslinks, and optionally can contain beta strands and other secondary structures such as an alpha helix. For example, knotted peptides can include small disulfide-rich proteins characterized by a disulfide through disulfide knot. This knot can be, e.g., obtained when one disulfide bridge crosses the macrocycle formed by two other disulfides and the interconnecting backbone. In some embodiments, the knotted peptides can include growth factor cysteine knots or inhibitor cysteine knots. Other possible peptide structures can include peptide having two parallel helices linked by two disulfide bridges without β-sheets (e.g., hefutoxin).

A knotted peptide can comprise at least one amino acid residue in an L configuration. A knotted peptide can comprise at least one amino acid residue in a D configuration. In some embodiments, a knotted peptide is 15-40 amino acid residues long. In other embodiments, a knotted peptide is 11-57 amino acid residues long. In still other embodiments, a knotted peptide is 11-81 amino acid residues long. In further embodiments, a knotted peptide is at least 20 amino acid residues long.

These kinds of peptides can be derived from a class of proteins known to be present or associated with toxins or venoms. In some cases, the peptide can be derived from toxins or venoms associated with scorpions or spiders. The peptide can be derived from venoms and toxins of spiders and scorpions of various genus and species. For example, the peptide can be derived from a venom or toxin of the Leiurus quinquestriatus hebraeus, Buthus occitanus tunetanus, Hottentotta judaicus, Mesobuthus eupeus, Buthus occitanus israelis, Hadrurus gertschi, Androctonus australis, Centruroides noxius, Heterometrus laoticus, Opistophthalmus carinatus, Haplopelma schmidti, Isometrus maculatus, Haplopelma huwenum, Haplopelma hainanum, Haplopelma schmidti, Agelenopsis aperta, Haydronyche versuta, Selenocosmia huwena, Heteropoda venatoria, Grammostola rosea, Ornithoctonus huwena, Hadronyche versuta, Atrax robustus, Angelenopsis aperta, Psalmopoeus cambridgei, Hadronyche infensa, Paracoelotes luctosus, or Chilobrachys jingzhao, or another suitable genus or species of scorpion or spider. As additional examples, the peptide can be derived from Pandinus imperator, Lychas mucronatus, Hadrurus gertschi, Centruroides elegans, Macrothele gigas, Centruroides limpidus limpidus, Mesobuthus tamulus, Pentadiplandra brazzeana, Heterometrus fulvipes, or Tachypleus tridentatus. In some cases, a peptide can be derived from a Buthus martensii Karsh (scorpion) toxin. In some embodiments, a peptide can be derived from a member of the pfam005453: Toxin_6 class.

In other embodiments, the present disclosure provides peptides that are not derived from knottins. In these embodiments, peptides can be designed or engineered using in silico techniques and/or random mutagenesis techniques. In some embodiments, a peptide of the disclosure is non-naturally occurring. Non-naturally occurring can refer to an article not caused by or existing in nature in its natural form.

TABLE 1 lists some exemplary peptides according to the present disclosure.

TABLE 1
Peptide Sequences

| | Amino Acid Sequence |
|---|---|
| SEQ ID NO: 1 | GSMCMPCFTTDHQMARRCDDCCGGRGRGRCYGPQCLCR |
| SEQ ID NO: 2 | GSEVIRCSGSKQCYGPCKQQTGCTNSKCMNKVCKCYGCG |
| SEQ ID NO: 3 | GSSEKDCIKHLQRCRENKDCCSKKCSRRGTNPEKRCR |
| SEQ ID NO: 4 | GSSCAKPRENCNRMNILCCRGECVCPTFGDCFCYGD |
| SEQ ID NO: 5 | GSGVPINVKCRGSRDCLDPCKKAGMRFGKCINSKCHCTP |
| SEQ ID NO: 6 | GSVRIPVSCKHSGQCLKPCKDAGMRFGKCMNGKCDCTPK |
| SEQ ID NO: 7 | GSDCVRFWGKCSQTSDCCPHLACKSKWPRNICVWDGSVG |
| SEQ ID NO: 8 | GSMCMPCFTTRPDMAQQCRACCKGRGKCFGPQCLCGYD |
| SEQ ID NO: 9 | GSGCFGYKCDYYKGCCSGYVCSPTWKWCVRPGPGR |
| SEQ ID NO: 10 | GSQVQTNVKCQGGSCASVCRREIGVAAGKCINGKCVCYRN |
| SEQ ID NO: 11 | GSGDCLPHLKRCKENNDCCSKKCKRRGANPEKRCR |
| SEQ ID NO: 12 | GSNFKVEGACSKPCRKYCIDKGARNGKCINGRCHCYY |
| SEQ ID NO: 13 | GSQKILSNRCNNSSECIPHCIRIFGTRAAKCINRKCYCYP |
| SEQ ID NO: 14 | GSDRDSCIDKSRCSKYGYYQECQDCCKKAGHNGGTCMFFKCKCA |
| SEQ ID NO: 15 | GSAVCNLKRCQLSCRSLGLLGKCIGDKCECVKHG |
| SEQ ID NO: 16 | GSQFCGTNGKPCVNGQCCGALRCVVTYHYADGVCLKMNP |
| SEQ ID NO: 17 | GSRPTDIKCSASYQCFPVCKSRFGKTNGRCVNGLCDCF |
| SEQ ID NO: 18 | GSNCAGYMRECKEKLCCSGYVCSSRWKWCVLPAPWRR |
| SEQ ID NO: 19 | GSQFTDVKCTGSKQCWPVCKQMFGKPNGKCMNGKCRCYS |
| SEQ ID NO: 20 | GSQIDTNVKCSGSSKCVKICIDRYNTRGAKCINGRCTCYP |
| SEQ ID NO: 21 | GSAEIIRCSGTRECYAPCQKLTGCLNAKCMNKACKCYGCV |
| SEQ ID NO: 22 | GSSDYCSNDFCFFSCRRDRCARGDCENGKCVCKNCHLN |
| SEQ ID NO: 23 | GSCIGEGVPCDENDPRCCFGLVCLKPTLHGIWYKSYYCYKK |
| SEQ ID NO: 24 | GSSCAKPGEMCMRIKCCDGQCGCNRGTGRCFCK |
| SEQ ID NO: 25 | GSACLAEYQKCEGSTVPCCPGLSCSAGRFRKTKLCTK |
| SEQ ID NO: 26 | GSVVIGQRCYRSPDCYSACKKLVGKATGKCTNGRCDC |
| SEQ ID NO: 27 | GSACQFWSCNSSCISRGYRQGYCWGIQYKYCQCQ |
| SEQ ID NO: 28 | GSRCPPCFTTNPNMEADCRKCCGGRGYCASYQCICPGG |
| SEQ ID NO: 29 | GSVFINVKCRGSPECLPKCKEAIGKSAGKCMNGKCKCYP |
| SEQ ID NO: 30 | GSQVSTNKKCSNTSQCYKTCEKVVGVAAGKCMNGKCICYP |
| SEQ ID NO: 31 | GSECLEIFKACNPSNDQCCKSSKLVCSRKTRWCKYQIG |
| SEQ ID NO: 32 | GSQDKCKKVYENYPVSKCQLANQCNYDCKLDKHARSGECFYDEKRNL |
| | QCICDYCEY |
| SEQ ID NO: 33 | GSGVPINVRCRGSRDCLDPCRRAGMREGRCINSRCHCTP |
| SEQ ID NO: 34 | GGYSRCQLQGFNCVVRSYGLPTIPCCRGLTCRSYFPGSTYGRCQRY |
| SEQ ID NO: 35 | GSMCMPCFTTDHQMARKCDDCCGGKGRGKCYGPQCLCR |
| SEQ ID NO: 36 | GSAQEPVKGPVSTKPGSCPIILIRCAMLNPPNRCLKDTDCPGIKKCCEGSC |
| | GMACFVPQ |
| SEQ ID NO: 37 | GSMCMPCFTTDPNMAKKCRDCCGGNGKCFGPQCLCNR |
| SEQ ID NO: 38 | GSGIGDPVTCLKSGAICHPVFCPRRYKQIGTCGLPGTKCCKKP |
| SEQ ID NO: 39 | GSGVPTDVKCRGSPQCIQPCKDAGMRFGKCMNGKCHCTPK |
| SEQ ID NO: 40 | GSRCPPCFTTNPNMEADCRKCCGGRGYCASYQCICPGG |
| SEQ ID NO: 41 | MCMPCFTTDHQMARRCDDCCGGRGRGRCYGPQCLCR |
| SEQ ID NO: 42 | EVIRCSGSKQCYGPCKQQTGCTNSKCMNKVCKCYGCG |
| SEQ ID NO: 43 | SEKDCIKHLQRCRENKDCCSKKCSRRGTNPEKRCR |
| SEQ ID NO: 44 | SCAKPRENCNRMNILCCRGECVCPTFGDCFCYGD |
| SEQ ID NO: 45 | GVPINVKCRGSRDCLDPCKKAGMRFGKCINSKCHCTP |
| SEQ ID NO: 46 | VRIPVSCKHSGQCLKPCKDAGMRFGKCMNGKCDCTPK |
| SEQ ID NO: 47 | DCVRFWGKCSQTSDCCPHLACKSKWPRNICVWDGSVG |
| SEQ ID NO: 48 | MCMPCFTTRPDMAQQCRACCKGRGKCFGPQCLCGYD |
| SEQ ID NO: 49 | GCFGYKCDYYKGCCSGYVCSPTVVKWCVRPGPGR |
| SEQ ID NO: 50 | QVQTNVKCQGGSCASVCRREIGVAAGKCINGKCVCYRN |
| SEQ ID NO: 51 | GDCLPHLKRCKENNDCCSKKCKRRGANPEKRCR |
| SEQ ID NO: 52 | NEKVEGACSKPCRKYCIDKGARNGKONGRCHCYY |
| SEQ ID NO: 53 | QKILSNRCNNSSECIPHCIRIFGTRAAKCINRKCYCYP |
| SEQ ID NO: 54 | DRDSCIDKSRCSKYGYYQECQDCCKKAGHNGGTCMFFKCKCA |
| SEQ ID NO: 55 | AVCNLKRCQLSCRSLGLLGKCIGDKCECVKHG |
| SEQ ID NO: 56 | QFCGTNGKPCVNGQCCGALRCVVTYHYADGVCLKMNP |
| SEQ ID NO: 57 | RPTDIKCSASYQCFPVCKSRFGKTNGRCVNGLCDCF |
| SEQ ID NO: 58 | NCAGYMRECKEKLCCSGYVCSSRWKWCVLPAPWRR |
| SEQ ID NO: 59 | QFTDVKCTGSKQCWPVCKQMFGKPNGKCMNGKCRCYS |
| SEQ ID NO: 60 | QIDTNVKCSGSSKCVKICIDRYNTRGAKCINGRCTCYP |
| SEQ ID NO: 61 | AEIIRCSGTRECYAPCQKLTGCLNAKCMNKACKCYGCV |
| SEQ ID NO: 62 | SDYCSNDFCFFSCRRDRCARGDCENGKCVCKNCHLN |
| SEQ ID NO: 63 | CIGEGVPCDENDPRCCFGLVCLKPTLHGIWYKSYYCYKK |
| SEQ ID NO: 64 | SCAKPGEMCMRIKCCDGQCGCNRGTGRCFCK |
| SEQ ID NO: 65 | ACLAEYQKCEGSTVPCCPGLSCSAGRFRKTKLCTK |
| SEQ ID NO: 66 | VVIGQRCYRSPDCYSACKKLVGKATGKCTNGRCDC |
| SEQ ID NO: 67 | ACQFWSCNSSCISRGYRQGYCWGIQYKYCQCQ |
| SEQ ID NO: 68 | RCPPCFTTNPNMEADCRKCCGGRGYCASYQCICPGG |
| SEQ ID NO: 69 | VFINVKCRGSPECLPKCKEAIGKSAGKCMNGKCKCYP |
| SEQ ID NO: 70 | QVSTNKKCSNTSQCYKTCEKVVGVAAGKCMNGKCICYP |
| SEQ ID NO: 71 | ECLEIFKACNPSNDQCCKSSKLVCSRKTRWCKYQIG |
| SEQ ID NO: 72 | QDKCKKVYENYPVSKCQLANQCNYDCKLDKHARSGECFYDEKRNLQCI |
| | CDYCEY |
| SEQ ID NO: 73 | GVPINVRCRGSRDCLDPCRRAGMRFGRCINSRCHCTP |
| SEQ ID NO: 74 | YSRCQLQGFNCVVRSYGLPTIPCCRGLTCRSYFPGSTYGRCQRY |
| SEQ ID NO: 75 | MCMPCFTTDHQMARKCDDCCGGKGRGKCYGPQCLCR |
| SEQ ID NO: 76 | AQEPVKGPVSTKPGSCPIILIRCAMLNPPNRCLKDTDCPGIKKCCEGSCG |
| | MACFVPQ |
| SEQ ID NO: 77 | MCMPCFTTDPNMAKKCRDCCGGNGKCFGPQCLCNR |
| SEQ ID NO: 78 | GIGDPVTCLKSGAICHPVFCPRRYKQIGTCGLPGTKCCKKP |
| SEQ ID NO: 79 | GVPTDVKCRGSPQCIQPCKDAGMRFGKCMNGKCHCTPK |
| SEQ ID NO: 80 | RCPPCFTTNPNMEADCRKCCGGRGYCASYQCICPGG |

In some embodiments, a peptide of present disclosure comprises a sequence of:

X⁰X¹X²X³X⁴X⁵X⁶X⁷X⁸X⁹X¹⁰X¹¹X¹²CX¹⁴X¹⁵X¹⁶CX¹⁸X¹⁹X²⁰X²¹X²²X²³X²⁴X²⁵X²⁶X²⁷ CX²⁹NX³¹X³²CX³⁴CX³⁶X³⁷X³⁸X³⁹ (SEQ ID NO: 83), wherein any one of X⁰ to X³⁹ is independently any amino acid or null. In some embodiments, a peptide of present disclosure comprises a sequence of:

X⁰VX²X³X⁴VKCX⁸GSX¹¹X¹²CLX¹⁵PCKX¹⁹X²⁰X²¹GX²³RX²⁵GKCMNGKCX³⁴CX³⁶PX³⁸X³⁹ (SEQ ID NO: 84), wherein X is any amino acid or no amino acid. In some embodiments, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, or all of the following conditions apply to SEQ ID NO: 83 or SEQ ID NO: 84: X⁰ is null, G, or Q; X¹ is R, Q, V, null, or I; X² is P, F, G, R, V, N, D, null, or A; X₃ is T, I, F, or E; X⁴ is D, N, P, G, K, V, or I; X⁵ is I, V, or Q; X⁶ is K, R, S, or E; X⁷ is C or G; X⁸ is S, T, R, K, Y or A; X⁹ is A, G, H, R or C; X¹⁰ is S or T; X¹¹ is Y, K, R, G, P, or S; X¹² is Q, D, E, P, or K; X¹⁴ is F, W, L, I, Y, R, or V; X¹⁵ is P, D, K, Q, S, G, or A; X¹⁶ is V, P, K, A, Y, or I; X¹⁸ is K, R, I, or Q; X¹⁹ is S, Q, K, R, D, or E; X²⁰ is R, M, A, L, K, or Q; X²¹ is F, null, I, V, Y, or T; X²² is G or N; X²³ is K, M, A, T, or C; X²⁴ is T, P, R, S, A, or L; X²⁵ is N, F, A, T, or G; X²⁶ is G, A, or S; X²⁷ is R or K; X²⁹ is V, M, I T, S or L; X³¹ is G, S, R, K; X³² is L, K, R, V, or A; X³⁴ is D, R, H, K, or T; X³⁶ is F, Y, T, or null; X³⁷ is null, S, P, Y, or G; X³⁸ is null, K, or C; and X³⁹ is null, G, or V. With respect to the residue positions above, it is understood that the positions and interacting residues describe different but corresponding positions within any peptide sequence described herein. For example, the first two N-terminal amino acids shown (GS) can be absent in SEQ ID NO: 41-SEQ ID NO: 80, or substituted by any other one or two amino acids, as shown in SEQ ID NO: 1-SEQ ID NO: 40, and in such peptides where the N-terminal amino acids (GS) are present, amino acid position X₁₀ in SEQ ID NO: 83 would correspond to position 12 in the peptide with GS added at the N-terminus. If 5 amino acid residues are added to the N-terminus, the corresponding positions for X₁₀ in SEQ ID NO: 83 is position 15. The position numbering used in SEQ ID NO: 83 or 84 represent relative positions. As contemplated herein, any number of amino acids can be added to the N-terminus or the C-terminus of SEQ ID NO: 83 or 84.

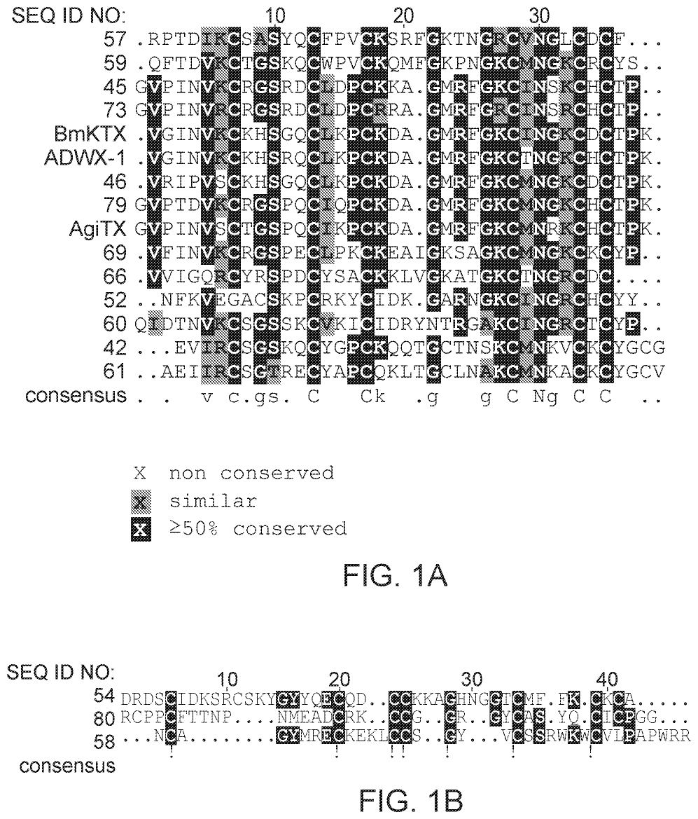

As illustrated in FIG. 1A, peptide sequences showing significant ion channel activity in the experiments described herein were aligned using an implementation of ClustalW from within the R package MSA (Multiple Sequence Alignment). The alignment was analyzed in the context of the ion channel activity data and published literature on known ion channel effectors to identify residues in the knotted peptides which are important or critical for interactions, variations that can be permitted at certain positions, and various possible binding interactions. This permits designing new peptide variants with increased or decreased binding to select ion channels, and/or enhance selectivity for a particular ion channel over other ion channel types or subtypes.

Several peptides that interacted with Kv1.1, Kv1.2, and Kv1.3 ion channels were analyzed in detail. These included SEQ ID NO: 42, 45, 46, 52, 57, 59, 60, 61, 66, 69, 73, and 79. The alignment of these sequences are shown in FIG. 1A, and were compared against published sequences for Agitoxin2, BmKTX, and the artificial sequence ADWX-1.

FIG. 1B illustrates the sequence alignment of three Kv1.2 binding peptide candidates: SEQ ID NO: 54, 58, and 80, which have more divergent structures compared to the other sequences and were not included in the alignment at FIG. 1A. From the multiple alignments of these sequences, several rules can be described in addition to the cysteine patterns for these peptides as knotted peptides described herein.

As illustrated in FIG. 1A, the residue at position 27 of the peptides in this alignment is often a Lysine (K), or in two instances an Arginine (R). The ion channels that these peptides bind to have a net negative charge at the opening, whereas the solvent exposed surfaces of the peptides comprise basic amino acid residues. Amino acid residue, K27, has been described as protruding into the ion channel, in a charge complementary way. Thus, either retaining in the knotted peptides, e.g., SEQ ID NO: 1 to SEQ ID NO: 80, of the present disclosure or optimizing knotted peptides by engineering positive charged amino acid residues (e.g., Lys or Arg) at position 27 can be used to enhance interaction with the ion channel Kv1.1, Kv1.2, or Kv1.3, or similar channels to affect or modulate its binding activity with the ion channel, such as a potassium ion channel.

In addition, several conserved amino acid residues shown in the alignment may similarly be retained or engineered into a peptide of this disclosure, e.g., SEQ ID NO: 1 to SEQ ID NO: 80, to enhance interaction with Kv1.1, 1.2, 1.3, or similar ion channels, thereby affecting activity on these ion channels. These include conserved residues at positions 6, 7, 10, 22, 26, 29, 30, and 32, as described further below.

Position 30 is 100% conserved as an Asparagine (N). This amino acid residue is important to retain or engineer as Asn at this relative position in the knotted peptide.

Position 10 is conserved in all but one sequence as a Serine (S); Threonine (T) is an iso-steric, aliphatic, alternative. This amino acid residue is optimally retained or engineered as Ser or Thr at this relative position in the knotted peptide.

Position 22 is conserved in all but one sequence as a Glycine (G). This amino acid residue is optimally retained or engineered as Gly at this relative position in the knotted peptide.

Position 26 is most commonly Glycine (G), with two instances of Alanine (A), and one of Serine (S). This amino acid residue is optimally retained or engineered as Gly, Ala or Ser at this relative position in the knotted peptide.

Position 29 is represented by two iso-steric subgroups with the amino acid residues Methionine (M) and Isoleucine (I) as one group, and Valine (V) and Threonine (T) as the other group. Serine (S) and Leucine (L) may also be tolerated here. This amino acid residue is optimally retained or engineered as Met or Ile, or Val or Thr at this relative position in the knotted peptide, or retained or engineered as Ser or Leu. Isoleucine is considered to be critical amino acid residue in some sequences.

Position 5 is either a Isoleucine (I) or a Valine (V), except for one Glutamine (Q). This amino acid residue is optimally retained or engineered as Ile, Val or Glu at this relative position in the knotted peptide.

Position 6 is either a Lysine (K) or an Arginine (R), both positively charged, in twelve of the fifteen sequences. This amino acid residue is optimally retained or engineered as Lys or Arg at this relative position in the knotted peptide.

Position 32 is similar in that it is either a Lysine (K) or an Arginine (R), both positively charged, in twelve of the fifteen sequences. This amino acid residue is optimally retained or engineered as Lys or Arg at this relative position in the knotted peptide.

In addition, this particular subgroup of CDPs may have different possible binding interactions with the ion channel, based largely on the distribution of their basic surface amino acids. One or more of residues at positions 7, 19, 24, and 34 may be part of a cluster of amino acid residues that helps define the binding interface of the peptide. The relative number of such basically charged amino acids at these relative positions can be used to optimize intereactions with an ion channel disclosed herein, such as Kv1.1, Kv1.2, and Kv1.3.

Position 34 is usually a basic amino acid such as Histidine (H) or Lysine (K), but in some circumstances, it is observed to be an acidic amino acid residue, such as Aspartic Acid (D). This amino acid residue at position 34 is optimally retained or engineered as His, Lys or Asp at this relative position in the knotted peptide. In this case, the binding interface of these proteins may change, e.g., rotate or re-orientate (e.g., due to altered electrostatic distribution or polarity of the binding interface), to place the Arginine at position 24, as described above, into the ion channel, thus enhancing the peptide's inhibition activity for the ion channel. The amino acid residue at position 34 is optimally retained or engineered as His, Lys, or Asp at this relative position in the knotted peptide.

Position 7 is typically a conserved cysteine, except in SEQ ID NO: 52 wherein the corresponding cysteine has been moved forward two positions, which suggests flexibility in the location of this cysteine in the hitchin sequence/structure motif.

In some embodiments, a peptide of this disclosure binds, inhibits, or activates a potassium ion channel, e.g., Kv1.1, Kv1.2, and Kv1.3, and comprises one or more, two or more, three or more, or four or more of the following residues at the corresponding position illustrated in FIG. 1A: K or R at position 27; N at position 30; S or T at position 10; G at position 22; G or A or S at position 26; M, I, V, T, S, or L at position 29; I, V, or Q at position 5; K or R at position 6; and K or R at position 32.

In some embodiments, a peptide of this disclosure comprises a positively charge amino acid residues at positions in the ion channel binding interface. In some embodiments, a peptide that selectively target a potassium ion channel, such as Kv1.1, Kv1.2, or Kv1.3, comprises 2 or more, 3 or more, 4 or more, or 5 or more disulfide bonds. In some embodiments, a peptide of this disclosure comprises a His or Lys at position 34. In some embodiments, a peptide of this disclosure comprises an acidic residue, such as Asp, at position 34. In some embodiments, a peptide of this disclosure comprises one or more acidic residues in the ion channel binding interface to alter the polarity or the electrostatic distribution of the binding interface, such that the peptide re-orientates the binding interface to result in enhanced selectivity or activity of the peptide for an ion channel over other ion channels or ion channel subtypes, e.g., enhanced selectivity for Kv1.3 over Kv1.1 and Kv1.2.

In some embodiments, the number of disulfide bonds within a peptide can be at least 1, at least 2, at least 3, at least 4, at least 5, at least 6, at least 7, at least 8, at least 9, or at least 10.

In some instances, the peptide can contain only one lysine residue, or no lysine residues. In some embodiments, the peptide comprises at least two lysine residues. In some embodiments, the peptide comprises at least two consecutive lysine residues. In some instances, some or all of the lysine residues in the peptide are replaced with arginine residues. In some instances, some or all of the methionine residues in the peptide are replaced by leucine or isoleucine. Some or all of the tryptophan residues in the peptide can be replaced by phenylalanine or tyrosine. In some instances, some or all of the asparagine residues in the peptide are replaced by glutamine. In some embodiments, some or all of the aspartic acid residues can be replaced by glutamic acid residues. In some cases, the N-terminus of the peptide is blocked, such as by an acetyl group. Alternatively or in combination, in some instances, the C-terminus of the peptide is blocked, such as by an amide group. In some embodiments, the peptide is modified by methylation on free amines. For example, full methylation can be accomplished through the use of reductive methylation with formaldehyde and sodium cyanoborohydride.

In some cases, the first two N-terminal amino acids can be GS as shown in SEQ ID NO: 1-SEQ ID NO: 40, or such N-terminal amino acids (GS) can be absent as shown in SEQ ID NO: 41-SEQ ID NO: 80, or can be substituted by any other one or two amino acids. In some cases, the first two N-terminal amino acids can be GS as shown in SEQ ID NO: 1-SEQ ID NO: 40, or such N-terminal amino acids (GS) can be absent as shown in SEQ ID NO: 41-SEQ ID NO: 80, or can be substituted by the amino acids GG.

In some cases, the C-terminal Arg residues of a peptide is modified to another residue such as Ala, Asn, Asp, Gln, Glu, Gly, His, Leu, Lys, Met, Phe, Pro, Ser, Thr, Trp, Tyr, or Val. For example, the C-terminal Arg residue of a peptide can be modified to Ile. Alternatively, the C-terminal Arg residue of a peptide can be modified to any non-natural amino acid. This modification can prevent clipping of the C-terminal residue during expression, synthesis, processing, storage, in vitro, or in vivo including during treatment, while still allowing maintenance of a key hydrogen bond. A key hydrogen bond can be the hydrogen bond formed during the initial folding nucleation and is critical for forming the initial hairpin.

In some cases the peptide comprises the sequence of any one of SEQ ID NO: 1-SEQ ID NO: 80. A peptide can be a fragment comprising a contiguous fragment of any one of SEQ ID NO: 1-SEQ ID NO: 80 that is at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46 at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76 residues long, at least 77, at least 78, at least 79, at least 80, or at least 81 residues long, wherein the peptide fragment is selected from any portion of the peptide. In some embodiments, the peptide sequence is flanked by additional amino acids. One or more additional amino acids can, for example, confer a desired in vivo charge, isoelectric point, chemical conjugation site, stability, or physiologic property to a peptide.

The peptides of the present disclosure can further comprise negatively charged amino acid residues. In some cases, the peptide has 2 or fewer negative amino acid residues. In other cases, the peptide has 4 or fewer negative amino acid residues, 3 or fewer negative amino acid residues, or 1 or fewer negative amino acid residues. The negative amino acid residues can be selected from any negatively charged amino acid residues. The negative amino acid residues can selected from either E or D, or a combination of both E and D.

The peptides of the present disclosure can further comprise basic amino acid residues. In some embodiments, basic residues are added to the peptide sequence to increase the charge at physiological pH. The added basic residues can be any basic amino acid. The added basic residues can be selected from K or R, or a combination of K or R.

In some embodiments, the peptide has a charge distribution comprising an acidic region and a basic region. An acidic region can be a nub. A nub is a portion of a peptide extending out of the peptide's three-dimensional structure. A basic region can be a patch. A patch is a portion of a peptide that does not designate any specific topology characteristic of the peptide's three-dimensional structure. In further embodiments, a knotted peptide can be 6 or more basic residues and 2 or fewer acidic residues.

The peptides of the present disclosure can further comprise positively charged amino acid residues. In some cases, the peptide has at least 1 positively charged residue. In some cases, the peptide has at least 2 positively charged residues. In some cases, the peptide has at least 3 positively charged residues. In other cases, the peptide has at least 4 positively charged residues, at least 5 positively charged residues, at least 6 positively charged residues, at least 7 positively charged residues, at least 8 positively charged residues or at least 9 positively charged residues. While the positively charged residues can be selected from any positively charged amino acid residues, in some embodiments, the positively charged residues are either K, or R, or a combination of K and R.

The peptides of the present disclosure can further comprise neutral amino acid residues. In some cases, the peptide has 35 or fewer neutral amino acid residues. In other cases, the peptide has 81 or fewer neutral amino acid residues, 70 or fewer neutral amino acid residues, 60 or fewer neutral amino acid residues, 50 or fewer neutral amino acid residues, 40 or fewer neutral amino acid residues, 36 or fewer neutral amino acid residues, 33 or fewer neutral amino acid residues, 30 or fewer neutral amino acid residues, 25 or fewer neutral amino acid residues, or 10 or fewer neutral amino acid residues.

The peptides of the present disclosure can further comprise negative amino acid residues. In some cases the peptide has 6 or fewer negative amino acid residues, 5 or fewer negative amino acid residues, 4 or fewer negative amino acid residues, 3 or fewer negative amino acid residues, 2 or fewer negative amino acid residues, or 1 or fewer negative amino acid residues. While negative amino acid residues can be selected from any neutral charged amino acid residues, in some embodiments, the negative amino acid residues are either E, or D, or a combination of both E and D.

At physiological pH, peptides can have a net charge, for example, of −5, −4, −3, −2, −1, 0, +1, +2, +3, +4, or +5. When the net charge is zero, the peptide can be uncharged or zwitterionic. In some embodiments, the peptide contains one or more disulfide bonds and has a positive net charge at physiological pH where the net charge can be +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10. In some embodiments, the peptide has a negative net charge at physiological pH where the net charge can be −0.5 or less than −0.5, −1 or less than −1, −1.5 or less than −1.5, −2 or less than −2, −2.5 or less than −2.5, −3 or less than −3, −3.5 or less than −3.5, −4 or less than −4, −4.5 or less than −4.5, −5 or less than −5, −5.5 or less than −5.5, −6 or less than −6, −6.5 or less than −6.5, −7 or less than −7, −7.5 or less than −7.5, −8 or less than −8, −8.5 or less than −8.5, −9 or less than −9.5, −10 or less than −10. In some cases, the engineering of one or more mutations within a peptide yields a peptide with an altered isoelectric point, charge, surface charge, or rheology at physiological pH. Such engineering of a mutation to a peptide derived from a scorpion or spider can change the net charge of the complex, for example, by decreasing the net charge by 1, 2, 3, 4, or 5, or by increasing the net charge by 1, 2, 3, 4, or 5. In such cases, the engineered mutation can facilitate the ability of the peptide to pass through the gastrointestinal tract intact, to have a longer half life in serum or other compartments of the body, or to maintain secondary or tertiary structure in intracellular environments. Suitable amino acid modifications for improving the rheology and potency of a peptide can include conservative or non-conservative mutations. A peptide can comprise at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to the sequence of the venom or toxin, component that the peptide is derived from. In some embodiments, mutations can be in a single loop between disulfide bonds or can be in multiple loops. In other embodiments, mutations can improve pharmacokinetic or biodistribution properties, or can add, enhance, or decrease biological activities. In other cases, a peptide, or a functional fragment thereof, comprises at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, or another suitable number as compared to the sequence of the venom, toxin, or native component that the peptide is derived from. In some embodiments, mutations can be engineered within a peptide to provide a peptide that has a desired charge or stability at physiological pH.

Generally, the NMR solution structures, the x-ray crystal structures, as well as the primary structure sequence alignment of related structural homologs can be used to inform mutational strategies that can improve the folding, stability, and/or manufacturability, while maintaining a particular biological function. They can be used to predict the 3D pharmacophore of a group of structurally homologous scaffolds, as well as to predict possible graft regions of related proteins to create chimeras with improved properties. The general strategy for producing homologs can include identification of a charged surface patch of a protein, mutation of critical amino acid positions and loops, and testing of sequences. This strategy can be used to design peptides with improved properties or to correct deleterious mutations that complicate folding and manufacturability. These key amino acid positions and loops can be retained while other residues in the peptide sequences can be mutated to improve, change, remove, or otherwise modify function, homing, and activity of the peptide. The crystal structure of several peptides of this disclosure were solved and can be used to modify peptide function as described herein.

Improved peptides can also be engineered based upon immunogenicity information, such as immunogenicity information predicted by TEPITOPE and TEPITOPEpan. TEPITOPE is a computational approach which uses position specific scoring matrix to provide prediction rules for whether a peptide will bind to 51 different HLA-DR alleles, and TEPITOPEpan is method that uses TEPITOPE to extrapolate from HLA-DR molecules with known binding specificities to HLA-DR molecules with unknown binding specificities based on pocket similarity. For example, TEPITOPE and TEPITOPEpan can be used to determine immunogenicity of peptides that have improved stability. Comparison of peptides with high immunogenicity to peptides with low immunogenicity can guide engineering strategies for designing stable variants with decreased immunogenicity.

Additionally, the comparison of the primary sequences and the tertiary sequences of two or more peptides can be used to reveal sequence and 3D folding patterns that can be leveraged to improve the peptides and parse out the biological activity of these peptides. For example, comparing two different peptide scaffolds that are reduction resistant or protease resistant can lead to the identification of conserved pharmacophores that can guide engineering strategies, such as designing variants with improved resistance and stability properties.

The present disclosure also encompasses multimers of the various peptides described herein. Examples of multimers include dimers, trimers, tetramers, pentamers, hexamers, heptamers, and so on. A multimer may be a homomer formed from a plurality of identical subunits or a heteromer formed from a plurality of different subunits. In some embodiments, a peptide of the present disclosure is arranged in a multimeric structure with at least one other peptide, or two, three, four, five, six, seven, eight, nine, ten, or more other peptides. In certain embodiments, the peptides of a multimeric structure each have the same sequence. In alternative embodiments, some or all of the peptides of a multimeric structure have different sequences.

The present disclosure further includes peptide scaffolds that, e.g., can be used as a starting point for generating additional peptides. In some embodiments, these scaffolds can be derived from a variety of knotted peptides or knottins. Some suitable peptide for scaffolds can include, but are not limited to, chlorotoxin, brazzein, circulin, stecrisp, hanatoxin, midkine, hefutoxin, potato carboxypeptidase inhibitor, bubble protein, attractin, α-GI, α-GID, p-PIIIA, ω-MVIIA, ω-CVID, χ-MrIA, ρ-TIA, conantokin G, contulakin G, GsMTx4, margatoxin, shK, toxin K, and EGF epiregulin core.

Two or more peptides can share a degree of sequence identity or homology and share similar properties in vivo. For instance, a peptide can share a degree of sequence identity or homology with any one of the peptides of SEQ ID NO: 1-SEQ ID NO: 80. In some cases, one or more peptides of the disclosure can have up to about 20% pairwise sequence identity or homology, up to about 25% pairwise sequence identity or homology, up to about 30% pairwise sequence identity or homology, up to about 35% pairwise sequence identity or homology, up to about 40% pairwise sequence identity or homology, up to about 45% pairwise sequence identity or homology, up to about 50% pairwise sequence identity or homology, up to about 55% pairwise sequence identity or homology, up to about 60% pairwise sequence identity or homology, up to about 65% pairwise sequence identity or homology, up to about 70% pairwise sequence identity or homology, up to about 75% pairwise sequence identity or homology, up to about 80% pairwise sequence identity or homology, up to about 85% pairwise sequence identity or homology, up to about 90% pairwise sequence identity or homology, up to about 95% pairwise sequence identity or homology, up to about 96% pairwise sequence identity or homology, up to about 97% pairwise sequence identity or homology, up to about 98% pairwise sequence identity or homology, up to about 99% pairwise sequence identity or homology, up to about 99.5% pairwise sequence identity or homology, or up to about 99.9% pairwise sequence identity or homology. Various methods and software programs can be used to determine the homology between two or more peptides, such as NCBI BLAST, Clustal W, MAFFT, Clustal Omega, AlignMe, Praline, or another suitable method or algorithm.

Pairwise sequence alignment is used to identify regions of similarity that may indicate functional, structural and/or evolutionary relationships between two biological sequences (protein or nucleic acid). By contrast, multiple sequence alignment (MSA) is the alignment of three or more biological sequences. From the output of MSA applications, homology can be inferred and the evolutionary relationship between the sequences assessed. One of skill in the art would recognize as used herein, “sequence homology” and “sequence identity” and “percent (%) sequence identity” and “percent (%) sequence homology” have been used interchangeably to mean the sequence relatedness or variation, as appropriate, to a reference polynucleotide or amino acid sequence.

In some instances, the peptide is any one of SEQ ID NO: 1-SEQ ID NO: 80 or a functional fragment thereof In other embodiments, the peptide of the disclosure further comprises a peptide with 99%, 98%, 97%, 96%, 95%, 94%, 93%, 92%, 91%, 90%, 89%, 88%, 87%, 86%, 85%, 84%, 83%, 82%, 81%, or 80% sequence identity or homology to any one of SEQ ID NO: 1-SEQ ID NO: 80 or fragment thereof.

In other instances, the peptide can be a peptide that is homologous to any one of SEQ ID NO: 1-SEQ ID NO: 80 or a functional fragment thereof. The term “homologous” is used herein to denote peptides having at least 70%, at least 80%, at least 90%, at least 95%, or greater than 95% sequence identity or homology to a sequence of any one of SEQ ID NO: 1-SEQ ID NO: 80 or a functional fragment thereof

In still other instances, the variant nucleic acid molecules of a peptide of any one of SEQ ID NO: 1-SEQ ID NO: 80 can be identified by either a determination of the sequence identity or homology of the encoded peptide amino acid sequence with the amino acid sequence of any one of SEQ ID NO: 1-SEQ ID NO: 80, or by a nucleic acid hybridization assay. Such peptide variants can include nucleic acid molecules (1) that remain hybridized with a nucleic acid molecule having the nucleotide sequence of any one of SEQ ID NO: 1-SEQ ID NO: 80 (or its complement) under stringent washing conditions, in which the wash stringency is equivalent to 0.5×-2×SSC with 0.1% SDS at 55-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 80. Alternatively, peptide variants of any one of SEQ ID NO: 1-SEQ ID NO: 80 can be characterized as nucleic acid molecules (1) that remain hybridized with a nucleic acid molecule having the nucleotide sequence of any one of SEQ ID NO: 1-SEQ ID NO: 80 (or its complement) under highly stringent washing conditions, in which the wash stringency is equivalent to 0.1×-0.2×SSC with 0.1% SDS at 50-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 80.

Percent sequence identity or homology is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid molecules using a ratio as disclosed above. For nucleotide sequence comparisons, the ktup value can range between one to six, preferably from three to six, most preferably three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., Chimera (University of California, San Francisco, Calif.), PyMol (Schrödinger, Inc. New York N.Y.), or Rosetta (University of Washington, Seattle Wash.)), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments, the determination of structure can typically be accompanied by evaluating activity of modified molecules.

Certain embodiments of the peptides described herein exhibit properties that enhance localization, binding, accumulation in, and/or internalization by target tissues, regions, compartments, or cells. Examples of peptide properties that can be relevant to target tissue/cell binding and internalization include but are not limited to isoelectric point, net charge, charge distribution, molecular weight, hydrodynamic radius, pH stability, hydrophilicity, and protein-protein binding.

For example, in various embodiments, the peptides of the present disclosure exhibit an isoelectric point (pI) favorable for target tissue localization, ion channel binding, and/or internalization. In certain embodiments, the pI of a peptide is less than or equal to about 2.0, less than or equal to about 2.5, less than or equal to about 3.0, less than or equal to about 3.5, 4.0, less than or equal to about 4.5, less than or equal to about 5.5, less than or equal to about 6.0, less than or equal to about 6.5, less than or equal to about 7.0, less than or equal to about 7.5, less than or equal to about 8.0, less than or equal to about 8.5, less than or equal to about 9.0, less than or equal to about 9.5, less than or equal to about 10.0, less than or equal to about 10.5, less than or equal to about 11.0, less than or equal to about 11.5, less than or equal to about 12.0, less than or equal to about 12.5, less than or equal to about 13.0, less than or equal to about 13.5, less than or equal to about 14.0, less than or equal to about 14.5, or less than or equal to about 15.0. In certain embodiments, the pI of a peptide is greater than or equal to about 2.0, greater than or equal to about 2.5, greater than or equal to about 3.0, greater than or equal to about 3.5, 4.0, greater than or equal to about 4.5, greater than or equal to about 5.5, greater than or equal to about 6.0, greater than or equal to about 6.5, greater than or equal to about 7.0, greater than or equal to about 7.5, greater than or equal to about 8.0, greater than or equal to about 8.5, greater than or equal to about 9.0, greater than or equal to about 9.5, or greater than or equal to about 10.0, greater than or equal to about 10.5, greater than or equal to about 11.0, greater than or equal to about 11.5, greater than or equal to about 12.0, greater than or equal to about 12.5, greater than or equal to about 13.0, greater than or equal to about 13.5, greater than or equal to about 14.0, greater than or equal to about 14.5, or greater than or equal to about 15.0. The pI of a peptide can be within a range from about 3.0 to about 10.0, within a range from about 3.0 to about 6.0, or within a range from about 4.0 to about 9.0.

In some embodiments, the pI (the pH at which the net charge of the peptide is zero) of the peptides of this disclosure can be calculated by the EMBOSS method. The pI value is the isoelectric point of fully reduced form of protein sequences. The value can be calculated with the Henderson-Hasselbalch equation using EMBOSS scripts and a pKa table provided by the European Bioinformatics Institute. The EMBOSS method of calculating pI has been described by Rice et al. (EMBOSS: the European Molecular Biology Open Software Suite. Trends Genet. 2000 June; 16(6):276-7) and Carver et al. (The design of Jemboss: a graphical user interface to EMBOSS. Bioinformatics. 2003 Sep. 22; 19(14):1837-43).

In some embodiments, the first two N-terminal amino acids of SEQ ID NO: 1-SEQ ID NO: 40 (GS or GG) serve as a spacer or linker in order to facilitate conjugation or fusion to another molecule, as well as to facilitate cleavage of the peptide from such conjugated or fused molecules. In some embodiments, the peptide may not include the first two N-terminal amino acids, as shown in SEQ ID NO: 41-SEQ ID NO: 80, or such N-terminal amino acids can be substituted by GG, or any other one or two amino acids.

The present disclosure encompasses various modifications to the peptides provided herein. In some embodiments, a peptide of the present disclosure contains or is modified to contain only one lysine residue, or no lysine residues. In some embodiments, some or all of the lysine residues in the peptide are replaced with arginine residues. In some embodiments, some or all of the methionine residues in the peptide are replaced by leucine or isoleucine. In some embodiments, some or all of the tryptophan residues in the peptide are replaced by phenylalanine or tyrosine. In some embodiments, some or all of the asparagine residues in the peptide are replaced by glutamine. In some embodiments, some or all of the cysteine residues in the peptide are replaced by serine to produce a linearized form of the peptide. In some embodiments, the N-terminus of the peptide is blocked, such as by an acetyl group. In some embodiments, the N-terminus of the peptide is blocked with pyroglutamic acid. Alternatively or in combination, in some instances, the C-terminus of the peptide is blocked, such as by an amide group. In some embodiments, the peptide is modified by methylation on free amines. For example, full methylation can be accomplished through the use of reductive methylation with formaldehyde and sodium cyanoborohydride.

In some embodiments, more than one peptide sequence is present on a particular peptide. For example, a peptide of the present disclosure can include sequences from at least 1, at least 2, at least 3, at least 4, at least 5, at least 6, at least 7, at least 8, at least 9, or at least 10 different peptides, or fragments thereof

In some embodiments, a peptide of present disclosure binds to an ion channel with high affinity. In some cases, the binding affinity is between 0.01 nM to 1000 nM or between 0.1 nM to 100 nM. In some embodiments, the binding affinity is about 1 μM. In some embodiments, the binding affinity is less than 0.1 nM, less than 0.5 nM, less than 1 nM, less than 5 nM, less than 10 nM, less than 30 nM, less than 50 nM, less than 70 nM, less than 100 nM, less than 200 nM, less than 300 nM, less than 400 nM, less than 500 nM, less than 600 nM, less than 700 nM, or less than 1000 nM.

In some embodiments, a peptide disclosed herein is derived from or comprises a cholera toxin, alpha-KTX 6.10, a potassium channel toxin, a hadrucalcin, a hainantoxin, alpha KTx 3.10, BoiTx1, kaliotoxin-2, alpha-KTx 3.5, KTX-2, omega theraptotoxin-Grla, Omega-grammotoxin, chloride channel toxin meuC114, U5-Theraptotoxin-Hs1b 2, PK+ channel toxin alpha-KTx 15.8, BmKKx1, Opicalcin-2, K+ channel blocker alpha-KTx 26.1, Neurotoxin BmK86, K+ toxin-like Tx-677, gamma KTx 1.8, CeErgTx5, alpha-KTx5.3, Neurotoxin BmP05, U10-hexatoxin Mg1a, neurotoxin magi-9, K+ channel gamma-KTx 2.2, BmKK7, U3-theraphotoxin-Hhn1n, Hainantoxin-VIII-6, K+ channel toxin alpha-KTx 1.5, BmTX1, K+ channel toxin alpha-KTx 15.6, Discrepin, K+ channel toxin alpha-KTx 6.9, OcKTx4, K+ channel toxin alpha-KTx, U11-theraptotoxin-Hhn1v, Hainantoxin-XVI-22, Hainantoxin HNTX-XX, U11-Hexatoxin Mg1a, neurotoxin magi-10, K+ channel toxin alpha KTx 4.2, Neurotoxin Ts-kappa, Defensin-1, Neurotoxin Bt1Tx3, Neurotoxin BTChl1, K+ channel toxin alpha-KTx 4.5, K+ channel toxin alpha-KTx 15.9, Neurotoxin KTx9, Mu-theraptotoxin-Hs2a, Huwentoxin-4, Brazzein, tachystatin, chlorotoxin, TEAD, meu14toxinA, human beta defensin-2, potassium channel toxin alpha-KTx 3.11, neurotoxin BtITx3, BTChl1, or a derivative, homologue, a functional fragment, or a combination thereof.

### Chemical Modifications

A peptide can be chemically modified one or more of a variety of ways. In some embodiments, the peptide can be mutated to add function, delete function, or modify the in vivo behavior. One or more loops between the disulfide linkages can be modified or replaced to include active elements from other peptides (such as described in Moore and Cochran, Methods in Enzymology, 503, p. 223-251, 2012). Amino acids can also be mutated, such as to increase half-life, modify, add or delete binding behavior in vivo, add new targeting function, modify surface charge and hydrophobicity, or allow conjugation sites. N-methylation is one example of methylation that can occur in a peptide of the disclosure. In some embodiments, the peptide is modified by methylation on free amines. For example, full methylation may be accomplished through the use of reductive methylation with formaldehyde and sodium cyanoborohydride.

A chemical modification can, for instance, extend the half-life of a peptide or change the biodistribution or pharmacokinetic profile. A chemical modification can comprise a polymer, a polyether, polyethylene glycol, a biopolymer, a polyamino acid, a fatty acid, a dendrimer, an Fc region, a simple saturated carbon chain such as palmitate or myristolate, or albumin. A polyamino acid can include, for example, a poly amino acid sequence with repeated single amino acids (e.g., poly glycine), and a poly amino acid sequence with mixed poly amino acid sequences (e.g., gly-ala-gly-ala (SEQ ID NO: 85)) that may or may not follow a pattern, or any combination of the foregoing.

The peptides of the present disclosure can be modified such that the modification increases the stability and/or the half-life of the peptides. The attachment of a hydrophobic moiety, such as to the N-terminus, the C-terminus, or an internal amino acid, can be used to extend half-life of a peptide of the present disclosure. The peptides can also be modified to increase or decrease the gut permeability or cellular permeability of the peptide. The peptide of the present disclosure can include post-translational modifications (e.g., methylation and/or amidation and/or glycosylation), which can affect, e.g., serum half-life. In some embodiments, simple carbon chains (e.g., by myristoylation and/or palmitylation) can be conjugated to the fusion proteins or peptides. The simple carbon chains can render the fusion proteins or peptides easily separable from the unconjugated material. For example, methods that can be used to separate the fusion proteins or peptides from the unconjugated material include, but are not limited to, solvent extraction and reverse phase chromatography. Lipophilic moieties can extend half-life through reversible binding to serum albumin. Conjugated moieties can, e.g., be lipophilic moieties that extend half-life of the peptides through reversible binding to serum albumin. In some embodiments, the lipophilic moiety can be cholesterol or a cholesterol derivative including cholestenes, cholestanes, cholestadienes and oxysterols. In some embodiments, the peptides can be conjugated to myristic acid (tetradecanoic acid) or a derivative thereof. In other embodiments, the peptides of the present disclosure can be coupled (e.g., conjugated) to a half-life modifying agent. Examples of half-life modifying agents can include, but is not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin.

In some embodiments, a peptide of this disclosure comprises amidation at the C-terminus for improved stability. In some cases, a peptide of this disclosure comprises phosphotyrosine (pTyr) residue at the N-terminus for enhancing selectivity of an ion channel over other ion channels or other subtypes. In some cases, a peptide of this disclosure comprises one or more non-hydrolyzable analogs of the N-terminal pTyr, such as para phosphophenylalanine for improving its stability.

In some embodiments, poly(ethylene glycol) (PEG) polymer is conjugated to a peptide disclosed herein to improve its potency or ion channel binding activity. In some cases, 30 kDa linear PEG or branched PEG consisting of two 10 kDa PEG arms can be conjugated to a peptide of this disclosure to enhance its ion channel binding properties. In some embodiments, a peptide disclosed herein is conjugated to a PEG polymer at the N-terminus to enhance its selectivity for a target ion channel over other ion channel or ion channel subtypes, such as Kv1.3 over Kv1.1 and Kv1.2, by at least 10 fold, at least 20 fold, at least 50 fold, at least 100 fold, at least 200 fold, at least 500 fold, or at least 1000 fold as compared to a peptide without any conjugation.

In some embodiments, the first two N-terminal amino acids (GS) of SEQ ID NO: 1-SEQ ID NO: 40 serve as a spacer or linker in order to facilitate conjugation or fusion to another molecule, as well as to facilitate cleavage of the peptide from such conjugated or fused molecules. In some embodiments, the fusion proteins or peptides of the present disclosure can be conjugated to other moieties that, e.g., can modify or effect changes to the properties of the peptides.

### Active Agent Peptide Conjugates

In some embodiments, a peptide of the present disclosure functions as an active agent that modulates ion channel activity, including as an ion channel agonist or antagonist. In some embodiments, a peptide of this disclosure binds to an ion channel to block, inhibit, or deactivate the ion channel or its activity. In some embodiments, a peptide of this disclosure binds to an ion channel to activate or open the channel, or increase the ion channel activity.

In other embodiments, a peptide of the present disclosure can be conjugated to another active agent, such as an ion channel activator, agonist, inhibitor, antagonist, co-factor, or an imaging molecule, a detectable label, a toxin or a derivative or analog thereof, immunomodulatory agent, antibody, steroid, a small molecule, interleukin, cytokine, chemokine, immunomodulatory imide drug, or any other active agent having a therapeutic effect. In some embodiments, a peptide is conjugated to any one of the active agents, detectable labels, and/or a moiety disclosed herein to target the such active agent, detectable label, and/or a moiety to a target organ, tissue, or cells, such as those from kidney, brain, nerve cells (both peripheral and central nervous system), immune system, heart, GI tract, skeletal muscle, smooth muscle, eye, retina, cartilage, and cancer.

Peptides according to the present disclosure can be conjugated or fused to an agent relevant for any disease disclosed herein for use in the treatment of tumors, cancers, brain disorders, cartilage disorders, skin disorders, lung disorders, cardiovascular diseases, gastrointestinal diseases and disorders, vaginal mucosal diseases, ocular diseases, oral diseases, pain or pain management, autoimmune disease, neurological disorders, renal diseases, or other mucosal diseases.

For example, in certain embodiments, the peptides described herein are fused to another molecule, such as an active agent that provides a functional capability. A peptide can be fused with an active agent through expression of a vector containing the sequence of the peptide with the sequence of the active agent. In various embodiments, the sequence of the peptide and the sequence of the active agent can be expressed from the same Open Reading Frame (ORF). In various embodiments, the sequence of the peptide and the sequence of the active agent can comprise a contiguous sequence. The peptide and the active agent can each retain similar functional capabilities in the fusion peptide compared with their functional capabilities when expressed separately. In certain embodiments, examples of active agents can include other peptides.

As another example, in certain embodiments, the peptides described herein are attached to another molecule, such as an active agent that provides a functional capability. In some embodiments, 1, 2, 3, 4, 5, 6, 7, 8, 9, or 10 active agents can be linked to a peptide. Multiple active agents can be attached by methods such as conjugating to multiple lysine residues and/or the N-terminus, or by linking the multiple active agents to a scaffold, such as a polymer or dendrimer and then attaching that agent-scaffold to the peptide (such as described in Yurkovetskiy, A. V., Cancer Res 75(16): 3365-72 (2015). Examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an oligonucleotide, an antibody, a single chain variable fragment (scFv), an antibody fragment, an aptamer, a cytokine, an interferon, a hormone, an enzyme, a growth factor, a checkpoint inhibitor, a PD-1 inhibitor, a PD-L1 inhibitor, a CTLA4 inhibitor, a CD antigen, a chemokine, a neurotransmitter, an ion channel inhibitor, an ion channel activator, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, a complement fixing peptide or protein, a tumor necrosis factor inhibitor, a tumor necrosis factor activator, a tumor necrosis factor receptor family agonist, a tumor necrosis receptor antagonist, a Tim-3 inhibitor, a protease inhibitor, an amino sugar, a chemotherapeutic, a cytotoxic molecule, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, polyethylene glycol, a lipid, a dendrimer, a fatty acid, or an Fc region, or an active fragment or a modification thereof. In some embodiments, the peptide is covalently or non-covalently linked to an active agent, e.g., directly or via a linker. For example, cytotoxic molecules that can be used include auristatins, MMAE, MMAF, dolostatin, auristatin F, monomethylaurstatin D, DM1, DM4, maytansinoids, maytansine, calicheamicins, N-acetyl-γ-calicheamicin, pyrrolobenzodiazepines, PBD dimers, doxorubicin, vinca alkaloids (4-deacetylvinblastine), duocarmycins, cyclic octapeptide analogs of mushroom amatoxins, epothilones, and anthracylines, CC-1065, taxanes, paclitaxel, cabazitaxel, docetaxel, SN-38, irinotecan, vincristine, vinblastine, platinum compounds, cisplatin, methotrexate, and BACE inhibitors. Additional examples of active agents are described in McCombs, J. R., AAPS J, 17(2): 339-51 (2015), Ducry, L., Antibody Drug Conjugates (2013), and Singh, S. K., Pharm Res. 32(11): 3541-3571 (2015). Exemplary linkers suitable for use with the embodiments herein are discussed in further detail below.

As compared to antibody-drug conjugates (e.g., Adcetris, Kadcyla, Mylotarg), in some aspects the peptide conjugated to an active agent as described herein can exhibit better penetration of solid tumors due to its smaller size. In certain aspects, the peptide conjugated to an active agent as described herein can carry different or higher doses of active agents as compared to antibody-drug conjugates. In still other aspects, the peptide conjugated to an active agent as described herein can have better site specific delivery of defined drug ratio as compared to antibody-drug conjugates. In other aspects, the peptide can be amenable to solvation in organic solvents (in addition to water), which can allow more synthetic routes for solvation and conjugation of a drug (which often has low aqueous solubility) and higher conjugation yields, higher ratios of drug conjugated to peptide (versus an antibody), and/or reduce aggregate/high molecular weight species formation during conjugation. Additionally, a unique amino acid residue(s) can be introduced into the peptide via a residue that is not otherwise present in the short sequence or via inclusion of a non-natural amino acid, allowing site specific conjugation to the peptide.

The peptides or fusion peptides of the present disclosure can also be conjugated to other moieties that can serve other roles, such as providing an affinity handle (e.g., biotin) for retrieval of the peptides from tissues or fluids. For example, peptides or fusion peptides of the present disclosure can also be conjugated to biotin. In addition to extension of half-life, biotin can also act as an affinity handle for retrieval of peptides or fusion peptides from tissues or other locations. In some embodiments, fluorescent biotin conjugates that can act both as a detectable label and an affinity handle can be used. Non limiting examples of commercially available fluorescent biotin conjugates can include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexa fluor 488 biocytin, Alexa flour 546, Alexa Fluor 549, lucifer yellow cadaverine biotin-X, Lucifer yellow biocytin, Oregon green 488 biocytin, biotin-rhodamine and tetramethylrhodamine biocytin. In some other examples, the conjugates can include chemiluminescent compounds, colloidal metals, luminescent compounds, enzymes, radioisotopes, and paramagnetic labels. In some embodiments, the peptide described herein can also be attached to another molecule. For example, the peptide sequence also can be attached to another active agent (e.g., small molecule, peptide, polypeptide, polynucleotide, antibody, aptamer, cytokine, growth factor, neurotransmitter, an active fragment or modification of any of the preceding agents, fluorophore, radioisotope, radionuclide chelator, acyl adduct, chemical linker, or sugar). In some embodiments, the peptide can be fused with, or covalently or non-covalently linked to an active agent.

Additionally, more than one peptide sequence derived from a toxin or venom knottin protein can be present on or fused with a particular peptide. A peptide can be incorporated into a biomolecule by various techniques. A peptide can be incorporated by a chemical transformation, such as the formation of a covalent bond, such as an amide bond. A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule, or can substitute for a subsequence of the sequence that encodes the biomolecule.

A peptide can be conjugated to an agent used in imaging, research, therapeutics, theranostics, pharmaceuticals, chemotherapy, chelation therapy, targeted drug delivery, and radiotherapy.

In some embodiments, active agents can be conjugated to any peptide of this disclosure according to the desired function or activity. For example, active agents can belong to the class of anti-inflammatory drugs, immunosuppressive (immune suppression) drugs, analgesics/pain relief drugs, cell depleting agents/apoptosis modifiers, and tissue normalization (disease modifying) drugs.

Anti-inflammatory active agents can include, but are not limited to, corticosteroids, nonsteroidal anti-inflammatory drugs (NSAIDs), biologics, and other small molecules. Examples of corticosteroid active agents that can be conjugated to any peptide of this disclosure for delivery to a target tissue, organ, or cells include triamcinolone. Examples of NSAID active agents that can be conjugated to any peptide of this disclosure for delivery to target tissue, organ, or cells include naproxen. NSAID active agents can be further classified into COX2 inhibitors. An example of a COX2 inhibitor active agent directed to a prostaglandin pathway that can be conjugated to any peptide of this disclosure for delivery includes celecoxib. An example of a COX2 inhibitor active agent with anti-leukotriene receptor antagonist that can be conjugated to any peptide of this disclosure for delivery includes montelukast. An example of a COX2 inhibitor active agent that can be conjugated to any peptide of this disclosure for delivery to a target tissue, organ, or cells includes iguratimod.

Biologic active agents can be further classified into active agents that are IL-1 family inhibitors, IL-17 or IL-23 pathway inhibitors, IL-6 family inhibitors, interferon receptor inhibitors, tumor necrosis factor (TNF) inhibitors, RANK pathway inhibitors, B cell inhibitors, anti-IgE active agents, and co-stimulation inhibitors. An example of an IL-1 family inhibitor active agent that can be conjugated to any peptide of this disclosure for delivery includes anakinra. An example of an IL-17/IL-23 pathway inhibitor active agent that can be conjugated to any peptide of this disclosure for delivery includes secukinumab. An example of an IL-6 family inhibitor active agent that can be conjugated to any peptide of this disclosure includes tocilizumab, siltuxima, and sirukumab. An example of an interferon receptor inhibitor active agent that can be conjugated to any peptide of this disclosure includes anifrolumab. An example of a TNF inhibitor active agent that can be conjugated to any peptide of this disclosure for delivery includes infliximab or etanercept. An example of a RANK pathway inhibitor active agent that can be conjugated to any peptide of this disclosure for delivery includes denosumab. An example of a B cell inhibitor active agent that can be conjugated to any peptide of this disclosure for delivery to target tissue, organ, or cells includes ocrelizumab, ofatumumab, obinutuzumab, and rituximab. An example of an anti-IgE active agent that can be conjugated to any peptide of this disclosure includes omalizumab. An example of a co-stimulation inhibitor active agent that can be conjugated to any peptide of this disclosure for delivery includes abatacept.

Pain relief active agents can include, but are not limited to analgesics, counter-irritants, and pain receptor blocking drugs. Analgesics can be further classified into non-narcotic agents and narcotic agents. An example of a non-narcotic active agent that can be conjugated to any peptide of this disclosure for delivery includes acetaminophen. An example of a narcotic active agent that can be conjugated to any peptide of this disclosure includes oxycodone. Counter-irritant active agents can be derived from natural products. Examples of a counter-irritant active agent can be conjugated to any peptide of this disclosure for delivery include capsaicin, piperine, mustard oil, eugenol, and curcumin, and capsaicin-like molecules like resiniferatoxin (RTX). Pain receptor blocking active agents can be further classified as TRPV4 inhibitors. An example of a TRPV4 inhibitor active agent that can be conjugated to any peptide of this disclosure for delivery includes GSK2193874.

Apoptosis modifier active agents can include, but are not limited to, biologics and small molecules. Biologic apoptosis modifier active agents can be further classified as Fas/FasL inhibitors, TNF/TNFR inhibitors, TRAIL/TRAILR inhibitors, TWEAK/Fn14 inhibitors, IL-1 inhibitors, IL-1 receptor antagonists, growth factors, and sclerostin inhibitors. An example of a TNF/TNFR inhibitor active agent that can be conjugated to any peptide of this disclosure for delivery includes infliximab. An example of a TRAIL/TRAILR inhibitor active agent that can be conjugated to any peptide of this disclosure for delivery includes osteoprotegrin. An example of a TWEAK/Fn14 inhibitor active agent that can be conjugated to any peptide of this disclosure includes BIIB023 An example of an IL-1 receptor antagonist that can be conjugated to any peptide of this disclosure for delivery includes anakinra. An example of a growth factor active agent that can be conjugated to any peptide of this disclosure for delivery includes IGF-1. An example of a growth factor active agent that can be conjugated to any peptide of this disclosure includes EGF. An example of a sclerostin inhibitor active agent that can be conjugated to any peptide of this disclosure for delivery includes romosozumab. Small molecule apoptosis modifier active agents can be further classified as caspase inhibitors, iNOS inhibitors, surfactants, and bisphosphonates. An example of a caspase inhibitor active agent that can be conjugated to any peptide of this disclosure for delivery includes ZVAD-fmk. An example of an iNOS inhibitor active agent that can be conjugated to any peptide of this disclosure for delivery include S-methylisothiourea.

An example of a surfactant active agent that can be conjugated to any peptide of this disclosure includes P188. Moreover, the known class of drugs called senotherapeutics, also referred to as senolytics or senolytic drugs or senolytic compounds, refers to small molecules that can selectively induce death of senescent cells and for example by directly or indirectly inducing apoptosis in senescent cells. In addition, senolytics may also act via non-apoptotic mechanisms of cell death including by necroptis, autophagic cell death, pyroptis and caspase-independent cell death (Journal of Cell Science 127; 2135-2144 (2014)). Such drugs can attenuate age-related deterioration of tissues or organs. Examples of drugs that can be conjugated to any peptide of this disclosure to induce apoptosis or induce cell death via non-apoptotic mechanisms include quercetin, dasatinib, bortezomib, carfilzomib, and navitoclax amongst other compounds disclosed herein. Additional examples are metformin, rapamycin, ABT-263, ABT-737, mTOR modulators, dasatinib, molecules that interact with FOXO, such as FOXO4 peptide (Everts, “Can we hit the snooze button” Chemical and Engineering News, 95(10), 30-35,2017, Molecules that perturb the FOXO4 interaction with p53, such as a FOXO4 peptide (Cell. 169(1): 132-147 (2017)). Other examples include dietary flavonols, small interfering RNA, or a rapamycin analog such as RAD001. A further example of an active agent that can be linked to any peptide of this disclosure is dimethyl fumarate. Additional active agents are described in the following references: Aging Cell. 2015 August; 14(4):644-58. doi: 10.1111/acel.12344. Epub 2015 Apr. 22. Kirkland J L (2013b) Translating advances from the basic biology of aging into clinical application. Exp. Gerontol. 48, 1-5, Kirkland J L, Tchkonia T (2014) Clinical strategies and animal models for developing senolytic agents. Exp. Gerontol. 2014 Oct. 28. pii: S0531-5565(14)00291-5, Tchkonia T, Zhu Y, van Deursen J, Campisi J, Kirkland J L (2013) Cellular senescence and the senescent secretory phenotype: therapeutic opportunities. J. Clin. Invest. 123, 966-972, WO2016118859, WO2016118859, Pharmgenomics Pers Med. 2015; 8: 23-33, Ren et al. Sci Rep. 2016 Apr. 7; 6:23968, Swanson et al. Nat Rev Rheumatol. 2009 June; 5(6): 317-324, Oh et al. PLoS One. 2012; 7(10): e45870, and Adebajo, Ade, Wolf-Henning Boehncke, Dafna D. Gladman, and P J. Mease. Psoriatic Arthritis and Psoriasis: Pathology and Clinical Aspects, 2016. Internet resource.

Another type of active agent that can be conjugated to a peptide of present disclosure includes tissue normalization (disease modifying) active agents, which can include, but are not limited to, biologics and small molecules. Biologic active agents can be further classified as chemokines (e.g. for stem cell recruitment) and growth factors. An example of a tissue normalization chemokine active agent that can be conjugated to any peptide of this disclosure includes MIP-3α. Small molecule active agents can be further classified as flavonoids (e.g., icariin), ACE inhibitors (e.g., captopril), and anti-proliferative active agents (e.g., methotrexate).

Further examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an RNAi, an oligonucleotide, an antibody, a single chain variable fragment (scFv or a single chain Fv), an antibody fragment, an aptamer, a cytokine, an interferon, a hormone, an enzyme, a growth factor, alpha-lipoic acid (to prevent nephrotoxicity to tubular cells after chemotherapy (e.g. cisplatin) or administration of an NSAID (e.g. indomethacin), a checkpoint inhibitor, nivolumab, pembrolizumab, ipilimumab (e.g., anti-CTLA4 antibodies), pidilizumab, bmx-936559, atezolizumab, avelumab, a PD-1 inhibitor, a PD-L1 inhibitor, a CTLA4 inhibitor, a CD antigen, aa chemokine, a neurotransmitter, an ion channel inhibitor, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, a complement fixing peptide or protein, a tumor necrosis factor inhibitor, a tumor necrosis factor activator, a tumor necrosis factor receptor family agonist, a tumor necrosis receptor antagonist, a tumor necrosis factor (TNF) soluble receptor or antibody, caspase protease activator or inhibitor, an NF-κB a RIPK1 and/or RIPK3 inhibitor or activator (e.g., through Toll-like receptors (TLRs) TLR-3 and/or TLR-4, or T-cell receptor (TCR) and the like), a death-receptor ligand (E.g., Fas ligand) activator or inhibitor, TNF receptor family (e.g., TNFR1, TNFR2, lymphotoxin β receptor/TNFRS3, OX40/TNFRSF4, CD40/TNFRSF5, Fas/TNFRSF6, decoy receptor 3/TNFRSF6B, CD27/TNFRSF7, CD30/TNFRSF8, 4-1BB/TNFRSF9, DR4 (death receptor 4/TNFRS10A), DR5 (death receptor 5/TNFRSF10B), decoy receptor 1/TNFRSF10C, decoy receptor 2/TNFRSF10D, RANK (receptor activator of NF-kappa B/TNFRSF11A), OPG (osteoprotegerin/TNFRSF11B), DR3 (death receptor 3/TNFRSF25), TWEAK receptor/TNFRSF12A, TACl/TNFRSF13B, BAFF-R (BAFF receptor/TNFRSF13C), HVEM (herpes virus entry mediator/TNFRSF14), nerve growth factor receptor/TNFRSF16, BCMA (B cell maturation antigen/TNFRSF17), GITR (glucocorticoid-induced TNF receptor/TNFRSF18), TAJ (toxicity and JNK inducer/TNFRSF19), RELT/TNFRSF19L, DR6 (death receptor 6/TNFRSF21), TNFRSF22, TNFRSF23, ectodysplasin A2 isoform receptor/TNFRS27, ectodysplasin 1, and anhidrotic receptor, a TNF receptor superfamily ligand including—TNF alpha, lymphotoxin-α, tumor necrosis factor membrane form, tumor necrosis factor shed form, LIGHT, lymphotoxin β₂α₁ heterotrimer, OX-40 ligand, compound 1 [PMID: 24930776], CD40 ligand, Fas ligand, TL1A, CD70, CD30 ligand, TRAF1, TRAF2, TRAF3, TRAIL, RANK ligand, APRIL, BAFF, B and T lymphocyte attenuator, NGF, BDNF, neurotrophin-3, neurotrophin-4, TL6, ectodysplasin A2, ectodysplasin A1—a TIMP-3 inhibitor, a BCL-2 family inhibitor, navitoclax (Aging Cell. 15(3): 428-435. (2016)), an IAP disruptor, a protease inhibitor, an amino sugar, a chemotherapeutic (whether acting through an apoptotic or non-apoptotic pathway) (Ricci et al. Oncologist 11(4):342-57 (2006)), a cytotoxic chemical, a toxin, a tyrosine kinase inhibitor (e.g. imatinib mesylate), protons, bevacuzimab (antivascular agent), erlotinib (EGFR inhibitor), QPI-1002, QM56, SVT016426 (QM31), 16/86 (third generation ferrostatin), BASP siRNA, CCX140, BIIB023, CXA-10, alkaline phosphatase, Dnmt1 inhibitor, THR-184, lithium, formoterol, IL-22, EPO and EPO derivatives, agents that stimulate erthyropoietin such as epoeitn alfa or darbepoietin alfa, PDGF inhibitors, CRMD-001, Atrasentan, Tolvaptan, RWJ-676070, Abatacept, Sotatercept, the binding site of the extracellular domain of the activing receptor 2A, an anti-infective agent, an antibiotic; such as gentamicin, vancomycin, minocin or mitomyclin, penicillins (such as amoxicillin), cephalosporins (such as cephalexin), macrolides (such as azithromycin), fluoroquinolones (such as ciprofloxacin), sulfonamides (such as co-trimoxazole), tetracyclines (such as doxycycline), aminoglycosides, an anti-infective agent, an antibiotic, an anti-viral agent, an anti-fungal agent, an aminoglycoside, a nonsteroidal anti-inflammatory drug (NSAID), a statin, a nanoparticle, a liposome, such as ketorolac or ibuprofen, an immunosuppresant such tacrolimus, mycophenolic acid (e.g., mycophenolate mofetil), cyclosporine A, or azathioprine, a diuretic drug including thiazides, loop diuretics, and potassium sparing diuretics, bumetanide, ethacrynic acid, furosemide, torsemide, glucose, mannitol, amiloride, spironolactone, eplerenone, triamterene, potassium canrenoate, bendroflumethiazide, chlorothiazide, chlorthalidone, metolazone, indapamide, hydrochlorothiazide, vasopressin, amphotericin B, acetazolamide, tovaptan, conivaptan, dopamine, dorzolamide, bendrolumethiazide, hydrochlorothiazide, caffeine, theophylline, or theobromine, a statin, a senolytic such as navitoclax or obatoclax, a corticosteroid such as prednisone, betamethasone, fludrocortisone, deoxycorticosterone, aldosterone, cortisone, hydrocortisone, belcometasone, dexamethasone, mometasone, fluticasone, prednisolone, methylprednisolone, triamcinolone acetonide or triamcinolone, a glucocorticoid, a mineralocorticoid, such as aldosterone and flucrocortisone, a liposome, renin, renin inhibitors such as aliskiren, pepstatin, statine, cgp2928, remikiren, enalkiren, zankiren, angiotensin, ACE inhibitors such as ramipril, captopril, lisinopril, benazepril, quinapril, fosinopril, trandolapril, moexipril, enalaprilat, enalapril maleate, or perindopril erbumine, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, retinoic acid, SGLT2 modulator, such as Dapagliflozin, canagliflozin, and empagliflozin, a polymer, a biopolymer, a polysaccharide, a proteoglycan, a glycosaminoglycan, polyethylene glycol, a lipid, a dendrimer, a fatty acid, or an Fc domain or an Fc region, or an active fragment or a modification thereof. Any combination of the above active agents can be co-delivered with peptides or peptide conjugates of this disclosure. Additionally, in some embodiments, other co-therapies such as proton therapy or ablative radiotherapy can be administered to a subject in need thereof along with peptides or peptide conjugates of this disclosure. In some embodiments, the peptide is covalently or non-covalently linked to an active agent, e.g., directly or via a linker. TNF blockers suppress the immune system by blocking the activity of TNF, a substance in the body that can cause inflammation and lead to immune-system diseases, such as lupus. The drugs in this class include Remicade (infliximab), Enbrel (etanercept), Humira (adalimumab), Cimzia (certolizumab pegol) and Simponi (golimumab). The peptide disclosed herein can be used to home, distribute to, target, directed to, is retained by, accumulate in, migrate to, and/or bind to a target tissue or cell, and thus also be used for localizing the attached or fused active agent. Furthermore, chlorotoxin peptide can be internalized in cells (Wiranowska, M., Cancer Cell Int., 11: 27 (2011)). Therefore, cellular internalization, subcellular localization, and intracellular trafficking after internalization of the peptide itself, or an active agent peptide conjugate or fusion peptide can be important factors in the efficacy of an active agent conjugate or fusion. (Ducry, L., Antibody Drug Conjugates (2013); and Singh, S. K., Pharm Res. 32(11): 3541-3571 (2015)).

Additionally, more than one peptide sequence can be present on or fused with a particular peptide. A peptide can be incorporated into a biomolecule by various techniques, for example by a chemical transformation, such as the formation of a covalent bond, such as an amide bond, or by solid phase or solution phase peptide synthesis, or by preparing a nucleic acid sequence encoding the biomolecule, wherein the nucleic acid sequence includes a subsequence that encodes the peptide. The subsequence can be in addition to the sequence that encodes the biomolecule, or can substitute for a subsequence of the sequence that encodes the biomolecule.

In some embodiments, the peptides of the present disclosure are coupled (e.g., conjugated) to other moieties that, e.g., can modify or effect changes to the properties of the peptides. For example, in certain embodiments, the peptides described herein are attached to another molecule, such as an active agent that provides a functional capability. Examples of active agents include but are not limited to: a peptide, an oligopeptide, a polypeptide, a polynucleotide, a polyribonucleotide, a DNA, a cDNA, a ssDNA, a RNA, a dsRNA, a micro RNA, an oligonucleotide, an antibody fragment, a single chain Fv, an aptamer, a cytokine, an enzyme, a growth factor, a chemokine, a neurotransmitter, a chemical agent, a fluorophore, a metal, a metal chelate, an X-ray contrast agent, a PET agent, a radioisotope, a photosensitizer, a radiosensitizer, a radionuclide chelator, a therapeutic small molecule, a steroid, a corticosteroid, an anti-inflammatory agent, an immune modulator, a protease inhibitor, an amino sugar, a chemotherapeutic, a cytotoxic chemical, a toxin, a tyrosine kinase inhibitor, an anti-infective agent, an antibiotic, an anti-viral agent, an anti-fungal agent, an aminoglycoside, a nonsteroidal anti-inflammatory drug (NSAID) such as ketorolac or ibuprofen, a statin, a nanoparticle, a liposome, a polymer, a biopolymer, a polysaccharide, a proteoglycan, a glycosaminoglycan, a dendrimer, a fatty acid, or an Fc region, or an active fragment or a modification thereof. In some embodiments, the peptide is covalently or non-covalently linked to an active agent, e.g., directly or via a linker. Exemplary linkers suitable for use with the embodiments herein are discussed in further detail below.

In some embodiments, the active agent interacts with an ion channel, inhibits a protease, has antimicrobial activity, has anticancer activity, has anti-inflammatory activity, induces ischemic preconditioning or acquired cytoresistance, produces a protective or therapeutic effect on a target tissue or cell of the subject, reduces a clearance rate of the composition, or a combination thereof. Optionally, the active agent is a therapeutic agent, such as a protective agent or prophylactic agent.

In some embodiments, the peptides of the present disclosure can be modified such that the modification increases the stability and/or the half-life of the peptides. In some embodiments, the attachment of a hydrophobic moiety, such as to the N-terminus, the C-terminus, or on an internal amino acid, can be used to extend half-life of a peptide of the present disclosure. In some embodiments, simple carbon chains (e.g., by myristoylation and/or palmitylation) can be conjugated to the fusion proteins or peptides. In some embodiments, the simple carbon chains can render the peptides easily separable from the unconjugated material. For example, methods that can be used to separate the peptides from the unconjugated material include, but are not limited to, solvent extraction and reverse phase chromatography. The lipophilic moieties can extend half-life through reversible binding to serum albumin. The conjugated moieties can, e.g., be lipophilic moieties that extend half-life of the peptides through reversible binding to serum albumin. In some embodiments, simple carbon chains (e.g., by myristoylation) can be conjugated to the peptides. In some embodiments, the lipophilic moiety can be cholesterol or a cholesterol derivative including cholestenes, cholestanes, cholestadienes and oxysterols. In some embodiments, the peptides can be conjugated to myristic acid (tetradecanoic acid) or a derivative thereof.

### Detectable Agent Peptide Conjugates

In some embodiments, a peptide is conjugated to or fused with detectable agents, such as an imaging label, fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, or 10 detectable agents can be linked to a peptide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Peptides can be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers can include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of diseased cells (e.g., cancer cells) using both a therapeutic agent (e.g., drug) and electromagnetic energy (e.g., radiation or light) concurrently. In some embodiments, the peptide is fused with, or covalently or non-covalently linked to the agent, e.g., directly or via a linker. Exemplary linkers suitable for use with the embodiments herein are discussed in further detail below.

### Linkers

Peptides according to the present disclosure can be attached to another moiety (e.g., an active agent or an detectable agent), such as a small molecule, a second peptide, a protein, an antibody, an antibody fragment, an aptamer, polypeptide, polynucleotide, a fluorophore, a radioisotope, a radionuclide chelator, a polymer, a biopolymer, a fatty acid, an acyl adduct, a chemical linker, or sugar or other active agent or detectable agent described herein through a linker, or directly in the absence of a linker. In the absence of a linker, for example, an active agent or an detectable agent can be fused to the N-terminus or the C-terminus of a peptide to create an active agent or detectable agent fusion peptide. In other embodiments, the link can be made by a peptidic fusion via reductive alkylation.

Direct attachment can be through covalent attachment of a peptide to a region of the other molecule. For example, an active agent or a detectable agent can be fused to the N-terminus or the C-terminus of a peptide to create an active agent or detectable agent fusion peptide. As an additional example, a peptidic linker can be inserted between the N-terminus or C-terminus of a peptide and an active agent or detectable agent, wherein the peptidic linker can be from 1 to 30 amino acid residues and can comprise (GGGS)ₓ (SEQ ID NO: 82), wherein X can be any integer from 1 to 7. As another example, the peptide can be attached at the N-terminus, an internal lysine, glutamic acid, or aspartic acid residue, or the C-terminus to a terminus of the amino acid sequence of the other molecule by a linker. If the attachment is at an internal lysine residue, the other molecule can be linked to the peptide at the epsilon amine of the internal lysine residue. In some further examples, the peptide can be attached to the other molecule by a side chain, such as the side chain of a lysine, serine, threonine, cysteine, tyrosine, aspartic acid, a non-natural amino acid residue, or glutamic acid residue. A linker can be an amide bond, an ester bond, an ether bond, a carbamate bond, a carbonate bond, a carbon-nitrogen bond, a triazole, a macrocycle, an oxime bond, a thioester bond, a thioether bond a hydrazone bond, a carbon-carbon single, double, or triple bond, a disulfide bond, a two carbon bridge between two cysteines, a three carbon bridge between two cysteines, or a thioether bond. In still other embodiments, the peptide can comprise a non-natural amino acid, wherein the non-natural amino acid can be an insertion, appendage, or substitution for another amino acid, and the peptide can be linked to the active agent at the non-natural amino acid by a linker. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single, double, or triple bond, a disulfide bond, a thioether bond, or other linker as described herein) can be used to link other molecules.

Attachment via a linker involves incorporation of a linker moiety between the other molecule and the peptide. The peptide and the other molecule can both be covalently attached to the linker. The linker can be cleavable, non-cleavable, self-immolating, hydrophilic, or hydrophobic. The linker has at least two functional groups, one bonded to the other molecule, and one bonded to the peptide, and a linking portion between the two functional groups. Some example linkers are described in Jain, N., Pharm Res. 32(11): 3526-40 (2015), Doronina, S. O., Bioconj Chem. 19(10): 1960-3 (2008), Pillow, T. H., J Med Chem. 57(19): 7890-9 (2014), Dorywalksa, M., Bioconj Chem. 26(4): 650-9 (2015), Kellogg, B. A., Bioconj Chem. 22(4): 717-27 (2011), and Zhao, R. Y., J Med Chem. 54(10): 3606-23 (2011).

Non-limiting examples of the functional groups for attachment can include functional groups capable of forming, for example, an amide bond, an ester bond, an ether bond, a carbonate bond, a carbamate bond, a carbon-nitrogen bond, a triazole, a macrocycle, an oxime bond, a hydrazone bond, a carbon-carbon single, double, or triple bond, a disulfide bond or a thioether bond. Non-limiting examples of functional groups capable of forming such bonds include amino groups; carboxyl groups; aldehyde groups; azide groups; alkyne and alkene groups; ketones; hydrazides; hydrazines; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; maleimides; linkers containing maleimide groups that are designed to hydrolyze; maleimidocaproyl; MCC ([N-maleimidomethyl]cyclohexane-1-carboxylate); N-ethylmaleimide; maleimide alkane; mc-vc-PABC; DUBA (DuocarmycinhydroxyBenzamide-Azaindole linker); SMCC Succinimidyl-4-(N-maleimidomethyl) cyclohexane-1-carboxylate; SPDP (N-succinimidyl-3-(2-pyridyldithio) propionate); SPDB N-succinimidyl-4-(2-pyridyldithio) butanoate; sulfo-SPDB N-succinimidyl-4-(2-pyridyldithio)-2-sulfo butanoate; SPP N-succinimidyl 4-(2-pyridyldithio)pentanoate; a dithiopyridylmaleimide (DTM); a hydroxylamine, a vinyl-halo group; haloacetamido groups; bromoacetamido; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion can include alkylene, alkenylene, alkynylene, polyether, such as polyethylene glycol (PEG), polyester, polyamide, polyamino acids, polypeptides, cleavable peptides, Val-Cit, Phe-Lys, Val-Lys, Val-Ala, other peptide linkers as given in Doronina et al., 2008, linkers cleavable by beta glucuronidase, linkers cleavable by a cathepsin or by cathepsin B, D, E, H, L, S, C, K, O, F, V, X, or W, Val-Cit-p-aminobenzyloxycarbonyl, glucuronide-MABC, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, charged groups, zwitterionic groups, and ester groups. Other non-limiting examples of reactions to link molecules together include click chemistry, copper-free click chemistry, HIPS ligation, Staudinger ligation, and hydrazine-iso-Pictet-Spengler.

Non-limiting examples of linkers include:

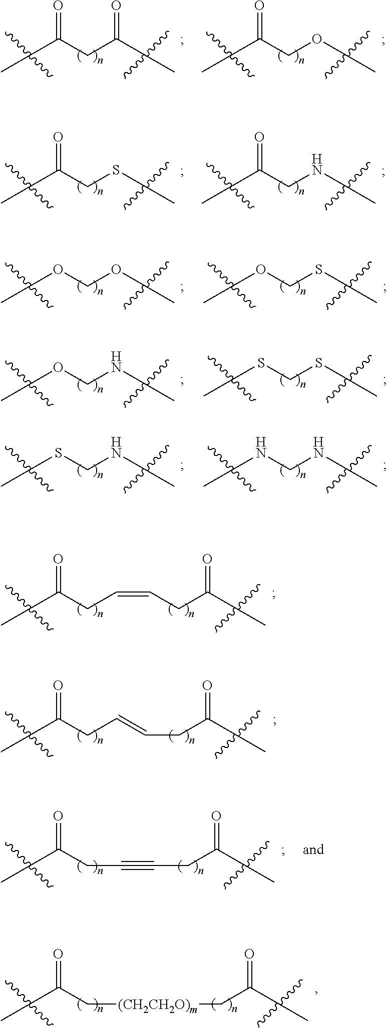

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50, or any linker as disclosed in Jain, N., Pharm Res. 32(11): 3526-40 (2015) or Ducry, L., Antibody Drug Conjugates (2013).

In some cases a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid.

In some embodiments, the linker can release the active agent in an unmodified form. In other embodiments, the active agent can be released with chemical modification. In still other embodiments, catabolism can release the active agent still linked to parts of the linker and/or peptide.

The linker may be a noncleavable linker or a cleavable linker. In some embodiments, the noncleavable linker can slowly release the conjugated moiety by an exchange of the conjugated moiety onto the free thiols on serum albumin. In some embodiments, the use of a cleavable linker can permit release of the conjugated moiety (e.g., a therapeutic agent) from the peptide, e.g., after administration to a subject in need thereof. In other embodiments, the use of a cleavable linker can permit the release of the conjugated therapeutic from the peptide. In some cases the linker is enzyme cleavable, e.g., a valine-citrulline linker. In some embodiments, the linker contains a self-immolating portion. In other embodiments, the linker includes one or more cleavage sites for a specific protease, such as a cleavage site for matrix metalloproteases (MMPs), thrombin, cathepsins, peptidases, or beta-glucuronidase. Alternatively or in combination, the linker is cleavable by other mechanisms, such as via pH, reduction, or hydrolysis.

The rate of hydrolysis or reduction of the linker can be fine-tuned or modified depending on an application. For example, the rate of hydrolysis of linkers with unhindered esters can be faster compared to the hydrolysis of linkers with bulky groups next to an ester carbonyl. A bulky group can be a methyl group, an ethyl group, a phenyl group, a ring, or an isopropyl group, or any group that provides steric bulk. In some cases, the steric bulk can be provided by the drug itself, such as by ketorolac when conjugated via its carboxylic acid. The rate of hydrolysis of the linker can be tuned according to the residency time of the conjugate in the target location. For example, when a peptide is cleared from a tumor, or the brain, relatively quickly, the linker can be tuned to rapidly hydrolyze. When a peptide has a longer residence time in the target location, a slower hydrolysis rate would allow for extended delivery of an active agent. “Programmed hydrolysis in designing paclitaxel prodrug for nanocarrier assembly” Sci Rep 2015, 5, 12023 Fu et al., provides an example of modified hydrolysis rates.

### Structures

In some embodiments, the secondary, tertiary, and/or quaternary structures of any peptide and/or ion channel of this disclosure can be solved in order to spatially map each atom in a given peptide. Such structure can be determined using crystallization techniques, electron microscopy, homology mapping, sequence analysis, and/or any other biophysical method known in the art. Solving the structure of the peptide and/or an ion channel can yield information on the spatial orientation, positioning, and interaction of amino acids between a peptide or peptide conjugate of this disclosure with an ion channel. Thus, in some embodiments, the structure of a peptide or ion channel can provide information on conserved structural elements or motifs that play a role in stability, ion channel binding activity, binding affinity between a peptide and residues of an ion channel, ion channel inhibition activity, in identifying residues that can be mutated, conserved, or internal or external to the surface of a folded peptide or a domain or subunit of an ion channel to effect ion channel binding, activation, or inhibition, or in identifying sites for conjugation with active agents or sites of modification that can affect binding specificity, affinity, or mode of binding or interaction between a peptide or peptide conjugate and an ion channel of this disclosure. This information can allow mutants to be designed that preserve a desired function (such as ion channel inhibition) while changing other aspects of the peptide, such as reduced off-target effects or higher specificity or selectivity for an ion channel in a target tissue or cell type.

One of skill can apply principles to each structure and the underlying coordinate data to identify conserved and internal residues, or determine which residues may be modified without likely affecting the overall structure. This information about a peptide based on its structure and the general sequences of knotted peptides can be used to enhance specific properties of the peptides of this disclosure such as, but not limited to, modifying specificity, affinity, and/or resistance to a variety of agents and conditions in order to make stable peptides of the disclosed sequences, enhancing physiologic activity, optimizing manufacturability, and identifying optimal sites for conjugating or linking the peptide to an active agent or detectable agent.

The NMR solution structures, x-ray crystallography, or crystal structures of related structural homologs can be used to inform mutational strategies that can improve the folding, stability, and manufacturability, while maintaining the ability of a peptide to bind to an ion channel. They can be used to predict the 3D pharmacophore of a group of structurally homologous scaffolds, as well as to predict possible graft regions of related proteins to create chimeras with improved properties. For example, this strategy can be used to identify critical amino acid positions and loops that can be used to design drugs with improved properties or to correct deleterious mutations that complicate folding and manufacturability for the peptides. These key amino acid positions and loops can be retained while other residues in the peptide sequences can be mutated to improve, change, remove, or otherwise modify function, homing, and activity of the peptide.

Additionally, the comparison of the primary sequences and the tertiary sequences of two or more peptides can be used to reveal sequence and 3D folding patterns that can be leveraged to improve the peptides and parse out the biological activity of these peptides. For example, comparing two different peptide scaffolds that target an ion channel can lead to the identification of conserved pharmacophores that can guide engineering strategies, such as designing variants with improved binding properties. Important pharmacophore, for example, can comprise aromatic residues or basic residues, which can be important for binding.

In some cases, amino acid sequence and structural alignments of various toxins that evolved to target specific ion channels are used to reveal conserved amino acid residues for targeting, modulating the activity of, e.g., activating or inhibiting, an ion channel, or to reveal amino acid residues that can orientate the binding interface of a peptide by adjusting its molecular polarity or electrostatic distribution. In some embodiments, one can start with a natural toxin as a template and engineer specific amino acid substitutions at the ion channel binding interface based on structural analysis of the target ion channel and/or the toxin-derived peptide to refine the specificity and binding properties of the peptide.

In some cases, acidic residues, such as aspartic acid, are engineered in a peptide to alter its acidic residue distribution and to orientate or shift the peptide binding interface relative to an ion channel, which can increase the peptide's specificity and/or inhibition of the ion channel. In some cases, alanine-scanning mutagenesis and computational simulation can be used to improve the targeting, binding, inhibition or activation properties of a peptide for a particular ion channel. In some cases, chemical modification of amino acid residues, sequence truncation, computer-aided design and phage display libraries can also be used to improve peptide selectivity for a particular ion channel and/or to minimize off-target effects or binding to non-target ion channel.

In some cases, evolution-guided drug design can be used to manufacture non-naturally occurring peptides, such as SEQ ID NO: 1 to SEQ ID NO: 80, or a derivative or analog thereof. Examples of evolution-guided drug design for making ion channel blockers are described in Ye, F., et al., Toxins (Basel), 2016 Apr. 19; 8(4):115, and Chen, Z. et al., Sci. Rep. 2015 May 8; 5:9881, each of which is incorporated by reference in its entirety. Venoms or toxins derived from various life forms, e.g., snakes, scorpions, spiders, insects, sea anemones, and cone snails, contain peptide-based toxins that target diverse ion channels, and provide useful templates, starting points, or disulfide framework for engineering ion channel inhibitors with improved binding properties and selectivity for a specific ion channel over non-target ion channels.

Examples of ion channel toxins for making peptides that bind selectively to a target ion channel, or for applying the evolution-guided ion channel binding peptide design approach, include, but are not limited to, ablomin, aconitine, AETX, agelenin, agitoxin, allopumiliotoxin, altitoxin, AmmTX3, androctonus australis hector insect toxin, anthopleurin, antillatoxin, anuroctoxin, apamin, azaspiracid, babycurus toxin, bactridines, batrachotoxin, BDS-1, BeKm-1 toxin, bestoxin, BgK, Birtoxin, BmKAEP, BmTx3, BotIT2, bukatoxin, bungarotoxin, alpha-bungarotoxin, beta-bungarotoxin, butantoxin, calcicludine, calciseptine, calitoxin, cangitoxin, cgna toxin, charybdotoxin, chlorotoxin, ciguatoxin, Cl11, cobatoxin, cobratoxin, conantokin, conotoxin, contryphan, crotamine, CssII, CSTX, dendrotoxin, discrepin, dortoxin, ergtoxin, grammotoxin, grayanotoxin, guangxitoxin, hadrucalcin, hainantoxin, halcurin, hanatoxin, hefutoxin, helothermine, hemitoxin, heteropodatoxin, heteroscodratoxin-1, HgeTx1, hongotoxin, HsTx1, huwentoxin, iberiotoxin, ikitoxin, imperatoxin, inhibitor cystine knot, iodoresiniferatoxin, isopimaric acid, jingzhaotoxin, kalicludine, kaliotoxin, kaliseptine, kalkitoxin, kurtoxin, latisemin, limbatustoxin, LmαTX3, lolitrem B, Lq2, maitotoxin, margatoxin, maurocalcine, maurotoxin, MCD peptide, myotoxin, omega-atracotoxin, ophanin, pandinotoxin, pandinus imperator (Pi3) toxin, pandinus imperator toxin (Pi4), parabutoxin, phaiodotoxin, philanthotoxin, PhTX-433, phrixotoxin, pinnatoxin, piscivorin, pompilidotoxin, poneratoxin, psalmotoxin, pumiliotoxin, raventoxin, RhTx, scyllatoxin, SHTX, slotoxin, SNX-482, soricidin, spinoxin, Ssm6a, stichodactyla toxin, stromatoxin, taicatoxin, tamapin, tamulotoxin, tertiapin, tetrodotoxin, tinyatoxin, tityustoxin, tityustoxin peptide 2, TLTx, triflin, Ts15, TsIV, vanillotoxin, and veratridine, or a combination, a structural analogue, or derivative thereof.

In some cases, a peptide of present disclosure comprises amino acid substitutions that alter the molecular polarity of the peptide at its binding interface. For example, positive ion channels, such as potassium ion channels or sodium ion channels, have negatively charged vestibules. Peptides that target positive ion channels have a positively charged binding interface. One can improve peptide binding properties for a particular ion channel by re-orientating the peptide binding interface by engineering specific acidic amino acid residues that can guide the orientation of the binding interfaces due to electrostatic repulsion forces between the acidic residues of the peptide and the ion channel. Thus, by adjusting the acidic residue distribution in a peptide, one can orient the peptide binding interface in a manner that improves selectivity for one particular ion channel over other ion channels. Such strategy can also increase the potency of the peptides as ion channel blockers or activators.

In some embodiments, a peptide of this disclosure comprises an amino acid substitution that re-orientate the binding interface by using a turn motif between an alpha helix and an anti-parallel beta sheet domains in the binding interface. In some cases, a peptide of this disclosure comprises one or more, two or more, three or more, four or more, or five or more positively charge residues, e.g., lysine, histidine, or arginine, and/or negatively charged residues, e.g., glutamic acid or aspartic acid, that adjust the electrostatic distribution or molecular polarities at the binding interfaces. Residues that result in electrostatic repulsion forces between the peptide and the ion channel can be removed or added to a peptide to rotate the peptide binding interface to impart a greater selectivity between the peptide and the ion channel. In some cases, an electrostatic repulsion force, e.g., an aspartic acid residue in the peptide is removed so that new electrostatic repulsion forces between the peptide and the ion channel are introduced or aligned to impart greater selectivity or improved binding properties between the peptide and the ion channel.

In some embodiments, structure of a peptide disclosed herein can be determined or predicted using NMR, crystallographic, or computational simulation methods. Using the structure, one can identify the amino acid residues of the peptide at the binding interface with an ion channel. Surface exposed residues, such as Arg, Lys, Asp, and Glu, can also be identified using mutatgenesis studies. Knowing which surface exposed residues are functionally important for ion channel binding allows one to make site-directed mutatgenesis to improve the binding properties or potency of the peptide as an ion channel blocker or activator.

In some embodiments, the CDP peptides selectively inhibit voltage-gated ion channels, such as voltage- and calcium-dependent potassion ion channels. Such peptides interact with the potassium ion channel and block the conduction pore by binding to an extracellular entryway of the ion channel. One such peptide comprises 37-39 amino acids in length and contains six cysteine residues. For example, agitoxin 2 (AgTx2) binding affinity and specificity depend on Arg24, Lys27, and Arg31 residues. Arg 24 and Arg 31 of AgTx2 can interact with a potassium channel, while Lys27 is positioned over the center of the channel. Corresponding residues in other toxin derived peptides may play a similar role in ion channel binding. For example, Arg19 of ChTx is equivalent to Asp20 of AgTx2. Such surface exposed residues that contribute to ion channel binding affinities can be mutated to alter the binding affinity and specificity of a peptide for an ion channel. Alternatively, spatially adjacent residues that interact with the surface exposed residues or residues that constrain the positioning of the solvent exposed residues can be mutated to improve the binding properties or selectivity of the peptide for an ion channel.

In some embodiments, peptides for inhibiting ion channels can be derived by systematically mapping the disulfide bonds of toxins that inhibit an ion channel family and investigating amino acid residues that are significant for ion channel binding using alanine scanning. An example of such approach is described in Pennington, M., et al Mar. Drugs 2015, 13, 529-542, which is incorporated herein in its entirety.

Other methods for determining residues of peptides for improving selectivity and/or binding properties of peptides include multi attribute positional scan analoging, as described in Murray et al., J. Med. Chem. 2015, 58, 6784-6802, which is incorporated herein in its entirety. High-throughput screening of large sets of peptides can be performed using automated electrophysiology methods and platforms, such as inhibition of Kv1.3 or Kv1.1 by electrophysiology and inhibition of IL-2 and IFN-γ secretion in whole blood. In such high-throughput screen, each position of the peptide can be substituted with amino acid residues with different physiological attributes, such as hydrophobic, basic, and acidic attributes, while keeping the disulfide bonds intact, to determine the amino acid attributes at various positions that enhance ion channel binding, inhibition, or activation.

### Peptide as a Delivery Scaffold

In certain embodiments, any peptide of this disclosure can be used as a delivery scaffold for an active agent. A peptide of this disclosure can be used as delivery scaffold for an active agent to various biological environments due to the peptide's enhanced stability in these environments, which can allow for access and treatment of disorders in these biological environments. For example, any peptide of SEQ ID NO: 1-SEQ ID NO: 80 can be stable in a biological environment with a low pH, a protease-rich environment, an acidic environment, a reducing environment, and/or environments with varying temperatures. Such biological environments can be found in the gastrointestinal (GI) tract (including, but not limited to, mouth, nasal cavities, throat, esophagus, stomach, small intestine, large intestine, and rectum), heart, brain, kidney, muscle, lung, skin, cartilage, vaginal mucosa, or nasal mucosa, or a cellular compartment, such as lysosomes, endosomes, or the cytosol. Therefore, using the peptides of this disclosure as delivery scaffold can be advantageous for delivery of therapeutics to various physiologic environments that can degrade other peptides.

### Pharmacokinetics of Peptides

The pharmacokinetics of any of the peptides of this disclosure can be determined after administration of the peptide via different routes of administration. For example, the pharmacokinetic parameters of a peptide of this disclosure can be quantified after intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, peritoneal, buccal, synovial, or topical administration. Peptides of the present disclosure can be analyzed by using tracking agents such as radiolabels or fluorophores. For example, a radiolabeled peptides of this disclosure can be administered via various routes of administration. Peptide concentration or dose recovery in various biological samples such as plasma, urine, feces, any organ, skin, muscle, and other tissues can be determined using a range of methods including HPLC, fluorescence detection techniques (TECAN quantification, flow cytometry, iVIS), or liquid scintillation counting.

The methods and compositions described herein relate to pharmacokinetics of peptide administration via any route to a subject. Pharmacokinetics can be described using methods and models, for example, compartmental models or noncompartmental methods. Compartmental models include but are not limited to monocompartmental model, the two compartmental model, the multicompartmental model or the like. Models are often divided into different compartments and can be described by the corresponding scheme. For example, one scheme is the absorption, distribution, metabolism and excretion (ADME) scheme. For another example, another scheme is the liberation, absorption, distribution, metabolism and excretion (LADME) scheme. In some aspects, metabolism and excretion can be grouped into one compartment referred to as the elimination compartment. For example, liberation includes liberation of the active portion of the composition from the delivery system, absorption includes absorption of the active portion of the composition by the subject, distribution includes distribution of the composition through the blood plasma and to different tissues, metabolism, which includes metabolism or inactivation of the composition and finally excretion, which includes excretion or elimination of the composition or the products of metabolism of the composition. Compositions administered intravenously to a subject can be subject to multiphasic pharmacokinetic profiles, which can include but are not limited to aspects of tissue distribution and metabolism/excretion. As such, the decrease in plasma or serum concentration of the composition is often biphasic, including, for example an alpha phase and a beta phase, occasionally a gamma, delta or other phase is observed.

Pharmacokinetics includes determining at least one parameter associated with administration of a peptide to a subject. In some aspects, parameters include at least the dose (D), dosing interval (τ), area under curve (AUC), maximum concentration (Cₘₐₓ), minimum concentration reached before a subsequent dose is administered (Cₘᵢₙ), minimum time (Tₘᵢₙ), maximum time to reach Cmax (Tₘₐₓ), volume of distribution (V_{d}), steady-state volume of distribution (Vₛₛ), back-extrapolated concentration at time 0 (C₀), steady state concentration (Cₛₛ), elimination rate constant (kₑ), infusion rate (kᵢₙ), clearance (CL), bioavailability (f), fluctuation (% PTF) and elimination half-life (t_{1/2}).

In certain embodiments, the peptides of any of SEQ ID NO: 1-SEQ ID NO: 80 exhibit optimal pharmacokinetic parameters after oral administration. In other embodiments, the peptides of any of SEQ ID NO: 1-SEQ ID NO: 80 exhibit optimal pharmacokinetic parameters after a any route of administration, such as oral administration, inhalation, intranasal administration, topical administration, intravenous administration, subcutaneous administration, intra-articular administration, intramuscular administration, intraperitoneal administration, intra-synovial, or any combination thereof.

In some embodiments any peptide of SEQ ID NO: 1-SEQ ID NO: 80 exhibits an average Tₘₐₓ of 0.5-12 hours, or 1-48 hours at which the Cₘₐₓ is reached, an average bioavailability in serum of 0.1%-10% in the subject after administering the peptide to the subject by an oral route, an average bioavailability in serum of less than 0.1% after oral administration to a subject for delivery to the GI tract, an average bioavailability in serum of 10-100% after parenteral administration, an average t_{1/2} of 0.1-168 hours, or 0.25-48 hours in a subject after administering the peptide to the subject, an average clearance (CL) of 0.5-100 L/hour or 0.5-50 L/hour of the peptide after administering the peptide to a subject, an average volume of distribution (V_{d}) of 200-20,000 mL in the subject after systemically administering the peptide to the subject, or optionally no systemic uptake, any combination thereof.

### Methods of Manufacture

Various expression vector/host systems can be utilized for the recombinant expression of peptides described herein. Non-limiting examples of such systems include microorganisms such as bacteria transformed with recombinant bacteriophage DNA, plasmid DNA or cosmid DNA expression vectors containing a nucleic acid sequence encoding peptides or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV) or transformed with recombinant plasmid expression vectors (e.g., Ti plasmid) containing the aforementioned nucleic acid sequence, or animal cell systems infected with recombinant virus expression vectors (e.g., adenovirus, vaccinia virus, lentivirus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secrete minimal amounts of proteolytic enzymes.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, peptides can also be synthesized in a cell-free system prior to extraction using a variety of known techniques employed in protein and peptide synthesis.

In some cases, a host cell produces a peptide that has an attachment point for a drug. An attachment point could comprise a lysine residue, an N-terminus, a cysteine residue, a cysteine disulfide bond, a glutamic acid or aspartic acid residue, a C-terminus, or a non-natural amino acid.

The peptide could also be produced synthetically, such as by solid-phase peptide synthesis, or solution-phase peptide synthesis. Peptide synthesis can be performed by fluorenylmethyloxycarbonyl (Fmoc) chemistry or by butyloxycarbonyl (Boc) chemistry. The peptide could be folded (formation of disulfide bonds) during synthesis or after synthesis or both. Peptide fragments could be produced synthetically or recombinantly. Peptide fragments can be then be joined together enzymatically or synthetically.

In other aspects, the peptides of the present disclosure can be prepared by conventional solid phase chemical synthesis techniques, for example according to the Fmoc solid phase peptide synthesis method (“Fmoc solid phase peptide synthesis, a practical approach,” edited by W. C. Chan and P. D. White, Oxford University Press, 2000).

In some embodiments, the peptides of this disclosure can be more stable during manufacturing. For example, peptides of this disclosure can be more stable during recombinant expression and purification, resulting in lower rates of degradation by proteases that are present in the manufacturing process, a higher purity of peptide, a higher yield of peptide, or any combination thereof. In some embodiments, the peptides can also be more stable to degradation at high temperatures and low temperatures during manufacturing, storage, and distribution. For example, in some embodiments peptides of this disclosure can be stable at 25° C., 30° C., 35° C., or 40° C. In other embodiments, peptides of this disclosure can be stable at 70° C. or higher than 70° C. In some embodiments, peptides of this disclosure can be stable at 100° C. or higher than 100° C.

In some embodiments, peptides of this disclosure can remain intact after exposure to a temperature of at least 25° C., 30° C., or 40° C. with at least 60%, 65% or 75% relative humidity for at least 3, 6, 12, 18, 24, 36, or 48 months. In some embodiments, peptides of this disclosure can remain intact after exposure to a temperature of at least 25° C. with at least 60% relative humidity for from 3 months to 48 months. In some embodiments, peptides of this disclosure can remain intact after exposure to a temperature of at least 25° C. with at least 60% relative humidity for from 3 months to 12 months. In some embodiments, peptides of this disclosure can remain intact after exposure to a temperature of at least 25° C. with at least 60% relative humidity for from 12 months to 24 months. In some embodiments, peptides of this disclosure can remain intact after exposure to a temperature of at least 25° C. with at least 65% relative humidity for from 3 months to 48 months. In some embodiments, peptides of this disclosure can remain intact after exposure to a temperature of at least 25° C. with at least 65% relative humidity for from 3 months to 12 months. In some embodiments, peptides of this disclosure can remain intact after exposure to a temperature of at least 25° C. with at least 65% relative humidity for from 12 months to 24 months. In some embodiments, peptides of this disclosure can remain intact after exposure to a temperature of at least 25° C. with at least 70% relative humidity for from 3 months to 48 months. In some embodiments, peptides of this disclosure can remain intact after exposure to a temperature of at least 25° C. with at least 70% relative humidity for from 3 months to 12 months. In some embodiments, peptides of this disclosure can remain intact after exposure to a temperature of at least 25° C. with at least 70% relative humidity for from 12 months to 24 months.

In some embodiments, peptides of this disclosure can remain intact after exposure to a temperature of at least 23° C. with at least 60% relative humidity for from 3 months to 48 months. In some embodiments, peptides of this disclosure can remain intact after exposure to a temperature of at least 23° C. with at least 60% relative humidity for from 3 months to 12 months. In some embodiments, peptides of this disclosure can remain intact after exposure to a temperature of at least 23° C. with at least 60% relative humidity for from 12 months to 24 months. In some embodiments, peptides of this disclosure can remain intact after exposure to a temperature of at least 23° C. with at least 65% relative humidity for from 3 months to 48 months. In some embodiments, peptides of this disclosure can remain intact after exposure to a temperature of at least 23° C. with at least 65% relative humidity for from 3 months to 12 months. In some embodiments, peptides of this disclosure can remain intact after exposure to a temperature of at least 23° C. with at least 65% relative humidity for from 12 months to 24 months. In some embodiments, peptides of this disclosure can remain intact after exposure to a temperature of at least 23° C. with at least 70% relative humidity for from 3 months to 48 months. In some embodiments, peptides of this disclosure can remain intact after exposure to a temperature of at least 23° C. with at least 70% relative humidity for from 3 months to 12 months. In some embodiments, peptides of this disclosure can remain intact after exposure to a temperature of at least 23° C. with at least 70% relative humidity for from 12 months to 24 months.

In some embodiments, peptides of this disclosure can remain intact after exposure to a temperature of at least 30° C. with at least 60% relative humidity for from 3 months to 48 months. In some embodiments, peptides of this disclosure can remain intact after exposure to a temperature of at least 30° C. with at least 60% relative humidity for from 3 months to 12 months. In some embodiments, peptides of this disclosure can remain intact after exposure to a temperature of at least 30° C. with at least 60% relative humidity for from 12 months to 24 months. In some embodiments, peptides of this disclosure can remain intact after exposure to a temperature of at least 30° C. with at least 65% relative humidity for from 3 months to 48 months. In some embodiments, peptides of this disclosure can remain intact after exposure to a temperature of at least 30° C. with at least 65% relative humidity for from 3 months to 12 months. In some embodiments, peptides of this disclosure can remain intact after exposure to a temperature of at least 30° C. with at least 65% relative humidity for from 12 months to 24 months. In some embodiments, peptides of this disclosure can remain intact after exposure to a temperature of at least 30° C. with at least 70% relative humidity for from 3 months to 48 months. In some embodiments, peptides of this disclosure can remain intact after exposure to a temperature of at least 30° C. with at least 70% relative humidity for from 3 months to 12 months. In some embodiments, peptides of this disclosure can remain intact after exposure to a temperature of at least 30° C. with at least 70% relative humidity for from 12 months to 24 months.

In some embodiments, peptides of this disclosure can remain intact after exposure to a temperature of at least 40° C. with at least 60% relative humidity for from 3 months to 48 months. In some embodiments, peptides of this disclosure can remain intact after exposure to a temperature of at least 40° C. with at least 60% relative humidity for from 3 months to 12 months. In some embodiments, peptides of this disclosure can remain intact after exposure to a temperature of at least 40° C. with at least 60% relative humidity for from 12 months to 24 months. In some embodiments, peptides of this disclosure can remain intact after exposure to a temperature of at least 40° C. with at least 65% relative humidity for from 3 months to 48 months. In some embodiments, peptides of this disclosure can remain intact after exposure to a temperature of at least 40° C. with at least 65% relative humidity for from 3 months to 12 months. In some embodiments, peptides of this disclosure can remain intact after exposure to a temperature of at least 40° C. with at least 65% relative humidity for from 12 months to 24 months. In some embodiments, peptides of this disclosure can remain intact after exposure to a temperature of at least 40° C. with at least 70% relative humidity for from 3 months to 48 months. In some embodiments, peptides of this disclosure can remain intact after exposure to a temperature of at least 40° C. with at least 70% relative humidity for from 3 months to 12 months. In some embodiments, peptides of this disclosure can remain intact after exposure to a temperature of at least 40° C. with at least 70% relative humidity for from 12 months to 24 months.

### Pharmaceutical Compositions of Peptides

A pharmaceutical composition of the disclosure can be a combination of any peptide described herein with other chemical components, such as carriers, stabilizers, diluents, dispersing agents, suspending agents, thickening agents, antioxidants, solubilizers, buffers including citric acid, osmolytes, salts, surfactants, amino acids, encapsulating agents, bulking agents, cryoprotectants, mucoadhesive agents, delayed release agents, enteric coatings, and/or excipients. The pharmaceutical composition facilitates administration of a peptide described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, buccal, intra-articular, intra-synovial, and topical administration. A pharmaceutical composition can be administered in a local or systemic manner, for example, via injection of the peptide described herein directly into an organ, optionally in a depot including biodegradable matrices, thermal gelling agents, and aqueous and non-aqueous solvents.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water soluble form. Suspensions of peptides described herein can be prepared as oily injection suspensions. Suitable lipophilic solvents or vehicles include fatty oils such as sesame oil, N-methyl pyrrolidone, propylene glycol, glycerol, alcohols, fatty acids or omega-3-fatty acids, or synthetic fatty acid esters, such as ethyl oleate or triglycerides, liposomes, micelles, or mixed micelles. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such peptides described herein to allow for the preparation of highly concentrated solutions. Alternatively, the peptides described herein can be lyophilized or in powder form for re-constitution with a suitable vehicle, e.g., sterile pyrogen-free water, 5% dextrose in water, isotonic saline solutions, or buffered solutions before use. In some embodiments, a purified peptide is administered intravenously.

A peptide of the disclosure can be applied directly to an organ, or an organ tissue or cells, such as brain or brain tissue or cancer cells, during a surgical procedure. The recombinant peptides described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide described herein described herein can be administered in pharmaceutical compositions to a subject suffering from a condition that affects the immune system. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the peptide described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Pharmaceutical compositions can also include permeation or absorption enhancers (Aungst et al. AAPS J. 14(1):10-8. (2012) and Moroz et al. Adv Drug Deliv Rev 101:108-21. (2016)). Permeation enhancers can facilitate uptake of molecules from the GI tract into systemic circulation. Permeation enhancers can include salts of medium chain fatty acids, sodium caprate, sodium caprylate, N-(8-[2-hydroxybenzoyl]amino)caprylic acid (SNAC), N-(5-chlorosalicyloyl)-8-aminocaprylic acid (5-CNAC), hydrophilic aromatic alcohols such as phenoxyethanol, benzyl alcohol, and phenyl alcohol, chitosan, alkyl glycosides, dodecyl-2-N,N-dimethylamino propionate (DDAIPP), chelators of divalent cations including EDTA, EGTA, and citric acid, sodium alkyl sulfate, sodium salicylate, lecithin-based, or bile salt-derived agents such as deoxycholates,

Compositions can also include protease inhibitors including soy bean trypsin inhibitor, aprotinin, sodium glycocholate, camostat mesilate, vacitracin, or cyclopentadecalactone.

Pharmaceutical compositions can also include excipients to release an agent in certain parts of the gastrointestinal (GI) tract. For example, but limited to, an excipient can be an enteric coating (e.g., fatty acids, waxes, shellac, plastics, and plant fibers), methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, or zein.

A pharmaceutical composition of the disclosure can be a combination of any peptide or peptide-conjugate described herein, or a salt thereof, with other chemical components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients. The pharmaceutical composition facilitates administration of a peptide or peptide-conjugate described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, intra-articular, topical administration, or combination thereof. A pharmaceutical composition can be administered in a local or systemic manner, for example, via injection of the peptide described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water soluble form. Suspensions of peptides described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduces the aggregation of such peptides described herein to allow for the preparation of highly concentrated solutions. Alternatively, the peptides described herein can be lyophilized or in powder form for re-constitution with a suitable vehicle, e.g., sterile pyrogen-free water, before use. In some embodiments, a purified peptide is administered intravenously.

A peptide or peptide-conjugate of the disclosure can be applied directly to an organ, or an organ tissue or cells, such as brain or brain tissue or cancer cells, during a surgical procedure. The recombinant peptides described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide described herein described herein can be administered in pharmaceutical compositions to a subject suffering from a condition that affects the immune system. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the peptide or peptide-conjugates described herein, or a salt thereof, with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Administration

A pharmaceutical composition of the disclosure can be a combination of any plant, venom, toxin, or artificially derived disulfide-rich peptide described herein with other chemical components, such as carriers, stabilizers, diluents, dispersing agents, suspending agents, thickening agents, and/or excipients. The pharmaceutical composition facilitates administration of a peptide described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, inhalation, dermal, intrathecal, intranasal, and topical administration. A pharmaceutical composition can be administered in a local or systemic manner, for example, via injection of the peptide described herein directly into an organ, optionally in a depot. In some cases, a particular route is advantageous as it reduces exposure of other non-target tissues to the peptide. For instance, oral route may be advantageous for some GI targets to avoid more systemic exposure to the peptide, or intrathecal route for pain treatment of the CNS to avoid more systemic exposure of the peptide. In some embodiments, localized administration of a peptide or composition thereof has the advantages of reducing adverse effects or off-target effects and/or lowering the dose used.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptides described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduces the aggregation of such peptides described herein to allow for the preparation of highly concentrated solutions. Alternatively, the peptides described herein can be lyophilized or in powder form for re-constitution with a suitable vehicle, e.g., sterile pyrogen-free water, before use. In some embodiments, a purified peptide is administered intravenously. A peptide described herein can be administered to a subject, home, target, migrates to, is retained by, and/or binds to, or be directed to an organ, e.g., the kidney.

A peptide of the disclosure can be applied directly to an organ, or an organ tissue or cells, such as the kidney, during a surgical procedure, such as kidney transplantation. The recombinant peptides described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide described herein described herein are administered in pharmaceutical compositions to a subject suffering from a condition. In some instances the pharmaceutical composition will affect the physiology of the animal, such as the immune system, inflammatory response, or other physiologic affect. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the peptide described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Use of Peptides in Treatments

In some embodiments, the method includes administering an effective amount of a peptide as described herein to a subject in need thereof.

In one embodiment, the method includes administering an effective amount of a peptide as described herein to a subject in need thereof.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case may be determined using techniques, such as a dose escalation study.

The methods, compositions, and kits of this disclosure may comprise a method to prevent, treat, arrest, reverse, or ameliorate the symptoms of a condition. The treatment may comprise treating a subject (e.g., an individual, a domestic animal, a wild animal, or a lab animal afflicted with a disease or condition) with a peptide of the disclosure. The disease may be a brain or spinal cord disease. In treating the disease, the peptide may cross the blood brain barrier or blood cerebrospinal fluid barrier of a subject. The subject may be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment may be provided to the subject before clinical onset of disease or after clinical onset of disease. Treatment may be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can comprise administering to a subject a pharmaceutical composition, such as one or more of the pharmaceutical compositions described throughout the disclosure. A treatment can comprise a once daily dosing. A treatment can comprise delivering a peptide of the disclosure to a subject, either intravenously, subcutaneously, intramuscularly, by inhalation, dermally, topically, by intra-articular injection, orally, sublingually, intrathecally, transdermally, intranasally, via a peritoneal route, directly into the brain, e.g., via and intracerebral ventrical route, or directly onto a joint, e.g. via topical, intra-articular injection route. A treatment can comprise administering a peptide-active agent complex to a subject, either intravenously, subcutaneously, intramuscularly, by inhalation, by intra-articular injection, dermally, topically, orally, intrathecally, transdermally, intransally, parenterally, orally, via a peritoneal route, nasally, sublingually, directly onto cancerous tissues, or directly onto or near cartilage.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat an ion channel related disorder. In certain embodiments, the peptide can be used as a delivery scaffold for an active agent or a detectable active agent. For example, a peptide or peptide-active agent conjugate can be used to access and treat disorders of the gastrointestinal (GI) tract, heart, brain, retina, cartilage, and kidney. In some embodiments, a peptide or peptide-active agent conjugate is used to treat cancer, pain, or an autoimmune disease.

The present disclosure provides peptides that can home, target, migrate to, accumulate in, are directed to, and/or bind to specific regions, tissues, structures, or cells and methods of using such peptides. In some embodiments, in addition to targeting a specific regions, tissues, structures, or cells, the peptides can also bind to an ion channel thereof. End uses of such peptides include, for example, imaging, research, therapeutics, diagnostics, theranostics, pharmaceuticals, chemotherapy, chelation therapy, targeted drug delivery, and radiotherapy. Such peptide can function as active agents alone, or conjugated to another active agent or a detectable label as described herein. Such peptides can also be modified to achieve a desired property, such as prolonged half-life, improved stability, and/or reduced adverse effects.

In some embodiments, peptides of present disclosure can home, distribute to, target, migrate to, accumulate in, or are directed to cancerous or diseased cells. In some embodiments, peptides of present disclosure can home, target, migrate to, accumulate in, or are directed to specific regions, tissues, structures, or cells within the body and methods of using such peptides. In some cases, these peptides have the ability to bind to cross the blood brain barrier or blood CSF barrier. These abilities make the peptides useful for a variety of applications. In particular, the peptides have applications in site-specific modulation of biomolecules to which the peptides are directed. End uses of such peptides include, for example, imaging, research, therapeutics, theranostics, pharmaceuticals, chemotherapy, chelation therapy, targeted drug delivery, and radiotherapy. Some uses can include targeted drug delivery and imaging.

Potassium Channels:

Voltage-gated potassium channels can be found in all excitable cells, and consist of four a protein chains surrounding a central pore, or homotetramer with four voltage sensors and one central pore domain. Each subunit comprises four transmembrane helices that form a voltage-sensor domain, and two transmembrane helices that form the central pore domain. Potassium channels are activated by depolarization after sodium channel activation, as the flow of potassium ions out of the cell allows for re-establishing resting cell potential. There are subtypes of potassium channels. For example KCNQ channels regulate critical physiological functions, such as regulation of heart beat or neuronal activity. Another class of potassium channels is inward rectifiers, comprising four identical subunits, having two membrane-spanning segments and one pore-lining segment.

hERG or KCHN2 is a gene that encodes Kᵥ 11.1, the alpha subunit of a potassium ion channel. hERG potassium channel comprises four identical a subunits that form the pore through the membrane. Each hERG subunit further comprises six transmembrane alpha helices numbered S1-S6, a pore helix located between S5 and S6, and N- and C-termini located in the cytoplasm. hERG forms the major portion of the ion channel proteins that conducts potassium ions out of the heart muscle cells, or cardiac myocytes. hERG contributes to the electrical activity of the heart by mediating the repolarizing current in the cardiac action potential. When the potassium ion channel's ability to conduct electrical current across the cardiac cells is inhibited or compromises, it can result in long QTs syndrome. hERG has also been associated with modulating functions of some cells in the nervous system and certain cancer cells, like leukemic cells. In some embodiments, a peptide of this disclosure binds to, interacts with, modulates, or functions as an antagonist or an agonist with any one of the potassium ion channels or subunits thereof described herein. In some embodiments, a peptide of this disclosure binds to, interacts with, modulates, or functions as an antagonist or an agonist with any one of the potassium ion channels described herein, e.g., hERG, or a variant or subunits thereof.

Ion channels are associated with various GI functional and motility disorders, including ion channels associated with central and peripheral (enteric) nervous systems and motor dysfunction in the GI wall. Interstitial cells of Cajal (ICCs) generate and propagate electrical activity, while smooth muscle cells (SMCs) are responsible for electromechanical coupling. These cells and other excitable cells have voltage-sensitive ion channels, including voltage-gated sodium (Nav), calcium (Cav), potassium (Kv, K_{Ca}), chloride and nonselective ion channels, such as transient receptor potentials (TRPs). See Beyder, A. and Farrugia, G., Ther. Adv. Gastroenterol., (2012) 5(1) 5-21 and Shoeb, F., et al., J Biol Chem. 2003 Jan. 24; 278(4):2503-14, which are incorporated in its entirety.

GI system has potassium channels that are voltage-sensitive and calcium-activated potassium selective channels. In the GI, Kv4 and Kv3 families of channels modulate A-type current in SMCc and ICCs. Multiple Kv1 family channels are expressed by GI SMCs, including Kv1.1, Kv1.2, Kv1.5. ERG channels are inwardly rectifying ion channels. Drug targets for hERG channels are complicated by the fact that they have a promiscuous binding pocket that leads to inhibition of cardiac hERG. Thus, there is a need for drugs, e.g., peptides that can selectively target GI hERG without affecting cardiac hERG, such as selective 5HT4 agonists. A key difference between cardiac and gut hERG is a truncation of a hundred residues in the gut hERG, which allows for selective targeting by a peptide of this disclosure. In some embodiments, a peptide of this disclosure selectively binds to gut hERG over cardiac hERG by at least 5 fold, by at least 10 fold, by at least 20 fold, by at least 50 fold, by at least 100 fold, or by at least 500 fold. In some embodiments, a peptide of this disclosure can distinguish gut hERG from cardiac hERG based on the truncation of a hundred residues in the gut hERG.

Elevated expression of Kv1.3 channels in autoreactive memory T lymphocytes has also been associated with T cell-mediated autoimmune diseases. Kv1.3 channel is homotetrameric and regulates membrane potential and calcium signaling in human T cells. See Beeton et al., Proc. Natl. Acad. Sci. 2006 Nov. 14; 103(46):17414-9. Elevated expression of Kv1.3 have been reported in activated CD4+ and CD8+ effector memory T cells of diabetes mellitus or rheumatoid arthritis patients. Specific Kv1.3 blockers, such as SLS (ShK analog, ShK-186) and 5-(4-phenoxybutoxy)psoralen (PAP-1), ameliorated disease in rat models of rheumatoid arthritis and diabetes mellitus. In some embodiments, a peptide of this disclosure binds to, interacts with, modulates, or functions as an antagonist or an agonist with any one of the potassium ion channels described herein, e.g., Kv1.3, or subunits thereof. In some embodiments, a peptide of this disclosure binds to, interacts with, modulates, or functions as an antagonist or an agonist with Kv1.3 or a variant or subunits thereof in T lymphocytes. In some embodiments, a peptide disclosed herein is conjugated to a Kv1.3 blocker, e.g., ShK analog, SLS, or PAP-1, or a derivative thereof. In some embodiments, a peptide disclosed herein suppresses or inactivates Kv1.3 channels in T lymphocytes, e.g., effector memory T cells, of a subject diagnosesd with or has a risk of developing autoimmune disease, such as rheumatoid arthritis.

In some embodiments, a peptide of this disclosure binds to potassium ion channels. In some cases, a peptide of this disclosure binds to any one of the following potassium channels: Kv1.1, 1.2, 1.3, Kv2.1, Kir2.1, and hERG channels. In some cases, a peptide of this disclosure binds to hERG channels. Potassium ion channels include but are not limited to hERG (cardiac), HCN, Kv, Kd (delayed rectifier), Kf (fast transient), KCa (calcium-activated), MaxiK (activated by Ca++), TASK-1, Shaker, Shal, Shab, Shaw, minK (miniature potassium channel, or IsK), KvLQT, and KCNK channels.

In some embodiments, a peptide of this disclosure binds to, activates, deactivates, or inhibits an inward-rectifier potassium channel. Inwardly rectifying potassium channels (or Kir) are a specific subset of potassium selective ion channels. In some cases, Kir channels are targets of a toxin. Kir channels typically have a pore domain that is homologous to the por domain of voltage-gated ion channels, and flanking transmembrane segments. Kir channels can exist in cellular membrane as homo- or heterooligomers and each monomer can have between 2 and 4 transmembrane segments. Kir channels play a role in potassium ion transport, with a greater tendency for potassium ion uptake than potassium ion export.

Kir channels can be found in multiple cell types, including macrophages, cardiac cells or cardiac myocytes, kidney cells, leukocytes, neurons, pancreatic beta cells, and endothelial cells. In various cells, Kir channels play a role in mediating small depolarizing potassium ion current at negative membrane potentials and can help establish resting membrane potential, and/or help mediate inhibitory neurotransmitter responses. In some embodiments, a peptide comprising the sequence of any of SEQ ID NO: 1-SEQ ID NO: 80 binds to a Kir channel expressed in any of macrophages, cardiac cells or cardiac myocytes, kidney cells, leukocytes, neurons, pancreatic beta cells, and endothelial cells.

In some cases, a peptide of this disclosure is used to activate, deactivate, or inhibit a Kir channel, such as Kir1.1, Kir2.1, Kir2.2, Kir2.3, Kir2.4, Kir3.1, Kir3.2, Kir3.3, Kir3.4, Kir4.1, Kir4.2, Kir5.1, Kir6.1, Kir6.2, and Kir7.1. In some embodiments, a peptide of at least 80% sequence identity to SEQ ID NO: 1-SEQ ID NO: 80 binds to any one of Kir1.1, Kir2.1, Kir2.2, Kir2.3, Kir2.4, Kir3.1, Kir3.2, Kir3.3, Kir3.4, Kir4.1, Kir4.2, Kir5.1, Kir6.1, Kir6.2, and Kir7.1 channels.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a potassium channel related disorder. In some embodiments, a peptide comprising the sequence of any of SEQ ID NO: 1-SEQ ID NO: 80 binds to a potassium channel to inhibit or activate its activity, including Kv1.1, Kv1.2, Kv1.3, hERG, and Kir channels.

In some embodiments, a peptide having at least 80% sequence identity to SEQ ID NO: 6, 19, 39, 46, 59, or 79 inhibits Kv1.1. In some embodiments, a peptide having at least 80% sequence identity to any one of SEQ ID NO: 1-4, 11-14, 17-19, 21, 22, 26, 29, 31, 40, 41-44, 51-54, 57-59, 61, 62, 66, 69, 71, and 80 inhibits Kv1.2. In some embodiments, a peptide having at least 80% sequence identity to any one of SEQ ID NO: 2, 5, 6, 12, 17, 18, 19, 20, 21, 29, 33, 38, 39, 40, 42, 45, 46, 52, 57, 58, 59, 60, 61, 69, 73, 78, 79, and 80 inhibits Kv1.3.

In some embodiments, a peptide having at least 80% sequence identity to SEQ ID NO: 7, 14, 17, 47, 54, or 57 binds to hERG. In some embodiments, a peptide having at least 80% sequence identity to SEQ ID NO: 7, 14, 17, 47, 54, or 57 inhibits hERG.

Voltage-gated Kv1.3 potassium channel is expressed in effector memory T cells, which play a role in various autoimmune diseases, e.g., rheumatoid arthritis, multiple sclerosis (MS), and type-1 diabetes mellitus (T1DM). In some embodiments, a peptide of this disclosure, SEQ ID NO: 1 to SEQ ID NO. 80, is used to target Kv1.3 channels expressed in effector memory T cells to treat an autoimmune disease. In some cases, peptides comprising one or more, two or more, three or more, four or more, or five or more amino acid residues that adjust the acidic residue distribution at the peptide-ion channel binding interface, such as aspartic acid and glutamic acid substitutions, are used to selectively bind and/or inhibit an ion channel, such as a potassium ion channel. Such engineered or non-naturally occurring peptides can function as Kv1.3 channel inhibitors that suppress cytokine secretion and thus alleviate T-cell mediated autoimmune diseases. Such non-naturally occurring peptides are better drug candidates than chemical molecules because peptides can be engineered to improve selectivity, specificity, and to decrease side effects. In some embodiments, a peptide of this disclosure or a derivative thereof comprises one or more, two or more, three or more, four or more, or five or more amino acid substitutions that alter the polarity or electrostatic distribution of the peptide at the binding interface.

In some embodiments, a peptide of this disclosure or a derivative thereof selectively blocks Kv1.3 channel to inhibit cytokine secretion by T cells, such as TNF-α, IL-2, and IFN-γ, to improve delayed-type hypersensitivity responses, or to decrease autoreactive T-cell mediated inflammation in vivo. Inhibition of Kv1.3 can inhibit the activation of T cells and secretion of cytokines via the calcineurin pathway by preventing potassium efflux necessary for calcium influx. In some embodiments, a peptide of this disclosure comprises one or more mutations, wherein the peptide has selective binding to Kv1.3 and inhibits any one of the following activities in vivo: cytokine secretion, proliferation of T cells, and delayed-type hypersensitivity reaction in vivo. Such peptide functions as an immunosuppressant, which can be used to treat an autoimmune disease.

In some embodiments, a peptide of this disclosure comprises a structural analog of BmKTX scorpion toxin. In some embodiments, the peptide comprises a mutation at one or more, or two or more of the following positions: Asp6, Asp19, and Asp33. In some embodiments, a peptide that inhibits Kv1.3 comprises a mutation at Asp33; or at Asp6 and Asp33. In some embodiments, a peptide that selectively inhibits Kv1.3 channel comprises a D33H mutation, or a similar mutation at a position corresponding to Asp33. See Ye, F., et al., Toxins (Basel), 2016 Apr. 19; 8(4):115.

In some embodiments, a peptide that targets a potassium ion channel, such as Kv1.3, comprises a mutation at position D33, e.g., D33H, D33R, D33K. In some embodiments, a peptide disclosed herein comprises a mutation that alters the electrostatic distribution of a domain of the peptide that interacts with the potassium ion channel.

In some embodiments, such mutation results in a peptide that selectively blocks one type of ion channel, e.g., Kv1.3 channel, with more than 10-fold, more than 50-fold, more than 100-fold, more than 500-fold, or more than 1000-fold selectivity as compared to other ion channels or subtypes, e.g., Kv1.1, Kv1.2, Kv1.7, Kv11.1, KCa2.2, KCa2.3, and KCa3.1.

In some embodiments, a peptide disclosed herein is selective for any one of Kv1.1, Kv1.2, Kv1.3, and hERG over at least 2, at least 3, at least 4, at least 5, at least 6, at least 7, at least 8, at least 9, or at least 10 other ion channel types or potassium ion channel subtypes.

In some embodiments, a peptide disclosed herein that blocks a potassium ion channel, such as Kv1.3, comprises one or more of the following mutations corresponding to positions G11R, I28T, and D33H in BmKTX. In some embodiments, a peptide disclosed herein comprises one or more mutations in the conserved anti-parallel beta-sheet domain in its binding interface with a potassium ion channel.

In some embodiments, a peptide of this disclosure is an analog of the ShK disulfide-rich peptide derived from sea anemone and blocks Kv1.3 and/or Kv1.1 ion channels. In some embodiments, a peptide that selectively blocks Kv1.3 comprises one or more of the following substitutions: p-phosphono-phenylalanine at the N-terminus, Gln16, Met21, and alanine extension at the C-terminus. In some embodiments, a peptide of this disclosure inhibits Kv1.4 or Kv1.6.

ShK petpides comprise two small α-helical segments, an extended N-terminal segment, and two interlocking turns that resemble a 3₁₀ helix. In some cases, the CDP peptides are characterized by a dominant three-stranded antiparallel beta-sheet domain with an alpha-helix across stands 2 and 3. ShK and kaliotoxin comprise three stabilizing disulfide bonds.

In some embodiments, a peptide of this disclosure blocks an ion channel using a Lys or Arg residue. Residues corresponding to Lys22 and Tyr23 in ShK peptide can be important for potassium ion channel binding. In some cases, a peptide of this disclosure selectively targets Kv1.1. channels in cardiac cells, or Kv1.4 channels in the brain, or Kv1.6 channels in the brain. In some embodiments, a peptide derived from ShK or an analog thereof comprises a substitution at Gln16, such as Gln16 to Lys substitution. In some embodiments, a peptide of this disclosure comprises N-terminal extension with a negatively charged moiety to enhance its selectivity for Kv1.3 over Kv1.1. In some embodiments, a peptide of this disclosure comprises C-terminal amidation and/or addition of a Lys at the C-terminus. In some cases, addition of Ala at the C-terminus can also enhance selectivity for Kv1.3. In some cases, substitution of a residue corresponding to Met21 in Shk with hydrophobic residues, such as Nle or Ile, can also enhance the selectivity for Kv1.3.

Sodium Channels:

Voltage-gated sodium channel is formed by the a subunit chain, which has four homologous but non-identical domains. Each domain has 6 transmembrane segments. The β subunit is a smaller polypeptide with a single transmembrane segment and an extra-cellular domain. The β subunit plays a role in the gating mechanism of the channel and the rate the channel closes or opens. Mutations in the a subunit of voltage-gated sodium channel (SCN4A) is associated with two types of autosomal dominant skeletal muscle disorders: periodic paralysis and myotonia.

Sodium ion channels include mH1, mH2, SCN4A (skeletal muscle), PN1, PN3, SkM1, RSMK, Kat1, EAG, ELK, Drk1. In some embodiments, a peptide of this disclosure binds to, interacts with, modulates, or functions as an antagonist or an agonist with any one of the sodium ion channels described herein or a variant or subunits thereof.

In some embodiments, a peptide of this disclosure binds to a sodium channel. In some embodiments, a peptide of this disclosure binds to Nav1.5 and/or Nav1.7 ion channels to inhibit or activate ion channel activity. In some embodiments, a peptide disclosed herein binds to any one of the following sodium channels: Nav1.5 (TP1), Nav1.5 (TP2), Nav 1.7 (TP1), and Nav1.7 (TP2).

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a sodium channel related disorder. In some embodiments, a peptide comprising the sequence of any of SEQ ID NO: 1-SEQ ID NO: 80 binds to a sodium channel to inhibit or activate its activity, including Nav1.5 (TP1), Nav1.5 (TP2), Nav1.7 (TP1), and Nav1.7 (TP2) channels. In some embodiments, a peptide having at least 80% sequence identity to SEQ ID NO: 7 or SEQ ID NO: 47 inhibits Nav1.5 (TP1) or Nav1.5 (TP2). In some embodiments, a peptide having at least 80% sequence identity to any one of SEQ ID NO: 7, 18, 31, 39, 47, 58, 71, and 79 inhibits Nav1.7 (TP1) and Nav1.7 (TP2).

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a sodium channel related disorder. In some embodiments, a peptide comprising the sequence of any of SEQ ID NO: 1-SEQ ID NO: 80 binds to a sodium channel to inhibit or activate its activity, including Nav1.5 (TP1), Nav1.5 (TP2), Nav1.7 (TP1), and Nav1.7 (TP2) channels.

Calcium Channels:

Voltage-gated calcium channels comprise a large α1 subunit, which contains the pore, and regulatory α2, β, χ, and δ subunit. The principal protein is the α1 subunit, which comprises four homologous domains with six transmembrane alpha-helix segments each. Various α1 subunits are divided into three sub-families or subtypes: Caᵥ1.1-Caᵥ1.4 (L-type); Caᵥ2.1 (P/Q-type); Caᵥ2.2 (N-type) and Caᵥ2.3 (R-type); Caᵥ3.1-Caᵥ3.3 (T-type). Voltage-gated calcium channels are commonly found in membranes of excitable cells, such as muscle cells, glial cells, and neurons. Caᵥ channels play a role in regulating membrane potential and intracellular calcium signaling pathways, and are thus implicated in various neurological and cardiovascular diseases. In some embodiments, a peptide of this disclosure binds to, interacts with, modulates, or functions as an antagonist or an agonist with any one of the calcium ion channels described herein, or a variant or subunits thereof.

In some cases, a peptide of this disclosure binds to a calcium channel. In some cases, a peptide of this disclosure binds to any one of Cav2.1 and Cav2.2.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a calcium channel related disorder. In some embodiments, a peptide comprising the sequence of any of SEQ ID NO: 1-SEQ ID NO: 80 binds to a calcium channel to inhibit or activate its activity, including Cav2.1 and Cav2.2 channels. In some embodiments, a peptide having at least 80% sequence identity to SEQ ID NO: 7 or SEQ ID NO: 47 inhibits Cav2.1 or Cav2.2 channel. In some embodiments, a peptide having at least 80% sequence identity to any one of SEQ ID NO: 7 or SEQ ID NO: 47 activates Cav2.1 or Cav2.2 channel.

TRP Channels:

Transient receptor potential (TRP) channels are a class of cation channels found mostly on plasma membrane of animal cells and perform functions ranging from pain perception to calcium and magnesium absorption. TRP channels can be classified into six subfamilies: TRPA (ankyrin), TRPV (vanilloid), TRPM (melastatin), TRPC (canonical), TRPP (polycystin), and TRPML (mucolipin). Group 1 of TRP genes are comprised of TRPC (canonical), TRPM (melastatin), TRPV (vanilloid), and TRPA (ankyrin). Group 2 includes TRPP (polycystin) and TRPML (mucolipin). See Pan et al., (2012). Transient Receptor Potential (TRP) Channels in the Eye, Advances in Ophthalmology, Dr Shimon Rumelt (Ed.).

TRP channels comprise six transmembrane spanning domains and a cation-permeable pore formed by a short hydrophobic region between transmembrane domains 5 and 6, and are typically configured as homo- or hetero-tetramers to form non-selective cation channels. TRP channels' permeability ratios to Ca2+/Na+ vary among individual members. TRPV5 and TRPV6 channels have a Ca2+/Na+ permeability ratio of greater than 100, indicating high Ca2+ selectivity. TRPM4 and TRPM5 channels are impermeable to Ca2+ but are selective for monovalent cations (Na+, K+). TRP channels play a role in ocular sensory and cellular function, including ocular disease, ocular surface disease, wound healing of an eye, glaucoma, cataract development, retinopathy, wet age-related macular degeneration (AMD). In some embodiments, a peptide of this disclosure binds to, interacts with, modulates, or functions as an antagonist or an agonist with any one of the TRP channels described herein or subunits or variants thereof. In some embodiments, such peptide is used to treat an ocular disease; ocular surface disease; wound, injury, or inflammation of an eye; glaucoma; cataract development; retinopathy; or wet AMD; or to treat pain, affect metabolic control, thermosensation and thermoregulation.

In some embodiments, a peptide of this disclosure binds to, interacts with, modulates, or functions as an antagonist or an agonist with any one of the TRP channels described herein, or a variant or subunits thereof.

In some cases, a peptide of this disclosure binds to a TRP ion channel. In some cases, a peptide of this disclosure binds to a TRPV1 channel, also known as the capsaicin receptor. TRPV1 functions to detect and regulate body temperature and plays a role in providing a sensation of heat and pain.

TRPV1 is a nonselective cation channel that can be activated by various exogenous and endogenous physical and chemical stimuli, including acidic conditions, capsaicin, allyl isothiocyanate, endocannabinoid anandamide, N-oleyl-dopamine, and N-arachidonoyl-dopamine. In some cases, a peptide of this disclosure functions as an antagonist that blocks TRPV1 activity and thus reducing pain. In some cases, such antagonists are used to reduce nociception from inflammatory and neuropathic pain, or to treat neuropathic pain associated with multiple sclerosis, chemotherapy, or amputation, as well as pain associated with the inflammatory response of damaged tissue, such as in osteoarthritis. In some cases, a peptide of this disclosure functions as an agonist, such as capsaicin and resiniferatoxin, which activate TRPV1. Prolonged application of a TRPV1 agonist can cause TRPV1 activity to decrease (desensitization), leading to alleviation of pain via the subsequent decrease in the TRPV1 mediated release of inflammatory molecules.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a TRP channel related disorder, such as pain management. In some embodiments, a peptide comprising the sequence of any of SEQ ID NO: 1-SEQ ID NO: 80 binds to a TRP channel to inhibit or activate its activity. In some embodiments, a peptide having at least 80% sequence identity to SEQ ID NO: 1-SEQ ID NO: 80 inhibits TRPA1 or TRPV1 channel. In some embodiments, a peptide having at least 80% sequence identity to any one of SEQ ID NO: 1-SEQ ID NO: 80 activates TRPA1 or TRPV1 channel.

GABA Channels:

GABA_{A} is a ligand-gated ion channel wherein γ-aminobutyric acid (GABA) is the endogenous ligand and a major neurotransmitter in the CNS. GABA is an inhibitory compound in the CNS. The subunits of GABA channels can be grouped into eight families: α1-6, β1-3, γ1-3, δ, ε, π, θ, and ρ. In some embodiments, a peptide of this disclosure binds to, interacts with, modulates, or functions as an antagonist or an agonist with any one of the GABA channels described herein, or a variant or subunits thereof. There are two classes of GABA receptors: GABA_{A} and GABA_{B} receptors. GABA_{A} receptors are ligand-gated ion channels (also known as ionotropic receptors). GABA_{B} receptors are G protein-coupled receptors, also called metabotropic receptors. Fast-responding GABA receptors are members of the Cys-loop ligand-gated ion channels. In ionotropic GABA_{A} receptors, binding of GABA molecules to binding sites in the extracellular part of the receptor opens a chloride ion-selective pore, which leads to an increased chloride conductance that drives the membrane potential towards the reversal potential of the chloride ion and inhibition of the firing of new action potentials. This mechanism is responsible for the sedative effects of GABA_{A} allosteric agonists. Activation of GABA receptors can also lead to shunting inhibition, which reduces the excitability of the cell independent of the changes in membrane potential. A slow response to GABA is mediated by GABA_{B} receptors.

In some cases, a GABA receptor agonist binds to one or more of the GABA receptors to produce a sedative effect, e.g., anxiolytic, anticonvulsant, and muscle relaxant effects. There are three types of receptors of the gamma-aminobutyric acid. GABA-α and GABA-ρ receptors are ion channels that signal chloride and diminish action potentials. The GABA-β receptor belongs to the class of G-Protein coupled receptors that inhibit adenylyl cyclase, leading to decreased cyclic adenosine monophosphate (cAMP). GABA-α and GABA-ρ receptors produce sedative and hypnotic effects and have anti-convulsion properties. GABA-β receptors can also produce sedative effects. GABA receptor antagonists function to inhibit action of GABA receptors, and can produce stimulant and/or convulsant effects.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a GABA channel/receptor related disorder, such as Alzheimer's disease. In some embodiments, a peptide comprising the sequence of any of SEQ ID NO: 1-SEQ ID NO: 80 binds to a GABA channel/receptor to inhibit or activate its activity. In some embodiments, a peptide having at least 80% sequence identity to SEQ ID NO: 1-SEQ ID NO: 80 inhibits GABA channel/receptor. In some embodiments, a peptide having at least 80% sequence identity to any one of SEQ ID NO: 1-SEQ ID NO: 80 activates GABA channel/receptor.

Ionotropic Glutamate Receptor Channels:

Ionotropic glutamate receptors (iGluRs) are ligand-gated ion channels that are activated by neurotransmitter glutamate, and mediate majority of excitatory neurotransmission and function of mammalian CNS. iGluRs are subdivided into subtypes based on ligand binding properties and sequence similarity: AMPA receptors, kainate receptors, NMDA receptors and delta receptors. Various neurological disorders are linked to aberrant activity of iGluRs, including epilepsy, stroke, and Alzheimer's disease. iGluRs are tetramers, formed of four subunits. Each subunit consists of extracellular N-terminal domain, ligand-binding domain, transmembrane domain that forms the ion channel, and an intracellular C-terminal domain. In some embodiments, a peptide of this disclosure binds to, interacts with, modulates, or functions as an antagonist or an agonist with any one of the iGluRs described herein, or a variant or subunits thereof.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a iGluR related disorder, such as Alzheimer's disease or epilepsy. In some embodiments, a peptide comprising the sequence of any of SEQ ID NO: 1-SEQ ID NO: 80 binds to a iGluR to inhibit or activate its activity. In some embodiments, a peptide having at least 80% sequence identity to SEQ ID NO: 1-SEQ ID NO: 80 inhibits iGluR. In some embodiments, a peptide having at least 80% sequence identity to any one of SEQ ID NO: 1-SEQ ID NO: 80 activates iGluR.

Acetylcholine Receptors:

Acetylcholine receptor (AChR) is an integral membrane protein that responds to acetylcholine neurotransmitter. Acetylcholine is an endogenous ligand for nicotinic acetylcholine receptor (nAChR). Examples of nicotinic receptor include α3β4 or α4β2. nAChRs are ligand-gated ion channels comprising five protein subunits symmetrically arranged to form a barrel-like channel. Each subunit contains four regions that span the membrane. Binding of acetylcholine to the N-termini of each of the two alpha subunits results in a conformational change that modulates the receptor activity. Reduced expression of nAChRs is associated with schizophrenia and Alzheimer's disease, suggesting that α7 and α4β2 receptor agonists could be useful for the treatment of cognitive impairment disorders. In some embodiments, a peptide of this disclosure binds to, interacts with, modulates, or functions as an antagonist or an agonist with any one of the AChR described herein, or a variant or subunits thereof.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat an AChR related disorder, such as Alzheimer's disease or a cognitive impairment. In some embodiments, a peptide comprising the sequence of any of SEQ ID NO: 1-SEQ ID NO: 80 binds to AChR, such as α3β4 or α4β2, to inhibit or activate its activity. In some embodiments, a peptide having at least 80% sequence identity to SEQ ID NO: 1-SEQ ID NO: 80 inhibits AChR. In some embodiments, a peptide having at least 80% sequence identity to any one of SEQ ID NO: 1-SEQ ID NO: 80 activates AChR.

5-HT3 Receptors:

5-HT3 is expressed in the CNS, the peripheral nervous system, and the gut, and plays a role in excitatory synaptic transmission. 5-HT3 receptors belong to the Cys-loop superfamily of ligand-gated ion channels, and are selective for cations and mediate neuronal depolarization and excitation within the CNS and peripheral nervous system. 5-HT3 receptor consists of five subunits arranged around a conducting pore, which is permeable to sodium, potassium, and calcium ions. Binding of neurotransmitter 5-hydroxytryptamine (serotonin) to the 5-HT3 receptor opens the channel and leads to an excitatory response in neurons. In some embodiments, a peptide of this disclosure binds to, interacts with, modulates, or functions as an antagonist or an agonist with any one of the 5-HT3 receptors described herein, or a variant or subunits thereof.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a 5-HT3 related disorder, such as GI disorder, pain disorder, or psychiatric disorder. In some embodiments, a peptide comprising the sequence of any of SEQ ID NO: 1-SEQ ID NO: 80 binds to 5-HT3a to inhibit or activate its activity. In some embodiments, a peptide having at least 80% sequence identity to SEQ ID NO: 1-SEQ ID NO: 80 inhibits 5-HT3a. In some embodiments, a peptide having at least 80% sequence identity to any one of SEQ ID NO: 1-SEQ ID NO: 80 activates 5-HT3a.

Chloride Channels:

Chloride channels are a class of ion channels specific for chloride ions. The CLC family of chloride channels contains 10 or 12 transmembrane helices, wherein each protein forms a single pore. Some members of the CLC family form homodimers. CLCN1 is involved in maintaining resting membrane potential in skeletal muscles, while other chloride channels play a role in maintaining solute concentrations in kidney. In some embodiments, a peptide of this disclosure binds to, interacts with, modulates, or functions as an antagonist or an agonist with any one of the chloride ion channels described herein, or a variant or subunits thereof. In some embodiments, a peptide of this disclosure binds to, interacts with, modulates, or functions as an antagonist or an agonist with any one of the chloride ion channels described herein, or a variant or subunits thereof in kidney cells.

Lubiprostone is a bicyclic fatty acid derivative of prostaglandin E₁ that increases small intestinal smooth muscle contractions through a prostaglandin E receptor 1 (EP1)-mediated pathway, and is used to treat chronic chronic idiopathic constipation and constipation predominant irritable bowel syndrome. See Jun J Y, J. Neurogastroenterol. Motil. 2013 July; 19(3):277-8. Lubiprostone works by stimulating chloride secretion by activating chloride channel type-2 (ClC-2) and cystic fibrosis transmembrane conductance regulator (CFTR) chloride channels in the apical membrane of the intestinal epithelial cells. Activation of ClC-2 channels or CFTR chloride channels in intestinal epithelial cells results in secretion of chloride ions from cells into the intestinal lumen, which increases the liquidity of the luminal contents. In some embodiments, a peptide of this disclosure is conjugated to lubiprostone to provide a more targeted delivery to chloride channel type-2. In some embodiments, a peptide of this disclosure is used to treat a disease and/or disorder associated with gastrointestinal motility, or to modulate gastrointestinal motility. In some embodiments, a peptide of this disclosure is used to activate ClC-2 channels or CFTR chloride channels.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a chloride channel related disorder, such as GI disorder. In some embodiments, a peptide comprising the sequence of any of SEQ ID NO: 1-SEQ ID NO: 80 binds to a chloride ion channel. In some embodiments, a peptide having at least 80% sequence identity to SEQ ID NO: 1-SEQ ID NO: 80 inhibits a chloride ion channel. In some embodiments, a peptide having at least 80% sequence identity to any one of SEQ ID NO: 1-SEQ ID NO: 80 activates a chloride ion channel.

Mechanosensitive Channels:

Mechanosensitive ion channels or stretch-gated ion channels are membrane proteins capable of responding to mechanical stress over a range of external mechanical stimuli. Mechanosensitive channels function like sensors for a number of systems including touch, hearing and balance, cardiovascular regulation and osmotic homeostasis. Such channels vary in ion selectivity, including from nonselective anions and/or cations channels, selectivity for Ca2+, K+ and Na+ ions, and high selectivity for K+ ions. Mechanosensitive channels include TRECK-1 channel, which plays a role in resting membrane potential.

Mechanosensitive ion channels include ENaC/DEG superfamily, comprising ASIC subunits (e.g., ASIC1a, ASIC1b, ASIC2a, ASIC2b, ASIC3, and ASIC4); TRP superfamily, comprising TRPC (canonical), TRPV (vanilloid), TRPM (melastatin), TRPP (polycystin), TRPML (mucolipin), TRPA (ankyrin), and TRPN (NOMPC-like) subfamilies; and K1-selective superfamily, wherein K2P channels consist of six subfamilies and contain four transmembrane domains.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a mechanosensitive ion channel related disorder, such as cardiovascular disease. In some embodiments, a peptide comprising the sequence of any of SEQ ID NO: 1-SEQ ID NO: 80 binds to a mechanosensitive ion channel. In some embodiments, a peptide having at least 80% sequence identity to SEQ ID NO: 1-SEQ ID NO: 80 inhibits a mechanosensitive ion channel. In some embodiments, a peptide having at least 80% sequence identity to any one of SEQ ID NO: 1-SEQ ID NO: 80 activates a mechanosensitive ion channel.

N-methyl-D-aspartate (NMDA) Receptors:

NR2A and NR2B are examples of NMDA receptors, which are glutamate receptors and ion channel proteins found in nerve cells. Activation of NMDA receptors results in the opening of an ion channel that is nonselective to cations, with a combined reversal potential near 0 mV. NR2B is predominant in early postnatal brain, while the number of NR2A subunits grows. Eventually NR2A subunits outnumber NR2B. This dynamic is referred to as the NR2B and NR2A switch. Greater ratios of the NR2B subunit leads to NMDA receptors remaining open longer compared to those with more NR2A.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a NMDA receptor related disorder, such as stroke or brain injury. In some embodiments, a peptide comprising the sequence of any of SEQ ID NO: 1-SEQ ID NO: 80 binds to a NMDA receptor. In some embodiments, a peptide having at least 80% sequence identity to SEQ ID NO: 1-SEQ ID NO: 80 inhibits a NMDA receptor, such as NR2B or NR2A receptor. In some embodiments, a peptide having at least 80% sequence identity to any one of SEQ ID NO: 1-SEQ ID NO: 80 activates a NMDA receptor, such as NR2B or NR2A receptor.

In some embodiments, a peptide of this disclosure, SEQ ID NO: 1 to SEQ ID NO. 80, is used to target Kv1.3 channels expressed in effector memory T cells to treat an autoimmune disease. Such peptides can function as Kv1.3 channel inhibitors that suppress cytokine secretion and thus alleviate T-cell mediated autoimmune diseases. See Chen, Z. et al., Sci. Rep. 2015 May 8; 5:9881, which is incorporated by reference in its entirety.

Unless otherwise defined, all technical terms used herein have the same meaning as commonly understood by one of ordinary skill in the art. As used in this specification and the appended claims, the singular forms “a,” “an,” and “the” include plural references unless the context clearly dictates otherwise. Any reference to “or” herein is intended to encompass “and/or” unless otherwise stated. As used herein, the term “about” a number refers to that number plus or minus 10% of that number. The term “about” a range refers to that range minus 10% of its lowest value and plus 10% of its greatest value.

As used herein, the term “disease”, “disorder”, or “condition” are used interchangeably.

### Ion Channel-Related Diseases

Many diseases are linked to proteins embedded in cell membranes, and ion channels are one class of such molecules. Such proteins form channels or pores in cell membranes and allow ions to pass through them. The gating mechanism of channels controls the movement of ions across a membrane. In general, there are three types of gating mechanisms: voltage-gated channels, which are modulated by a change in membrane potential; ligand-gated channels, which are modulated by molecules that bind to specific sites of the channels; and channels that are modulated or activated by certain mechanical stimuli. In some cases, an ion channel related disease is associated with an inward rectifier channel or a mechanosensitive ion channel. In some cases, a disease state can be improved by activating, blocking, or modulating one or more ion channels, even if the ion channels are not the source of the disease.

Mutations in muscle voltage-gated channels, such as sodium, potassium, calcium, and chloride channels, acetylcholine-gated or glycine-gated channels are implicated in a variety of diseases and disorders, including hyper- and hypokalemic periodic paralysis, myotonias, long QY syndrome, Brugada syndrome, malignant hyperthermia and myasthenia. Neuronal disorders or diseases include, but are not limited to, epilepsy, episodic ataxia, familial hemiplegic migraine, Lambert-Eaton myasthenic syndrome, Alzheimer's disease, Parkinson's disease, schizophrenia, hyperekplexia. Kidney disorders or conditions involving ion channels include Bartter's syndrome, polycystic kidney disease, Dent's disease, secretion disorders, e.g., hyperinsulinemic hypoglycemia of infancy and cystic fibrosis, and vision disorders.

In some cases, a peptide disclosed herein is used to treat an ion channel-related disorder, including a neurological disorder, cancer, autoimmune disease, GI motility disorder, irritable bowel syndrome, inflammatory bowel disease, constipation, dyspepsia, acquired neuromyotonia, renal disorder, ocular disorder, retinal disease, epilepsy, migraine, ataxia, polycystic kidney disease, seizure, long or short QT syndrome, paralysis, pain, neuropathic pain, severe pain, need for local anesthesia, migraine, cystic fibrosis, Bartter syndrome, endocrine disorder, rheumatoid arthritis, type 1 diabetes mellitus, multiple sclerosis, psoriasis, lupus, asthma, obesity, insulin resistance, hypertension, hypotension, stroke, Alzheimer's disease, arrhythmia, or bone disease. In some cases, a peptide is used to treat an ion channel-related disorder that is a cancer, including breast, cervical, neuoroblastoma, hepatocellular, prostate, colon, squamous lung cell, mammary gland, adenocarcinoma, leukemia, glioma, endometrial, chronic lymphocytic leukemia (CLL), acute myeloid leukemia (AML), or glioblastoma cancer or tumor.

In some cases, a peptide of the disclosure is used to treat a Kir channel related disease, including persistent hyperinsulinemic hypoglycemia of infancy; hypoglycemia, Bartter's syndrome, Andersen's syndrome; atherosclerosis; heart disease; loss of Kir currents in endothelial cells leading to atherogenesis or heart disease; thyrotoxic hypokalaemic periodic paralysis; and EAST/SeSAME syndrome.

In some cases, a peptide of the disclosure is used to treat a disease associated with a stretch-actived ion channel, such as cardiac arrhythmia, atrial fibrillation, cardiac hypertrophy, Duchenne muscular dystrophy, and other cardiovascular diseases. In some cases, a peptide of the disclosure is used to treat a disease associated with a mechanosensitive channel, such as polycystic kidney disease, atrial fibrillation, a neuronal disease, muscular degeneration, cardiac arrhythmia, or hypertension.

In some cases, a peptide disclosed herein is used to activate or deactivate an ion channel associated with a disease state in a particular cell type, tissue, or organ, such as a tissue or cell from heart, kidney, brain, eye, gastrointestinal tract, or the peripheral nervous system. In some cases, a peptide of the disclosure activates or deactivates an ion channel from heart, renal tissue, retina, cancer or tumor, gastrointestinal tract, epithelia, neural tissue, central and peripheral nervous system, cartilage, immune system (e.g., T lymphocyte, memory T cells), smooth muscle, skeletal muscle, cancer or tumor.

In some cases, commercial product ziconatide, which is a synthetic form of a w-conotoxin, targets and inhibits Cav2.2 for pain management. Ziconatide can also target and inhibit Nav1.7 to decrease sensitivity to pain or target NR2A to exert a neuroprotective effect. In some embodiments, ziconatide can be co-administered with a peptide of this disclosure. In some embodiments, conserved residues and/or key residues at the ion channel binding interface of ziconatide are incorporated in peptides of this disclosure to yield peptides that can target Cav2.2, Nav1.7, or NR2A selectively.

In some embodiments, a peptide disclosed herein is used to activate or inhibit an ion channel associated with autoimmune disease, such as inhibition of Kv1.3 channels in T cells to treat autoimmune diseases such as rheumatoid arthritis, multiple sclerosis, and type-1 diabetes mellitus. In some embodiments, such peptide is used to treat Lambert-Eaton Myasthenic Syndrome (LEMS), which is a disorder of neuromuscular transmission wherein antibodies are directed to presynaptic voltage-gated calcium channels resulting in muscle weakness. LEMS patient-derived IgGs have been shown to result in inhibiting P- and Q-type calcium currents. A peptide of this disclosure can be used to activate calcium ion channels or to counteract the effects of abnormal antibodies found in LEMS patients, or to increase the calcium influx in cells aftected by abnormal IgGs found in LEMS patients.

Antibodies directed to voltage-gated potassium channels are associated with various autoimmune diseases involving the CNS and peripheral nervous system, including, but not limited to, acquired neuromyotonia (NMT), also known as Isaac's syndrome, wherein antibodies against voltage-gated potassium channels prevent membrane re-polarization, increase acetylcholine release, and prolong action potentials. Hyperexcitability of peripheral nervous system can co-exist with CNS effects, and can results in seizures, sleep disturbances, and behavior changes. Other autoimmune diseases associated with voltage-gated potassium channels include crampfasciculation syndrome, limbic encephalitis (LE) and Morvan's syndrome (MoS). Antibodies against Kv1.2 and Kv1.6 can result in NMT and MoS, whereas antibodies against Kv1.1 can result in LE. Antibodies that target Kir4.1, such as the extracellular loop of Kir4.1, are associated with MS and, in some cases, neurologic diseases. In such patients, antibodies against Kir4.1 can result in impaired axon myelination and tissue damage. In some embodiments, a peptide of this disclosure selectively targets the E3 domain of Kv1.2 or Kv3.1 to activate or inhibit the ion channel in vivo, such as neuronal cells. In some embodiments, a peptide of this disclosure selectively targets Nav1.5, or an extracellular loop or domain in Nav1.5.

In some embodiments, a peptide disclosed herein is effective in modulating ion channel activity. Mechanisms of action for modulating ion channel activity can include blocking ion permeation pathway and/or modulating ion channel gating.

Diseases associated with Kv ion channels include autoimmune disease in the CNS and peripheral nervous system, acquired neuromyotonia, peripheral nerve hyperexcitability, seizures, sleep disturbance, behavioral changes, crampfasciculation syndrome, limbic encelphalitis, and Morvan's syndrome. Kv1.1 can be associated with episodic ataxia type 1, epilepsy, neuropathic pain, irritable bowl syndrome (IBS), and limbic encephalitis. Kv1.2 can be associated with IBS, (anti-) restore consciousness during anesthesia, acquired neuromyotonic, Morvan's syndrome. Kv1.3 can be implicated in immunosuppression, T cell-mediated autoimmune diseases, e.g., type-1 diabetes mellitus, rheumatoid arthritis, multiple sclerosis, asthma, obesity, insulin resistance, longevity, metabolism, bone resorption, psoriasis, Hashimoto's thyroiditis, Sjorgen's syndrome, systemic lupus erythematosis, autoimmune glumerlonephritis, contact dermatitis, and tranpslantation. Cav can be associated with hypertension, pain, epilepsy, and neuropathic pain. Such Kv-ion channel related diseases can be treated using any one of the following peptides: SEQ ID NO: 2, 6, 12, 19, 20, 21, 26, 29, 33, 39, 40, 42, 46, 52, 59, 60, 61, 66, 69, 73, 79, and 80. Any one of the following peptides can be used to treat a disease associated with Kv1.3: SEQ ID NO: 2, 6, 12, 19, 20, 21, 26, 29, 33, 39, 40, 42, 46, 52, 59, 60, 61, 66, 69, 73, 79, and 80. In some embodiments, a peptide disclosed herein is more selective for Kv1.3 over Kv1.1 and Kv1.2 by at least 10 fold, at least 50 fold, at least 100 fold, at least 500 fold, or at least 1000 fold.

Cav2.2 can be associated with episodic ataxia type 2, familial hemiplegic migraine, spinocerebellar ataxia type 6, ataxia, migraine, and epilepsy. Cav2.2 can be associated with severe pain and neuropathic pain. Such calcium ion channel related diseases can be treated using any one of the following peptides: SEQ ID NO: 7 and 47.

hERG (Kv11.1) can be associated with long QT syndrome, short QT syndrome, arrhythmia, metastasis, or cancer (e.g., breast, cervical, neuroblastoma, hepatocellular, prostate, colon, squamous cell lung, mammaary gland adenocarcinoma, leukemia, glioma, endometrial, CLL, AML, glioblastoma), immunomodulation, angina, arrhythmia, autoimmune disease, and IBS. Nav channels can be implicated in local anesthesia, epilepsy, bipolar, amyotrophic lateral sclerosis, seizures, pain, antidysrhythmics, anticoonvulsants, partial onset seizures, diabetic neuropathic pain. Such hERG ion channel related diseases can be treated using any one of the following peptides: SEQ ID NO: 7, 14, 17, 47, 54, and 57.

Nav1.5 (TP1 or TP2) can be associated with long QT syndrome, arrhythmia, progressive familiar heart block type I, Brugada syndrome (idiopathic ventricular arrhythmia), metastasis, cancer, breast cancer, prostate cancer; use for analgesic, angina, GI motility disorders, IBS, GI functional disorders, achalasia, gastroparesis, intestinal pseudo-obstruction, slow transit constipation, functional dyspepsia, and diabetic gastroparesis. Such Nav1.5 ion channel related diseases can be treated using any one of the following peptides: SEQ ID NO: 7 and 47.

Nav1.7 (TP1 or TP2) can be associated with erythromelalgia, paroxysmal extreme pain disorder, congenital indifference to pain, pain in patients with congenital rythromelalgia, cancer, metastasis, breast cancer, prostate cancer; use for analgesic, and angina. NR2A or NMDA receptors/channels can be associated with epilepsy, stroke, Alzheimer's disease, Parkinson's disease, stroke, and neuropathic painc. Such Nav1.7 ion channel related diseases can be treated using any one of the following peptides: SEQ ID NO: 7, 18, 31, 47, 57, and 71.

In some embodiments, a peptide of this disclosure can bind to, interact with, modulate, antagonize, or agonize any of the ion channels in TABLE 2, adapted from Table 1 of Sun, H. and Li, M., Acta Pharmacologica Sinica (2013) 34: 199-204, which is incorporated herein by reference. In some cases, the peptide can interact with the ion channel directly or in conjugation with an active agent described herein.

TABLE 2
Ion Channel Targets Associated With Autoimmune Diseases from Sun et al.

| Ion Channel | | |
|---|---|---|
| Family | Ion Channel | Cell Types Tested |
| Voltage-gated | Kv1.2; | HEK-293; NG108-15; Central medial thalamus |
| potassium | E3 extracellular loop in domain I | |
| channels | Kv3.1; | HEK-293; NG108-15; Primary oligodendrocyte |
| | E3 extracellular loop in domain I | progenitor cell |
| | Kv10.1; | HEK-293; Neuroblastoma; SH-SY5Y; Breast |
| | Fusion of E3 extracellular loop in | carcinoma; MDA-MB-435s; NCI-ADR; |
| | domain I and tetramerizing | Melanoma; HT144; C8161; SKMel2; Ovarian |
| | coiled-coil | carcinoma; SKOV3; SKOV6; OVCAR-3; |
| | | OVCAR-8; Cervical carcinoma HeLa; Pancreas |
| | | carcinoma; BxPC3; Colon carcinoma; HT29; |
| | | Fibrosarcoma; HT1080; Breast cancer; MDA- |
| | | MB-435s; xenograft; Pancreatic cancer; |
| | | xenograft; PAXF1657 Acute myeloid; HEL; |
| | | leukemia; UT-7; K562; PLB-985; Primary cells |
| Voltage-gated | Nav; | Sciatic nerve fibers; optic nerve fibers; cardiac |
| sodium channels | Extracellular domain | purkinje fibers; myosacs |
| | Nav; | Sciatic nerve firbers; optic nerve fibers; HEK- |
| | E2 or E3 extraceullary loop in | 293; EBNA-293 |
| | domain I | |
| Voltage-gated | L-type | BC3H1 myocytes |
| calcium channels | Extracellular domain; | |
| | α_{1D}; | Dorsal root ganglion; |
| | C-terminal to the pore-forming | Cardiac myocytes |
| | region between S1 and S2 in | |
| | domain IV | |
| | N and P/Q-type; | Cerebellar granule neurons; HEK293; Purkinje |
| | E3 extracellular loop | cell soma; Cerebellum |
| | P type; | Small-cell lung carcinoma |
| | E3 extracellular loop | |
| TRP Channels | TRPC1; | Platelets and vascular endothelial |
| | E3 extracellular loop | cells; Vascular smooth muscle cells; Bovine |
| | | aortic endothelial cells |
| | TRPC5; | HEK293; CHO; Cerebral arterioles; Pial |
| | E3 extracellular loop | arterioles |
| | TRPM3; | HEK-293 |
| | E3 extracellular loop | |
| | TRPV1; | CHO; HEK-293 |
| | E3 extracellular loop | |

In some embodiments, a peptide of this disclosure can bind to, interact with, modulate, antagonize, or agonize any of the ion channel regions listed in TABLE 2, or any of the ion channels in the diseased cell types in TABLE 2, such as neuroblastoma, breast carcinoma, melanoma, ovarian carcinoma, cervical carcinoma, pancreas carcinoma, breast cancer, pancreatic cancer, acute myeloid, small cell lung carcinoma, or leukemia. In some embodiments, a peptide of this disclosure can bind to, interact with, modulate, antagonize, or agonize Kv10.1 ion channel to reduce tumor growth, reduce proliferation and migration of cancerous cells, or increase cell death. In some cases, such peptides bind to E3 extracellular loop in domain I and/or the tetramerizing coiled-coil of Kv10.1 to reduce tumor growth. In some embodiments, a peptide of this disclosure can bind to, interact with, modulate, antagonize, or agonize a calcium ion channel to reduce tumor growth associated with small cell lung carcinoma. In some cases, such peptides bind to E3 extracellular loop in a P-type calcium ion channel to slow cancer cell growth associated with small cell lung carcinoma.

In some embodiments, a peptide of this disclosure can bind to, interact with, modulate antagonize, or agonize any of the below ion channels in TABLE 3 reproduced from Table 1 of Bagal et al. (J Med Chem. 2013 Feb. 14; 56(3):593-624.), which is incorporated herein by reference.

TABLE 3
Ion Channel-Related Diseases from Bagal et al.

| Ion Channel | | Gain (G) or Loss (L) | |
|---|---|---|---|
| Family | Channel | of Function | Ion-Channel Related Disease |
| Kᵢᵣ | Kir1.1 | L | Bartter's syndrome |
| | Kir2.1 | L | Andersen's syndrome |
| | Kir6.2 | L | Congenital hyperinsulinism |
| | | G | Neonatal diseases |
| Kᵥ | SUR2 | L | Dilated cardiomyopathy |
| | Kv1.1 | L | Episodic ataxia type 1 |
| | KCNQ1 | L | Long QT syndrome |
| | | G | Short QT syndrome |
| | KCNQ2 | L | Benign neonatal febrile convulsions |
| | KCNQ4 | L | Nonsyndromic deafness |
| | hERG | L | Long QT syndrome |
| | | G | Short QT syndrome |
| TRP | TRRP2 | | Polycystic kidney disease |
| | TRPA1 | G | Familial episodic pain syndrome |
| | TRPC6 | G | Focal segmental glomerulosclerosis |
| CNG | CNGA1 | L | Retinitis pigmentosa |
| K_{Ca} | BK | G | Epilepsy |
| Naᵥ | Naᵥ1.1 | G | Epilepsy |
| | | L | Severe myoclonic epilepsy |
| | Naᵥ1.5 | G | Long QT syndrome |
| | Naᵥ1.6 | L | Cerebellar ataxia |
| | Naᵥ1.7 | G | Erythromelalgia, paroxysmal extreme |
| | | | pain disorder |
| | | L | Congenital indifference to pain |
| | Naᵥ2.1 | G | Benign familial neonatal seizures |
| Caᵥ | Caᵥ1.2 | G | Timothy syndrome |
| | Caᵥ2.1 | L | Episodic ataxia type 2 |
| Glycine receptors | GLRA1 | L | Stiff baby syndrome |
| GABA | GABA_{A} | L | Juvenile myoclonic epilepsy |
| AChR | CHRNA4 | L | Autosomal dominant nocturnal frontal |
| | | | lobe epilepsy |

In some embodiments, a peptide of this disclosure can bind to, interact with, modulate antagonize, or agonize any of the ion channels in TABLE 3. In some embodiments, a peptide or composition comprising a peptide of this disclosure is used to treat, ameliorate, and/or prevent any one of the diseases listed in TABLE 3.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a disease listed in TABLE 3 by modulating the activity of an ion channel associated with the disease.

In some embodiments, a peptide of this disclosure is conjugated to any one of the ion channel-targeting drugs in TABLE 4 reproduced from Table 2 of Bagal et al. (J Med Chem. 2013 Feb. 14; 56(3):593-624.), which is incorporated herein by reference. In some embodiments, a peptide of this disclosure binds to, interacts with, modulates, or functions as an antagonist or an agonist with any one of the target ion channels of TABLE 4.

TABLE 4
Ion Channel Targeting Drugs from Bagal et al.

| Ion Channel- | | |
|---|---|---|
| Targeting Drug | Target Channel | Disease Target |
| Verapamil | L-type Caᵥ | Hypertension |
| Diltiazem | L-type Caᵥ | Hypertension |
| Amlodipine | L-type Caᵥ | Hypertension |
| Nifedipine | L-type Caᵥ | Hypertension |
| Gabapentin | Caᵥ (α2δ) | Pain |
| Pregabalin | Caᵥ (α2δ) | Pain |
| Sotalol | hERG | Arrhythmia |
| Flecainide | Naᵥ1.5 | Arrythmia |
| Ziconotide | Caᵥ2.2 | Severe Pain |
| Lidocaine | Naᵥ | Local Anesthetic |
| Bupivacaine | Naᵥ | Local Anesthetic |
| Lamotrigine | Naᵥ | Epilepsy, bipolar |
| Riluzole | Naᵥ | Amyotrophic lateral sclerosis |
| Phenytoin | Naᵥ | Epilepsy |
| Lacosamide | Naᵥ | Seizure and Pain |
| Carbamazepine | Naᵥ | Epilepsy |
| Varenicline | nACHR | Smoking cessation |
| Flupirtine | KCNQ2/3 | Epilepsy |
| Retigabine | KCNQ2/3 | Epilepsy |
| Diazepam | GABAₐ | Depression |

In some embodiments, a peptide of this disclosure is conjugated to any one of the ion channel-targeting drugs or an analogue or derivative thereof in TABLE 4. In some embodiments, a peptide of this disclosure is used in a combination therapy and/or is co-administered with any one of the drugs or an analogue or derivative thereof in TABLE 4. In some embodiments, a peptide of this disclosure is used to treat or ameliorate any one of the disease targets in TABLE 4. In some embodiments, a peptide comprising the sequence of any of SEQ ID NO: 1-SEQ ID NO: 80, and any peptide derivative or peptide conjugates, such as a peptide-active agent conjugate, as described herein, can be used to treat a disease listed in TABLE 4 by modulating the activity of an ion channel associated with the disease.

In some embodiments, a peptide of this disclosure, e.g., SEQ ID NO: 1-SEQ ID NO: 80, is used to target an ion channel in vivo to treat and/or ameliorate any of the following diseases: Bartter's syndrome, Andersen's syndrome, congenital hyperinsulinism, neonatal diseases, dilated cardiomyopathy, episodic ataxia type 1 or type 2, long QT syndrome, short QT syndrome, benign neonatal febrile convulsions, nonsyndromic deafness, polycystic kidney disease, familial episodic pain syndrome, focal segmental glomerulosclerosis, retinitis pigmentosa, epilepsy, severe myoclonic epilepsy, cerebellar ataxia, erythromelalgia, paroxysmal extreme pain disorder, congenital indifference to pain, benign familial neonatal seizures, timothy syndrome, stiff baby syndrome, juvenile myoclonic epilepsy, autosomal dominant nocturnal frontal lobe epilepsy, seizure and pain, depression, hypertension, arrhythmia, and amyotrophic lateral sclerosis. In some embodiments, a patient is diagnosed or has a risk of developing any one of the ion channel-related diseases, disorders, or conditions, and is treated or prescribed or administered a pharmaceutical composition comprising a peptide of the disclosure that selectively binds to, interacts with, modulates, or functions as an antagonist or agonist to the ion channel relevant to the disease, or the ion channel in a target organ, tissue, or cell types, e.g., brain, CNS, kidney, cartilage, heart, GI system, lymphocytes, memory T cells, or a cell type relevant to an autoimmune disease.

In some embodiments, peptides of this disclosure that home, target, are directed to, migrate to, are retained by, accumulate in, or bind to specific regions, tissues, structures or cells of the kidney, eye (e.g., retina), CNS, brain, peripheral nervous system, skeletal muscle, gastrointestinal (GI) system, cartilage, and the heart with different degrees of efficiency.

In some embodiments, a peptide of this disclosure binds to, interacts with, modulates, or functions as an antagonist or an agonist with any one of the target ion channels in TABLE 5 reproduced from Table 1 of Kale et al. (Biochim. Biophys. Acta. 2015 October; 1848(10 Pt B):2747-55), which is incorporated herein by reference.

TABLE 5
Ion Channels Involved in Cancers f rom Kale et al.

| | Ion Channels/Transporters Involved in Cancers |
|---|---|
| Hallmarks of Cancer | (I)-Increase; (D)-Decrease |
| Cell growth | Cys-loop, cationic Ca2+ permeable: nAChR α7 (I) |
| | Voltage gated Ca2+ (Cav): Cav1 L-type (I), Cav2.3 R-type (I), |
| | Cav3.2 T-type (I) |
| | Voltage gated K+ (Kv): Kv10.1 (I), Kv11.1 (I), Kv1.3 (I) |
| | Ca2+ activated K+ (KCa): KCa3.1 |
| | Inwardly rectifying K+ (Kir): Kir3.1 (I), Kir6.1 (I) |
| | Background K+ (K2P): K2P2.1 (I), K2P9.1 (I) |
| | TRP: TRPC6 (I), TRPV6 (I), TRPM7 (I) |
| Insensitivity to antigrowth signals | Purinergic, cationic Ca2+ permeable: P2X5/11 (D) |
| | Voltage gated K+ (Kv): Kv11.1 (I) |
| | Ca2+ activated K+ (KCa): KCa1.1 |
| | TRP, cationic Ca2+ permeable: TRPC1, TRPC4 (D) |
| Evasion of apoptosis | Background K+ (K2P): K2P9.1 (D) |
| | SOC, Ca2+ selective: Orai1 (D) |
| | TRP, cationic Ca2+ permeable: TRPV6 (D), TRPM2 (D) |
| | Cl− channels: CIC-3 (I) |
| Limitless replicative potential | Voltage gated Ca2+ (Cav): Cav1 (L-type) (I) |
| Angiogenesis | Voltage gated K+ (Kv): Kv10.1 (I), Kv11.1 (I) |
| | Ca2+ activated K+ (KCa): KCa1.1 and KCa3.1 (I) |
| | SOC, Ca2+ selective: Orai1 (I) |
| | TRP, cationic Ca2+ permeable: TRPC3 (I), TRPC4 (I), TRPC6 (I) |
| Metastasis | Voltage gated Na+ (Nav): Nav1.5 (I), Nav1.7 (I) |
| | Voltage gated Ca2+ (Cav): Cav3.1 (I) |
| | Voltage gated K+ (Kv): Kv11.1 (I) |
| | Ca2+ activated K+ (KCa): KCa1.1 (I), KCa2.3 (I), KCa3.1 (I) |
| | Inwardly rectifying K+ (Kir): Kir3.1 |
| | SOC, Ca2+ selective: Orai1 (I) |
| | TRP, cationic Ca2+permeable: TRPC1 (I) |
| | TRP, cationic Ca2+ permeable: TRPM1 (D) |
| | Na+ non-voltage-gated, DEG-related: ENaCα, ENaCγ, ASIC1 (I), |
| | ASIC2 (D) |
| | Cl− channels: CIC-3 (I) |

In some embodiments, a peptide of this disclosure is used to treat a cancer having any one of the hallmarks described in TABLE 5. In some embodiments, a peptide of this disclosure is used to modulate any one of the ion channels in TABLE 5 to treat cancer.

In some embodiments, a peptide of this disclosure binds to, interacts with, modulates, or functions as an antagonist or an agonist with any one of the target ion channels in TABLE 6 reproduced from Table 2 of Kale et al. (Biochim. Biophys. Acta. 2015 October; 1848(10 Pt B):2747-55), which is incorporated herein by reference.

TABLE 6
Ion Channels Involved in Cancers and Potential Drug Modulators from Kale et al.

| Ion Channel | Class of Channels | | |
|---|---|---|---|
| Family | Involved in Cancer | Cancer Type | Potential Drugs |
| Cys-loop, | nAChR α7 | Small cell lung carcinoma | Memantine |
| cationic Ca2+ | | | α Conotoxin |
| permeable | | | α Bungarotoxin |
| Purinergic, | P2X5/11, P2X7 | Bladder, prostate | ATP |
| cationic Ca2+- | | | |
| permeable | | | |
| Voltage-gated | Nav1.5, Nav1.7 | Breast, prostate | CNV1014802, TV-45070, PF- |
| Na+ (Nav) | | | 05089771, DSP-2230, |
| | | | Ranolazin, Riluzole |
| Voltage-gated | Cav1 (L-type), Cav2.3 | Lung, ovarian, | T-type: Mibefradil (T-Type), |
| Ca2+ (Cav) | (R-type) | esophageal carcinoma, | Pimozide, Penfluridol, |
| | Cav3.1 (T-Type), | prostate, fribrosarcoma, | KYS05041, TH-1177, L-type: |
| | Cav3.2 (T-Type) | breast, glioma, | Derivatives of dihydropyridine |
| | | neuroblastoma, | (amlodipine, felodipine) |
| | | colorectal, gastric, | |
| | | acutemyelogenous | |
| | | leukemia, retinoblastoma | |
| Voltage-gated | Kv10, Kv11, Kv1.3 | Breast, neuroblastoma, | Margatoxin, Verapamil, TEA, |
| K+ (Kv) | | cervical, breast, | Tamoxifen, E-4031, |
| | | hepatocellular carcinoma, | Kaliotoxin, ShK-186, |
| | | prostate carcinoma, colon | Astemizole |
| | | carcinoma, squamous cell | |
| | | lung carcinoma, colon | |
| | | carcinoma, mammary | |
| | | gland adenocarcinoma, | |
| | | neuroblastoma | |
| Ca2+ activated | KCa1.1, KCa2.3, | Breast, cervical, ovarian, | Iberiotoxin, Charybdotoxin, |
| K+ (KCa) | KCa3.1 | glioma, melanoma | Clotrimazole |
| Inwardly | Kir3.1, Kir6.1 | Breast, lung | Quinidine, linogliride, Barium |
| rectifying K+ | | | |
| (Kir) | | | |
| Background K+ | K2P2.1, K2P9.1 | Prostate, breast, glioma | Vernakalant |
| (K2P) | | | |
| SOC, Ca2+ | Orai1/STIM1 | Prostate, breast | Ophiobolin |
| selective | | | |
| TRP, cationic | TRPC1, TRPC3, | Prostate, fibrosarcoma, | Activators: TRPM2:ADP- |
| Ca2+ permeable | TRPC4, TRPC6, | hepatocellular carcinoma, | Ribose |
| | TRPV1, TRPV6, | gastric, breast | TRPM8: Menthol, icilin, |
| | TRPM1, TRPM2, | | geraniol, eucalyptol |
| | TRPM7, TRPM8 | | TRPV1: Capsaicin, |
| | | | anandamide, resiniferatoxin, |
| | | | ethanol |
| | | | Inhibitors: TRPM2: |
| | | | Clotrimazole |
| | | | TRPM8: SB-452533 |
| | | | TRPV1: Iodoresiniferatoxin, |
| | | | capsazepine |
| Na+ non-voltage- | ENaCα, ENaCγ, | Glioblastoma, Glioma | Amiloride |
| gated, DEG- | ASIC1, ASIC2 | | Triamterene |
| related | | | |
| Cl− channels | ClC-3 | Prostate | Chlorotoxin |
| | | Gliomas | Tamoxifen |

In some embodiments, a peptide of this disclosure is used to treat a cancer described in TABLE 6. In some embodiments, a peptide of this disclosure is conjugated to or used in conjunction with one of the anti-cancer drugs in TABLE 6.

In some embodiments, a peptide of this disclosure, e.g., SEQ ID NO: 1-SEQ ID NO: 80, is used to target an ion channel in vivo to treat and/or ameliorate any one of the following cancers: prostate cancer, glioma, glioblastoma, gastric cancer, hepatocellular carcinoma, breast cancer, lung cancer, cervical cancer, fibrosarcoma, lung cancer, ovarian cancer, esophageal carcinoma, neuroblastoma, colorectal cancer, acutemyelogenous leukemia, retinoblastoma, small cell lung cancer, and carcinoma. In some embodiments, a peptide of this disclosure, e.g., SEQ ID NO: 1-SEQ ID NO: 80, bind to any one of the channels listed in TABLE 6 and modulates its activity, including inhibition or activation.

In some embodiments, a peptide of this disclosure binds to, interacts with, modulates, or functions as an antagonist or an agonist with any one of the target ion channels in TABLE 7 reproduced from Table 1 of Hubner et al. (Hum Mol Genet. 2002 Oct. 1; 11(20):2435-45.), which is incorporated herein by reference.

TABLE 7
Ion Channel Diseases from Hübner et al.

| | | |
|---|---|---|
| | Channel-Forming | |
| Channel | Unit/Ligand | Disease |

Cation Channels:

| | | |
|---|---|---|
| CHRNA1/ACHRA | α, ACh | Myasthenia congenita |
| CHRNA4 | α, ACh | Autosomal dominant nocturnal frontal lobe epilepsy |
| CHRNB2 | β, ACh | Autosomal dominant nocturnal frontal lobe epilepsy |
| Polycystin-2 | α | Autosomal dominant polycystic kidney disease |
| | | (ADPKD) |
| CNGA3 | α, cGMP | Achromatopsia 2 (color blindness) |
| CNGB1 | β, cGMP | Autosomal recessive retinitis pigmentosa |
| CNGB3 | β, cGMP | Achromatopsia 3 |

Sodium Channels:

| | | |
|---|---|---|
| Nav1.1 | α | Generalized epilepsy with febrile seizures (GEFS+) |
| Nav1.2 | α | Generalized epilepsy with febrile and afebrile |
| | | seizures |
| Nav1.4 | α | Paramyotonia congenita, potassium aggressive |
| | | myotonia, hyperkalemic periodic paralysis |
| Nav1.5 | α | Long-QT syndrome, progressive familial heart block |
| | | type I, |
| | | Brugada syndrome (idiopathic ventricular |
| | | arrhythmia) |
| SCN1B | β | Generalized epilepsy with febrile seizures (GEFS+) |
| ENaCα | α | Pseudohypoaldosteronism type 1 (PHA1) |
| ENaCβ | β | PHA1, Liddle syndrome (dominant hypertension) |
| ENaCγ | γ | PHA1, Liddle syndrome (dominant hypertension) |

Potassium channels:

| | | |
|---|---|---|
| Kv1.1 | α | Episodic ataxia with myokymia |
| KCNQ1/KvLQT1 | α | Autosomal dominant long-QT syndrome (Romano- |
| | | Ward); Autosomal recessive long-QT syndrome with |
| | | deafness (Jervell-Lange-Nielsen) |
| KCNQ2 | α | BFNC (epilepsy), also with myokymia |
| KCNQ3 | α | BFNC (epilepsy) |
| KCNQ4 | α | DFNA2 (dominant hearing loss) |
| HERG/KCNH2 | α | Long-QT syndrome |
| Kir1.1/ROMK | α | Bartter syndrome (renal salt loss, hypokalemic |
| | | alkalosis) |
| Kir2.1/IRK/KCNJ2 | α | Long-QT syndrome with dysmorphic features |
| | | (Andersen syndrome) |
| Kir6.2/K_{ATP} | α | Persistent hyperinsulinemic hypoglycemia of infancy |
| | | (PHHI) |
| SUR1 | β | PHHI |
| KCNE1/MinK/ISK | β | Autosomal dominant long-QT syndrome (Romano- |
| | | Ward); Autosomal recessive long-QT syndrome with |
| | | deafness (Jervell-Lange-Nielsen) |
| KCNE2/MiRP1 | β | Long-QT syndrome |
| KCNE3/MiRP2 | β | Periodic paralysis |

Calcium channels:

| | | |
|---|---|---|
| Cav1.1 | α | Hypokalemic periodic paralysis, malignant |
| | | hyperthermia |
| Cav1.4 | α | X-linked congenital stationary night blindness |
| Cav2.1 | α | Familial hemiplegic migraine, episodic ataxia, |
| | | spinocerebellar ataxia type 6 |
| RyR1 | α | Malignant hyperthermia, central core disease |
| RyR2 | α | Catecholaminergic polymorphic ventricular |
| | | tachycardia, arrhythmogenic right ventricular |
| | | dysplasia type 2 |

Chloride channels:

| | | |
|---|---|---|
| CFTR | α | Cystic fibrosis, congenital bilateral aplasia of vas |
| | | deferens |
| ClC-1 | α | Autosomal recessive (Becker) or dominant |
| | | (Thomsen) myotonia |
| ClC-5 | α | Dent's disease (X-linked proteinuria and kidney |
| | | stones) |
| ClC-7 | α | Osteopetrosis (recessive or dominant) |
| ClC-Kb | α | Bartter syndrome type III |
| Barttin | β | Bartter syndrome type IV (associated with |
| | | sensorineural deafness) |
| GLRA1 | α, glycine | Hyperekplexia (startle disease) |
| GABAα1 | α, GABA | Juvenile myoclonus epilepsy |
| GABAγ2 | γ, GABA | Epilepsy |

Gap junction channels:

| | |
|---|---|
| Cx26 | DFNA3 (autosomal dominant hearing loss); DFNB1 |
| | (autosomal recessive hearing loss) |
| Cx30 | DFNA3 |
| Cx31 | DFNA2 |
| Cx32 | CMTX (X-linked Charcot-Marie-Tooth neuropathy) |

Administration of any one of the compositions or peptides disclosed herein to a subject of interest, e.g., a human patient or an animal, can be done parenterally (e.g., intravenously, subcutaneously, intramuscularly), directly into the relevant tissue or organ, by inhalation, dermally, topically, or orally. Therapeutic agents or pharmaceutical compositions for any one of the diseases, conditions, and/or disorders disclosed herein can be injected directly locally into an affected area or tissue, or into specific structures within the affected area or tissue. In some embodiments, the lack of access to the target tissue can also lead to administration of doses that are higher than would be necessary if a drug could home, target, or be directed to, is retained by, and/or binds to a target region, tissue, structure or cell. Thus, treatment of disease or conditions can require the use of high concentrations of non-specific drugs.

In some embodiments, a pharmaceutical composition comprising a peptide of the disclosure herein is a patch, ointment, an oral formulation, or an injection. In some embodiments, a peptide of the disclosure herein functions as an agonist or an antagonist.

In some embodiments, attaching or conjugating a payload, such as a therapeutic agent, to an ion-channel-binding peptide that selectively targets an ion channel of interest with little or minimal off-target effects can decrease the concentration of the therapeutic agent necessary to achieve a therapeutic effect.

Specific and potent ion channel-binding peptides that are capable of targeting a tissue or cell type of interest can counteract the non-specificity of many treatments by selectively targeting and delivering compounds to specific regions, tissues, cells and structures. Such drugs can also be useful to modulate ion channels, protein-protein interactions, extracellular matrix remodeling (e.g., protease inhibition), and the like. Such targeted therapy can allow for lower dosing, reduced side effects or off-target effects, improved patient compliance, and improvement in therapeutic outcomes, which are advantageous for treatment of patients, especially for chronic or long-term use.

In some embodiments, a peptide or a composition comprising a peptide of this disclosure can target an ion channel selectively in a target tissue or cell type, including kidney cells; nerve cells across the blood brain barrier; nerve cell of the peripheral nervous system; cells of gastrointestinal tract, such as epithelial cells; cartilage, immune cells, such as T and B lymphocytes, and effector memory T cells; muscle cells, such as smooth muscle or skeletal muscle cells, and cancer cells.

In some embodiments, peptides can have a higher concentration in the relevant organ, tissue, or cell type than in other locations, such as blood or a non-target or off-target organ, tissue, or cell type. Such targeting by a peptide of this disclosure has one or more of the advatanges of reducing adverse side effect, allowing systemic administration, higher tolerance in subjects (e.g., a human, an animal, or a patient), and higher tolerance for chronic or long-term use or administration of the peptide or a composition thereof.

In some embodiments, a method of treating a disease comprises delivering a peptide having at least 80% sequence identity to SEQ ID NO: 1-SEQ ID NO: 80, or a functional fragment thereof, that binds to any one of the ion channels listed in TABLE 2-7 to a target tissue or cell. In some embodiments, a method of treating a disease comprises administering to a subject in need thereof a peptide having at least 80% sequence identity to SEQ ID NO: 1-SEQ ID NO: 80, or a functional fragment thereof, that inhibits or activates any one of the ion channels listed in TABLE 2-7. In some embodiments, a method of treating a disease comprises delivering a peptide that binds to an ion channel of a diseased cell. In some embodiments, a method of treating a disease comprising delivering a peptide having at least 80% sequence identity to SEQ ID NO: 1-SEQ ID NO: 80, or a functional fragment thereof, that binds to a voltage-gated channel, a ligand-gated channel, a ligand-activated channel, an inward rectifier channel, or a mechanosensitive ion channel; or a potassium channel, sodium channel, calcium channel, TRP channel, GABA channel, ionotrophic glutamate receptor channel, acetylcholine receptor, nicotinic receptor, 5-HT3 receptor, or chloride channel; or any one of Kv1.1, Kv1.2, Kv1.3, Cav2.1, Cav2.2, Nav1.5 (TP1), Nav1.5 (TP2), Nav1.7 (TP1), Nav1.7 (TP2), hERG, 5-HT3a, alpha-4 beta-2 nicotinic receptor, Kv2.1, TPV1, GABA, and Kir2.1 channels. In some embodiments, the peptide blocks or activates such channels in a target cell or tissue. In some embodiments, such peptide is administered to a subject in need thereof. In some embodiments, such peptide is conjugated to an active agent or a detectable label. In some embodiments, such peptide is administered to a subject to image or detect diseased cells or tissue. In some embodiments, detection of diseased cells or tissue is used in a surgical procedure to remove or to apply a therapeutic agent to the diseased cells or tissue. In some embodiments, such peptides are administered to a subject in need thereof before, after, and/or during a medical procedure to manage pain. In some embodiments, such peptides are conjugated to an active agent disclosed herein and deliver the active agent to a target cell or tissue. In some embodiments, the disease treated using such peptides can be any one of the ion channel-related diseases disclosed herein, including a neurological disorder, cancer, autoimmune disease, GI motility disorder, irritable bowel syndrome, inflammatory bowel disease, constipation, dyspepsia, acquired neuromyotonia, renal disorder, ocular disorder, retinal disease, epilepsy, migraine, ataxia, polycystic kidney disease, seizure, long or short QT syndrome, paralysis, pain, neuropathic pain, severe pain, need for local anesthesia, migraine, cystic fibrosis, Bartter syndrome, endocrine disorder, rheumatoid arthritis, type 1 diabetes mellitus, multiple sclerosis, psoriasis, lupus, asthma, obesity, insulin resistance, hypertension, stroke, Alzheimer's disease, arrhythmia, or bone disease, or cancer, including breast, cervical, neuroblastoma, hepatocellular, prostate, colon, squamous lung cell, mammary gland, and endometrial cancers, and adenocarcinoma, leukemia, glioma, CLL, AML, glioblastoma. In some embodiments, target tissue or cells, or diseased tissues or cells, that are targeted by such peptides include a tissue or cell from heart, kidney, retina, cancer, gastrointestinal tract, epithelia, neural tissue, cartilage, immune system, such as B and T lymphocytes, smooth muscle, skeletal muscle, and cancerous cells. Examples of neural tissue/cells include nerve cells of the central nervous system, nerve cells of the peripheral nervous system, motor neuron, Purkinje cells, GABAergic neurons, excitatory neurons, sensory neurons, and interneurons. In some embodiments, cells of the CNS include glial cells, oligodendrocytes, astrocytes, ependymal cells and microglia.

In some embodiments, a peptide having at least 80% sequence identity to SEQ ID NO: 1, 3, 4, 6, 7, 11-14, 16-20, 22, 31, 33, 39, 40, 41, 43, 44, 46, 47, 51-54, 56-60, 62, 71, 73, 79, and 80 can inhibit an ion channel selected from a group consisting of: Kv1.1, Kv1.2, Kv1.3, NR2A, Nav1.5, Nav1.7 (TP1), Nav1.7 (TP2), and hERG.

In some embodiments, a peptide of SEQ ID NO: 2, 14, 18, 19, 21, 26, 29, 39, 40, 42, 54, 58, 59, 61, 66, 69, 79, and 80 can inhibit an ion channel selected from a group consisting of Kv1.1, Kv1.2, and Kv1.3. In some embodiments, these peptides bind to the ion channel to result in <50% activity at peptide concentration of 20 μM and <80% activity at peptide concentration of 0.2 μM according to a ChanTest assay.

In some embodiments, a peptide of SEQ ID NO: 6, 19, 39, 46, 59, and 79 inhibits Kv1.1. In some embodiments, a peptide of SEQ ID NO: 1-4, 11-14, 18, 22, 31, 40, 41-44, 51-54, 58, 62, and 80 inhibits Kv1.2. In some embodiments, a peptide of SEQ ID NO: 2, 17, 20, 33, 40, 42, 57, 60, 73, and 80 inhibits Kv1.3. In some embodiments, a peptide of SEQ ID NO: 7 and 47 inhibits hERG. In some embodiments, a peptide of SEQ ID NO: 7 and 47 inhibits Nav1.5 or Nav1.7. In some embodiments, a peptide of SEQ ID NO: 7, 16, 18, 47, 58, and 57 inhibits Nav1.7 (TP1) or Nav1.7 (TP2). In some embodiments, a peptide of SEQ ID NO: 40 and 80 inhibits NR2A.

In some embodiments, a peptide having at least 80%, 85%, 90%, 95%, 97%, or 99% sequence identity to any one of SEQ ID NO: 6, 19, 39, 46, 59, and 79 inhibits Kv1.1. In some embodiments, the peptide having at least 80%, 85%, 90%, 95%, 97%, or 99% sequence identity to any one of SEQ ID NO: 1-4, 11-14, 18, 22, 31, 40, 41-44, 51-54, 58, 62, and 80 inhibits Kv1.2. In some embodiments, a peptide having at least 80%, 85%, 90%, 95%, 97%, or 99% sequence identity to any one of SEQ ID NO: 2 17, 20, 33, 40, 42, 57, 60, 73, and 80 inhibits Kv1.3. In some embodiments, peptide having at least 80%, 85%, 90%, 95%, 97%, or 99% sequence identity to any one of SEQ ID NO: 7 and 47 inhibits hERG. In some embodiments, a peptide having at least 80%, 85%, 90%, 95%, 97%, or 99% sequence identity to any one of SEQ ID NO: 7 and 47 inhibits Nav1.5 or Nav1.7. In some embodiments, a peptide having at least 80%, 85%, 90%, 95%, 97%, or 99% sequence identity to any one of SEQ ID NO: 7, 16, 18, 47, 58, and 57 inhibits Nav1.7 (TP1) or Nav1.7 (TP2). In some embodiments, a peptide having at least 80%, 85%, 90%, 95%, 97%, or 99% sequence identity to any one of SEQ ID NO: 40 and 80 inhibits NR2A.

In some embodiments, peptides disclosed herein can rescue phenotype of an ion channel having a gain of function mutation or a loss of function mutation. In some cases, peptides disclosed herein can rescue phenotype of an ion channel that is overexpressed in a target tissue or cell type. In some embodiments, peptides disclosed herein can rescue phenotype of an ion channel that is under-expressed in a target tissue or cell type. In some embodiments, peptides disclosed herein bind to an ion channel wherein activation or deactivation is associated with a disease state. In some cases, peptides disclosed herein can modulate an ion channel by inhibiting or activating the ion channel. In some cases, the peptide binds to the ion channel to induce a conformational change in the ion channel. In some cases, peptide binding to the ion channel blocks ion movement through the channel. In other cases, peptide binding to the ion channel enhances ion movement through the channel. In some cases, peptide binding to the ion channel blocks the ion channel from ligand interaction.

In some embodiments, the peptide binds to the ion channel to result in an inhibition of ion channel activity by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% as measured by a ScreenPatch Assay. In some embodiments, the peptide binds to the ion channel to result in an activation of ion channel activity by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% as measured by a ScreenPatch Assay.

In some embodiments, these peptides bind to the ion channels to result in <1%, <5%, <10%, <20%, <30%, <40%, <50%, <60%, <70%, <80%, <90%, or <95% ion channel activity at a peptide concentration of 20 μIV as measured by ChanTest assay. In some embodiments, a peptide of this disclosure binds to an ion channel to result in at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or at least 100% inhibition of ion channel activity. In some embodiments, a peptide of this disclosure binds to an ion channel to result in at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or at least 100% or more increase in ion channel activity.

In some embodiments, a peptide comprising the sequence of any of SEQ ID NO: 1-SEQ ID NO: 80, and any peptide derivative or peptide conjugates, such as a peptide-active agent conjugate, as described herein, can be used to treat a disorder of a region of the body or tissue or an intracellular compartment. In certain embodiments, the peptide can be used as a delivery scaffold for an active agent. For example, a peptide or peptide-active agent conjugate can be used to access and treat disorders of the gastrointestinal (GI) tract, lung, skin, cartilage, vaginal mucosa, or nasal mucosa. Peptides of this disclosure can be used to access and treat these disorders due to their enhanced stability in various biological environments, including low pH, protease-rich environments, acidic environments, reducing environments, or environments with varying temperatures.

A peptide comprising the sequence of any of SEQ ID NO: 1-SEQ ID NO: 80, and any peptide derivative or peptide-active agent conjugate as described herein, can be used to treat upper GI disease, cancers (e.g., throat, oral, esophageal cancer, salivary glands, tonsils, pharynx, adenosarcomas, oral malignant melanoma head, neck cancer, or sarcomas). A peptide comprising the sequence of any of SEQ ID NO: 1-SEQ ID NO: 80, and any peptide derivative or peptide-active agent conjugate as described herein, can be used to target diseases of the esophagus, stomach, the small intestine, the duodenum, the large intestine, and other parts of the GI tract. These diseases can include Crohn's disease, inflammatory bowel disease, irritable bowel syndrome, cancers such as colorectal cancer and stomach cancer, gastroesophageal reflux disease, ulcerative colitis, constipation, opioid-induced constipation, and infections, such as an infection caused by Helicobacter pylori.

A peptide comprising the sequence of any of SEQ ID NO: 1-SEQ ID NO: 80, and any peptide derivative or peptide-detectable agent conjugate as described herein, can be used to diagnose or image upper GI disease and cancers (e.g., throat, oral, esophageal cancer, salivary glands, tonsils, pharynx, adenosarcomas, oral malignant melanoma head, neck cancer, or sarcomas). A peptide comprising the sequence of any of SEQ ID NO: 1-SEQ ID NO: 80, and any peptide derivative or peptide-detectable agent conjugate as described herein, can be used to diagnose or image diseases of the esophagus, stomach, the small intestine, the duodenum, the large intestine, and other parts of the GI tract. These diseases can include Crohn's disease, inflammatory bowel disease, irritable bowel syndrome, cancers such as colorectal cancer and stomach cancer, gastroesophageal reflux disease, ulcerative colitis, constipation, opioid-induced constipation.

A peptide comprising the sequence of any of SEQ ID NO: 1-SEQ ID NO: 80, and any peptide derivative or peptide-active agent conjugate as described herein, can be used to treat upper chronic inflammatory lung diseases such as cystic fibrosis, chronic obstructive pulmonary disease (COPD), and emphysema, which are characterized by higher than normal levels of pulmonary proteases (e.g., neutrophil elastase, alpha-1 antitrypsin, secretory leucoprotease inhibitor, or elafin).

A peptide comprising the sequence of any of SEQ ID NO: 1-SEQ ID NO: 80, and any peptide derivative or peptide-detectable agent conjugate as described herein, can be used to diagnose or image upper chronic inflammatory lung diseases such as cystic fibrosis, chronic obstructive pulmonary disease (COPD), and emphysema, which are characterized by higher than normal levels of pulmonary proteases (e.g., neutrophil elastase, alpha-1 antitrypsin, secretory leucoprotease inhibitor, or elafin).

A peptide comprising the sequence of any of SEQ ID NO: 1-SEQ ID NO: 80, and any peptide derivative or peptide-active agent conjugate as described herein, can be used to treat eye or ocular diseases, disorders, or infections such as asacanthamoeba keratitis, blepharitis, CMV retinitis, conjunctivitis, corneal abrasion, dry eye syndrome, ocular herpes, fungal keratitis, trachoma, endophthalmitis, dacryostenosis, uveitis, Sjogren's syndrome, stye, ocular histoplasmosis syndrome, mycosis, toxoplasmosis, chlamydia, gonorrhea, bacterial keratitis, tuberculosis, leprosy, syphilis, hepatitis B, or infections caused by herpes simplex virus, epstein-barr virus, or Candida. In some embodiments, peptide comprising the sequence of any of SEQ ID NO: 1-SEQ ID NO: 80, and any peptide derivative or peptide-active agent conjugate as described herein, can be used to treat ocular surface disease, inflammation or injury of the eye, AMD, wet AMD, glaucoma, or retinopathy, or to promote wound healing of an eye.

A peptide comprising the sequence of any of SEQ ID NO: 1-SEQ ID NO: 80, and any peptide derivative or peptide-detectable agent conjugate as described herein, can be used to diagnose or image eye diseases such asacanthamoeba keratitis, blepharitis, CMV retinitis, conjunctivitis, corneal abrasion, dry eye syndrome, ocular herpes, fungal keratitis, trachoma, endophthalmitis, dacryostenosis, uveitis, Sjogren's syndrome, stye, ocular histoplasmosis syndrome, mycosis, toxoplasmosis, chlamydia, gonorrhea, bacterial keratitis, tuberculosis, leprosy, syphilis, hepatitis B, or infections caused by herpes simplex virus, epstein-barr virus, or Candida.

In therapeutic applications, the compositions can be administered to a subject already suffering from a disease or condition, in an amount sufficient to cure or at least partially arrest the symptoms of the disease or condition, or to cure, heal, improve, or ameliorate the condition. Such peptides described herein can also be administered to prevent (either in whole or in part), lessen a likelihood of developing, contracting, or worsening a condition. Amounts effective for this use can vary based on the severity and course of the disease or condition, previous therapy, the subject's health status, weight, and response to the drugs, and the calculations of the treating physician.

Multiple peptides described herein can be administered in any order or simultaneously. In some cases, multiple functional fragments of peptides derived from toxins or venom can be administered in any order or simultaneously. If simultaneously, the multiple peptides described herein can be provided in a single, unified form, such as an intravenous injection, or in multiple forms, such as subsequent intravenous dosages.

Types of cartilage diseases or conditions that can be treated with a peptide or peptide-active agent conjugate of the disclosure can include inflammation, pain management, anti-infective, pain relief, anti-cytokine, cancer, injury, degradation, genetic basis, remodeling, hyperplasia, or the like. Examples of cartilage diseases or conditions that can be treated with a peptide of the disclosure include Costochondritis, Spinal disc herniation, Relapsing polychondritis, Injury to the articular cartilage, any manner of rheumatic disease (e.g., Rheumatoid Arthritis (RA), ankylosing spondylitis (AS), Systemic Lupus Erythematosus (SLE or “Lupus”), Psoriatic Arthritis (PsA), Osteoarthritis, Gout, and the like), Herniation, Achondroplasia, Benign or non-cancerous chondroma, Malignant or cancerous chondrosarcoma, Chondriodystrophies, Chondromalacia patella, Costochondritis, Halus rigidus, Hip labral tear, Osteochondritis dssecans, Osteochondrodysplasias, Torn meniscus, Pectus carinatum, Pectus excavatum, Chondropathy, Chondromalacia, Polychondritis, Relapsing Polychondritis, Slipped epiphysis, Osteochondritis Dissecans, Chondrodysplasia, Costochondritis, Perichondritis, Osteochondroma, Knee osteoarthritis, Finger osteoarthritis, Wrist osteoarthritis, Hip osteoarthritis, Spine osteoarthritis, Chondromalacia, Osteoarthritis Susceptibility, Ankle Osteoarthritis, Spondylosis, Secondary chondrosarcoma, Small and unstable nodules as seen in osteoarthritis, Osteochondroses, Primary chondrosarcoma, Cartilage disorders, scleroderma, collagen disorders, Chondrodysplasia, Tietze syndrome, Dermochondrocorneal dystrophy of Francois, Epiphyseal dysplasia multiple 1, Epiphyseal dysplasia multiple 2, Epiphyseal dysplasia multiple 3, Epiphyseal dysplasia multiple 4, Epiphyseal dysplasia multiple 5, Ossified Ear cartilages with Mental deficiency, Muscle Wasting and Bony Changes, Periosteal chondrosarcoma, Carpotarsal osteochondromatosis, Achondroplasia, Genochondromatosis II, Genochondromatosis, Chondrodysplasia—disorder of sex development, Chondroma, Chordoma, Atelosteogenesis, type 1, Atelosteogenesis Type III, Atelosteogenesis, type 2, Pyknoachondrogenesis, Osteoarthropathy of fingers familial, Dyschondrosteosis-nephritis, Coloboma of Alar-nasal cartilages with telecanthus, Alar cartilages hypoplasia-coloboma-telecanthus, Pierre Robin syndrome-fetal chondrodysplasia, Dysspondyloenchondromatosis, Achondroplasia regional-dysplasia abdominal muscle, Osteochondritis Dissecans, Familial Articular Chondrocalcinosis, Tracheobronchomalacia, Chondritis, Dyschondrosteosis, Jequier-Kozlowski-skeletal dysplasia, Chondrodystrophy, Cranio osteoarthropathy, Tietze's syndrome, Hip dysplasia-ecchondromata, Bessel-Hagen disease, Chondromatosis (benign), Enchondromatosis (benign), Chondrocalcinosis due to apatite crystal deposition, Meyenburg-Altherr-Uehlinger syndrome, Enchondromatosis-dwarfism-deafness, premature growth plate closure (e.g., due to dwarfism, injury, therapy such as retinoid therapy for adolescent acne, or ACL repair), Astley-Kendall syndrome, Synovial osteochondromatosis, Severe achondroplasia with developmental delay and acanthosis nigricans, Chondrocalcinosis, Stanescu syndrome, Familial osteochondritis dissecans, Achondrogenesis type 1A, Achondrogenesis type 2, Achondrogenesis, Langer-Saldino Type, Achondrogenesis type 1B, Achondrogenesis type 1A and 1B, Type II Achondrogenesis-Hypochondrogenesis, Achondrogenesis, Achondrogenesis type 3, Achondrogenesis type 4, Chondrocalcinosis 1, Chondrocalcinosis 2, Chondrocalcinosis familial articular, Diastrophic dysplasia, Fibrochondrogenesis, Hypochondroplasia, Keutel syndrome, Maffucci Syndrome, Osteoarthritis Susceptibility 6, Osteoarthritis Susceptibility 5, Osteoarthritis Susceptibility 4, Osteoarthritis Susceptibility 3, Osteoarthritis Susceptibility 2, Osteoarthritis Susceptibility 1, Pseudoachondroplasia, Cauliflower ear, Costochondritis, Growth plate fractures, Pectus excavatum, septic arthritis, gout, pseudogout (calcium pyrophosphate deposition disease or CPPD), gouty arthritis, bacterial, viral, or fungal infections in or near the joint, bursitis, tendinitis, arthropathies, or another cartilage or joint disease or condition.

Types of cartilage diseases or conditions that can be diagnosed or imaged with a peptide-detectable agent conjugate of the disclosure can include inflammation, pain management, anti-infective, pain relief, anti-cytokine, cancer, injury, degradation, genetic basis, remodeling, hyperplasia, or the like. Examples of cartilage diseases or conditions that can be treated with a peptide of the disclosure include Costochondritis, Spinal disc herniation, Relapsing polychondritis, Injury to the articular cartilage, any manner of rheumatic disease (e.g., Rheumatoid Arthritis (RA), ankylosing spondylitis (AS), Systemic Lupus Erythematosus (SLE or “Lupus”), Psoriatic Arthritis (PsA), Osteoarthritis, Gout, and the like), Herniation, Achondroplasia, Benign or non-cancerous chondroma, Malignant or cancerous chondrosarcoma, Chondriodystrophies, Chondromalacia patella, Costochondritis, Halus rigidus, Hip labral tear, Osteochondritis dssecans, Osteochondrodysplasias, Torn meniscus, Pectus carinatum, Pectus excavatum, Chondropathy, Chondromalacia, Polychondritis, Relapsing Polychondritis, Slipped epiphysis, Osteochondritis Dissecans, Chondrodysplasia, Costochondritis, Perichondritis, Osteochondroma, Knee osteoarthritis, Finger osteoarthritis, Wrist osteoarthritis, Hip osteoarthritis, Spine osteoarthritis, Chondromalacia, Osteoarthritis Susceptibility, Ankle Osteoarthritis, Spondylosis, Secondary chondrosarcoma, Small and unstable nodules as seen in osteoarthritis, Osteochondroses, Primary chondrosarcoma, Cartilage disorders, scleroderma, collagen disorders, Chondrodysplasia, Tietze syndrome, Dermochondrocorneal dystrophy of Francois, Epiphyseal dysplasia multiple 1, Epiphyseal dysplasia multiple 2, Epiphyseal dysplasia multiple 3, Epiphyseal dysplasia multiple 4, Epiphyseal dysplasia multiple 5, Ossified Ear cartilages with Mental deficiency, Muscle Wasting and Bony Changes, Periosteal chondrosarcoma, Carpotarsal osteochondromatosis, Achondroplasia, Genochondromatosis II, Genochondromatosis, Chondrodysplasia—disorder of sex development, Chondroma, Chordoma, Atelosteogenesis, type 1, Atelosteogenesis Type III, Atelosteogenesis, type 2, Pyknoachondrogenesis, Osteoarthropathy of fingers familial, Dyschondrosteosis-nephritis, Coloboma of Alar-nasal cartilages with telecanthus, Alar cartilages hypoplasia-coloboma-telecanthus, Pierre Robin syndrome-fetal chondrodysplasia, Dysspondyloenchondromatosis, Achondroplasia regional-dysplasia abdominal muscle, Osteochondritis Dissecans, Familial Articular Chondrocalcinosis, Tracheobronchomalacia, Chondritis, Dyschondrosteosis, Jequier-Kozlowski-skeletal dysplasia, Chondrodystrophy, Cranio osteoarthropathy, Tietze's syndrome, Hip dysplasia-ecchondromata, Bessel-Hagen disease, Chondromatosis (benign), Enchondromatosis (benign), Chondrocalcinosis due to apatite crystal deposition, Meyenburg-Altherr-Uehlinger syndrome, Enchondromatosis-dwarfism-deafness, premature growth plate closure (e.g., due to dwarfism, injury, therapy such as retinoid therapy for adolescent acne, or ACL repair), Astley-Kendall syndrome, Synovial osteochondromatosis, Severe achondroplasia with developmental delay and acanthosis nigricans, Chondrocalcinosis, Stanescu syndrome, Familial osteochondritis dissecans, Achondrogenesis type 1A, Achondrogenesis type 2, Achondrogenesis, Langer-Saldino Type, Achondrogenesis type 1B, Achondrogenesis type 1A and 1B, Type II Achondrogenesis-Hypochondrogenesis, Achondrogenesis, Achondrogenesis type 3, Achondrogenesis type 4, Chondrocalcinosis 1, Chondrocalcinosis 2, Chondrocalcinosis familial articular, Diastrophic dysplasia, Fibrochondrogenesis, Hypochondroplasia, Keutel syndrome, Maffucci Syndrome, Osteoarthritis Susceptibility 6, Osteoarthritis Susceptibility 5, Osteoarthritis Susceptibility 4, Osteoarthritis Susceptibility 3, Osteoarthritis Susceptibility 2, Osteoarthritis Susceptibility 1, Pseudoachondroplasia, Cauliflower ear, Costochondritis, Growth plate fractures, Pectus excavatum, septic arthritis, gout, pseudogout (calcium pyrophosphate deposition disease or CPPD), gouty arthritis, bacterial, viral, or fungal infections in or near the joint, bursitis, tendinitis, arthropathies, or another cartilage or joint disease or condition.

In some embodiments, a peptide or peptide-active agent conjugate of this disclosure can be administered to a subject in order to target an arthritic joint. In other embodiments, a peptide or peptide-active agent conjugate of this disclosure can be administered to a subject in order to treat an arthritic joint.

In some embodiments, a peptide or peptide-detectable agent conjugate of this disclosure can be administered to a subject in order to diagnose or image an arthritic joint.

In some embodiments, the peptides of the present disclosure can be used to treat chondrosarcoma. Chondrosarcoma is a cancer of cartilage producing cells and is often found in bones and joints. It falls within the family of bone and soft-tissue sarcomas. In certain embodiments, administration of a peptide, peptide-active agent conjugate, or peptide-detectable agent conjugate of the present disclosure can be used to image and diagnose or target and treat a subject with chondrosarcoma. The subject can be a human or an animal.

In some embodiments, the peptides of the present disclosure are conjugated to one or more therapeutic agents. In further embodiments, the therapeutic agent is a chemotherapeutic, anti-cancer drug, or anti-cancer agent selected from, but are not limited to: anti-inflammatories, such as for example a glucocorticoid, a corticosteroid, a protease inhibitor, such as for example collagenase inhibitor or a matrix metalloprotease inhibitor (i.e., MMP-13 inhibitor), an amino sugar, vitamin (e.g., Vitamin D), and antibiotics, antiviral, or antifungal, a statin, and an immune modulator, In other embodiments, the therapeutic agent is any nonsteroidal anti-inflammatory drug (NSAID). The NSAID can be any heterocyclic acetic acid derivatives such as ketorolac, indomethacin, etodolac, or tolemetin, any propionic acid derivatives such as naproxen, any enolic acid derivatives, any anthranilic acid derivatives, any selective COX-2 inhibitors such as celecoxib, any sulfonanilides, any salicylates, aceclofenac, nabumetone, sulindac, diclofenac, or ibuprofen. In other embodiments, the therapeutic agent is any steroid, such as dexamethasone, budesonide, triamcinolone, cortisone, prednisone, rednisolone, triamcinolone hexacetonide, or methylprednisolone. In some embodiments, a treatment consists of administering a combination of any of the above therapeutic agents and a peptide-active agent conjugate, such as a treatment in which both a dexamethasone-peptide conjugate and an NSAID are administered to a patient. Peptides and peptide-active agent conjugates of the current disclosure that target the cartilage can be used to treat the diseases conditions as described herein, for example, any diseases or conditions including tears, injuries (i.e., sports injuries), genetic factors, degradation, thinning, inflammation, cancer or any other disease or condition of the cartilage or to target therapeutically-active substances to treat these diseases amongst others. In other cases, a peptide or a peptide-active agent conjugate of the disclosure can be used to treat traumatic rupture, detachment, chostochondritis, spinal disc herniation, relapsing and non-relapsing polychondritis, injury to the articular cartilage, osteoarthritis, arthritis or achondroplasia. In some cases, the peptide or peptide-active agent conjugate can be used to target cancer in the cartilage, for example benign chondroma or malignant chondrosarcoma, by contacting the cartilage by diffusion into chondrocytes and then having antitumor function, targeted toxicity, inhibiting metastases, etc. Additionally, a peptide-detectable agent conjugate can be used to label, detect, or image such cartilage lesions, including tumors and metastases amongst other lesions, which may be removed through various surgical techniques.

Peptides of the current disclosure that target the cartilage can be used to treat or manage pain associated with a cartilage injury or disorder, or any other cartilage or joint condition as described herein. The peptides can be used either directly or as carriers of active drugs, peptides, or molecules. For example, since ion channels are associated with pain and may be activated in disease states such as arthritis, peptides that interact with ion channels can be used directly to reduce pain. In another embodiment, the peptide is conjugated to an active agent with anti-inflammatory activity, in which the peptide acts as a carrier for the local delivery of the active agent to reduce pain.

In some embodiments, the peptides described herein provide a method of treating a cartilage condition of a subject, the method comprising administering to the subject a therapeutically-effective amount of a peptide comprising the sequence SEQ ID NO: 1-SEQ ID NO: 80, or any fragment thereof.

Venom or toxin derived peptide(s), peptides, modified peptides, labeled peptides, peptide-active agent conjugates and pharmaceutical compositions described herein can be administered for prophylactic and/or therapeutic treatments. In therapeutic applications, the composition can be administered to a subject already suffering from a disease or condition, in an amount sufficient to cure or at least partially arrest the symptoms of the disease or condition, or to cure, heal, improve, or ameliorate the condition. Such peptides described herein can also be administered to prevent (either in whole or in part), lessen a likelihood of developing, contracting, or worsening a condition. Amounts effective for this use can vary based on the severity and course of the disease or condition, previous therapy, the subject's health status, weight, response to the drugs, and the judgment of the treating physician. Venom or toxin derived peptide(s), peptides, modified peptides, labeled peptides, peptide-active agent conjugates and pharmaceutical compositions described herein can allow for targeted homing of the peptide and local delivery of any conjugate. For example, a peptide conjugated to a steroid allows for local delivery of the steroid, which is significantly more effective and less toxic than traditional systemic steroids. A peptide conjugated to an NSAID is another example. In this case, the peptide conjugated to an NSAID allows for local delivery of the NSAID, which allows for administration of a lower NSAID dose and is subsequently less toxic. By delivering an active agent to the joint, pain relief can be more rapid, may be more long lasting, and can be obtained with a lower systemic dose and off-site undesired effects than with systemic dosing without targeting.

In some embodiments, the present disclosure provides a method for treating a cancer, the method comprising administering to a subject in need thereof an effective amount of a peptide of the present disclosure.

Peptides of the current disclosure can be used to treat or manage pain associated with an ion channel-related disease or disorder. The peptides can be used either directly or as carriers of active drugs, peptides, or molecules. For example, since ion channels can be associated with pain and can be activated in disease states, peptides that interact with ion channels can be used directly to reduce pain. In another embodiment, the peptide is conjugated to an active agent with anti-inflammatory activity, in which the peptide acts as a carrier for the local delivery of the active agent to reduce pain.

In some embodiments, the peptides described herein provide a method of treating a kidney condition of a subject, the method comprising administering to the subject a therapeutically-effective amount of a peptide disclosed herein.

In some embodiments, the peptides described herein provide a method of treating a disease, the method comprising administering to the subject a peptide of any one of SEQ ID NO: 1-SEQ ID NO: 80 or a functional fragment thereof.

Gastrointestinal (GI) Diseases.

In some cases, a peptide can block, inhibit, or deactivate any of the chloride, potassium, sodium, voltage-gated, ligand-gated, mechanosensitive, and TRP ion channels in a cell or tissue of the GI tract. The peptide can block potassium channels and/or sodium channels. The peptide can block calcium or magnesium channels. In some embodiments, a peptide can binding to an ion channel to reduce activity of an ion channel that is overexpressed in a GI cell or tissue. In some embodiments, a peptide of this disclosure functions as an antagonist of an ion channel listed in any of TABLE 2-7. In some cases, a peptide can activate or increase ion channel activity of any one of the chloride, potassium, sodium, voltage-gated, ligand-gated, mechanosensitive, and TRP ion channels in a GI cell or tissue. The peptide can activate potassium channels and/or sodium channels. The peptide can activate calcium or magnesium channels. In some embodiments, a peptide can binding to an ion channel to increase activity of an ion channel that is under-expressed in a GI cell or tissue. In some embodiments, a peptide of this disclosure functions as an agonist of an ion channel listed in any of TABLE 2-7. GI cells or tissues include intestinal epithelium, smooth muscle cells, intestinal myofibroblasts, and peripheral nerve cells. In some embodiments, a peptide, a peptide conjugate, or a composition thereof interacts with an ion channel, including gap junction, to effect a change in gastrointestinal motility.

The peptides of the present disclosure can be used to treat, diagnose, or image a gastrointestinal (GI) disease, disorder, or infection, or to modulate gastrointestinal motility. Any one of the peptides of SEQ ID NO: 1-SEQ ID NO: 80 can be used to treat a gastrointestinal disease, disorder, or infection, or to modulate gastrointestinal motility. For example, any one of the peptides of SEQ ID NO: 1-SEQ ID NO: 80 can be administered alone or as a conjugate with an active agent or a detectable label.

The peptide can be recombinantly expressed or chemically synthesized. In some embodiments, the peptide can be fused with of chemically conjugated to an active agent or detectable agent to produce a peptide-active agent conjugate or peptide-detectable agent conjugate.

Because the peptides of this disclosure can be resistant to proteases, low pH, and/or reduction conditions found the environment of the GI tract, the peptides of this disclosure can remain intact in the GI tract long enough to have a therapeutic effect, to target a tissue, to accumulate in a tissue or cell, to deliver an active agent, to bind to, antagonize or agonize a receptor or enzyme or ion channel, to activate or block a biological pathway, to allow imaging, or to have another therapeutic or diagnostic effect. For example, linaclotide, which is a knotted peptide that is resistant to low pH and pepsin, can be orally administered and can agonize guanylate cyclase-C (for treatment of irritable bowel syndrome with constipation or chronic idiopathic constipation) in the gastrointestinal tract prior to being degraded in the intestinal lumen. As a result, linoclatide is not significantly systemically absorbed into the plasma, and therefore, sytemic side effects are avoided (see FDA label for Linzess (approved 2012)). Similarly to linaclotide, peptides of the disclosure can be used for treating diseases of the GI tract. The peptides or peptide-active agent conjugates can be orally administered to prevent or treat a GI infection, or a GI cancer. For example, peptides and/or peptide-active agent conjugates can be used to treat any one of the following gastrointestinal diseases, cancer, disorders or infections: infections caused norovirus, rotavirus, intestinal parasites (e.g., Entamoeba hystolytica, Trichomonas, Giardia, Bacteroides, Clostridium peptococcus, pinworm, Strongyloidiasis, Plasmodium falciparum, Cryptosporidium parvum, Cyclospora cayetanensis, Diphyllobothrium latum, Ascaris lumbricoides, Trichuris trichiura, Taenia solium, or Taenia saginata), Campylobacter, Clostridium botulinum, Clostridium perfringens, Escherichia coli, (including Shiga toxin-producing (STEC) strains of E. coli, E. coli O157:H7, E. coli O145, and E. coli O121:H19), Listeria, Salmonella, Shigella, Staphylococcal food poisoning, Typhoid fever, Vibrio, Yersinia, infections of enteric bacteria that can result in secretory or watery diarrhea (e.g., Vibrio cholera, ETECs (Enterotoxigenic E. coli), EPECs (Enteropathogenic E. coli)), invasive/tissue damaging enteric pathogens that can result in bloody diarrhea and dysentery (e.g., EIECs (Enteroinvasive E. coli), Shigella spp, Salmonella spp, EHECs (Enterohemmorhagic E. coli)), and slow bacterial infection pathogens (e.g., Helicobacter pylori), Balantidium, Cryptosporidium, Toxoplasma, Cyclospora, Micropsoridia, Trichomona, Candida, Staphylococcus, Streptococcus pyogenes, Staphylococcus aureus, Bacillus cereus, Yersinia enterocoliticia, Clostridium difficile, Vibrio parahaemolyticus, Aeromona hydrophila, Plesiomonas sp, norwalk virus, astrovirus, adenovirus, caliciviruses, or parvoviruses; chancoroid; granuloma inguinale; anal cancer; attenuated familial adenomatous polyposis; blumer's shelf; carcinoid; digestive system neoplasm; duodenal cancer; esophageal cancer; familial adenomatous polyposis; gardner's syndrome; gastric lymphoma; gastroinestinal stromal tumor; goblet cell carcinoid; hepatoblastoma; inflammatory myeloblastic tumor; intraductal papillary mucinous neoplasm; juvenile polyposis syndrome; krukenberg tumor; linitis plastica; MALT lymphoma; oesophagogastric juntional adenocarinoma; small intestine cancer; tonsil carcinoma; colon cancer; rectal cancer; gastric cancer; stromal tumors; lipomas; hamartomas; carcinoid syndromes; gastrointestinal carcinoid tumors; adenocarcinomas; sarcoma; gastro intestinal stromal tumors; bile duct cancer; colorectal cancer; nasopharyngeal cancer; oropharyngeal cancer; oral cancer; hypopharyngeal cancer; inflammatory bowel disease; irritable bowel syndrome; constipation; diarrhea; infection; ulcers; pain; metabolic disorders; obesity; immune disorders; autoimmune diseases; nausea; vomiting; bloating; motility disorders; achalasia; gastroparesis; dyspepsia; bleeding; gastroesophogeal reflux disease; Barret's esophagus; gastroenteritis; pyloric stenosis; anemia; pernicious anemia; Crohn's disease; ulcerative colitis; enterocolitis; ischemic colitis; radiation colitis; polyps; enteritis; celiac disease; malabsorption; appendicitis; colitis; diverticulitis; hemorrhoids; anal fissure; perianal absecesses; anal fistula; diverticulosis; acid reflex; hirschsprung disease; fecal incontinence, cyclic vomiting syndrome; dumping syndrome; gallstones; gas; gastritis; gastrointestinal bleeding; inguinal hernia; menetrier's disease; peptic ulcers; liver disease; pancreatitis; short bowel syndrome; viral gastroenteritis; whipple disease; zollinger-ellison syndrome; and proctitis.

In some embodiments, probiotic or commensal bacteria can be genetically engineered to produce a peptide of the present disclosure for use in GI disease treatment. In some embodiments, the peptide or peptide-active agent conjugate can be added to food. In other embodiments, the peptide, peptide-active agent, or genetically engineered probiotic or commensal bacteria that expresses the peptide can be taken as a pill similarly to conventional probiotics. Thus, the stable peptides or peptide-active agent conjugates of this disclosure can provide ongoing prophylaxis or treatment of a GI disease in extreme conditions (e.g., temperature) and can be used in settings without requiring additional storage equipment. This can be advantageous for use in developing countries that lack readily available refrigeration or for use by the armed services.

Peptide-active agent conjugates that can be used to treat gastrointestinal diseases, disorders or infections can comprise any one of the following active agents fused or chemically conjugated to any peptide of SEQ ID NO: 1-SEQ ID NO: 80: antibiotics (e.g., clindamycin, fusidic acid, muprirocin, oritavancin, tedizolid, tigecycline, animoglycosides (e.g., amikacin, gentamicin, kanamycin, neomycin, netilmicin, tobramycin, paromomycin, streptomycin, spectinomycin, loncosamides, clincamycin, linkomycin), ansamycin (e.g., geldanamycin, herbimycin, rifaximin), cabapenems (e.g., ertapenem, doripenem, imipenem/cilastatin, meropenem), quinolines/fluorquinolones (e.g., ciprofloxacin, enoxacin, gatifloxacin, gemifloxacin, levofloxacin, moxifloxacin, nalidixic acid, norfloxacin, ofloxacin, trovaloxacin, grepafloxacin, sparfloxacin, temafloxacin, lomefloxacin), piperacillin/tzaobatam, ticarcillin/clavulanic acid, amoxicillin/clavulanate, ampicillin/sulbactam, streptogramins, cephalosporins (e.g., cefadroxil, cefazolin, cefalotin, ceflexin, cefactor, cefamandole, cefoxitin, cefprozil, cefuroxime, cefixime, cefdinir, cefoperazone, cefotaxime, cefpodoxime, ceftazidime, ceftibuten, ceftizoxime, ceftriazone, cefepime, ceftaroline fosamil, ceftobiprole), glycopeptides (e.g., teicoplanin, vancomycin, telavancin, dalbavancin), lipeptide (e.g., daptomycin), macrolides (e.g., azithromycin, clarithromycin, dirithromycin, erthyromycin, roxithromycin, troleandomycin, telithromycin, spiramycin), monobactams (e.g., aztreonam), nitrofurans (e.g., furazolidone, nitrofurantoin), oxazolidinones (e.g., linezolid, posizolid, radezolid, torezolid), pencillin (e.g., amoxicillin, ampicillin, azlocillin, carbencillin, cloxacillin, dicloxacillin, flucloxacillin, mezlocillin, methicillin, nafcillin, oxaillin, penicillin g, temocillin, ticarcillin), polypetides (e.g., bacitracin, colistin, polymyxin B), sulfonamides (e.g., mafenide, sulfacetamide, sulfadiazine, silver sulfadiazine, sulfamethizole, sulfamethoxazole, sulfanilimide, sulfasalazine, sulfisoxazole, trimethoprim-sulfamethoxazole, sulfonamidochrysoidine), tetracyclines (e.g., demclocyline, doxycycline, minocycline, oxytetracycline, tetracycline), clofazimine, dapsone, capreomycin, clycoserine, ethambutol, ethionamede, isoniazid, pyriazinamide, rifampicin, sterptomycin, arsphenamine, chloramphenicol, fosomycin, metronidazole, platensimycin, quinupristin/dalfopristin, thiamphenicol, trimethoprim, extended spectrum penicillins (e.g., ticaracillin, piperacillin), nitrofurantoin, antiparasitics (e.g., nitazoxanide, melarsoprol, eflorinithine, metronidazole, mebendazole, praziquantel, thiobendazole, ivermectin, tinidazole, miltefosine, pyrantel pamoate, thiabendazole, diethylcarbamizine, niclosamide, albendazole, rifampin, amphoteicin B), antifungals (e.g., fumagillin, amphotericin B, candicidin, filipin, hamycin, nataycin, nystatin, rimocidin, bifonazole, butoconazole, clotrimazole, econazole, fenticonazole, isoconazole, ketoconazole, luiconzole, miconazole, omoconazole, sertaconazole, sulconazole, tioconazole, albaconazole, efinaconazole, epoxiconazole, fluconazole, isavuconazole, itraconazole, posaconazole, propiconazole, ravuconazole, teronazole, voriconazole, abafungin, amorolfin, butenafine, naftifine, terbinafine, anidulafungin, caspofungin, micafungin, aurones, benzoic acid, ciclopirox, flucytosine, griseofulvin, haloprogin, tolnafrtate, undecylenic acid, crystal violet, balsam of Peru), antiviral agents (e.g., abacavir, acyclovir, adefovir, amantadine, amprenavir, ampligen, arbidol, atazanavir, atripla, balavir, cidofovir, combivir, dolutegravir, darunavir, delavirdine, delavirdine, didanosine, docosanol, edoxudine, efavirenz, emtricitabine, enfuvirtide, entecavir, ecoliever, famciclovir, fomivirsen, fosamprenavir, foscarnet, fosonet, fusion inhibitor, ganciclovir, ibacitabine, imunovir, idoxuridine, imiquimod, indinavir, inosine, integrase inhibitor, interferon, lamivudine, lopinaivir, loviride, maraviroc, moroxydine, methisazone, nelfinavir, nevirapine, nexavir, nitazoxanide, nucleoside analogs, novir, oseltamivir, perginterone alfa-2a, penciclovir, peramivir, pleconaril, pdodphyllotoxin, raltegravir, reverse transcriptase inhibitor, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine), anti-inflammatory agents (e.g., naproxen, diclofenac, ibuprofen, indomethacin, piroxicam, nabumetone, etodolac, celecoxib, sulindac, oxaprozin, meloxicam, aspirin, fenoprofen, diflunisal, tolmetin, ketorolac, flurbiprofen, mefenamic acid, ketoprofen, salsalate, valdecoxib, loxoprofen, phenylbutazone) immune modulators (e.g., azathiorpine, mercaptopurine, methotrxate, alefacept, anakinra, certolizumab pegol, etanercept, golimumab, infliximab, natalizumab, rituximab, tocilizumab, ustekinumab), cancer therapeutics (e.g., actinomycin, azacitidine, azathioprine, bleomycin, bortezomib, carboplatin, capecitabine, cisplatin, chlorambucil, cyclophosphamide, cytarabine, daunorubicin, doxifluridine, doxifluridine, doxorubicin, epirubicin, epothilone, etoposide, fluorouracil, gemicitabine, hyroxyurea, idarucibin, imatinib, irinotecan, mechlorethamine, mercaptopurine, methotrexate, mitoxantrone, oxaliplatin, paclitaxel, pemetrexed, teniposide, tioguanine, topetecan, valrubicin, vemurafenib, vinblastine, vincritine, vindesine, vinorelbine), gastric motility modulators (e.g., benzamide, cisapride, domperidone, erythomycin, itopride, mosapride, metoclopramide, prucalopride, renzapride, tegaserod, mitemcinal, levosulpiride, cinitapride), anti-diarrheals (e.g., attapulgite, bismuth subsalicylate, crofelemer, diaraid, diasorb, difenoxin hcl/atropine, diphenoxylate hcl/atropine, imodium, k-pek, kaopectate, lomotil, lonox, loperamide, loperamide/simethicone, mallox, motofen, mytesi, neodiaral, octretoide, opium paregoric, opium tincture, paregoric, rifximin, sandostatin, xifaxan), constipation inhibitors (inaclotide, lactulose, lubiprostone, plecanatide, polyethylene glycol), or ion channel modulators.

Peptide-detectable agent conjugates that can be used to treat gastrointestinal diseases, disorders or infections (as disclosed above) can comprise any detectable agent as disclosed herein or any of the following detectable agents fused or chemically conjugated to any peptide of SEQ ID NO: 1-SEQ ID NO: 80: imaging agents, fluorescent dyes, or radioisotopes.

Other materials can also be conjugated to or formulated (such as in a tablet or capsule) with the peptide, peptide-active agent conjugate, or peptide-detectable agent conjugate to increase residence time in the gut mucosa after oral administration. For example, mucoadhesive polymers can be conjugated to peptides, peptide-active agent conjugates, or peptide-detectable agent conjugates. Mucoadhesive polymers can include hydroxypropyl methylcellulose, hydroxypropyl cellulose (HPC), methylcellulose (MC), and carboxymethyl cellulose (CMC), and insoluble cellulose derivatives such as ethylcellulose and microcrystalline cellulose (MCC), polyacrylates, starch, chitosan, or any polymer described in Chaturvedi et al. (J Adv Pharm Technol Res., 2(4): 215-222 (2011)). Mucoadhesive polymers can also include Carbopol®, Polycarbophil®, sodium alginate, sodium carboxymethylcellulose, hydroxypropylmethylcellulose (HPMC), polyethylene glycol, polyvinylpyrrolidone, hydroxyethycellulose, poloxamer, or any polymer described by Yu et al. in Chapter 2 of das Neves, Jose, and Bruno Sarmento, eds. Mucosal Delivery of Biopharmaceuticals: Biology, Challenges and Strategies. Springer Science & Business Media, 2014. In some embodiments, peptides and/or peptide-active agent conjugates and/or peptide-detectable agent conjugates further coupled to mucoadhesive polymers can enhance the mucoadhesivity of the peptide-polymer and/or peptide-active agent-polymer conjugates and/or peptide-detectable agent-polymer conjugates. By increasing the residence time in the gastrointestinal tract, these mucoadhesive polymers can facilitate sustained therapeutic efficacy of peptides, peptide-active agent conjugates, and peptide-detectable agent conjugates to treat, diagnose, or image gastrointestinal diseases, disorders, and infections. Additionally, a peptide, peptide-active agent conjugate, or peptide-detectable agent conjugate of this disclosure can be formulated to target delivery of the peptide, peptide-active agent conjugate, or peptide-detectable agent conjugate to a specific part of the GI tract or release of the active agent or detectable agent in a specific part of the GI tract, such as with polymer coatings or with other known formulations in the art.

Any peptide or peptide-conjugate of the present disclosure can also be modified to reduce breakdown and/or degradation of the conjugated active agent, which can thereby prevent degradation of active agents that are sensitive to low pH or intestinal enzymes. For example, in some embodiments, peptide-active agent conjugates can be administered to treat disease in the colon. For the treatment of a disease in the colon, a peptide or peptide-active agent conjugate can be formulated, such as, but limited to, in a suppository, tablet, or capsule, with polymer coatings, or any formulation as described herein or known in the art, to prevent premature release of the active agent in the small intestine or stomach, which can allow the peptide or peptide-active agent conjugate to remain intact until it reaches the colon. Alternatively, a peptide-active agent conjugate can be linked to the active agent via a linker that can be cleaved by enzymes or conditions that are specific to the colon, which can also prevent premature release of the active agent in the small intestine or stomach.

Renal Diseases.

In some embodiments, charge can play a role in kidney homing. Positively charged residues can increase binding of peptides to proximal tubule cells, to megalin (which is negatively charged), or can otherwise increase retention in the kidney (Janzer et al. Bioconjug Chem. 2016 Oct. 4, Geng et al. Bioconjug Chem. 2012 Jun. 20; 23(6):1200-10, Wischnjow et al. Bioconjug Chem. 2016 Apr. 20; 27(4):1050-7). The interaction of a peptide of this disclosure in solution and in vivo can be influenced by the isoelectric point (pI) of the peptide and/or the pH of the solution or the local environment it is in. The charge of a peptide in solution can impact the solubility of the protein as well as parameters such as biodistribution, bioavailability, and overall pharmacokinetics. Additionally, positively charged molecules can interact with negatively charged molecules. Positively charged molecules such as the peptides disclosed herein can interact and bind with molecules such as megalin and cubilin, or another cell surface receptor expressed by a cell of the proximal tubule, or a combination thereof. Positively charged residues can also interact with specific regions of other proteins and molecules, such as negatively charged residues of receptors or electronegative regions of an ion channel pore on cell surfaces.

As such, the pI of a peptide can influence whether a peptide of this disclosure can efficiently home to the kidney. Identifying a correlation between pI and kidney homing can be an important strategy in identifying lead peptide candidates of the present disclosure. The pI of a peptide can be calculated using a number of different methods including the Expasy pI calculator and the Sillero method. The Expasy pI can be determined by calculating pKa values of amino acids as described in Bjellqvist et al., which were defined by examining polypeptide migration between pH 4.5 to pH 7.3 in an immobilized pH gradient gel environment with 9.2M and 9.8M urea at 15° C. or 25° C. (Bjellqvist et al. Electrophoresis. 14(10):1023-31 (1993)). The Sillero method of calculating pI can involve the solution of a polynomial equation and the individual pKas of each amino acid. This method does not use denaturing conditions (urea) (Sillero et al. 179(2): 319-35 (1989)) Using these pI calculation methods and quantifying the kidney to blood ratio of peptide signal after administration to a subject can be a strategy for identifying a trend or correlation in charge and kidney homing. In some embodiments, a peptide with a pI above biological pH (˜pH 7.4) can exhibit efficient homing to kidney. In some embodiments, a peptide with a pI of at least 8, at least 9, at least 10, or at least 11 can efficiently home to kidney. In other embodiments, a peptide with a pI of 11-12 can home most efficiently to kidney. In certain embodiments, a peptide can have a pI of about 9. In other embodiments, a peptide can have a pI of 8-10. In some embodiments, more basic peptides can home more efficiently to kidney. In other embodiments, a high pI alone may not be sufficient to cause kidney homing of a peptide.

In some embodiments, the tertiary structure and electrostatics of a peptide of the disclosure can impact kidney homing. Structural analysis or analysis of charge distribution can be a strategy to predict residues important in biological function, such as kidney homing. For example, several peptides of this disclosure that home to kidney can be grouped into a structural class defined herein as “hitchins,” and can share the properties of disulfide linkages between C1-C4, C2-05, and C3-C6. The folding topologies of peptides knotted through three disulfide linkages (C1-C4, C2-C5, and C3-C6), can be broken down into structural families based on the three-dimensional arrangement of the disulfides. Knottins have the C3-C6 disulfide linkage passing through the macrocycle formed by the C1-C4 and C2-05 disulfide linkages, hitchins have the C2-05 disulfide linkage passing through the macrocycle formed by the C1-C4 and C3-C6 disulfide linkages, and yet other structural families have the C1-C4 disulfide linkage passing through the macrocycle formed by the C2-C5 and C3-C6 disulfide linkages. Variants of “hitchin” class peptides with preserved disulfide linkages at these cysteine residues, primary sequence identity, and/or structural homology can be a method of identifying or predicting other potential peptide candidates that can home to kidney. Additionally, members and related members of the calcin family of peptides can also home to kidney, despite having a distinct tertiary structure from the “hitchin” class of peptides. Calcin peptides are structurally a subset of the knottin peptides, with knottin disulfide connectivity and topology, but are further classified on the basis of functioning to bind and activate ryanodine receptors (RyRs). These receptors are calcium channels that act to regulate the influx and efflux of calcium in muscle (Schwartz et al. Br J Pharmacol 157(3):392-403. (2009)). Variants of the calcin family of peptides with preserved key residues can be one way to predict promising candidates that can home to kidney. In some embodiments, structural analysis of a peptide of this disclosure can be determined by evaluating peptides for resistance to degradation in buffers with various proteases or reducing agents. Structural analysis of the distribution of charge density on the surface of a peptide can also be a strategy for predicting promising candidates that can home to kidney. Peptides with large patches of positive surface charge (when at pH 7.5) can home to kidney.

In some embodiments, a peptide of this disclosure can bind to interact with, modulate, antagonize, or agonize any of the below renal ion channels in TABLE 8 reproduced from Table 1 of Kuo et al. (Chem Rev. 2012 Dec. 12; 112(12): 6353-6372), which is incorporated herein by reference.

TABLE 8
Renal Ion Channels from Kuo et al.

| Protein (gene) name | Distribution in kidney | Ion affected | Disease associated |
|---|---|---|---|
| TRPC6 (Trpc6) | Glomerulus | Ca²⁺ | Focal Segmental |
| | | | Glomerulosclerosis |
| TRPM6 (Trpm6) | Distal convoluted tubule | Mg²⁺ | Hypomagnesemia |
| ClC-5 (CLCN5) | Convoluted proximal tubule | Cl⁻/H+ | Dent's disease |
| ClC-Kb (CLCNKB) | Thick ascending loop of Henle | Cl⁻ | Bartter syndrome |
| ROMK (KCNJ1) | Thick ascending loop of | K⁺ | Bartter syndrome |
| | Henle; Distal nephron | | |
| Kir4.1 (KCNJ10) | Collecting duct | K⁺ | EAST syndrome |
| ENaC (Scnn1a) | Collecting duct | Na⁺ | Pseudohypoaldosteronism |
| ENaC (Scnn1a) | Collecting duct | Na⁺ | Liddle's syndrome |
| Polycystin 2 (PKD2) | Convoluted tubule | Ca²⁺ | Polycystic kidney disease |

In some embodiments, a peptide of this disclosure can bind to, interact with, modulate antagonize, or agonize any of the renal ion channels disclosed herein, and further in any of the renal cell types and/or structures, such as renal corpuscle, glomerulus, bowman's capsule, parietal cells, podocytes, mesangial cells, columnar and cuboidal epithelial cells.

In some embodiments, a peptide of this disclosure can bind to, interact with, modulate antagonize, or agonize any of the below renal ion channels in TABLE 9 reproduced from Table 1 of Zhou et al (Am J Physiol Renal Physiol. 2016 Jun. 1; 310(11): F1157-F1167.), which is incorporated herein by reference.

TABLE 9
Renal Ion Channels from Zhou et al.

| | | | | | |
|---|---|---|---|---|---|
| Protein | Human Gene | Expression in | Agonists and | Function in the | Relevant Kidney |
| Name | Name | the Kidney | Activators | Kidney | Diseases |

TRP Family

| TRPC | | | | | |
|---|---|---|---|---|---|
| TRPC1 | TRPC1, TRP1 | MC | PLC | Regulates | DN |
| | | | | mesangial cell | |
| | | | | contractility | |
| TRPC3 | TRPC3, TRP3 | P, DCT, CD | DAG, PLC | Regulates SOCE | Williams-Beuren |
| | | | | in podocytes, | syndrome |
| | | | | Ca²⁺ reabsorption | hypercalcemia, renal |
| | | | | in DCT and CD | fibrosis |
| TRPC5 | TRPC5, TRP5 | P, JGC | Intracellular | Dysregulates | Podocyte injury, |
| | | | Ca²⁺, | podocyte actin | glomerular disease |
| | | | lysophospholipids, | cytoskeleton, | |
| | | | oxidative | degrades | |
| | | | stress, | synaptopodin, and | |
| | | | rosiglitazone, | activates Rac1 | |
| | | | riluzole, PLC | | |
| TRPC6 | TRPC6, TRP6, | P, CD | PLC, DAG, | Regulates | FSGS, DN |
| | FSGS2 | | hyperforin, | podocyte slit | |
| | | | lysophosphatidyl- | diaphragm | |
| | | | choline, 20- | | |
| | | | HETE | | |
| TRPV | | | | | |
| TRPV4 | TRPV4, VR- | ATL, TAL, | Mechanical | Regulates renal | |
| | OAC, OTRPC4 | DCT, CNT | stress, warm | osmolality and | |
| | | | (<33° C.), 4α- | water reabsorption | |
| | | | PDD, | | |
| | | | GSK1016790A | | |
| TRPV5 | TRPV5, CAT2, | DCT, CNT | Constitutively | Ca²⁺ reabsorption | |
| | ECaC1 | | active, PKA- | | |
| | | | dependent | | |
| | | | phosphorylation, | | |
| | | | sheer stress, | | |
| | | | PIP₂ | | |
| TRPP | | | | | |
| PKD1/ | PKD1/PKD2 | Epithelial cells | Mechanical | Activates G | ADPKD |
| PKD2 | | of TAL, DCT | stress, | protein signaling | |
| complex | | | intracellular | cascades, | |
| | | | Ca²⁺ (?) | mechanosensor | |

Voltage-dependent calcium channels (VGCC) Family

| | | | | | |
|---|---|---|---|---|---|
| T-type | | | | | |
| VGCC | | | | | |
| Caᵥ3.1 | CACNA1G | Afferent and | Low voltage | Regulates blood | DN, fibrosis, |
| | | efferent | | flow | glomerular |
| | | arterioles, MC, | | | hypertension |
| | | DCT, CD | | | |
| Caᵥ3.2 | CACNA1H | Afferent and | Low voltage | Regulates | |
| | | efferent | | glomerular | |
| | | arterioles, MC, | | filtration rate | |
| L-type | | | | | |
| VGCC | | | | | |
| Caᵥ1.2 | CACNA1C | Afferent and | High voltage, | Vasoconstriction, | Glomerular |
| | | efferent | 1,4- | modifies the | hypertension, PKD |
| | | arterioles, MC, | dihydropyridines, | formation of | (?) |
| | | DCT | FPL-64176 | kidney cysts | |
| P-/Q-type | | | | | |
| VGCC | | | | | |
| Caᵥ2.1 | CACNA1A | Afferent | High voltage | Depolarization- | |
| | | arterioles, MC | | mediated | |
| | | | | contraction in | |
| | | | | renal afferent | |
| | | | | arterioles | |

TRP, transient receptor potential; VGCC, voltage-gated calcium channels; P, podocyte; MC, mesangial cell; PCT, proximal convoluted tubule; ATL, ascending thin limb; TAL, thick ascending limb; DCT, distal convoluted tubule; CNT, connecting tubule; CD, collecting duct; SOCE, store-operated Ca2+ entry; PIP₂, phosphatidylinositol 4,5-bisphosphate; JGC, juxtaglomerular cell; ADPKD, autosomal dominant polycystic kidney disease; DN, diabetic nephropathy; NDI, nephrogenic diabetes insipidus; FSGS, focal segmental glomerulosclerosis.

In some embodiments, a peptide of this disclosure can bind to, inter act with, modulate antagonize, or agonize any of the renal ion channels in Loudon et al. (Ann Clin Biochem. 2014 July; 51(Pt 4):441-58), which is incorporated herein by reference. Such renal ion channels include NKCC2, ROMK, ClC-Kb, ClC-Ka, NCCT, TRPM6, TRPM7, Kv1.1, Kir4.1, ROMK1, Maxi-K, ENaC, PC1, PC2, and CLC-5, or any combination thereof.

In some embodiments, a peptide can bind to any of the ion channels disclosed herein in a kidney cell or tissue. A peptide of this disclosure can bind to any one of chloride, potassium, sodium, voltage-gated, ligand-gated, mechanosensitive, and TRP ion channels. In some cases, the peptide can also bind to calcium or magnesium channels.

In some cases, a peptide can block, inhibit, or deactivate any of the chloride, potassium, sodium, voltage-gated, ligand-gated, mechanosensitive, and TRP ion channels in a kidney cell or tissue. The peptide can block potassium channels and/or sodium channels. The peptide can block calcium or magnesium channels. In some embodiments, a peptide can binding to an ion channel to reduce activity of an ion channel that is overexpressed in a kidney cell or tissue. In some embodiments, a peptide of this disclosure functions as an antagonist of an ion channel listed in any of TABLE 2-9.

In some cases, a peptide can activate or increase ion channel activity of any one of the chloride, potassium, sodium, voltage-gated, ligand-gated, mechanosensitive, and TRP ion channels in a kidney cell or tissue. The peptide can activate potassium channels and/or sodium channels. The peptide can activate calcium or magnesium channels. In some embodiments, a peptide can binding to an ion channel to increase activity of an ion channel that is under-expressed in a kidney cell or tissue. In some embodiments, a peptide of this disclosure functions as an agonist of an ion channel listed in any of TABLE 2-9. In some embodiments, a peptide of this disclosure can activate any one or more of such channels, or increase the activity of one or more such channels. In other embodiments, the peptide can be an agonist of potassium, sodium, chloride, or calcium channel, a hadrucalcin, a theraphotoxin, a huwentoxin, a kaliotoxin, a cobatoxin or a lectin. In some embodiments, the lectin can be SHL-Ib2. In some embodiments, the peptide can interact with, bind, inhibit, inactivate, or alters expression of ion channels. In some embodiments, the peptide can interact with a Nav1.7 ion channel. In some embodiments, the peptide can interact with a Kv 1.3 ion channel.

Identifying sequence homology can be important for determining key residues that preserve kidney targeting function. For example, conservation of hydrophilic residues, such as N, Q, S, T, D, E, K, R, and H, can be important for preserving peptide kidney targeting function by keeping the peptide from sticking to albumin. Additionally, basic amino acids such as Lys and/or Arg can important to binding and retention of a peptide in the kidney. Other lysine residues can be mutated out, such as by substitution with arginine, to provide a single site for amine conjugation.

The present disclosure provides peptides that can distribute to, home, target, be directed to, accumulate in, migrate to, be retained in, and/or bind to one or more specific regions, tissue, structures, regions, compartments, or cells of the kidney, collectively referred to herein as “renal tissue.” Examples of regions, tissue, structures, or cells of the kidney applicable to the embodiments presented herein include but are not limited to: the cortex region, the glomerulus, the glomerular filtrate (Bowman's space) tubular lumina, the proximal tubule, the S1, S2, and S3 segments, the medulla region, the descending tubule, the ascending tubule, the distal tubule, the loop of Henle, the Bowman's capsule, the renal interstitium, the renal microvasculature, vasa rectae, or any cells or cell types thereof.

In some embodiments, the peptides of the present disclosure interact with renal tissue of the subject, e.g., by binding to the renal tissue. The binding between the peptide and the renal tissue can be a specific binding interaction (e.g., a receptor-ligand interaction) or non-specific binding interaction (e.g., electrostatic interaction). For example, in certain embodiments, upon administration to a subject, a peptide of the present disclosure binds to a proximal tubule of the subject, e.g., a cell of the proximal tubule. As another example, in certain embodiments, upon administration to a subject, a peptide of the present disclosure binds to a glomerulus of the subject, e.g., a cell of the glomerulus. As another example, in certain embodiments, a peptide of the present disclosure binds to podocytes. In various embodiments, the peptides bind to receptors expressed by a renal cell. For instance, a peptide can bind to a cell surface receptor expressed by a cell of the proximal tubule, a megalin receptor, a cubulin receptor, or a combination thereof.

In some embodiments, the peptides are internalized by a cell of the renal tissue of the subject. The present disclosure encompasses various types of internalization mechanisms, including but not limited to pinocytosis, phagocytosis, endocytosis, receptor-mediated endocytosis, scavenging mechanisms, membrane penetration or translocation mechanisms, or combinations thereof. For example, a peptide can be internalized following binding to the cell or a receptor thereof, e.g., via receptor-mediated endocytosis.

In some embodiments, peptides of the present disclosure with a pI value greater than 9 can have higher accumulation in the kidney. In some embodiments, the pI of the peptide influences its localization within the kidney. For example, in certain embodiments, higher pI values (e.g., greater than or equal to about 7.5) promote localization and/or binding to the glomerulus, while lower pI values (e.g., lower than 7.5) promote localization and/or binding to the proximal tubule. Accordingly, different localization patterns within the kidney can be achieved by varying the pI of the peptide. In certain embodiments, the osmotic concentration of the urine and/or urine flow rates have an impact on intratubular localization.

As another example, in various embodiments, the peptides of the present disclosure exhibit a charge distribution at neutral pH favorable for renal localization, binding, and/or internalization. In certain embodiments, the peptide exhibits a substantially uniform charge distribution. In alternative embodiments, the peptide exhibits a non-uniform charge distribution, e.g., including one or more regions of concentrated positive charge and/or one or more regions of concentrated negative charge. The charge distribution can impact the localization, binding and/or internalization of the peptide. For example, the glomerular capillary wall and/or slit processes are negatively charged, which in certain embodiments influences glomerular localization of middle sized positively charged molecules (e.g., having a mass-average molecular weight (Mw) within a range from about 30 kDa to about 60 kDa), while being less likely to influence localization of smaller molecules (e.g., having a Mw less than 30 kDa) such as the peptides of the present disclosure. In certain embodiments, the charge distribution of the peptide influences electrostatic interactions with a target, e.g., the megalin/cubulin receptor.

In yet another example, in various embodiments, the peptides of the present disclosure exhibit a molecular weight favorable for renal targeting, localization, binding, accumulation, and/or internalization. In certain embodiments, the peptide comprises a mass-average molecular weight (Mw) less than or equal to about 1 kDa, less than or equal to about 2 kDa, less than or equal to about 3 kDa, less than or equal to about 4 kDa, less than or equal to about 5 kDa, less than or equal to about 6 kDa or less than or equal to about 10 kDa, less than or equal to about 20 kDa, less than or equal to about 30 kDa, less than or equal to about 40 kDa, less than or equal to about 50 kDa, less than or equal to about 60 kDa, or less than or equal to about 70 kDa. In certain embodiments, the peptide comprises a Mw within a range from about 0.5 kDa to about 50 kDa, or within a range from about 0.5 kDa to about 60 kDa.

In some embodiments, molecules (e.g., proteins or peptides) having relatively low Mw (e.g., less than or equal to about 1 kDa, less than or equal to about 2 kDa, less than or equal to about 3 kDa, less than or equal to about 4 kDa, less than or equal to about 5 kDa, less than or equal to about 10 kDa, less than or equal to about 20 kDa, less than or equal to about 30 kDa, or less than or equal to about 60 kDa) are rapidly targeted to, localized, bound, accumulated, and/or internalized by the kidney. In certain embodiments, low Mw molecules are freely filtered, presented to the proximal tubules of the kidney, and optionally taken up by megalin/cubulin receptors. In certain embodiments, low molecular weight molecules undergo endocytic reabsorption via the megalin/cubulin pathway and are then trafficked to renal tubular lysosomes for processing. In some embodiments, molecules (e.g., proteins or peptides) having higher Mw (e.g., greater than about 70 kDa) are generally excluded from glomerular filtration, but can still be able to achieve interstitial localization via the microcirculation.

In various embodiments, the peptides of the present disclosure exhibit stability at pH values favorable for renal localization, binding, and/or internalization. A peptide can be considered to be stable at a certain pH if it is capable of performing its functional or therapeutic effect, is soluble, is resistant to protease degradation, is resistant to reduction, retains secondary or tertiary structure, or a combination thereof.

In some embodiments, the disulfide knot structure of knotted peptides confers improved stability over a wide range of pH values, which can be advantageous for renal applications. For example, stability at low pH values can be advantageous in order to avoid cast formation leading to intratubular obstruction. In some embodiments, cast formation occurs via co-precipitation of proteins with an endogenously produced glycoprotein known as Tamm Horsall protein. In certain embodiments, this precipitation is affected by urinary pH and osmolality, as precipitation typically occurs under acidic conditions (e.g., pH less than about 5) and high salt concentrations and/or osmolality. Alternatively or in combination, stability at low pH value can reduce or prevent lysosomal degradation, which can improve delivery precision and avoid broader cellular or systemic toxicity.

In some embodiments, the peptides and peptide-conjugates of the present disclosure are used to treat a condition of the kidney, or a region, tissue, structure, or cell thereof. In certain embodiments, the condition is associated with a function of a subject's kidneys. The present disclosure encompasses various acute and chronic renal diseases, including glomerular, tubule-interstitial, and microvascular diseases. Examples of conditions applicable to the present disclosure include but are not limited to: hypertensive kidney damage, acute kidney diseases and disorders (AKD), acute kidney injury (AKI) due to ischemia-reperfusion injury, drug treatment such as chemotherapy, cardiovascular surgery, surgery, medical interventions or treatment, radiocontrast nephropathy, or induced by cisplatin or carboplatin, which can be treated prophylactically, established AKI including ischemic renal injury, endotoxemia-induced AKI, endotoxemia/sepsis syndrome, or established nephrotoxic AKI (e.g. rhabdomyolysis, radiocontrast nephropathy, cisplatin/carboplatin AKI, aminoglycoside nephrotoxicity), end stage renal disease, acute and rapidly progressive glomerulonephritis, acute presentations of nephrotic syndrome, acute pyelonephritis, acute renal failure, chronic glomerulonephritis, chronic heart failure, chronic interstitial nephritis, graft versus host disease after renal tranplant, chronic kidney disease (CKD) such as diabetic nephropathy, hypertensive nephrosclerosis, idiopathic chronic glomerulonephritis (e.g. focal glomerular sclerosis, membranous nephropathy, membranoproliferative glomerulonephritis, minimal change disease transition to chronic disease, anti-GBM disease, rapidly progressive cresentic glomerulonephritis, IgA nephropathy), secondary chronic glomerulonephritis (e.g. systemic lupus, polyarteritis nodosa, scleroderma, amyloidosis, endocarditis), hereditary nephropathy (e.g. polycystic kidney disease, Alport's syndrome), interstitial nephritis induced by drugs (e.g. Chinese herbs, NSAIDs), multiple myeloma or sarcoid, or renal transplantation such as donor kidney prophylaxis (treatment of donor kidney prior to transplantation), treatment post transplantation to treat delayed graft function, acute rejection, or chronic rejection, chronic liver disease, chronic pyelonephritis, diabetes, diabetic kidney disease, fibrosis, focal segmental glomerulosclerosis, Goodpasture's disease, hypertensive nephrosclerosis, IgG4-related renal disease, interstitial inflammation, lupus nephritis, nephritic syndrome, partial obstruction of the urinary tract, polycystic kidney disease, progressive renal disease, renal cell carcinoma, clear cell renal cell carcinoma, papillary renal cell carincoma, chromophobe renal cell carinoma, kidney cancer, transitional cell carcinoma, nephroblastoma, renal sarcoma, renal adenoma, oncocytoma, angiomyolipoma, renal fibrosis, kidney stones, hypertension, hypotension, disorders of sodium, water, acid-base, potassium, calcium, magnesium, or phosphate balance, infections, urinary tract infections, kidney failure, hematuria, renal cysts, uremia, shock, uretal obstruction, proteinuria, Fanconi's syndrome, Bartter's syndrome, chronic renal insufficiency, renal fibrosis, and vasculitis. For example, in certain embodiments, the peptides and peptide-conjugates of the present disclosure are used to reduce acute kidney injury in order to prevent it from progressing to chronic kidney disease.

Alternatively or in combination, in some embodiments, the peptide and peptide-conjugates of the present disclosure are used to elicit a protective response such as ischemic preconditioning and/or acquired cytoresistance in a kidney of the subject. In some embodiments, ischemic preconditioning and/or acquired cytoresistance is induced by administering an agent (e.g., a peptide or peptide-conjugate of the present disclosure) that upregulates the expression of protective stress proteins, such as antioxidants, anti-inflammatory proteins, or protease inhibitors. In certain embodiments, the induced response protects the kidney by preserving kidney function in whole or in part and/or by reducing injury to renal tissues and cells, e.g., relative to the situation where no protective response is induced. The peptides and peptide-conjugates of the present disclosure can provide certain benefits compared to other agents for inducing ischemic preconditioning and/or acquired cytoresistance, such as a well-defined chemical structure and avoidance of low pH precipitation.

In some embodiments, the peptide itself exhibits a renal therapeutic effect. For example, in certain embodiments, the peptide interacts with a renal ion channel, inhibits a protease, has antimicrobial activity, has anticancer activity, has anti-inflammatory activity, induces ischemic preconditioning or acquired cytoresistance, or produces a protective or therapeutic effect on a kidney of the subject, or a combination thereof. Optionally, the renal therapeutic effect exhibited by the peptide is a renal protective effect or renal prophylactic effect (e.g., ischemic preconditioning or acquired cytoresistance) that protects the kidney or a tissue or cell thereof from an upcoming injury or insult. Such effects based upon the peptide in and of itself can be used to enhance the therapeutic effect of active agents that may be conjugated or grafted to the peptides disclosed herein.

For example, in certain embodiments, a peptide of the present disclosure activates protective pathways and/or upregulates expression of protective stress proteins in the kidney or tissues or cells thereof. As another example, in certain embodiments, a peptide of the present disclosure accesses and suppresses intracellular injury pathways. In yet another example, in certain embodiments, a peptide of the present disclosure inhibits interstitial inflammation and prevents renal fibrosis. As a further example, in certain embodiments, a peptide of the present disclosure is administered prior to or currently with the administration of a nephrotoxic agent (e.g., aminoglycoside antibiotics such as gentamicin and minocycline, chemotherapeutics such as cisplatin, immunoglobulins or fragments thereof, mannitol, NSAIDs such as ketorolac or ibuprofen, cyclosporin, cyclophosphamide, radiocontrast dyes) in order to minimize its damaging effects, e.g., by blocking megalin-cubulin binding sites so that the nephrotoxic agent passes through the kidneys.

Alternatively or in combination, in some embodiments, the peptide is conjugated to a renal therapeutic agent that exhibits a renal therapeutic effect. In certain embodiments, the renal therapeutic agent is used to treat a condition of the kidney, or a region, tissue, structure, or cell thereof, such as the conditions provided herein. Examples of such renal therapeutic agents include but are not limited to: dexamethasone, a steroid, an anti-inflammatory agent, an antioxidant (e.g., glutathione, N acetyl cysteine), deferoxamine, feroxamine, iron, tin, a metal, a metal chelate, ethylene diamine tetraacetic acid (EDTA), an EDTA-Fe complex, dimercaptosuccinic acid (DMSA), 2,3-dimercapto-1-propanesulfonic acid (DMPS), penicillamine, an antibiotic such as gentamicin, vancomycin, minocin or mitomyclin, an iron chelator, a porphyrin, hemin, vitamin B12, a chemotherapeutic, an Nrf2 pathway activator such as bardoxolone, angiotensin-converting-enzyme (ACE) inhibitors such as ramipril, captopril, lisinopril, benazepril, quinapril, fosinopril, trandolapril, moexipril, enalaprilat, enalapril maleate, or perindopril erbumine, glycine polymers, or a combination thereof.

In various embodiments, a peptide of present disclosure is conjugated to any one of the active agents described herein for treating a renal disease in a subject in need thereof.

For example, in some embodiments, a peptide of the present disclosure is conjugated to an anti-inflammatory agent such as dexamethasone in order to treat lupus affecting the kidney, vasculitis, Goodpasture's disease, focal segmental glomerulosclerosis, nephritic syndrome, or other renal disorders caused by inflammatory processes. As another example, in some embodiments, a peptide of the present disclosure is conjugated to chemotherapeutic for treating renal cell carcinoma. As a further example, in some embodiments, a peptide of the present disclosure is conjugated to a steroid for treating polycystic renal disease.

For example, in certain embodiments, a peptide of the present disclosure is conjugated to hemin, which signals through the heat shock/heme reactive element pathway in order to upregulate a set of diverse cytoprotective proteins. As another example, in certain embodiments, a peptide of the present disclosure is conjugated to an iron chelate or iron complex in order to deliver iron to the kidney to alter gene expression profiles and induce expression of cytoprotective proteins.

The peptides of the present disclosure enable specific targeting of renal therapeutic agents and other agents to the kidneys, which in some embodiments is beneficial for reducing undesirable effect associated with systemic delivery and/or delivery to non-target tissues. For example, patients with inflammation-driven renal diseases that are currently treated with systemic steroids can benefit from peptide-steroid conjugates of the present disclosure that would deliver the therapeutic specifically to the kidneys at sufficiently high concentrations to elicit a targeted therapeutic effect, while reducing acute systemic side effects. In patients suffering from chronic disease, this approach can advantageously spare much of the rest of the body from side effects associated with long-term use of steroidal compounds. As another example, the peptide-conjugates of the present disclosure can be used for targeted delivery of iron for kidney preconditioning, thus reducing or preventing toxicity associated with systemic iron delivery.

In some embodiments, a method of treating a condition in a subject in need thereof comprises administering to the subject a composition or pharmaceutical composition comprising any of the peptides or peptide-conjugates described herein. For example, in certain embodiments, the composition comprises any of the peptides described herein. Optionally, the composition comprises a moiety coupled to the peptide, such as an active agent (e.g., a renal therapeutic agent) or any other moiety described herein. In various embodiments, the pharmaceutical composition comprises any composition of the present disclosure or a salt thereof, and any of pharmaceutically acceptable carriers described herein. In various embodiments, the composition or pharmaceutical composition homes, targets, is directed to, accumulates in, migrates to, is retained by, or binds to the renal tissue of the subject following administration. The composition or pharmaceutical composition can provide a therapeutic effect on the renal tissue in order to treat the condition, as discussed above and herein.

In some embodiments, a method of protecting a kidney of a subject from injury comprises administering to the subject a composition or pharmaceutical composition comprising any of the peptides or peptide-conjugates described herein. For example, in certain embodiments, the composition comprises any of the peptides described herein. Optionally, the composition comprises a moiety coupled to the peptide, such as an active agent (e.g., a renal therapeutic agent) or any other moiety described herein. In various embodiments, the pharmaceutical composition comprises any composition of the present disclosure or a salt thereof, and any of pharmaceutically acceptable carriers described herein.

In some embodiments, the method further comprises inducing ischemic preconditioning and/or acquired cytoresistance in the kidney of the subject. The ischemic preconditioning and/or acquired cytoresistance can protect the kidney from an injury or insult. The methods of the present disclosure allow such protective responses to be preemptively induced in order to protect the kidney from an upcoming injury or insult (e.g., an upcoming medical procedure). Alternatively or in combination, the present disclosure includes methods for inducing a protective response in order to treat an injury or insult that has already occurred. For example, in certain embodiments, the composition or pharmaceutical composition is administered at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 60 hours, at least 72 hours, or at least 96 hours prior to a predicted occurrence of the injury or insult.

In some embodiments, the present disclosure provides that any peptide of the disclosure including SEQ ID NO: 1-SEQ ID NO: 80 can as a peptide conjugate with an active agent for treatment of a kidney disorder.

In some embodiments, homing of a peptide of this disclosure to the kidneys can be assessed in an animal model such as those described in Zager et al. (Am J Physiol Renal Physiol. 2016 Sep. 1; 311(3):F640-51), Zager et al. (Kidney Int. 2013 October; 84(4):703-12), Zager et al. (Transl Res. 2015 November; 166(5):485-501), Bremlage et al. (BMC Nephrol. 2010 Nov. 16; 11:31), Zager et al. (Am J Physiol Renal Physiol. 2011 December; 301(6):F1334-45), all of which are incorporated herein by reference.

Cancer.

In one embodiment, a method of treating cancer includes administering an effective amount of a peptide of the present disclosure to a subject in need thereof.

In some embodiments, the present disclosure provides a method for treating a cancer or tumor, the method comprising administering to a subject in need thereof an effective amount of a peptide of the present disclosure. One example of cancers or conditions that can be treated with a peptide of the disclosure is solid tumors. Further examples of cancers or conditions that can be treated with a peptide of the disclosure include triple negative breast cancer, breast cancer, breast cancer metastases, metastases of any cancers described herein, colon cancer, colon cancer metastases, sarcomas, acute lymphoblastic leukemia, acute myeloid leukemia, adrenocortical carcinoma, AIDS-related cancers such as Kaposi sarcoma, AIDS-related lymphoma, primary CNS lymphoma, anal cancer, appendix cancer, childhood astrocytomas, astrocytomas, childhood atypical teratoid/rhabdiod tumor, CNS atypical teratoid/rhabdiod tumor, atypical teratoid/rhabdiod tumor, basal cell carcinoma, skin cancer, bile duct cancer, bladder cancer, bone cancer, Ewing sarcoma family of tumors, osteosarcoma, chondroma, chondrosarcoma, primary and metastatic bone cancer, malignant fibrous histiocytoma, childhood brain stem glioma, brain stem glioma, brain tumor, brain and spinal cord tumors, central nervous system embryonal tumors, childhood central nervous system embryonal tumors, central nervous system germ cell tumors, childhood central nervous system germ cell tumors, craniopharyngioma, childhood craniopharyngioma, ependymoma, childhood ependymoma, breast cancer, bronchial tumors, childhood bronchial tumors, burkitt lymphoma, carcinoid tumor, gastrointestinal cancer, carcinoma of unknown primary, cardiac tumors, childhood cardiac tumors, primary lymphoma, cervical cancer, cholangiocarcinoma, chordoma, childhood chordoma, chronic lymphocytic leukemia, chronic myelogenous leukemia, chronic myeloproliferative neoplasms, colon cancer, colorectal cancer, cutaneous T cell lymphoma, ductal carcinoma in situ, endometrial cancer, esophageal cancer, esthesioneuroblastoma, childhood esthesioneuroblastoma, ewing sarcoma, extracranial germ cell tumor, childhood extracranial germ cell tumor, extragonadal germ cell tumor, eye cancer, intraocular melanoma, retinoblastoma, fallopian tube cancer, fibrous histiocytoma of bone, gallbladder cancer, gastric cancer, gastrointestinal carcinoid tumor, gastrointestinal stromal tumors, ovarian cancer, testicular cancer, gestational trophoblastic disease, glioma, hairy cell leukemia, head and neck cancer, hepatocellular cancer, histiocytosis, Langerhans cell histiocytosis, hodgkin lymphoma, hypopharyngeal cancer, intraocular melanoma, melanoma, melanoma metastases, islet cell tumors, pancreatic neuroendocrine tumors, kidney cancer, renal cell tumors, Wilms tumor, childhood kidney tumors, lip and oral cavity cancer, liver cancer, lung cancer, nonhodgkin lymphoma, macroglodulinemia, Waldenstrom macroglodulinemia, male breast cancer, merkel cell carcinoma, metastatic squamous neck cancer with occult primary, midline tract carcinoma involving NUT gene, mouth cancer, multiple endocrine neoplasia syndromes, childhood multiple endocrine neoplasia syndromes, multiple myeloma/plasma cell neoplasm, mycosis fungoides, myelodysplastic syndromes, myelodysplastic/myeloproliferative neoplasms, multiple myeloma, myloproliferative neoplasms, chronic myeloproliferative neoplasms, nasal cavity and paranasal sinus cancer, nasopharyngeal cancer, neuorblastoma, non-small cell lung cancer, oropharyngeal cancer, low malignant potential tumor, pancreatic cancer, pancreatic neuroendocrine tumors, papillomatosis, childhood papillomatosis, paraganglioma, paranasal sinus and nasal cavity cancer, parathyroid cancer, penile cancer, pheochromocytoma, pharyngeal cancer, pituitary tumor, pleuropulmonary blastoma, childhood pleuropulmonary blastoma, primary peritoneal cancer, prostate cancer, rectal cancer, pregnancy-related cancer, rhabdomyosarcoma, childhood rhabdomyosarcoma, salivary gland cancer, Sezary syndrome, small cell lung cancer, small intestine cancer, soft tissue sarcoma, squamous cell carcinoma, testicular cancer, throat cancer, thymoma, thymic carcinoma, thyroid cancer, transitional cell cancer of the renal, pelvis, and ureter, uterine cancer, urethral cancer, endometrial cancer, uterine sarcoma, vaginal cancer, vascular tumors, and vulvar cancers.

In some embodiments, a peptide binds to an ion channel, such as those listed in TABLE 2-9, to modulate activity of the ion channel. In some embodiments, a peptide disclosed herein binds to an ion channel of a cancer cell to inhibit its activity or to deliver a cytotoxic agent to the cancer cell. In some embodiments, a peptide disclosed herein binds to an ion channel of a cancer cell to activate an ion channel or to deliver an active agent.

In some embodiments, a method of treating cancer involves delivering a peptide that binds to any one of to a voltage-gated channel, a ligand-gated channel, a ligand-activated channel, an inward rectifier channel, or a mechanosensitive ion channel; or a potassium channel, sodium channel, calcium channel, TRP channel, GABA channel, ionotrophic glutamate receptor channel, acetylcholine receptor, nicotinic receptor, 5-HT3 receptor, or chloride channel; or any one of Kv1.1, Kv1.2, Kv1.3, Cav2.1, Cav2.2, Nav1.5 (TP1), Nav1.5 (TP2), Nav1.7 (TP1), Nav1.7 (TP2), hERG, 5-HT3a, alpha-4 beta-2 nicotinic receptor, Kv2.1, TPV1, GABA, and Kir2.1 channels. In some embodiments, the peptide blocks or activates such channels in a target cell or tissue. In some embodiments, the peptide blocks or activates such ion channel in a cancer cell.

In some embodiments, the peptide interacts with nicotinic acetyl choline receptors, transient receptor potential channels, NMDA receptors, serotonin receptors, KIR channels, GABA channels, glycine receptors, glutamate receptors, acid sensing ion channels, K2P channels, Nav1.7, or purinergic receptors. In some embodiments, the peptide interacts with matrix metalloproteinase, inhibits cancer cell migration or metastases, or has antitumor activity. In some embodiments, the peptide interacts with calcium activated potassium channels. In some embodiments, the peptide has antibacterial, antifungal, or antiviral activity. In some embodiments, the peptide inhibits proteases. In some embodiments, the peptide interacts with channels that influence pain. In some embodiments, the peptide has other therapeutic effects on the tissue of an effected organ or structures thereof.

In some embodiments, the peptides of the present disclosure are used to treat cancers. For example, in certain embodiments, the peptides provided herein are used to directly inhibit critical cancer-associated pathways such as RAS, MYC, PHF5A, BubR1, PKMYT1, or BuGZ.

In some aspects, the peptides of the present disclosure are conjugated to one or more therapeutic agents. In certain aspects, the therapeutic agent is a chemotherapeutic, anti-cancer drug, or anti-cancer agent selected from, but are not limited to: radioisotopes, toxins, enzymes, sensitizing drugs, nucleic acids, including interfering RNAs, antibodies, anti-angiogenic agents, cisplatin, platinum compounds, anti-metabolites, mitotic inhibitors, growth factor inhibitors, taxanes, paclitaxel, cabazitaxel, temozolomide, topotecan, fluorouracil, vincristine, vinblastine, 4-deacetylvinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, vinca alkaloids, cyclic octapeptide analogs of mushroom amatoxins, epothilones, and anthracylines, CC-1065, SN-38, and BACE inhibitors, and their equivalents, as well as photo-ablation agents. For example, in certain embodiments, a peptide of the present disclosure is conjugated to palbociclib, a CDK 4/6 inhibitor with limited ability to cross the blood brain barrier. As another example, in certain embodiments, a peptide of the present disclosure is conjugated to monomethyl auristatine E (MMAE), MMAF, auristatin, dolostatin, auristatin F, monomethylauristatin D, maytansinoid (e.g., DM-1, DM4, maytansine), pyrrolobenzodiazapine dimer, calicheamicin, N-acetyl-γ-calicheamicin, duocarmycin, anthracycline, a microtubule inhibitor, or a DNA damaging agent.

Optionally, certain embodiments of the present disclosure provide peptides conjugated to a radiosensitizer or photosensitizer disclosed herein. In certain embodiments, the peptide of the disclosure is mutated to change the binding affinity or specificity of its function as agonizing or antagonizing an ion channel.

In some embodiments, the present disclosure provides a method for treating a cancer, the method comprising administering to a subject in need thereof an effective amount of a peptide of the present disclosure. In some embodiments, the present disclosure provides a method for treating a cancer, the method comprising administering to a patient in need thereof an effective amount of a pharmaceutical composition comprising a peptide of the present disclosure and a pharmaceutically acceptable carrier. In some embodiments, the present disclosure provides a method for inhibiting invasive activity of cells, the method comprising administering an effective amount of a peptide of the present disclosure to a subject.

A peptide comprising the sequence of any of SEQ ID NO: 1-SEQ ID NO: 80, and any peptide derivative or peptide-active agent as described herein, can be used to target upper GI disease and cancers (e.g., throat, oral, esophageal cancer, salivary glands, tonsils, pharynx, adenosarcomas, oral malignant melanoma head and neck cancer). A peptide comprising the sequence of any of SEQ ID NO: 1-SEQ ID NO: 80, and any peptide derivative or peptide-active agent as described herein, can be used to additionally target gall bladder disease and cancers. In some embodiments, the present disclosure provides a method of treating a tumor or cancerous cells of a subject, the method comprising administering to the subject a peptide comprising the sequence of any of SEQ ID NO: 1-SEQ ID NO: 80, or a functional fragment thereof.

Multiple peptides described herein can be administered in any order or simultaneously. In some cases, multiple functional fragments of peptides derived from toxins or venom can be administered in any order or simultaneously. If simultaneously, the multiple peptides described herein can be provided in a single, unified form, such as an intravenous injection, or in multiple forms, such as subsequent intravenous dosages.

Peptides can be packaged as a kit. In some embodiments, a kit includes written instructions on the use or administration of the peptides.

Brain Diseases:

In one embodiment, the method includes administering an effective amount of a peptide of the present disclosure to a subject in need thereof, such as a subject diagnosed with a brain disease or a brain cancer.

The activity of a plurality of brain regions, tissues, structures or cells can be modulated by a peptide of the disclosure. Some of the brain regions, tissues, structures include: a) the cerebrum, including cerebral cortex, basal ganglia (striatum), and olfactory bulb; b) the cerebellum, including dentate nucleus, interposed nucleus, fastigial nucleus, and vestibular nuclei; c) diencephalon, including thalamus, hypothalamus, and the posterior portion of the pituitary grand; and d) the brain-stem, including pons, substantia nigra, medulla oblongata; e) the temporal lobe, including the hippocampus and the dentate gyrus (including the subgranular zone); f) the ventricular system, including the lateral ventricles (right and left ventricles), third ventricle, fourth ventricle, intraventricular foramina, cerebral aqueduct, median aperture, right and left lateral apertures, choroid plexus, and the subventricular zone; g) the CSF and associated tissues, including the subarachnoid space, cisterns, sulci; h) the meninges, including the dura mater, arachnoid mater, and pia mater; i) the rostral migratory stream; j) neural stem cells, neural progenitor cells, and new neural cells; and k) any cells or cell types in (a)-(j) above. In some embodiments, the peptides of the present disclosure are capable of crossing the BBB or blood CSF barrier and accumulating in one or more specific brain regions, tissue, structures, or cells. For example, in certain embodiments, the peptides described herein bind, target, or are directed to an ion channel in the hippocampus, the CSF, the ventricular system, the meninges, or the rostral migratory stream, or combinations thereof.

In some embodiments, the present disclosure provides a method for treating a brain disease or condition, the method comprising administering to a subject in need thereof an effective amount of a peptide of the present disclosure. A brain disease or condition can be any neurodegenerative disease or lysosomal storage disease. A neurodegenerative disease can be any disease, state, or condition relating to the loss of structure or function of the central nervous system, including any disease, state or condition relating to the loss of structure or function of the central nervous system, including without limitation Alzheimer's disease, Parkinson's disease, Huntington's disease, Amyotrophic Lateral Sclerosis, Frontotemporal Dementia, Progressive Supranuclear Palsy and Corticobasal Degeneration. A lysosomal storage disease can be any disease, state, or condition relating to defects in lysosomal function, including, without limitation, Krabbe disease, Gaucher disease, Tay-Sachs disease, Niemann-Pick disease, Pompe disease, Hurler syndrome, and Hunter syndrome. Further examples of brain diseases or conditions that can be treated with a peptide of the disclosure include Acoustic Neuroma (Vestibular Schwannoma), Acute Subdural Hematomas, Addictions (e.g., alcoholism, drug addiction, nicotine or tobacco, etc.), Alzheimer's disease, Amyotrophic Lateral Sclerosis (ALS, or Lou Gehrig's Disease), Anaplastic Astrocytoma (AA), Anxiety and related disorders, Anorexia, Antisocial Personality Disorder, Aqueductal Stenosis, Arachnoid Cysts, Arnold Chiari Malformation, Arteriovenous Malformation (AVM), Astrocytoma, Autism, Ballism, bipolar disorders, Brain Aneurysm, Brain Attack, Brain Metastases, Brainstem Glioma, Bulimia, Carotid Stenosis, Catastrophic Epilepsy in Children, Cavernous Angioma, Cerebral Aneurysms, Cerebral Contusion and Intracerebral Hematoma, Cerebral Hemorrhage, Chiari Malformation, Chordomas, Chorea, Choroid Plexus Cyst, Chronic Subdural Hematomas, Colloid Cyst, Coma, Concussion, Cranial Gun Shot Wounds, Corticobasal Degeneration, Craniopharyngioma, Craniosynostosis, Cushing's Disease, Cyst (Epidermoid Tumor), Dementia, Depression and related disorders, eating disorders, weight loss and satiety, Diabetes, Dravet Syndrome, Ependymoma, Epilepsy, Epidural Hematomas Epilepsy, Essential Tremor, Extratemporal Lobe Epilepsies, Facet Joint Syndrome, Frontotemporal Dementia, Ganglioglioma, Gaucher disease, Germinoma, Glioblastoma Multiforme (GBM), Glioma, Glomus Jugulare Tumor, Glossopharyngeal Neuralgia, Hemangioblastomas, Hemi-Facial Spasm, Hydrocephalus, Huntington's disease, immune system disorders, Intracerebral Hemorrhage, Hurler syndrome, Hunter syndrome, Intracranial Hypotension, JPA (Juvenile Pilocytic Astrocytoma), Krabbe disease, Lennox-Gestaut Syndrome, Lipomyelomeningocele, Low-Grade Astocytoma (LGA), Lymphocytic Hypophysitis, Lymphoma, Medulloblastoma, Meningioma, Meningitis, Mesial Temporal Lobe Epilepsy, Metastatic Brain Tumors, Migraine, Mitochondrial Disease, Moyamoya Disease, multiple sclerosis, Multiple system atrophy (MSA), Niemann-Pick disease, Nelson's Syndrome, Neurocysticercosis, Neurodegenerative Disorders, Neurofibroma, neuropathic pain, Nonfunctional Pituitary Adenoma, Normal Pressure Hydrocephalus, obsessive-compulsive disorders, Oligodendroglioma, Optic Nerve Glioma, Osteomyelitis, Parkinson's disease, Paranoia and related disorders, Pediatric Hydrocephalus, Phantom Limb Pain, Pilocytic Astrocytoma, Pineal Tumor, Pineoblastoma, Pineocytoma, Pituitary Adenoma (Tumor), Pituitary Apoplexy, Pituitary Failure, Pompe disease, Postherpetic Neuralgia, Post-Traumatic Seizures, Post-Traumatic Stress Disorder, Primary CNS Lymphoma, Prolactinoma, Pseudotumor Cerebri, Progressive Supranuclear Palsy, Rathke's Cleft Cyst, Recurrent Adenomas, Rheumatoid Arthritis, Schizophrenia, Schwannomas, Scoliosis, Skull Fracture, Slit Ventricle Syndrome, Spasticity, Spontaneous Intracranial Hypotension, Stroke (Brain Attack, TIA), Subarachnoid Hemorrhage, Syrinx, Tay-Sachs disease, Thyrotroph (TSH) Secreting Adenomas, Torticollis, Transient Ischemic Attacks (TIA), Traumatic Brain Injury, Traumatic Hematomas, Trigeminal Neuralgia, Ventriculitis, Vestibular Schwannoma, depression, mood disorders, lysosomal storage diseases, memory disorders, learning disorders, disorders of spatial memory or navigation, stress-related disorders, post-traumatic stress disorder, pain, aging, hippocampal atrophy, brain infections including fungal infections and progressive Multifocal Leukoencepalopathy, or another brain disease or condition. In other cases, a peptide of the disclosure can be used to treat alcoholism, cigarette addiction, drug addiction, or anxiety.

In some embodiments, the peptide binds to any one of voltage-gated channel, a ligand-gated channel, a ligand-activated channel, an inward rectifier channel, or a mechanosensitive ion channel; or a potassium channel, sodium channel, calcium channel, TRP channel, GABA channel, ionotrophic glutamate receptor channel, acetylcholine receptor, nicotinic receptor, 5-HT3 receptor, or chloride channel; or any one of Kv1.1, Kv1.2, Kv1.3, Cav2.1, Cav2.2, Nav1.5 (TP1), Nav1.5 (TP2), Nav1.7 (TP1), Nav1.7 (TP2), hERG, 5-HT3a, alpha-4 beta-2 nicotinic receptor, Kv2.1, TPV1, GABA, and Kir2.1 channels in the brain. In some embodiments the peptide blocks or activates one of these channels in the brain. In some embodiments, the peptide interacts or modulates activity of an ion channels in the brain, including chloride channels, calcium channels, nicotinic acetyl choline receptors, transient receptor potential channels, NMDA receptors, serotonin receptors, KIR channels, GABA channels, glycine receptors, glutamate receptors, acid sensing ion channels, K2P channels, Nav1.7, or purinergic receptors. In some embodiments, the peptide binds to an ion channel to inhibit cancer cell migration or metastases, or reduces tumor activity. In some embodiments, the peptide interacts with channels that influence pain. In some embodiments, the peptide has other therapeutic effects on the brain or structures thereof.

In some embodiments, the peptides of the present disclosure are used to diagnose or treat a disease or condition associated with the hippocampus. The hippocampus is a critical brain structure involved in learning, memory, mood, and cognition. Changes in the hippocampus, including reduced volume and cellularity, reduced neuronal density, and defects in neurotransmitter function, are associated with initiation, persistence, and/or progression of disorders including late-life depression (Taylor); major depression and bipolar disorder (Drevets); post-traumatic stress disorder (PTSD) (Schmidt); Alzheimer disease (Nava-Mesa); and schizophrenia (Perez). Peptides of the current invention that target the hippocampus can be used to treat these diseases or to target therapeutically-active substances to treat these diseases amongst others. In some embodiments, the peptides are used to treat these diseases by acting on receptors such as GABA, NMDA, AMPA, dopamine, or serotonin receptors. The dentate gyrus in the hippocampus can also be a site of neurogenesis.

In some embodiments, the peptides of the present disclosure are used to diagnose or treat a disease or condition associated with the CSF or ventricular system. The CSF is a fluid that surrounds and circulates in the brain and spine that provides mechanical protection for the brain and plays a role in the homeostasis and metabolism of the central nervous system. CSF is produced by and circulated within the ventricular system. Diseases and conditions that are associated with the CSF or ventricular system include but are not limited to: antisocial personality disorder, cerebral hemorrhage, choroid plexus cyst, dementia, ependymoma, hydrocephalus, meningitis, multiple system atrophy (MSA), neurodegenerative disease (such as amyotrophic lateral sclerosis, Parkinson's disease, Alzheimer's disease, Huntington's disease) post-traumatic stress disorder, schizophrenia, subarachnoid hemorrhage, traumatic brain injury, and ventriculitis.

Peptides of the current disclosure that target the CSF or ventricular system can be used to treat these diseases or to target therapeutically-active substances to treat these diseases, amongst others. For example, in certain embodiments, the peptides of the present disclosure are used to modulate targets associated with a disease, such as mitochondrial deubiquitinase USP30 (e.g., for the treatment of Parkinsons' disease) or dual leucine zipper kinase (e.g., for the treatment of neurodegeneration). As another example, in certain embodiments, the peptides are conjugated to a therapeutic agent used to treat a neurodegenerative disease, such as Alzheimer's disease. Such drugs could also include galantamine, donzepil, tacrine, or even neurotoxins generally thought to be too toxic, such as sarin. Examples of therapeutic agents useful for treating neurodegenerative disease include but are not limited to: acetylcholinesterase inhibitors (e.g., rivastigimine), galantamine, donzepil, tacrine, and neurotoxins (e.g., sarin). This approach allows for treatment with lower dosages and reduced side effects in the periphery, compared to prior methods which utilize untargeted systemic delivery. In yet another example, in certain embodiments, peptides that home, distribute to, target, migrate to, accumulate in, or are directed to the ventricular space are used as radioprotectant (e.g., alone or as a conjugate to a radioprotective compound such as amifostine) during treatment of brain metastases with radiation.

In some embodiments, the peptides of the present disclosure are used to inhibit small-conductance, calcium-activated potassium channels (SK channels). Peptides that inhibit SK channels include members of the Toxin_6 class, for example. Optionally, such peptides may exhibit homing to specific brain regions, such as the ventricles. In certain embodiments, the peptides of the present disclosure have specificity for one or more SK channel subtypes, such as one or more of the SK1, SK2, SK3, or SK4 channel subtypes. In certain embodiments, inhibition of the SK3 subtype increases the frequency of firing in dopaminergic neurons, thus increasing levels of dopamine, which may ameliorate the physical symptoms of Parkinson's disease.

In some embodiments, the peptides of the present disclosure are used to affect (e.g., reduce, slow, or inhibit) the aggregation of proteins associated with neurodegenerative disease, such as tau, prion protein, amyloid beta, alpha synuclein, parkinin, or huntingtin.

In some embodiments, the peptides of the present disclosure are used to inhibit or activate one or more specific ion channels, and the inhibition or activation of the ion channels alleviates the symptoms of a range of diseases. TABLE 2-9 above illustrates exemplary ion channels and associated diseases that may be treated in accordance with the compositions and methods presented herein.

In some embodiments, the peptides of the present disclosure can be modified to bind to an ion channel that plays a role in inflammation in the brain and thus result in an anti-inflammatory effect. Alternatively or in combination, peptides of the present disclosure can be conjugated to immune regulatory molecules to reverse, reduce, or limit inflammation.

In some aspects, the peptides of the present disclosure are conjugated to one or more therapeutic agents or an active agent, including anti-inflammatory molecules (e.g., dexamethasone, prednisone, prednisolone, methyl prednisolone, or traimcinolone), antifungal agents (e.g., fluconazole, amphotericin B, ketoconazole, or abafungin), antiviral agents (e.g., acyclovir, cidofovir), growth factors (e.g., NGF or EGF), or anti-infective agents (e.g., ciprofloxacin, tetracycline, erythromycin, or streptomycin).

In some embodiments, the peptides of the present disclosure are used to treat brain cancer. For example, in certain embodiments, the peptides provided herein are used to directly inhibit critical cancer-associated pathways such as RAS, MYC, PHF5A, BubR1, PKMYT1, or BuGZ. Alternatively or in combination, the peptides of the present disclosure are used to carry a conjugated therapeutic agent across the BBB in order to treat brain cancer.

In further aspects, a peptide that targets or binds to an ion channel in the brain can be conjugated to a therapeutic agent, including a chemotherapeutic agent, anti-cancer drug, or anti-cancer agent selected from, but are not limited to: radioisotopes, toxins, enzymes, sensitizing drugs, nucleic acids, including interfering RNAs, antibodies, anti-angiogenic agents, cisplatin, platinum compounds, anti-metabolites, mitotic inhibitors, growth factor inhibitors, taxanes, paclitaxel, cabazitaxel, temozolomide, topotecan, fluorouracil, vincristine, vinblastine, 4-deacetylvinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, vinca alkaloids, cyclic octapeptide analogs of mushroom amatoxins, epothilones, and anthracylines, CC-1065, SN-38, and BACE inhibitors, and their equivalents, as well as photo-ablation agents. For example, in certain embodiments, a peptide of the present disclosure is conjugated to palbociclib, a CDK 4/6 inhibitor with limited ability to cross the BBB. As another example, in certain embodiments, a peptide of the present disclosure is conjugated to monomethyl auristatine E (MMAE), MMAF, an auristatin, dolostatin, auristatin F, monomethylauristatin D, a maytansinoid (e.g., DM-1, DM4, maytansine), a pyrrolobenzodiazapine dimer, N-acetyl-γ-calicheamicin, a calicheamicin, a duocarmycin, an anthracycline, a microtubule inhibitor, or a DNA damaging agent.

Optionally, certain embodiments of the present disclosure provide peptides conjugated to a radiosensitizer or photosensitizer disclosed herein.

In certain embodiments, the peptide of the disclosure is mutated to retain ability to cross the BBB or the blood CSF barrier and target or bind an ion channel in a cell in the brain.

In some embodiments, the present disclosure provides a method for treating a cancer, the method comprising administering to a subject in need thereof an effective amount of a peptide of the present disclosure, or an effective amount of a pharmaceutical composition comprising a peptide of the present disclosure and a pharmaceutically acceptable carrier. In some embodiments, the present disclosure provides a method for treating ion channel-related disease in the brain, the method comprising administering an effective amount of a peptide of the present disclosure to a subject.

In some aspects, the present disclosure provides a method for detecting a cancer, cancerous tissue, or tumor tissue, the method comprising the steps of contacting a tissue of interest with a peptide of the present disclosure, wherein the peptide is conjugated to a detectable agent and measuring the level of binding of the peptide, wherein an elevated level of binding, relative to normal tissue, is indicative that the tissue is a cancer, cancerous tissue or tumor tissue.

The present invention provides methods for intraoperative imaging and resection of a cancer, cancerous tissue, or tumor tissue using a peptide of the present disclosure conjugated with a detectable agent. In some aspects, the cancer, cancerous tissue, or tumor tissue is detectable by fluorescence imaging that allows for intraoperative visualization of the cancer, cancerous tissue, or tumor tissue using a peptide of the present disclosure. In some aspects, the peptide of the present disclosure is conjugated to one or more detectable agents. In a further embodiment, the detectable agent comprises a fluorescent moiety coupled to the peptide. In another embodiment, the detectable agent comprises a radionuclide. In some aspects, imaging is achieved using open surgery. In further aspects, imaging is accomplished using endoscopy or other non-invasive surgical techniques.

In some cases, the peptide or peptide-active agent can be used to target an ion channel associated with a cancer in the brain by crossing the BBB or blood CSF barrier and then modulating the ion channel to inhibit or to activate its activity. In other cases, the peptide or peptide-active agent can be used to label, detect, or image brain lesions or cancerous cells, including tumors and metastases amongst other lesions, which may be removed through various surgical techniques.

In some embodiments, the present disclosure provides a method of treating a brain condition of a subject, the method comprising administering to the subject a peptide comprising the sequence of any of SEQ ID NO: 1-SEQ ID NO: 80, or a functional fragment thereof.

Cartilage Diseases:

In some embodiments, a method of treating a cartilage disease or disorder comprises delivering a peptide of present disclosure that binds to a cartilage-specific ion channel, such as mechanosensory ion channel TRPV4. In some embodiments, binding of such peptides to an ion channel inhibits or activates the ion channel. Such targeted therapy can allow for lower dosing, reduced side effects, improved patient compliance, and improvement in therapeutic outcomes, which would be advantageous not only in acute disease of the cartilage, but in chronic conditions, such as osteoarthritis.

In some embodiments, peptides derived from toxins and venoms, e.g., knottins, can interact with ion channels associated with the cartilage, such as ion channels in chondrocytes, to modulate (e.g., inhibit or activate) ion channel activity, proliferation, mechanotransduction, and other functions (Potassium Ion Channels in Articular Chondrocytes, Ali Mobasheri, in Mechanosensitive Ion Channels Mechanosensitivity in Cells and Tissues Volume 1, 2008, pp 157-178).

In some embodiments, charge can play a role in targeting an ion channel in the cartilage. The interaction of a peptide of this disclosure in solution and in vivo can be influenced by the isoelectric point (pI) of the knottin peptide and/or the pH of the solution or the local environment it is in. The charge of a peptide in solution can impact the solubility of the protein as well as parameters such as biodistribution, bioavailability, and overall pharmacokinetics. Additionally, positively charged residues can also interact with specific regions of ion channels, such as negatively charged residues of receptors or electronegative regions of an ion channel pore on cell surfaces. As such, the pI of a peptide can influence whether a peptide of this disclosure can efficiently an ion channel. Identifying a correlation between pI and cartilage-specific ion channel targeting can be an important strategy in identifying lead peptide candidates of the present disclosure. The pI of a peptide can be calculated using a number of different methods described herein.

In some embodiments, the peptide can activate or block a cartilage specific ion channel, including a voltage-gated channel, a ligand-gated channel, a ligand-activated channel, an inward rectifier channel, or a mechanosensitive ion channel; or a potassium channel, sodium channel, calcium channel, TRP channel, ionotrophic glutamate receptor channel, acetylcholine receptor, nicotinic receptor, 5-HT3 receptor, or chloride channel; or any one of Kv1.1, Kv1.2, Kv1.3, Cav2.1, Cav2.2, Nav1.5 (TP1), Nav1.5 (TP2), Nav1.7 (TP1), Nav1.7 (TP2), hERG, 5-HT3a, alpha-4 beta-2 nicotinic receptor, Kv2.1, TRPV1, TRPV4, and Kir2.1 channels. In still other embodiments, the peptide can be an agonist to a cartilage-specific ion channel, an antagonist to a cartilage-specific ion channel, a hadrucalcin, a theraphotoxin, a huwentoxin, a kaliotoxin, a cobatoxin or a lectin. In some embodiments, the lectin can be SHL-Ib2. In some embodiments, the peptide can interact with, binds, inhibits, inactivates, or alters expression of ion channels or chloride channels. In some embodiments, the peptide can interact with a Nav1.7 ion channel. In some embodiments, the peptide can interact with a Kv 1.3 ion channel.

In some embodiments, the peptide interacts with or binds to a cartilage-specific ion channel to result in a therapeutic effect on the cartilage or structures thereof or nearby. In some embodiments, a peptide binds to an ion channel in immune cells associated with an autoimmune disease, such as osteoarthritis, autoimmune rheumatic disorders, such as systemic lupus erythematosus and rheumatoid arthritis (Vordenbaumen and Schneider 2011, Varoga 2004 and Varoga 2005).

Identifying sequence homology can be important for determining key residues that preserve cartilage homing function. For example, in some embodiments identification of conserved positively charged residues can be important in preserving cartilage homing in any homologous peptide variants that are made. In other embodiments, identification of basic or aromatic dyads, can be important in preserving interaction and activity with Kv ion channels in homologous variants.

Peptides of the current disclosure that target the cartilage can be used to treat or manage pain associated with a cartilage injury or disorder, or any other cartilage or joint condition as described herein. The peptides can be used either directly by binding to cartilage-specific ion channels or as carriers of active drugs, peptides, or molecules. For example, since ion channels can be associated with pain and can be activated in disease states such as arthritis, peptides that interact with ion channels can be used directly to reduce pain. In another embodiment, the peptide is conjugated to an active agent with anti-inflammatory activity, in which the peptide acts as a carrier for the local delivery of the active agent to reduce pain.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment can also include treating a human in a clinical trial. A treatment can comprise administering to a subject a pharmaceutical composition, such as one or more of the pharmaceutical compositions described throughout the disclosure. A treatment can comprise a once daily dosing. A treatment can comprise delivering a peptide of the disclosure to a subject, either intravenously, subcutaneously, intramuscularly, by inhalation, dermally, intra-articular injection, orally, intrathecally, transdermally, intranasally, via a peritoneal route, or directly onto or into a joint, e.g., via topical, intra-articular injection route or injection route of application. A treatment can comprise administering a peptide-active agent complex to a subject, either intravenously, intra-articular injection, parenterally, orally, via a peritoneal route, or directly onto, near or into the cartilage.

Types of cartilage diseases or conditions that can be treated with a peptide of the disclosure can include inflammation, pain management, anti-infective, pain relief, anti-cytokine, cancer, injury, degradation, genetic basis, remodeling, hyperplasia, surgical injury/trauma, or the like. Examples of cartilage diseases or conditions that can be treated with a peptide of the disclosure include Costochondritis, Spinal disc herniation, Relapsing polychondritis, Injury to the articular cartilage, any manner of rheumatic disease (e.g., Rheumatoid Arthritis (RA), ankylosing spondylitis (AS), Systemic Lupus Erythematosus (SLE or “Lupus”), Psoriatic Arthritis (PsA), Osteoarthritis, Gout, and the like), Herniation, Achondroplasia, Benign or non-cancerous chondroma, Malignant or cancerous chondrosarcoma, Chondriodystrophies, Chondromalacia patella, Costochondritis, Halus rigidus, Hip labral tear, Osteochondritis dssecans, Osteochondrodysplasias, Torn meniscus, Pectus carinatum, Pectus excavatum, Chondropathy, Chondromalacia, Polychondritis, Relapsing Polychondritis, Slipped epiphysis, Osteochondritis Dissecans, Chondrodysplasia, Costochondritis, Perichondritis, Osteochondroma, Knee osteoarthritis, Finger osteoarthritis, Wrist osteoarthritis, Hip osteoarthritis, Spine osteoarthritis, Chondromalacia, Osteoarthritis Susceptibility, Ankle Osteoarthritis, Spondylosis, Secondary chondrosarcoma, Small and unstable nodules as seen in osteoarthritis, Osteochondroses, Primary chondrosarcoma, Cartilage disorders, scleroderma, collagen disorders, Chondrodysplasia, Tietze syndrome, Dermochondrocorneal dystrophy of Francois, Epiphyseal dysplasia multiple 1, Epiphyseal dysplasia multiple 2, Epiphyseal dysplasia multiple 3, Epiphyseal dysplasia multiple 4, Epiphyseal dysplasia multiple 5, Ossified Ear cartilages with Mental deficiency, Muscle Wasting and Bony Changes, Periosteal chondrosarcoma, Carpotarsal osteochondromatosis, Achondroplasia, Genochondromatosis II, Genochondromatosis, Chondrodysplasia—disorder of sex development, Chondroma, Chordoma, Atelosteogenesis, type 1, Atelosteogenesis Type III, Atelosteogenesis, type 2, Pyknoachondrogenesis, Osteoarthropathy of fingers familial, Dyschondrosteosis-nephritis, Coloboma of Alar-nasal cartilages with telecanthus, Alar cartilages hypoplasia-coloboma-telecanthus, Pierre Robin syndrome-fetal chondrodysplasia, Dysspondyloenchondromatosis, Achondroplasia regional-dysplasia abdominal muscle, Osteochondritis Dissecans, Familial Articular Chondrocalcinosis, Tracheobronchomalacia, Chondritis, Dyschondrosteosis, Jequier-Kozlowski-skeletal dysplasia, Chondrodystrophy, Cranio osteoarthropathy, Tietze's syndrome, Hip dysplasia-ecchondromata, Bessel-Hagen disease, Chondromatosis (benign), Enchondromatosis (benign), Chondrocalcinosis due to apatite crystal deposition, Meyenburg-Altherr-Uehlinger syndrome, Enchondromatosis-dwarfism-deafness, premature growth plate closure (e.g., due to dwarfism, injury, therapy such as retinoid therapy for adolescent acne, or ACL repair), Astley-Kendall syndrome, Synovial osteochondromatosis, Severe achondroplasia with developmental delay and acanthosis nigricans, Chondrocalcinosis, Stanescu syndrome, Familial osteochondritis dissecans, Achondrogenesis type 1A, Achondrogenesis type 2, Achondrogenesis, Langer-Saldino Type, Achondrogenesis type 1B, Achondrogenesis type 1A and 1B, Type II Achondrogenesis-Hypochondrogenesis, Achondrogenesis, Achondrogenesis type 3, Achondrogenesis type 4, Chondrocalcinosis 1, Chondrocalcinosis 2, Chondrocalcinosis familial articular, Diastrophic dysplasia, Fibrochondrogenesis, Hypochondroplasia, Keutel syndrome, Maffucci Syndrome, Osteoarthritis Susceptibility 6, Osteoarthritis Susceptibility 5, Osteoarthritis Susceptibility 4, Osteoarthritis Susceptibility 3, Osteoarthritis Susceptibility 2, Osteoarthritis Susceptibility 1, Pseudoachondroplasia, Cauliflower ear, Costochondritis, Growth plate fractures, Pectus excavatum, septic arthritis, gout, pseudogout (calcium pyrophosphate deposition disease or CPPD), gouty arthritis, bacterial, viral, or fungal infections in or near the joint, bursitis, tendinitis, arthropathies, or another cartilage or joint disease or condition.

In some embodiments, a peptide or peptide conjugate of this disclosure can be administered to a subject in order to target, an arthritic joint. In other embodiments, a peptide or peptide conjugate of this disclosure can be administered to a subject in order to treat an arthritic joint.

In some embodiments, the peptides described herein provide a method of treating a cartilage condition of a subject, the method comprising administering to the subject a therapeutically-effective amount of a peptide comprising any one of sequence SEQ ID NO: 1-SEQ ID NO: 80, or fragment thereof.

### Use of Peptides in Imaging and Surgical Methods

In some embodiments, a peptide of the disclosure is conjugated to and/or delivers a detectable label as described above, including a metal, a radioisotope, a dye, fluorophore, a fluorescent agent, or another suitable material that can be used in imaging.

The present invention provides methods for intraoperative imaging and resection of a cancer, cancerous tissue, tumor tissue, cancerous cells, or diseased tissue using a peptide of the present disclosure conjugated with a detectable agent. In some aspects, the cancer, cancerous tissue, tumor tissue, or diseased tissue or cells of the foregoing is detectable by fluorescence imaging that allows for intraoperative visualization of the cancer, cancerous tissue, tumor tissue, cancerous cells, or diseased tissue using a peptide of the present disclosure. In some aspects, the peptide of the present disclosure is conjugated to one or more detectable agents. In a further embodiment, the detectable agent comprises a fluorescent moiety coupled to the peptide. In another embodiment, the detectable agent comprises a radionuclide. In some aspects, imaging is pre-operative imaging. In other aspects, imaging is achieved during open surgery. In further aspects, imaging is accomplished while using endoscopy or other non-invasive surgical techniques. In yet further aspects, imaging is performed after surgical removal of the cancer, cancerous tissue, tumor tissue, or diseased tissue or cells of the foregoing.

Multiple peptides or peptide conjugates described herein can be administered in any order or simultaneously. In some cases, multiple functional fragments of peptides derived from toxins or venom, or such fragments conjugated to active agents, can be administered in any order or simultaneously. If simultaneously, the multiple peptides or peptide conjugates described herein can be provided in a single, unified form, or in multiple forms, such as by intravenous injection, intravitreal injection, subretinal injection, ophthalmic drops, topical formulation, patch, transdermal patch, oral formulation, or subcutaneous administration. In some cases, peptides or peptide conjugated can be administered directly at or near an area of diseased or target tissue, such as a joint or cartilage. In some cases, such compositions are used for imaging neuronal function, heart function, cancer cells, GI motility, retinal, and eye health and disease.

### Peptide Kit

In one aspect, peptides described herein can be provided as a kit. In another embodiment, peptide conjugates described herein can be provided as a kit. In another embodiment, a kit comprises amino acids encoding a peptide described herein, a vector, a host organism, and an instruction manual. In some embodiments, a kit includes written instructions on the use or administration of the peptides.

As used herein the term “and/or” is used as a functional word to indicate that two words or expressions are to be taken together or individually. For example, A and/or B encompasses A alone, B alone, and A and B together.

All features discussed in connection with any embodiment or embodiment herein can be readily adapted for use in other embodiments and embodiments herein. The use of different terms or reference numerals for similar features in different embodiments does not necessarily imply differences other than those expressly set forth. Accordingly, the present disclosure is intended to be described solely by reference to the appended claims, and not limited to the embodiments disclosed herein.

Unless otherwise specified, the presently described methods and processes can be performed in any order. For example, a method describing steps (a), (b), and (c) can be performed with step (a) first, followed by step (b), and then step (c). Or, the method can be performed in a different order such as, for example, with step (b) first followed by step (c) and then step (a), or any combinations thereof. Furthermore, such steps can be performed in combination with additional steps or methods. Furthermore, those steps can be performed simultaneously or separately unless otherwise specified with particularity.

The particulars shown herein are by way of example and for purposes of illustrative discussion of the preferred embodiments of the present disclosure only and are presented in the cause of providing what is believed to be the most useful and readily understood description of the principles and conceptual embodiments of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

While preferred embodiments of the present disclosure have been shown and described herein, it is to be understood that the disclosure is not limited to the particular embodiments of the disclosure described, as variations of the particular embodiments can be made and still fall within the scope of the appended claims. It is also to be understood that the terminology employed is for the purpose of describing particular embodiments of the disclosure, and is not intended to be limiting. Instead, the scope of the present disclosure is established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the disclosure provided herein.

All features discussed in connection with an embodiment or embodiment herein can be readily adapted for use in other embodiments and embodiments herein. The use of different terms or reference numerals for similar features in different embodiments does not necessarily imply differences other than those expressly set forth. Accordingly, the present disclosure is intended to be described solely by reference to the appended claims, and not limited to the embodiments disclosed herein.

### EXAMPLES

The following examples are included to further describe some embodiments of the present disclosure, and should not be used to limit the scope of the disclosure.

### Example 1

### Manufacture of Peptides

This example describes the manufacture of the peptides described herein. Peptides derived from knottin proteins were generated in mammalian cell culture using a published methodology. (A. D. Bandaranayke, C. Correnti, B. Y. Ryu, M. Brault, R. K. Strong, D. Rawlings. 2011. Daedalus: a robust, turnkey platform for rapid production of decigram quantities of active recombinant proteins in human cell lines using novel lentiviral vectors. Nucleic Acids Research. (39)21, e143).

The peptide sequence was reverse-translated into DNA, synthesized, and cloned in-frame with siderocalin using standard molecular biology techniques. (M. R. Green, Joseph Sambrook. Molecular Cloning. 2012 Cold Spring Harbor Press.). The resulting construct was packaged into a lentivirus, transduced into HEK-293 cells, expanded, isolated by immobilized metal affinity chromatography (IMAC), cleaved with tobacco etch virus protease, and purified to homogeneity by reverse-phase chromatography. Following purification, each peptide was lyophilized and stored frozen.

### Example 2

### Peptide Expression Using a Mammalian Expression System

This example describes expression of the peptides using a mammalian expression system. Peptides were expressed according to the methods described in in Bandaranayake et al., Nucleic Acids Res. 2011 November; 39(21): e143. Peptides were cleaved from siderocalin using tobacco etch virus protease and purified by FPLC on a hydrophobic columns using a gradient of acetonitrile and 0.1% TFA. Peptides were then lyophilized and stored frozen.

### Example 3

### Peptide Radiolabeling

This example describes the radiolabeling of peptides. Several peptides were radiolabeled by reductive methylation with ¹⁴C formaldehyde and sodium cyanoborohydride with standard techniques (such as those described in Jentoft et al. J Biol Chem. 254(11):4359-65. 1979). The sequences were engineered to have the amino acids, “G” and “S” at the N terminus. See Methods in Enzymology V91:1983 p. 570 and JBC 254(11):1979 p. 4359. An excess of formaldehyde was used to drive complete methylation (dimethylation of every free amine). The labeled peptides were isolated via solid-phase extraction on Strata-X columns (Phenomenex 8B-S100-AAK), rinsed with water with 5% methanol, and recovered in methanol with 2% formic acid. Solvent was subsequently removed in a blowdown evaporator with gentle heat and a stream of nitrogen gas.

### Example 4

### Ion Channel Inhibitory Activity of Select Peptides

This example describes a method for assaying ion channel activity in cells. The selectivity profile of 40 peptides, SEQ ID NO: 1-40, were expressed and tested against a panel of 20 ion channels using a commercial electrophysiological assay platform (IonWorks Barracuda, Charles River).

Peptides were screened in duplicate at final concentrations of 20 μM and 200 nM, as determined by amino acid analysis, and diluted into HEPES buffered physiological saline (HBPS). HEK293 or CHO cells from the American Type Culture Collection (ATCC) were transfected with the appropriate ion channel before use. HEK293 cells were grown in Dulbecco's Modified Eagle Medium/Nutrient Mixture F-12, and CHO cells were cultured in Ham's F-12 media. All cultures were supplemented with 10% fetal bovine serum, 100 U/ml penicillin G, 100 μg/ml streptomycin sulfate, and the appropriate selection antibiotics. Prior to use, cells were washed twice with Hank's Balanced Salt Solution, and treated with Accutase cell detachment solution (Sigma-Aldrich). Cells were then washed. Peptides and controls were incubated with cells for at least five minutes. Controls, matched to particular channels, include ondansetron, mecamylamine, mibefradil dihydrochloride, picrotoxin, E-4031, BaCl2, 4-aminopyridine, verapamil, lidocaine, memantine, mustard oil, and capsazepine (sourced from Sigma-Aldrich or Tocris).

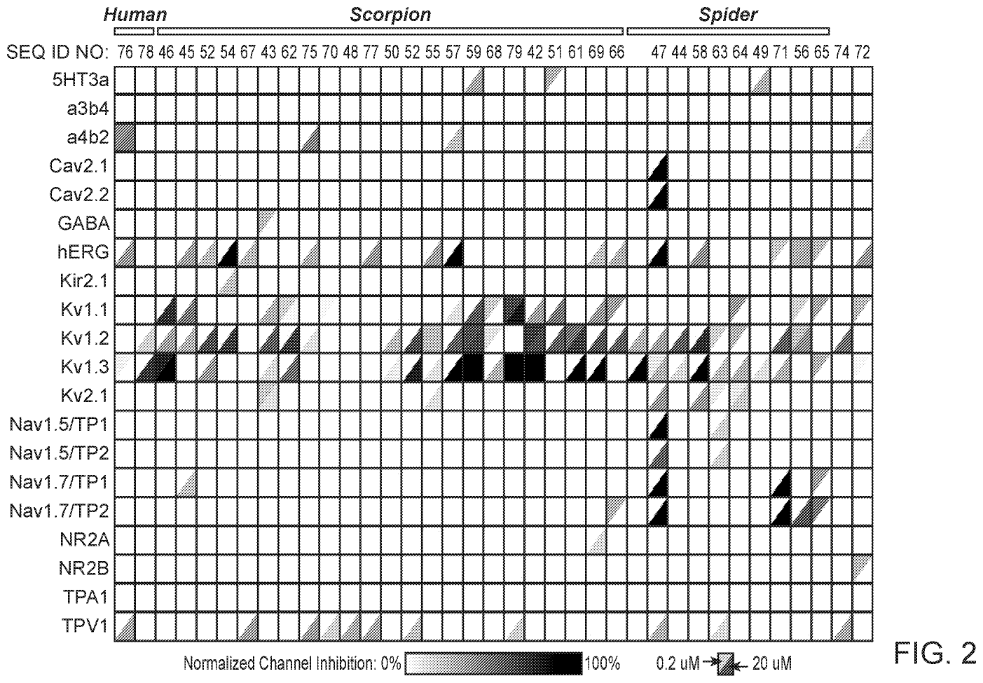

The effect of 40 select peptides on 20 ion channels at two dosages (0.2 or 20 μM) were examined and shown with triangular gnomons, with grayscale intensity indicating the degree of the ion channel inhibitory effect (darker shading referring to greater inhibition of ion channel). Percent inhibition was defined as: (1-(I_{peptide}/I_{control}))×100%). Individual assays were run in duplicates, and only effects greater than 3σ are illustrated in FIG. 2.

SEQ ID NO: 5 and 6 were active on a subset of potassium channels. SEQ ID NO: 6 is related to Kaliotoxin-1, a known Kv channel inhibitor. SEQ ID NO: 5 showed additional activity against hERG (Kv11.1) and Nav1.7 channels. SEQ ID NO: 14 had been previously reported to inhibit hERG channels, but showed activity against additional potassium channels. SEQ ID NO: 3 (or Hadrucalcin) and SEQ ID NO: 11 (or Opicalcin-2) had been reported to inhibit calcium channels (ryanodine receptors), but also showed activity against additional potassium channels or serotonin receptors (e.g., 5-HT3a). SEQ ID NO: 35, CTX, was a known inhibitor of chloride channels, but also showed activity against the α4β2 nicotinic receptor, and the hERG and Kv1.2 potassium channels. SEQ ID NO: 12 had been previously reported to inhibit Kv1.3, consistent with these results. CDP #47 had been reported to inhibit small conductance, calcium-activated K channels, but not Kv channels as observed here. SEQ ID NO: 17 had previously been demonstrated to bind to hERG (see U.S. Pat. No. 7,326,772 B2), but FIG. 2 confirmed hERG inhibition. SEQ ID NO: 19 and 39 showed strong Kv1.3 inhibition in these results, consistent with prior studies. SEQ ID NO: 20 and 26 showed activity on various subsets of potassium channels, while SEQ ID NO: 7 inhibited voltage-sensitive calcium channels, and SEQ ID NO: 31 showed activity on tetrodotoxin-sensitive sodium channels (e.g., Nav1.7).

Percent values were calculated using the following formula: % activation=(I_{Peptide}/I_{control})×100%, where I_{Peptide} is the current produced by co-application a peptide with agonist, and I_{control} refers to the current produced by stimulation in control. Control used for each ion channel is the reference compound matched to a particular channel, as illustrated in TABLE 10 below.

TABLE 10 illustrates the IC₅₀ values of receptor/channel inhibition with the reference compound (or control).

TABLE 10
IC₅₀ Values of Receptor/Channel Inhibition
With Reference Compounds

| Receptor/Channel | Reference Compound | IC₅₀, μM |
|---|---|---|
| 5HT3a | Ondansetron | 0.005 |
| α3β4 | Mecamylamine | 1.77 |
| α4β2 | Mecamylamine | 1.14 |
| Cav2.1 | Mibefradil dihydrochloride | 2.12 |
| Cav2.2 | Mibefradil dihydrochloride | 8.28 |
| GABA | Picrotoxin | 1.72 |
| hERG | E-4031 | 0.03 |
| Kir2.1 | BaCl2 | 5.25 |
| Kv1.1 | 4-aminopyridine | 156 |
| Kv1.2 | 4-aminopyridine | 76 |
| Kv1.3 | 4-aminopyridine | 154 |
| Kv2.1 | Verapamil | 78 |
| Nav1.5 (TP1) | Lidocaine | 572 |
| Nav1.5 (TP2) | Lidocaine | 33 |
| Nav1.7 (TP1) | Lidocaine | 465 |
| Nav1.7(TP2) | Lidocaine | 285 |
| NR2A | Memantine | 3.77 |
| NR2B | Memantine | 1.71 |
| TRPA1 | Mustard Oil | 220 |
| TRPV1 | Capsazepine | 0.09 |

A summary of the results for each peptide assayed (SEQ ID NO: 1-SEQ ID NO: 40) is illustrated at TABLE 11 and TABLE 12 below.

TABLE 11 illustrates the activity of receptors/channels in the presence of 0.2 μM of each peptide, wherein the values are expressed as a percentage of the reference compound. Bolded numbers indicate inhibition by a peptide that is greater than 3 standard deviations from the reference compound. A value of 100% means no change to ion channel activity by application of test compound. A value less than 100% indicates the channel was partially or completely blocked or inhibited by application of the test compound.

TABLE 12 illustrates the activity of receptors/channels in the presence of 20 μM of each peptide, wherein the values are expressed as a percentage of the reference compound. Bolded numbers indicate inhibition by a peptide that is greater than 3 standard deviations from the reference compound. A value of 100% means no change to ion channel activity by application of test compound. A value less than 100% indicates the channel was partially or completely blocked or inhibited by application of the test compound.

TABLE 11
Activity of Receptors/Channels With Peptides at 0.2 μM

| SEQ | | | | | | | | | | | |
|---|---|---|---|---|---|---|---|---|---|---|---|
| ID NO: | 5HT3a | a3b4 | a4b2 | Cav2.1 | Cav2.2 | GABA | hERG | Kir2.1 | Kv1.1 | Kv1.2 | Kv1.3 |
| 1 | 90 | 100 | 113 | 101 | 96 | 105 | 110 | 109 | 87 | 85 | 106 |
| 2 | 92 | 85 | 95 | 103 | 104 | 104 | 97 | 101 | 114 | 34 | 8 |
| 3 | 109 | 99 | 93 | 102 | 103 | 77 | 121 | 107 | 99 | 88 | 92 |
| 4 | 103 | 107 | 86 | 111 | 105 | 121 | 110 | 109 | 106 | 90 | 109 |
| 5 | 94 | 104 | 83 | 100 | 94 | 109 | 106 | 103 | 86 | 89 | 98 |
| 6 | 85 | 100 | 71 | 109 | 103 | 104 | 107 | 96 | 96 | 81 | 25 |
| 7 | 98 | 104 | 97 | 118 | 87 | 118 | 95 | 103 | 92 | 111 | 92 |
| 8 | 97 | 108 | 106 | 98 | 106 | 108 | 109 | 100 | 111 | 109 | 98 |
| 9 | 107 | 105 | 96 | 111 | 109 | 99 | 104 | 105 | 103 | 120 | 107 |
| 10 | 90 | 97 | 96 | 104 | 98 | 129 | 97 | 100 | 117 | 135 | 101 |
| 11 | 73 | 95 | 111 | 104 | 100 | 96 | 97 | 107 | 99 | 103 | 101 |
| 12 | 96 | 103 | 97 | 109 | 115 | 102 | 89 | 97 | 107 | 88 | 68 |
| 13 | 96 | 103 | 97 | 109 | 121 | 107 | 89 | 97 | 107 | 88 | 68 |
| 14 | 96 | 99 | 106 | 118 | 129 | 107 | 82 | 95 | 79 | 73 | 87 |
| 15 | 86 | 91 | 71 | 106 | 105 | 122 | 79 | 107 | 69 | 77 | 86 |
| 16 | 114 | 100 | 83 | 113 | 124 | 102 | 74 | 98 | 84 | 78 | 92 |
| 17 | 81 | 98 | 93 | 107 | 117 | 97 | 85 | 103 | 106 | 97 | 96 |
| 18 | 95 | 110 | 109 | 97 | 132 | 113 | 98 | 104 | 98 | 67 | 87 |
| 19 | 89 | 101 | 99 | 106 | 116 | 93 | 98 | 107 | 100 | 41 | 4 |
| 20 | 96 | 103 | 113 | 100 | 110 | 109 | 90 | 89 | 94 | 115 | 80 |
| 21 | 87 | 102 | 102 | 103 | 98 | 111 | 109 | 105 | 106 | 52 | 65 |
| 22 | 77 | 98 | 92 | 98 | 130 | 99 | 84 | 94 | 83 | 82 | 100 |
| 23 | 74 | 94 | 87 | 95 | 110 | 100 | 92 | 105 | 95 | 83 | 81 |
| 24 | 85 | 101 | 97 | 92 | 120 | 96 | 88 | 104 | 65 | 74 | 94 |
| 25 | 82 | 105 | 73 | 89 | 111 | 111 | 76 | 79 | 69 | 72 | 74 |
| 26 | 82 | 88 | 85 | 115 | 103 | 117 | 80 | 102 | 66 | 79 | 94 |
| 27 | 105 | 98 | 90 | 108 | 105 | 112 | 90 | 101 | 81 | 84 | 106 |
| 28 | 74 | 104 | 96 | 112 | 94 | 116 | 98 | 104 | 82 | 89 | 92 |
| 29 | 122 | 108 | 108 | 100 | 116 | 123 | 95 | 103 | 97 | 77 | 21 |
| 30 | 71 | 91 | 89 | 111 | 96 | 102 | 95 | 102 | 98 | 110 | 89 |
| 31 | 75 | 96 | 91 | 98 | 109 | 92 | 81 | 94 | 94 | 128 | 98 |
| 32 | 95 | 100 | 81 | 98 | 102 | 98 | 90 | 95 | 76 | 89 | 98 |
| 33 | 97 | 96 | 81 | 92 | 116 | 103 | 104 | 85 | 94 | 92 | 79 |
| 34 | 114 | 103 | 81 | 86 | 130 | 107 | 84 | 97 | 109 | 96 | 108 |
| 35 | 84 | 98 | 77 | 108 | 112 | 118 | 88 | 108 | 98 | 89 | 113 |
| 36 | 81 | 95 | 62 | 96 | 114 | 118 | 84 | 108 | 82 | 91 | 96 |
| 37 | 82 | 104 | 89 | 108 | 111 | 95 | 89 | 97 | 99 | 92 | 93 |
| 38 | 97 | 99 | 107 | 95 | 117 | 121 | 86 | 88 | 97 | 100 | 101 |
| 39 | 67 | 99 | 98 | 96 | 117 | 111 | 103 | 110 | 37 | 96 | 7 |
| 40 | 114 | 101 | 97 | 98 | 103 | 114 | 80 | 79 | 100 | 4 | 89 |
| SEQ | | | | | | | | | | | |
| ID NO: | Kv2.1 | Nav1.5 | Nav1.5 | Nav1.7 | Nav1.7 | NR2A | NR2B | TRPA1 | TRPV1 | | |
| 1 | 99 | 91 | 85 | 84 | 67 | 81 | 113 | 116 | 108 | | |
| 2 | 80 | 95 | 93 | 89 | 74 | 102 | 118 | 106 | 95 | | |
| 3 | 80 | 113 | 111 | 107 | 113 | 97 | 93 | 105 | 107 | | |
| 4 | 112 | 117 | 118 | 95 | 93 | 106 | 117 | 135 | 110 | | |
| 5 | 110 | 107 | 107 | 98 | 109 | 109 | 112 | 119 | 101 | | |
| 6 | 103 | 105 | 104 | 98 | 95 | 86 | 92 | 126 | 86 | | |
| 7 | 105 | 121 | 118 | 101 | 90 | 107 | 117 | 151 | 118 | | |
| 8 | 108 | 105 | 105 | 92 | 75 | 99 | 107 | 95 | 101 | | |
| 9 | 112 | 104 | 103 | 108 | 110 | 89 | 77 | 131 | 112 | | |
| 10 | 88 | 93 | 90 | 105 | 98 | 84 | 82 | 108 | 111 | | |
| 11 | 121 | 107 | 107 | 107 | 107 | 103 | 103 | 100 | 100 | | |
| 12 | 104 | 98 | 95 | 61 | 66 | 94 | 122 | 129 | 97 | | |
| 13 | 104 | 98 | 95 | 61 | 66 | 94 | 122 | 129 | 97 | | |
| 14 | 109 | 85 | 82 | 83 | 89 | 107 | 123 | 106 | 133 | | |
| 15 | 91 | 90 | 91 | 91 | 97 | 97 | 103 | 114 | 124 | | |
| 16 | 114 | 107 | 104 | 86 | 91 | 101 | 88 | 99 | 107 | | |
| 17 | 88 | 96 | 95 | 66 | 61 | 83 | 90 | 176 | 99 | | |
| 18 | 93 | 104 | 106 | 85 | 84 | 98 | 107 | 130 | 93 | | |
| 19 | 95 | 112 | 114 | 91 | 92 | 90 | 117 | 126 | 94 | | |
| 20 | 99 | 103 | 101 | 92 | 59 | 98 | 104 | 114 | 109 | | |
| 21 | 110 | 108 | 105 | 112 | 120 | 105 | 113 | 119 | 106 | | |
| 22 | 110 | 90 | 90 | 79 | 81 | 107 | 94 | 90 | 102 | | |
| 23 | 93 | 85 | 87 | 89 | 93 | 88 | 95 | 126 | 117 | | |
| 24 | 91 | 94 | 95 | 84 | 97 | 102 | 102 | 132 | 131 | | |
| 25 | 88 | 76 | 74 | 66 | 54 | 94 | 99 | 124 | 117 | | |
| 26 | 98 | 92 | 91 | 81 | 74 | 113 | 86 | 109 | 116 | | |
| 27 | 91 | 94 | 93 | 104 | 114 | 98 | 79 | 85 | 99 | | |
| 28 | 100 | 96 | 96 | 85 | 81 | 104 | 100 | 155 | 104 | | |
| 29 | 91 | 110 | 114 | 97 | 111 | 93 | 101 | 89 | 109 | | |
| 30 | 107 | 105 | 102 | 103 | 125 | 111 | 92 | 118 | 107 | | |
| 31 | 111 | 87 | 84 | 97 | 98 | 93 | 104 | 81 | 107 | | |
| 32 | 92 | 108 | 101 | 96 | 97 | 95 | 76 | 130 | 99 | | |
| 33 | 87 | 101 | 100 | 74 | 50 | 91 | 101 | 124 | 101 | | |
| 34 | 98 | 111 | 113 | 102 | 104 | 112 | 127 | 91 | 110 | | |
| 35 | 109 | 104 | 100 | 74 | 89 | 87 | 104 | 145 | 110 | | |
| 36 | 104 | 110 | 109 | 108 | 118 | 115 | 109 | 106 | 103 | | |
| 37 | 108 | 102 | 99 | 107 | 102 | 101 | 103 | 88 | 107 | | |
| 38 | 100 | 124 | 124 | 83 | 143 | 100 | 83 | 138 | 98 | | |
| 39 | 118 | 108 | 105 | 42 | 39 | 106 | 102 | 136 | 95 | | |
| 40 | 97 | 107 | 108 | 68 | 74 | 121 | 71 | 130 | 114 | | |

TABLE 12
Activity of Receptors/Channels With Peptides at 20 μM

| SEQ | | | | | | | | | | | |
|---|---|---|---|---|---|---|---|---|---|---|---|
| ID NO: | 5HT3a | a3b4 | a4b2 | Cav2.1 | Cav2.2 | GABA | hERG | Kir2.1 | Kv1.1 | Kv1.2 | Kv1.3 |
| 1 | 72 | 96 | 102 | 109 | 116 | 91 | 84 | 104 | 113 | 43 | 56 |
| 2 | 88 | 88 | 87 | 108 | 104 | 97 | 76 | 89 | 68 | 39 | 1 |
| 3 | 98 | 92 | 91 | 131 | 110 | 111 | 81 | 93 | 73 | 45 | 87 |
| 4 | 76 | 97 | 81 | 130 | 101 | 108 | 80 | 90 | 91 | 45 | 87 |
| 5 | 80 | 90 | 93 | 121 | 117 | 114 | 67 | 86 | 55 | 73 | 28 |
| 6 | 84 | 95 | 96 | 111 | 107 | 96 | 71 | 94 | 30 | 85 | 0 |
| 7 | 84 | 87 | 84 | −6 | −2 | 121 | 1 | 101 | 102 | 68 | 74 |
| 8 | 78 | 103 | 93 | 126 | 123 | 121 | 84 | 90 | 93 | 104 | 93 |
| 9 | 73 | 103 | 81 | 108 | 102 | 98 | 98 | 103 | 97 | 104 | 89 |
| 10 | 104 | 104 | 94 | 96 | 94 | 111 | 80 | 92 | 84 | 77 | 93 |
| 11 | 85 | 96 | 88 | 110 | 103 | 105 | 99 | 97 | 55 | 46 | 87 |
| 12 | 97 | 94 | 84 | 104 | 115 | 93 | 81 | 102 | 84 | 41 | 23 |
| 13 | 84 | 83 | 80 | 100 | 100 | 93 | 67 | 98 | 79 | 40 | 64 |
| 14 | 78 | 93 | 81 | 111 | 95 | 120 | 1 | 79 | 99 | 34 | 82 |
| 15 | 74 | 86 | 95 | 86 | 106 | 97 | 67 | 88 | 72 | 76 | 90 |
| 16 | 103 | 95 | 72 | 92 | 104 | 109 | 71 | 92 | 67 | 61 | 92 |
| 17 | 85 | 97 | 78 | 90 | 100 | 108 | 8 | 84 | 89 | 47 | 6 |
| 18 | 79 | 89 | 92 | 98 | 124 | 106 | 62 | 93 | 78 | 30 | 7 |
| 19 | 69 | 97 | 93 | 109 | 94 | 111 | 86 | 93 | 44 | 37 | −1 |
| 20 | 91 | 94 | 92 | 116 | 105 | 106 | 87 | 95 | 95 | 74 | 7 |
| 21 | 91 | 93 | 85 | 106 | 105 | 123 | 72 | 101 | 98 | 36 | 5 |
| 22 | 95 | 92 | 83 | 99 | 86 | 124 | 79 | 97 | 97 | 34 | 51 |
| 23 | 101 | 93 | 79 | 102 | 95 | 103 | 78 | 82 | 102 | 106 | 77 |
| 24 | 84 | 87 | 88 | 121 | 102 | 114 | 78 | 90 | 69 | 83 | 73 |
| 25 | 94 | 91 | 75 | 109 | 111 | 98 | 79 | 98 | 77 | 85 | 87 |
| 26 | 96 | 91 | 89 | 85 | 121 | 106 | 74 | 93 | 85 | 47 | 77 |
| 27 | 78 | 93 | 82 | 103 | 116 | 105 | 75 | 86 | 89 | 107 | 100 |
| 28 | 114 | 98 | 90 | 109 | 114 | 114 | 76 | 95 | 95 | 102 | 73 |
| 29 | 91 | 98 | 101 | 111 | 100 | 104 | 78 | 97 | 64 | 42 | 0 |
| 30 | 91 | 101 | 92 | 103 | 101 | 114 | 95 | 93 | 102 | 92 | 71 |
| 31 | 84 | 90 | 88 | 83 | 97 | 115 | 68 | 101 | 106 | 45 | 74 |
| 32 | 105 | 96 | 86 | 92 | 101 | 105 | 65 | 79 | 114 | 112 | 98 |
| 33 | 56 | 90 | 59 | 104 | 93 | 107 | 77 | 97 | 55 | 94 | 26 |
| 34 | 97 | 97 | 51 | 111 | 94 | 117 | 66 | 50 | 111 | 51 | 100 |
| 35 | 71 | 95 | 58 | 104 | 102 | 116 | 72 | 84 | 99 | 89 | 91 |
| 36 | 83 | 87 | 57 | 102 | 87 | 106 | 65 | 91 | 101 | 87 | 93 |
| 37 | 87 | 96 | 81 | 97 | 105 | 111 | 64 | 91 | 98 | 90 | 100 |
| 38 | 98 | 92 | 88 | 93 | 89 | 112 | 71 | 126 | 73 | 84 | 31 |
| 39 | 55 | 93 | 91 | 96 | 98 | 108 | 84 | 98 | 21 | 84 | 1 |
| 40 | 115 | 95 | 77 | 86 | 93 | 104 | 55 | 103 | 105 | 48 | 18 |
| SEQ | | | | | | | | | | | |
| ID NO: | Kv2.1 | Nav1.5 | Nav1.5 | Nav1.7 | Nav1.7 | NR2A | NR2B | TRPA1 | TRPV1 | | |
| 1 | 119 | 111 | 109 | 85 | 72 | 99 | 88 | 112 | 78 | | |
| 2 | 87 | 102 | 101 | 61 | 91 | 95 | 72 | 92 | 81 | | |
| 3 | 81 | 114 | 115 | 90 | 68 | 100 | 74 | 117 | 95 | | |
| 4 | 102 | 98 | 101 | 96 | 104 | 90 | 93 | 119 | 98 | | |
| 5 | 109 | 103 | 105 | 79 | 66 | 86 | 95 | 75 | 101 | | |
| 6 | 124 | 116 | 120 | 116 | 119 | 88 | 75 | 92 | 98 | | |
| 7 | 65 | 9 | 46 | 1 | 7 | 65 | 53 | 85 | 73 | | |
| 8 | 112 | 100 | 99 | 97 | 110 | 87 | 107 | 115 | 72 | | |
| 9 | 99 | 109 | 110 | 94 | 114 | 87 | 85 | 112 | 79 | | |
| 10 | 109 | 114 | 116 | 106 | 90 | 65 | 98 | 107 | 98 | | |
| 11 | 110 | 113 | 118 | 96 | 87 | 106 | 78 | 109 | 100 | | |
| 12 | 113 | 131 | 136 | 74 | 63 | 91 | 83 | 80 | 78 | | |
| 13 | 116 | 97 | 101 | 71 | 62 | 108 | 117 | 113 | 88 | | |
| 14 | 93 | 94 | 97 | 72 | 65 | 95 | 72 | 66 | 92 | | |
| 15 | 88 | 78 | 79 | 75 | 58 | 76 | 88 | 76 | 99 | | |
| 16 | 98 | 93 | 93 | 66 | 43 | 95 | 91 | 116 | 88 | | |
| 17 | 93 | 95 | 94 | 75 | 60 | 96 | 77 | 129 | 82 | | |
| 18 | 65 | 87 | 112 | 43 | 55 | 79 | 78 | 89 | 61 | | |
| 19 | 108 | 117 | 117 | 121 | 140 | 99 | 130 | 109 | 90 | | |
| 20 | 97 | 102 | 105 | 81 | 95 | 104 | 92 | 114 | 102 | | |
| 21 | 108 | 96 | 91 | 105 | 123 | 103 | 105 | 95 | 86 | | |
| 22 | 93 | 112 | 116 | 94 | 87 | 129 | 97 | 128 | 67 | | |
| 23 | 103 | 85 | 83 | 65 | 63 | 89 | 82 | 83 | 87 | | |
| 24 | 81 | 99 | 104 | 80 | 83 | 100 | 92 | 107 | 102 | | |
| 25 | 107 | 88 | 87 | 90 | 83 | 106 | 94 | 129 | 113 | | |
| 26 | 102 | 90 | 94 | 73 | 60 | 114 | 79 | 115 | 92 | | |
| 27 | 94 | 104 | 109 | 97 | 97 | 88 | 89 | 114 | 67 | | |
| 28 | 109 | 100 | 102 | 104 | 102 | 90 | 100 | 84 | 86 | | |
| 29 | 121 | 103 | 99 | 67 | 62 | 87 | 82 | 109 | 86 | | |
| 30 | 106 | 94 | 96 | 84 | 54 | 98 | 88 | 111 | 84 | | |
| 31 | 111 | 107 | 110 | −2 | −5 | 98 | 79 | 88 | 71 | | |
| 32 | 93 | 90 | 90 | 87 | 61 | 110 | 106 | 108 | 84 | | |
| 33 | 103 | 102 | 103 | 99 | 112 | 87 | 70 | 67 | 86 | | |
| 34 | 97 | 101 | 101 | 86 | 80 | 77 | 66 | 97 | 71 | | |
| 35 | 125 | 110 | 112 | 88 | 83 | 91 | 83 | 118 | 66 | | |
| 36 | 92 | 107 | 112 | 91 | 71 | 103 | 96 | 92 | 69 | | |
| 37 | 95 | 111 | 110 | 105 | 97 | 83 | 82 | 105 | 68 | | |
| 38 | 125 | 107 | 112 | 86 | 86 | 90 | 88 | 108 | 76 | | |
| 39 | 102 | 111 | 114 | 108 | 110 | 82 | 85 | 92 | 83 | | |
| 40 | 105 | 79 | 73 | 80 | 79 | 50 | 54 | 129 | 79 | | |

As indicated by the bolded numbers in TABLE 11 and TABLE 12, some peptides showed marked inhibition of ion channels at both concentrations tested, that is: <50% activity at 20 μM and <80% activity at 0.2 μM, which are summarized in TABLE 13 below. Some peptides showed marked activity on one Kv1.x channel but less or none on other channels.

TABLE 13
Marked Peptide Inhibition of Ion Channels
at both 20 μM and 0.2 μM

| Ion Channel Inhibition: | |
|---|---|
| (<50% activity at 20 μM | |
| and <80% activity at 0.2 μM) | Peptide |
| Kv1.1 | SEQ ID NO 39 |
| Kv1.2 | SEQ ID NO: 2, 14, 18, 19, 21, 26, |
| | 29, 40 |
| Kv1.3 | SEQ ID NO: 2, 6, 12, 19, 20, 21, |
| | 29, 33, 39 |

As indicated by the bolded numbers in TABLE 12, some peptides showed marked inhibition of ion channels tested, that is: <50% activity at 20 μM, which are summarized in TABLE 14 below. Some peptides show this marked inhibition on only one channel whereas other peptides show this marked inhibition on multiple channels.

TABLE 14
Peptide Inhibition of Ion Channels at 20 μM

| Ion Channel Inhibition | |
|---|---|
| (<50% activity at 20 μM) | Peptide |
| Cav2.1 | SEQ ID NO: 7 |
| Cav2.2 | SEQ ID NO: 7 |
| Kv1.1 | SEQ ID NO: 6, 19, 39 |
| Kv1.2 | SEQ ID NO: 1, 2, 3, 4, 11, 12, 13, 14, 17, 18, |
| | 19, 21, 22, 26, 29, 31,40 |
| Kv 1.3 | SEQ ID NO: 2, 5, 6, 12, 17, 18, 19, 20, 21, 29, |
| | 33, 38, 39, 40 |
| Nav1.5 (TP1) | SEQ ID NO: 7 |
| Nav1.5 (TP2) | SEQ ID NO: 7 |
| Nav 1.7 (TP1) | SEQ ID NO: 7, 18, 31 |
| Nav 1.7 (TP2) | SEQ ID NO: 7, 16, 31 |
| NR2A | SEQ ID NO: 40 |
| hERG | SEQ ID NO: 7, 14, 17 |

### Example 5

### Effects of Three Peptides on Human Kv1.3, Kv1.5, Nav1.3, Nav1.4, Nav1.5 and Nav1.7 Channels Expressed in Mammalian Cells

The example illustrates the effects of three peptides, SEQ ID NO: 5, SEQ ID NO: 6, and SEQ ID NO: 10, on human Kv1.3, Kv1.5, Nav1.3, Nav1.4, Nav1.5 and Nav1.7 ion channels expressed in mammalian cells. Nav1.3 sodium channel (human SCN3A gene), Nav1.4 sodium channel (human SCN4A gene), Nav1.5 cardiac sodium channel (human SCN5A gene), Nav1.7 sodium channel (human SCN9A gene), Kv1.3 potassium channel (human KCNA3 gene), and Kv1.5 potassium channel (human KCNA5 gene) were expressed in CHO cells. Effects of SEQ ID NO: 5, SEQ ID NO: 6, and SEQ ID NO: 10 peptides on these ion channels expressed in CHO cells were evaluated in 8-point concentration-response format (4 replicate wells/concentration). All test and control solutions contained 1% PBS and 0.1% BSA. The peptide formulations were loaded in a 384-well compound plate using an automated liquid handling system (SciClone ALH3000, Caliper LifeScienses).

TABLE 15 below describes the peptides assayed in vivo.

TABLE 15
Peptides Assayed In Vivo

| | Test Concentrations | Test Concentrations |
|---|---|---|
| SEQ ID | (Kv1.3 and Kv1.5) | (Nav1.3, Nav1.4, Nav1.5 |
| NO: | (μM) | and Nav1.7) (μM) |
| 5 | 0.0003, 0.001, 0.003, 0.01, | 0.001, 0.01, 0.1, 1 |
| | 0.03, 0.1, 0.3, 1 | |
| 6 | 0.0003, 0.001, 0.003, 0.01, | 0.001, 0.01, 0.1, 1 |
| | 0.03, 0.1, 0.3, 1 | |
| 10 | 0.0003, 0.001, 0.003, 0.01, | 0.001, 0.01, 0.1, 1 |
| | 0.03, 0.1, 0.3, 1 | |

Positive controls used in the experiment were: Tetrodotoxin (TTX) at four concentrations (0.001, 0.01, 0.1 and 1 μM) for Nav1.x channels; and 4-aminopyridine (4-AP) at eight concentrations (1, 3, 10, 30, 100, 300, 1000, 3000 μM) for Kv1.x channels

Cells were maintained in tissue culture incubators per standard ChanTest procedure. Before testing, cells in culture dishes were washed twice with Hank's Balanced Salt Solution, treated with accutase and re-suspended in the culture media (8-10×10⁶ cells in 20 ml). Cells in suspension were allowed to recover for 10 minutes in tissue culture incubator set at 37° C. in a humidified 95% air: 5% CO₂ atmosphere. Immediately before use in the IonWorks Barracuda™ system, the cells were washed twice in HEPES buffered physiological saline (HBPS) to remove the accutase/culture medium and re-suspended in 5 mL of HBPS.

The peptide effects were evaluated using IonWorks™ Barracuda systems (Molecular Devices Corporation, Union City, Calif.). HEPES-buffered intracellular solution (ChanTest proprietary) used for whole cell recordings was loaded into the intracellular compartment of the Population Patch Clamp™ (PPC) planar electrode. Extracellular buffer (HBPS) was loaded into PPC planar electrode plate wells (11 μL per well). The cell suspension was pipetted into the wells of the PPC planar electrode (9 μL per well). After establishment of a whole-cell configuration (the perforated patch), membrane currents were recorded using patch clamp amplifier in the IonWorks™ Barracuda system. The current recordings were performed one time before peptide application to the cells (baseline) and one time after application of the peptide. Peptide concentrations were applied to naïve cells (n=4, where n=replicates/concentration).

Nav1.x Voltage Protocol:

Inhibition of Nav1.x channels was measured using a stimulus voltage pattern. The pulse pattern (PP) was repeated before (baseline) and for five minutes after adding either the peptide or the reference compound control was added. Peak current amplitudes were measured for test pulses TP1 (tonic inhibition) and TP2 (inactivated state inhibition).

Kv1.x Voltage Protocol:

Kv1.x currents were elicited using a stimulus voltage pattern.

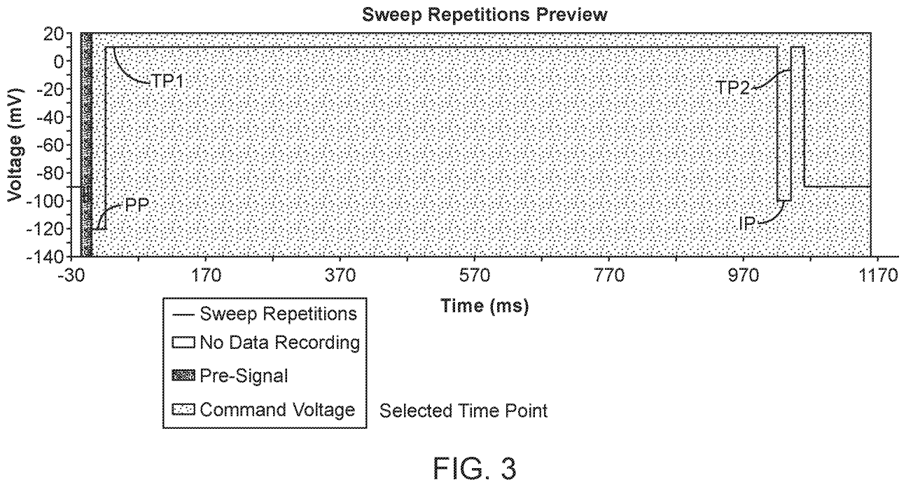

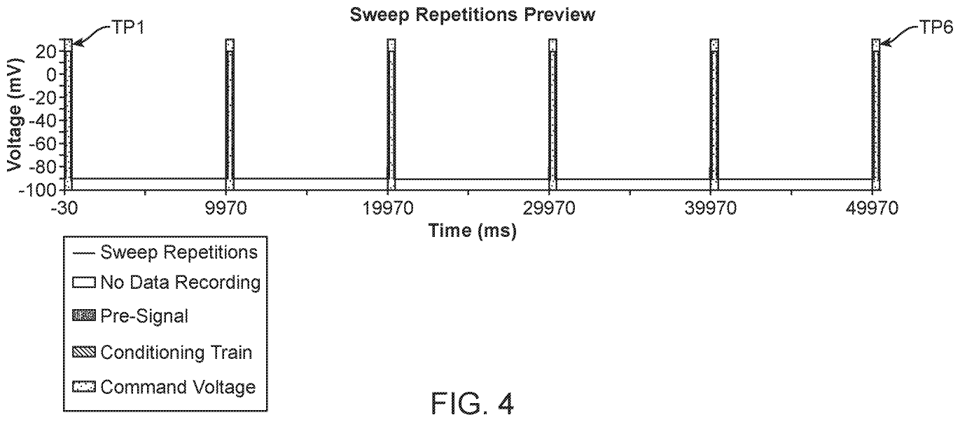

The decrease in current amplitude after peptide application was used to calculate the percent inhibition relative to the positive control. See FIG. 3 to FIG. 10.

The inhibition effect of the peptide was calculated as follows:

% Block=(1−I_{Peptide}/I_{Control})×100%,

where I_{Control} and I_{Peptide} were the currents measured before adding the peptide or in the presence of the peptide, respectively. The data were corrected for run-down according to the following formula:

% Block=100%−((% Block−% PC)*(100%/(% VC−% PC)),

where % VC and % PC are the mean values of the current inhibition with the vehicle and positive controls, respectively.

Concentration-response data were fit to an equation of the following formula:

% Block=(% 100/[1+([Test]/IC₅₀)^{N}],

where [Test] was the concentration of peptide, IC₅₀ was the concentration of the peptide producing half-maximal inhibition, N was the Hill coefficient and % Block was the percentage of ion channel current inhibited at each concentration of a peptide. Nonlinear least squares fits were solved with the XLfit add-in for Excel (Microsoft, Redmond, Wash.).

Kv1.x Channel Results:

IC₅₀ values for ion channel inhibition for each peptide are summarized in TABLE 16 below. Z′ factor values are presented in TABLE 17. In cases where the maximal inhibition effect was less than 50%, the IC₅₀ value was not calculated. SEQ ID NO: 6 may show an IC₅₀ against Kv1.3 less than 1 uM.

TABLE 16
IC₅₀ Values for Kv1.3 and Kv1.5 Ion Channel
Inhibition with Peptides and Positive Control

| IC₅₀, μM | |
|---|---|
| Kv1.3 | Kv1.5 |

| Peptide/Control | TP1 | TP6 | TP1 | TP6 |
|---|---|---|---|---|
| SEQ ID NO: 5 | >1 | >1 | >1 | >1 |
| SEQ ID NO: 6 | 0.895 | 0.894 | >1 | >1 |
| SEQ ID NO: 10 | >1 | >1 | >1 | >1 |
| 4-AP | 1370.9 | 516.9 | 1913.1 | 1529.2 |

TABLE 17
Z′ Values for Kv1.3 and Kv1.5 Ion Channel Assay

| Z′ | |
|---|---|
| Kv1.3 | Kv1.5 |

| | TP1 | TP6 | TP1 | TP6 |
|---|---|---|---|---|
| 4-AP | 0.794 | 0.860 | 0.674 | 0.799 |

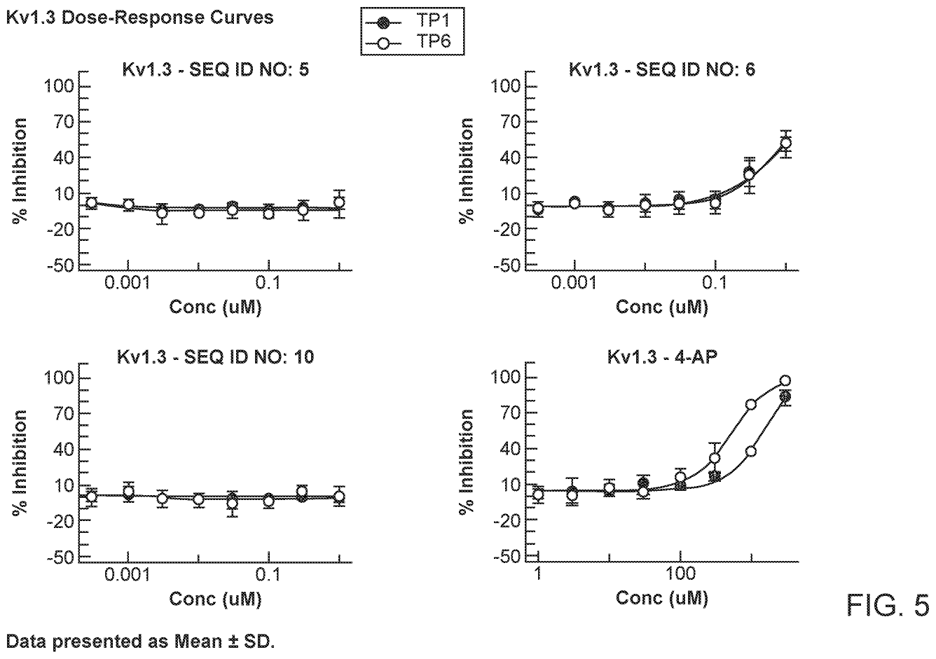

FIG. 5 illustrates the Kv1.3 dose-response curves for each peptide and 4-AP control.

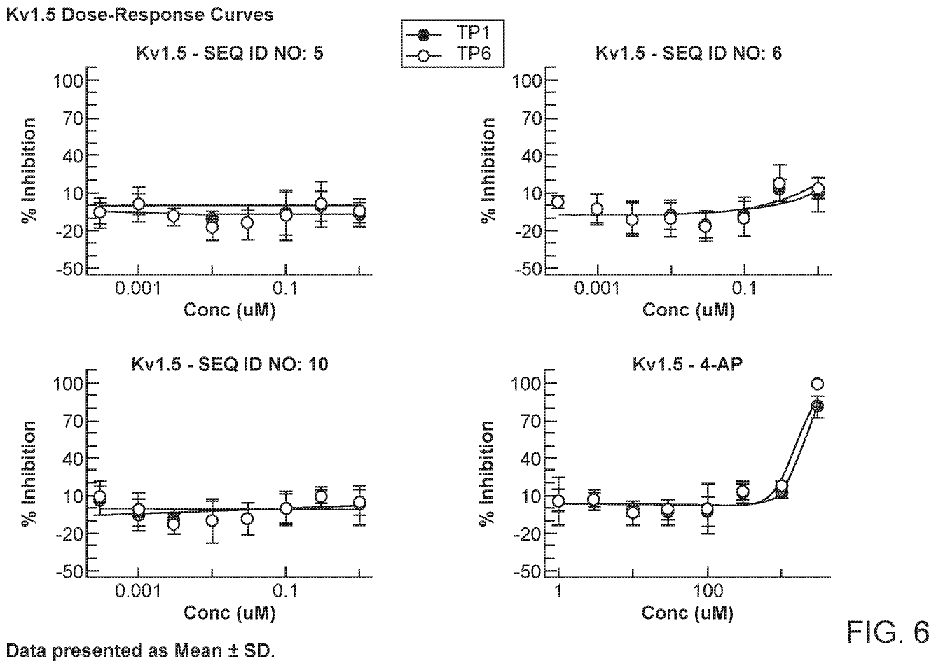

FIG. 6 illustrates the Kv1.5 dose-response curves for each peptide and 4-AP control.

Nav1.x Channel Results:

IC₅₀ values for ion channel inhibition for each peptide are presented TABLE 18 below. Z′ factor values are presented in TABLE 19. In cases where the maximal inhibition effect was less than 50%, the IC₅₀ value was not calculated.

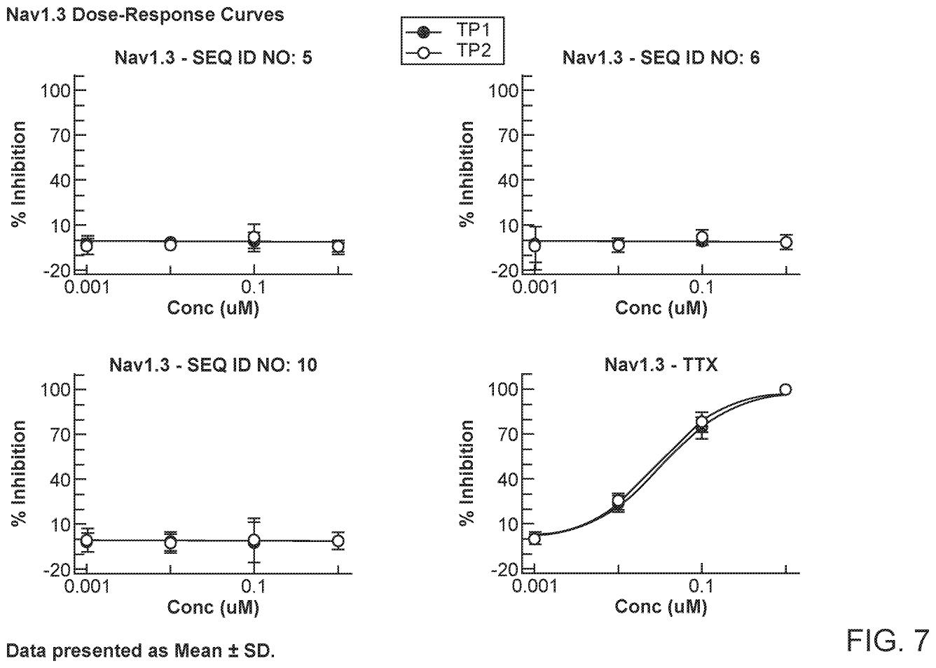

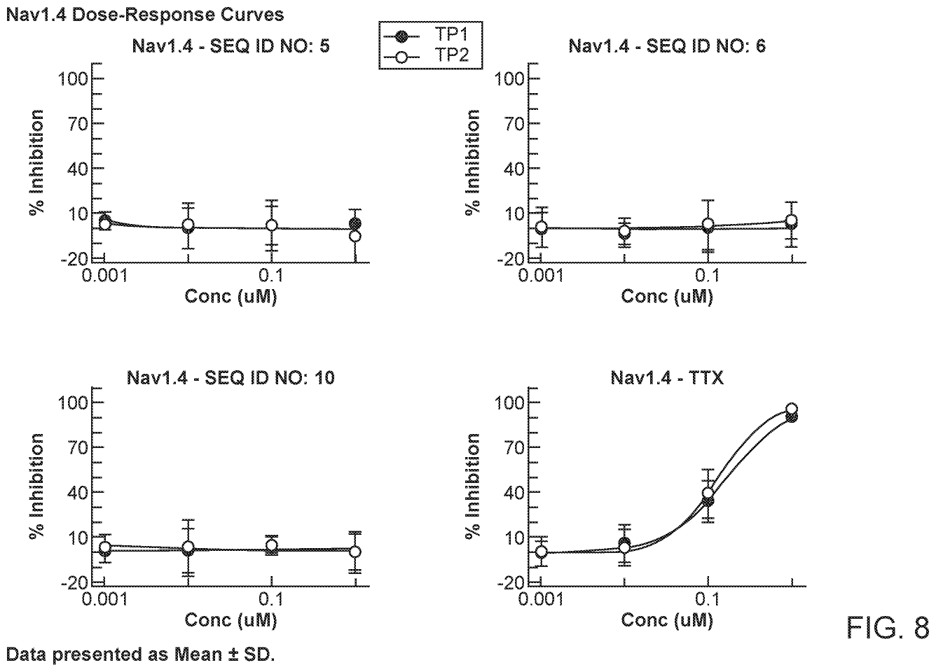

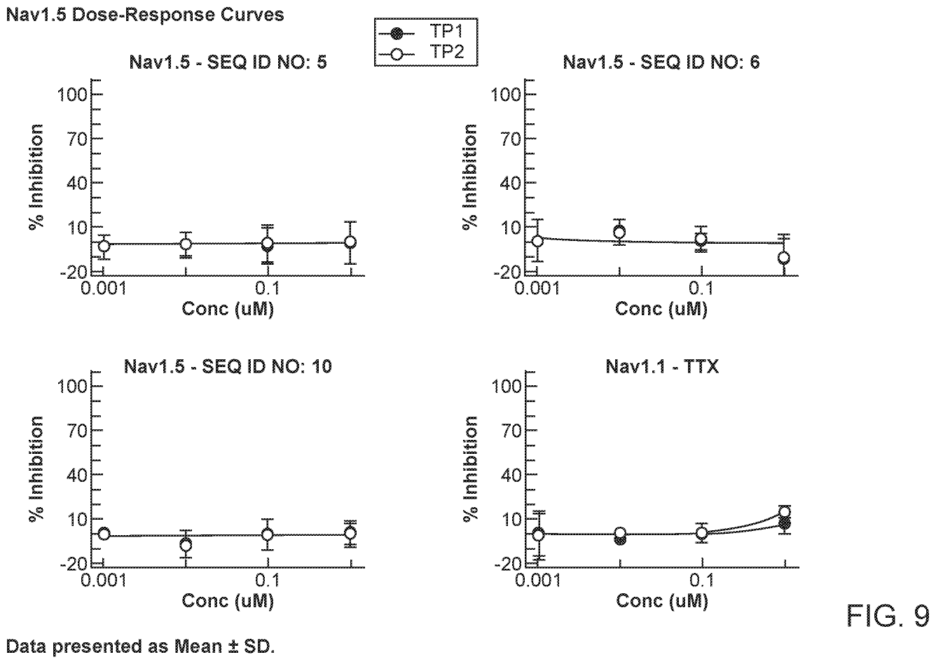

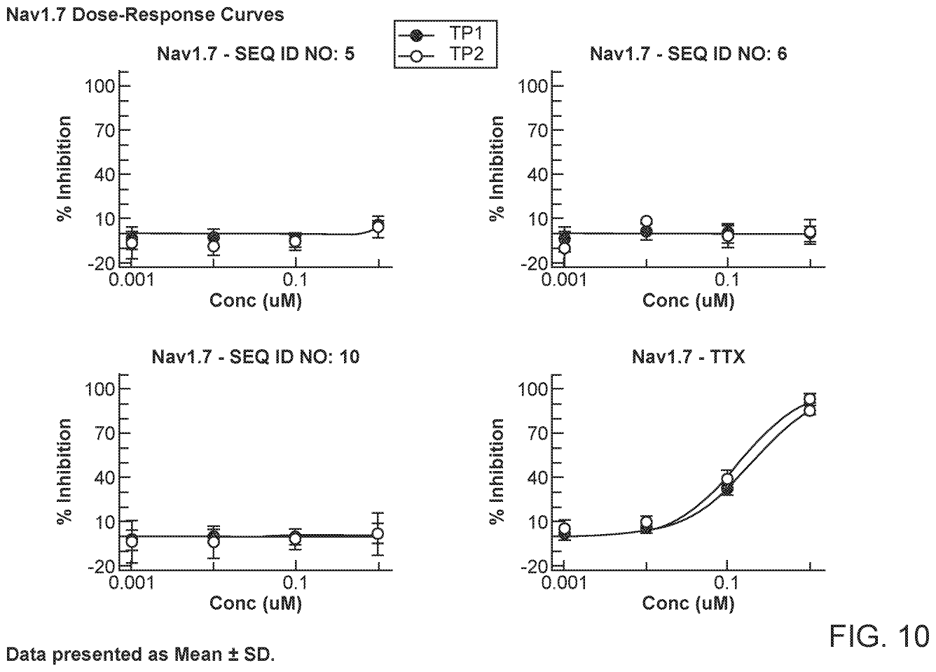

FIG. 7, FIG. 8, FIG. 9, and FIG. 10 each illustrates the Nav1.3, Nav1.4, Nav1.5, and Nav1.7 dose-response curves for each peptide and TTX positive control, respectively.

TABLE 18
IC₅₀ Values for Nav1.3, Nav1.4, Nav1.5 and Nav1.7 Ion
Channel Inhibition with Peptides and TTX Control

| | IC₅₀, μM | | | | | | | |
|---|---|---|---|---|---|---|---|---|
| | Nav1.3 | | Nav1.4 | | Nav1.5 | | Nav1.7 | |
| Peptide | TP1 | TP2 | TP1 | TP2 | TP1 | TP2 | TP1 | TP2 |
| SEQ ID NO: 5 | >1 | >1 | >1 | >1 | >1 | >1 | >1 | >1 |
| SEQ ID NO: 6 | >1 | >1 | >1 | >1 | >1 | >1 | >1 | >1 |
| SEQ ID NO: 10 | >1 | >1 | >1 | >1 | >1 | >1 | >1 | >1 |
| TTX | 0.033 | 0.028 | 0.165 | 0.132 | >1 | >1 | 0.191 | 0.134 |

TABLE 19
Z′ Values for Nav1.3, Nav1.4, Nav1.5 and
Nav1.7 Ion Channel Assay

| | Z′ | | | | | | | |
|---|---|---|---|---|---|---|---|---|
| | Nav1.3 | | Nav1.4 | | Nav1.5 | | Nav1.7 | |
| Control | TP1 | TP2 | TP1 | TP2 | TP1 | TP2 | TP1 | TP2 |
| TTX | 0.730 | 0.719 | 0.531 | 0.539 | ND | ND | 0.657 | 0.577 |

### Example 6

### Effects of Peptide on Ion Channels

This example describes the interaction between peptides of the present disclosure and ion channels. Ion channels can be associated with disease states in the kidney, including variations in ion channels that cause disease or modulation of ion channels in order to treat diseases (Kuo et al. Chem Rev. 2012 Dec. 12; 112(12):6353-72). A peptide of the disclosure, e.g., SEQ ID NO: 7 or 47, is expressed and administered in a pharmaceutical composition to a patient to treat a kidney condition or disease associated with a calcium ion channel and treatable by binding, blocking, modulating, or interacting with the ion channel. Ion channels, such as Cav2.1 and Cav2.2, are inhibited by peptides of the present disclosure. A given peptide is expressed recombinantly or chemically synthesized, wherein the peptide selected from any of SEQ ID NO: 1-SEQ ID NO: 80. Following expression or synthesis, the peptide is used directly or conjugated to a therapeutic compound, such as those described herein. A peptide of the present disclosure selectively interacts with ion channels, or is mutated in order to interact, bind, agonize, or antagonize, with ion channels. For example, a peptide of this disclosure modulates Cav1.1, Cav2.2, Kv1.1, Kv1.2, Kv1.3, Kv2.1, Nav1.5, Nav1.7, hERG, or NR2A. When the peptide is administered to a human subject, kidney TRPC6 function is modulated, Ca2+ influx is more normalized and focal segmental glomerulosclerosis is treated.

The peptide can be any one of SEQ ID NO: 1-7, 11-14, 17-22, 26, 29, 31, 39, and 40. Such peptide-drug conjugates can be made using either a cleavable or non-cleavable linker as described herein.

### Example 7

### Intra-Kidney Administration of Peptides and Peptide Conjugates

This example illustrates direct introduction into kidney by administration of peptides or peptide conjugates of this disclosure. A peptide of this disclosure is expressed recombinantly or chemically synthesized. In some cases, the peptide is subsequently conjugated to a detectable agent or an active agent. The peptide or peptide conjugate is administered to a subject in need thereof via administration by injection or placement directly into the kidney. Alternately, the peptide is administered systemically, such as intravenously or subcutaneously, but accumulate in the kidney. The kidney is penetrated by the peptide or peptide conjugate due to the small size of the peptide or peptide conjugate, and due to binding of kidney components by the peptide or peptide conjugate. The peptide or peptide conjugate is bound to or retained by the kidney and the residence time in the kidney is longer due to this binding. Optionally, the injected material is aggregated, is crystallized, or complexes are formed, further extending the depot effect and contributing to longer residence time.

The peptide can be any one of SEQ ID NO: 1-SEQ ID NO: 80. Such peptide-drug conjugates can be made using either a cleavable or non-cleavable linker as described herein.

### Example 8

### Treatment or Management of Pain

This example describes a method for treating or managing pain, including neuropathic pain. This method is used as a treatment for acute and/or chronic symptoms associated with any one of voltage-gated sodium and calcium channels, including Cav2.1, Cav2.2, Nav1.7, or NR2A, Kv1.1 and TRP, ASIC, ligand-gated ion channels in the brain, central nervous system, peripheral nervous system or elsewhere. A peptide of the disclosure is expressed and administered in a pharmaceutical composition to a patient as a therapeutic for pain management. The peptide of the present disclosure inhibits ion channels, such as Cav2.2 or Nav1.7 channel. The peptide is expressed recombinantly or chemically synthesized, wherein the peptide selected from any of SEQ ID NO: 1-SEQ ID NO: 80. The peptide is optionally modified to increase its affinity to the ion channel and to add or increase ion channel inhibition, such as Nav1.7. Following expression or synthesis, the peptide is used directly or conjugated to a narcotic (e.g. oxycodone), a non-narcotic analgesic, a counter-irritant (capsaicin), or a pain receptor channel inhibitor (such as the TRPV4 inhibitor GSK2193874). Following administration of the peptide, the peptide is exposed to the area of the central or peripheral nervous system affected by pain. One or more peptides are administered to a human or animal subcutaneously, intravenously, or orally, or is injected directly into the brain or intrathecally. The pain is reduced in the patient.

The peptide can be any one of SEQ ID NO: 7, 14, 17, 18, 31, 47, 54, and 57. Such peptide-drug conjugates can be made using either a cleavable or non-cleavable linker as described herein.

### Example 9

### Treatment of a Kidney Condition with a Peptide of the Disclosure

This example describes treatment of a kidney condition with peptides of this disclosure. The peptide is administered to a human or animal, where it binds to renal tissue and exhibits a therapeutic effect, e.g., via antioxidant or anti-inflammatory actions or affecting ion flux and thus blood pressure. A peptide of the disclosure (e.g., any of the peptides of SEQ ID NO: 1-SEQ ID NO: 80) is expressed recombinantly or chemically synthesized. For example, a peptide of the present disclosure is taken up by the proximal tubules, which results in the suppression of intracellular injury pathways by targeting ion channels in the pathways. As another example, a peptide of the present disclosure migrates to the renal interstitium and inhibits interstitial inflammation and prevents renal fibrosis.

### Example 10

### Treatment of a Gastrointestinal (GI) Disorder

This example describes treatment of a GI disorder with a peptide or peptide conjugate of this disclosure. An efficacious amount of the intact peptide or peptide conjugate is administered, which binds to an ion channel expressed by cells of the GI tract to modulate GI motility, which provides a method for treating colorectal cancer, inflammatory bowel disease, constipation, motility disorders, Crohn's disease, lupus, inflammation, or irritable bowel syndrome. The peptide binds, antagonizes, agonizes, or modulates Kv1.1, 1.2, and Nav1.5 ion channels. A peptide of the disclosure (e.g., any of the peptides of SEQ ID NO: 1-SEQ ID NO: 80) is expressed recombinantly or chemically synthesized. The peptide is administered as the therapeutic or it can be conjugated to an active agent, such as an antibiotic (e.g., carbapenems, penicillins, quinolines, fluorquinolines, etc.), a chemotherapeutic, an anti-apoptotic agent (e.g., a BCL2 inhibitor), a senolytic, or an anti-inflammatory agent (e.g., a steroid). The peptide or peptide conjugate of this disclosure is formulated and orally administered to a subject. The peptide can be formulated in a pharmaceutical composition. The subject can be an animal or a human. Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the gastrointestinal disease is relieved.

The peptide can be any one of SEQ ID NO: 6, 7, 14, 17, 19, 39, 40, 47, 54, and 57. Such peptide-drug conjugates can be made using either a cleavable or non-cleavable linker as described herein.

### Example 11

### Treatment of Colon Cancer

This example shows treatment of colon cancer with any peptide (SEQ ID NO: 1-SEQ ID NO: 80) or peptide-active agent conjugate of this disclosure. A peptide of interest is recombinantly expressed or chemically synthesized either alone or as a fusion or conjugate with an active agent. The peptide or the peptide-active agent conjugate is orally, intravenously, or subcutaneously administered to a subject in need thereof. The subject in need thereof is a human or a non-human animal. The subject in need thereof has colon cancer. In the case of the peptide-active agent conjugate, the active agent is any anti-cancer drug, such as fluorouracil, gemcitabine, or mafosphamide (a cyclophosphamide pro drug). The peptide itself is adapted for binding to an ion channel expressed by cancer cells. Upon binding to the ion channel, the peptide inhibits ion channel activity, which leads to cell death of cancer cells.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the colon cancer is treated.

The peptide modulates Kv1.3 or gut hERG. The peptide can be any one of SEQ ID NO: 2, 5, 6, 7, 12, 14, 17, 19, 20, 21, 29, 33, and 39. Such peptide-drug conjugates can be made using either a cleavable or non-cleavable linker as described herein

### Example 12

### Agonism or Antagonism of Ion Channels to Treat Irritable Bowel Syndrome

This example shows treatment of irritable bowel syndrome by agonizing or antagonizing ion channels in the gastrointestinal tract with any peptide (SEQ ID NO: 1-SEQ ID NO: 80) or peptide-active agent conjugate of this disclosure. A peptide of interest is recombinantly expressed or chemically synthesized either alone or as a fusion or conjugate with an active agent. The peptide or the peptide-active agent conjugate is orally administered to a subject in need thereof. The subject in need thereof is a human or a non-human animal. The subject in need thereof has irritable bowel syndrome. The voltage gated sodium (NaV) ion channels, calcium (CaV) ion channels, potassium (KV, KCa) ion channels, Kv1.1, Kv1.2, chloride (Cl—) ion channels, nonselective ion channels (transient receptor potentials (TRPs)), chloride channel type-2 (ClC-2)3 ion channels, or cystic fibrosis transmembrane conductance regulator (CFTR) chloride channels in the gastrointestinal tract are agonized or antagonized by the peptide or peptide-active agent conjugate ((Beyder, A., Therap Adv Gastroenterol., 5(1): 5-21 (2012); Jun, J. Y. J Neurogastroenterol Motil., 19(3): 277-8 (2013). Alternatively, the peptide itself binds to these ion channels to block or activate the ion channels.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the irritable bowel is relieved.

### Example 13

### Treatment of a Neurodegenerative Disease with a Peptide of the Disclosure

This example describes the treatment of a neurodegenerative disease with a peptide. A peptide of the disclosure (e.g., any of the peptides of SEQ ID NO: 1-SEQ ID NO: 80) is expressed recombinantly or chemically synthesized, and administered to a human or animal. The peptide crosses the BBB and agonizes or antagonizes an ion channel, such as potassium sodium channels, chloride channels, calcium channels, nicotinic acetyl choline receptors, transient receptor potential channels, NMDA receptors, serotonin receptors, KIR channels, GABA channels, glycine receptors, glutamate receptors, acid sensing ion channels, K2P channels, Nav1.7, Kv1.1, NR2A, or purinergic receptors, thereby providing a therapeutic effect.

### Example 14

### Treatment of Rheumatoid Arthritis

This example describes a method for treating rheumatoid arthritis. This method is used as a treatment for acute and/or chronic symptoms associated with rheumatoid arthritis. A peptide of the disclosure (e.g., any of the peptides of SEQ ID NO: 1-SEQ ID NO: 80) is expressed recombinantly or chemically synthesized, and then is used directly, or is conjugated to an anti-inflammatory compound, such as triamcinolone and dexamethasone. When the peptide is used directly, the peptide can, for example, bind or inhibit ion channels such as Kv 1.3 or TRPV4 in the cartilage. The resulting peptide or peptide-drug conjugate is administered in a pharmaceutical composition to a patient and is targeted to cartilage. The peptide is selected from SEQ ID NO: 1-SEQ ID NO: 80. One or more anti-inflammatory compound-peptide conjugates are administered to a human or animal subcutaneously, intravenously, or orally, or is injected directly into a joint.

The peptide can be any one of SEQ ID NO: 2, 5, 6, 12, 17, 18, 19, 20, 21, 29, 33, 38, 39, and 40.

### Example 15

### Treatment of Breast Cancer

This example describes a method for treating breast cancer. A peptide of the disclosure (e.g., any of the peptides of SEQ ID NO: 1-SEQ ID NO: 80) is expressed recombinantly or chemically synthesized, and then is used directly, or is conjugated to an active agent, such as docetaxel or an antibody. When the peptide is used directly, the peptide can, for example, bind or inhibit ion channels, such as Nav1.5 or Nav1.7. The resulting peptide or peptide-drug conjugate is administered in a pharmaceutical composition to a patient and is targeted to breast cancer cells. The peptide is selected from SEQ ID NO: 1-SEQ ID NO: 80. One or more breast cancer peptide conjugates are administered to a human or animal intravenously or orally.

The peptide can be any one of SEQ ID NO: 7, 16, 18, 31, 47, 56, 58, and 71. Such peptide is administered to to a patient to inhibit Nav1.5 or Nav1.7 channels associated with breast cancer cells. Peptide-drug conjugates can be made using either a cleavable or non-cleavable linker as described.

### Example 16

### Treatment of Autoimmune Diseases

This example describes a method for treating autoimmune diseases, including rheumatoid arthritis, type 1 diabetes, multiple sclerosis, Hashimoto's thyroiditis, Sjorgen's syndrome, systemic lupus erythematosus, autoimmune glomerulonephritis, and psoriasis. A peptide of the disclosure (e.g., any of the peptides of SEQ ID NO: 1-SEQ ID NO: 80) is expressed recombinantly or chemically synthesized, and then is used directly, or is conjugated to an immunomodulatory agent, such as TNF-α antagonists, e.g., lenalidomide and pomalidomide. When the peptide is used directly, the peptide can, for example, bind or inhibit ion channels such as Kv 1.3 in effector memory T cells. The resulting peptide or peptide-drug conjugate is administered in a pharmaceutical composition to a patient and is targeted to Kv1.3 ion channels expressed in T cells, especially memory T cells. The peptide is selected from SEQ ID NO: 1-SEQ ID NO: 80. One or more immunomodulator-peptide conjugates are administered to a human or animal subcutaneously, intravenously, or orally.

The peptide can be a peptide or any of SEQ ID NO: 2, 6, 12, 17, 19, 20, 21, 29, 33, 39 and 40 that selectively inhibits Kv1.3 in memory T cells.

### Example 17

### Treatment of Seizure

This example describes a method for treating ion channel related diseases. This method is used as a treatment for seizure. A peptide of the disclosure (e.g., any of the peptides of SEQ ID NO: 1-SEQ ID NO: 80) is expressed recombinantly or chemically synthesized, and then is used directly, or is conjugated to an active agent, such as lacosamide. When the peptide is used directly, the peptide can, for example, bind or inhibit ion channels such as Kv1.1, Cav2.1, Nav1.1, Nav1.2, Nav2.1, or NR2A in the CNS. The resulting peptide or peptide-drug conjugate is administered in a pharmaceutical composition to a patient and is targeted to the CNS. The peptide is selected from SEQ ID NO: 1-SEQ ID NO: 80. One or more anti-convulsant peptide conjugates are administered to a human or animal subcutaneously, intravenously, or orally.

The peptide can be any one of SEQ ID NO: 6, 7, 16, 18, 19, 31, and 40 that selectively inhibits Kv1.1, Cav2.1, Nav1.1, Nav1.2, Nav2.1, or NR2A.

### Example 18

### Treatment of Asthma, Obesity, and Insulin Resistance

This example describes a method for treating potassium ion channel related diseases, such as asthma, obesity, and insulin resistance. In particular, Kv7.1 (KCNQ1), Kir6.1 (K_{ATP}), and K_{Ca}3.1 (KCNN4) potassium channels are associated with asthma. Kv1.1, Kv1.5, Kv2.1, Kv7.1, Kv10.1, and Kv11.1 of adipose cells are associated with obesity, while ATP sensitive and calcium activated potassium channels are associated with insulin resistance. A peptide of the disclosure (e.g., any of the peptides of SEQ ID NO: 1-SEQ ID NO: 80) is expressed recombinantly or chemically synthesized, and then is used directly, or is conjugated to an active agent. When the peptide is used directly, the peptide can, for example, bind or inhibit potassium ion channels, such as Kv 1.1, in the diseased tissue. The resulting peptide or peptide-drug conjugate is administered in a pharmaceutical composition to a patient subcutaneously, intravenously, or orally.

The peptide can be any one of SEQ ID NO: 2, 17, 20, 33, and 40, that selectively targets and inhibits a potassium ion channel.

### Example 19

### Method of Engineering a Selective Peptide

This example describes a method for engineering a peptide with improved selectivity for Kv1.3, but not Kv1.2 or Kv1.1. This method is used to improve the binding properties of a peptide selected from SEQ ID NO: 1-SEQ ID NO: 80. Sequence alignments with other potassium ion channels in the family and native toxins are used to determine the conserved amino acid residues and amino acid attributes at certain positions in a peptide sequence, such as solvent-exposed residues at the binding interface. Keeping the disulfide bonds intact, residues at the binding interface are modified individually using site-directed mutagenesis and screened for binding properties, such as inhibition of a panel of ion channels. A peptide having the highest inhibition activity is selected for further analysis and verification in mammalian cells. Such peptide is conjugated to a PEG polymer at the N-terminus to enhance selectivity for Kv1.3 over Kv1.1 or Kv1.2.

What is claimed:

1. A composition comprising:
a peptide that modulates an ion channel activity, the peptide comprising an amino acid sequence according to:
X⁰X¹X²X³X⁴X⁵X⁶X⁷X⁸X⁹X¹⁰X¹¹X¹²CX¹⁴X¹⁵X¹⁶CX¹⁸X¹⁹X²⁰X²¹X²²X²³X²⁴X²⁵X²⁶X²⁷CX²⁹NX³¹X³²CX³⁴CX³⁶X³⁷X³⁸X₃₉ (SEQ ID NO: 83), wherein any one of X⁰ to X³⁹ is independently any amino acid or null.

2. The composition of claim 1, wherein the peptide comprises an amino acid sequence according to:
X⁰X¹X²X³X⁴VKCX⁸GSX¹¹X¹²CLX¹⁵PCKX¹⁹X²⁰X²¹GX²³RX²⁵GKCMNGKCX³⁴CX³⁶PX³⁸X³⁹ (SEQ ID NO: 84), wherein any one of X⁰ to X³⁹ is independently any amino acid or null, and wherein the peptide further comprises one or more of:
(a) X⁰ is G, Q, or null;
(b) X¹ is R, Q, V, I, or null;
(c) X² is P, F, G, R, V, N, D, A, or null;
(d) X³ is T, I, F, or E;
(e) X⁴ is D, N, P, G, K, V, or I,
(f) X⁵ is I, V, or Q;
(g) X⁶ is K, R, S, or E;
(h) X⁷ is C or G;
(i) X⁸ is S, T, R, K, Y or A;
(j) X⁹ is A, G, H, R or C;
(k) X¹⁰ is S or T,
(l) X¹¹ is Y, K, R, G, P, or S;
(m) X¹² is Q, D, E, P, or K;
(n) X¹⁴ is F, W, L, I, Y, R, or V;
(o) X¹⁵ is P, D, K, Q, S, G, or A;
(p) X¹⁶ is V, P, K, A, Y, or I;
(q) X¹⁸ is K, R, I, or Q;
(r) X¹⁹ is S, Q, K, R, D, or E;
(s) X²⁰ is R, M, A, L, K, or Q;
(t) X²¹ is F, I, V, Y, T, or null;
(u) X²² is G or N;
(v) X²³ is K, M, A, T, or C;
(w) X²⁴ is T, P, R, S, A, or L;
(x) X²⁵ is N, F, A, T, or G;
(y) X²⁶ is G, A, or S;
(z) X²⁷ is R or K;
(aa) X²⁹ is V, M, I, T, S or L;
(bb) X³¹ is G, S, R, or K;
(cc) X³² is L, K, R, V, or A;
(dd) X³⁴ is D, R, H, K, or T;
(ee) X³⁶ is F, Y, T, or null;
(ff) X³⁷ is S, P, Y, G, or null;
(gg) X³⁸ is K, C, null; and
(hh) X³⁹ is G, V, or null.

3. The composition of any one of claims 1-2, wherein any one of X⁰ to X³⁹ is independently any amino acid or null, and wherein the peptide further comprises one or more of:
(a) X⁵ is I or V;
(b) X⁶ is K or R;
(c) X⁷ is C;
(d) X¹⁰ is S;
(e) X²² is G;
(f) X²⁶ is G;
(g) X²⁷ is K;
(h) X²⁹ is V, M, I, or T;
(i) X³² is K or R; and
(j) X³⁴ is H, K, or D.

4. The composition of any one of claims 1-3, wherein the peptide comprises an amino acid sequence according to:
X⁰VX²X³X⁴VKCX⁸GSX¹¹X¹²CLX¹⁵PCKX¹⁹X²⁰X²¹GX²³RX²⁵GKCMNGKCX³⁴CX³⁶PX³⁸X³⁹ (SEQ ID NO: 84), wherein any one of X⁰ to X³⁹ is independently any amino acid or null.

5. The composition of any one of claims 1-4, wherein X⁰ is G, Q, or null.

6. The composition of any one of claims 1-5, wherein X¹ is R, Q, V, I, or null.

7. The composition of any one of claims 1-6, wherein X² is P, F, G, R, V, N, D, A, or null.

8. The composition of any one of claims 1-7, wherein X³ is T, I, F, or E.

9. The composition of any one of claims 1-8, wherein X⁴ is D, N, P, G, K, V, or I.

10. The composition of any one of claims 1-9, wherein X⁵ is I, V, or Q.

11. The composition of any one of claims 1-10, wherein X⁶ is K, R, S, or E.

12. The composition of any one of claims 1-11, wherein X⁷ is C or G.

13. The composition of any one of claims 1-12, wherein X⁸ is S, T, R, K, Y or A.

14. The composition of any one of claims 1-13, wherein X⁹ is A, G, H, R or C.

15. The composition of any one of claims 1-14, wherein X¹⁰ is S or T.

16. The composition of any one of claims 1-15, wherein X¹¹ is Y, K, R, G, P, or S.

17. The composition of any one of claims 1-16, wherein X¹² is Q, D, E, P, or K.

18. The composition of any one of claims 1-17, wherein X¹⁴ is F, W, L, I, Y, R, or V.

19. The composition of any one of claims 1-18, wherein X¹⁵ is P, D, K, Q, S, G, or A.

20. The composition of any one of claims 1-19, wherein X¹⁶ is V, P, K, A, Y, or I.

21. The composition of any one of claims 1-20, wherein X¹⁸ is K, R, I, or Q.

22. The composition of any one of claims 1-21, wherein X¹⁹ is S, Q, K, R, D, or E.

23. The composition of any one of claims 1-22, wherein X²⁰ is R, M, A, L, K, or Q.

24. The composition of any one of claims 1-23, wherein X²¹ is F, I, V, Y, T, or null.

25. The composition of any one of claims 1-24, wherein X²² is G or N.

26. The composition of any one of claims 1-25, wherein X²³ is K, M, A, T, or C.

27. The composition of any one of claims 1-26, wherein X²⁴ is T, P, R, S, A, or L.

28. The composition of any one of claims 1-27, wherein X²⁵ is N, F, A, T, or G.

29. The composition of any one of claims 1-28, wherein X²⁶ is G, A, or S.

30. The composition of any one of claims 1-29, wherein X²⁷ is R or K.

31. The composition of any one of claims 1-30, wherein X²⁹ is V, M, I, or T.

32. The composition of any one of claims 1-31, wherein X³¹ is G, S, R, or K.

33. The composition of any one of claims 1-32, wherein X³² is L, K, R, V, or A.

34. The composition of any one of claims 1-33, wherein X³⁴ is D, R, H, K, or T.

35. The composition of any one of claims 1-34, wherein X³⁶ is F, Y, T, or null.

36. The composition of any one of claims 1-35, wherein X³⁷ is S, P, Y, G, or null.

37. The composition of any one of claims 1-36, wherein X³⁸ is K, C, null.

38. The composition of any one of claims 1-37, wherein X³⁹ is G, V, or null.

39. The composition of any one of claims 1-38, wherein the peptide comprises an anti-parallel beta sheet domain that interacts with the ion channel.

40. The composition of any one of claims 1-39, wherein the peptide comprises 2 or more, 3 or more, 4 or more, 5 or more, 6 or more, 7 or more, 8 or more, 9 or more, or 10 or more solvent-exposed basic residues that interact with the ion channel.

41. The composition of any one of claims 1-40, wherein the peptide comprises a R or K that blocks an entryway of the ion channel upon binding.

42. The composition of any one of claims 1-41, wherein the peptide has at least 80%, 85%, 90%, 95%, 96%, 97%, 98%, 99%, or 100% sequence identity to any one of SEQ ID NO: 1-SEQ ID NO: 80 or a functional fragment thereof.

43. The composition of any one of claims 1-42, wherein the peptide is non-naturally occurring.

44. The composition of any one of claims 1-43, wherein the peptide comprises GS amino acid residues at the N-terminus.

45. The composition of any one of claims 1-44, wherein the peptide is a knotted peptide.

46. The composition of any one of claims 1-45, wherein the peptide comprises 6 or more cysteine residues.

47. The composition of any one of claims 1-46, wherein the peptide comprises three or more disulfide bridges formed between cysteine residues, wherein one of the disulfide bridges passes through a loop formed by two other disulfide bridges.

48. The composition of any one of claims 1-47, wherein the peptide comprises a plurality of disulfide bridges.

49. The composition of any one of claims 1-48, wherein the peptide is a cystine-dense peptide (CDP).

50. The composition of claim 49, wherein the CDP comprises independent folding domains, wherein the independent folding domains comprise a high density of at least six cysteines.

51. The composition of any one of claims 49-50, wherein the CDP is a non-knotted CDP.

52. The composition of any one of claims 1-51, wherein the peptide comprises a topology of a Cysu-Cysv disulfide bond, a Cysw-Cysx disulfide bond, and a Cysy-Cysz disulfide bond, wherein the Cysw-Cysx disulfide bond passes through a macrocycle comprising the Cysu-Cysv disulfide bond and the Cysy-Cysz disulfide bond.

53. The composition of claim 52, wherein the Cysw-Cysx cysteine-cysteine bond is a knotting cysteine.

54. The composition of any one of claims 52-53, wherein the peptide is a hitchin, and wherein the hitchin comprises a topology wherein the Cysu-Cysy disulfide bond is between cysteine 1 and cysteine 4, the Cysw-Cysx disulfide bond is between cysteine 2 and cysteine 5, and wherein the Cysy-Cysz disulfide bond is between cysteine 3 and cysteine 6.

55. The composition of any one of claims 1-54, wherein at least one amino acid residue of the peptide is in an L configuration or, wherein at least one amino acid residue of the knotted peptide is in a D configuration.

56. The composition of any one of claims 1-55, wherein the peptide is at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 amino acid residues long.

57. The composition of any one of claims 1-56, wherein any one or more K residues are replaced by an R residue or wherein any one or more R residues are replaced by for a K residue.

58. The composition of any one of claims 1-57, wherein the knotted peptide has a charge distribution comprising an acidic region and a basic region.

59. The composition of any of claims 1-58, wherein the knotted peptide comprises 6 or more basic residues and 2 or fewer acidic residues.

60. The composition of any of claims 1-59, wherein the knotted peptide comprises a 4-19 amino acid residue fragment containing at least 2 cysteine residues, and at least 2 positively charged amino acid residues.

61. The composition of any of claims 1-60, wherein the knotted peptide comprises a 20-70 amino acid residue fragment containing at least 2 cysteine residues, no more than 2 basic residues and at least 2 positively charged amino acid residues.

62. The composition of any of claims 1-61, wherein the knotted peptide comprises at least 3 positively charged amino acid residues.

63. The composition of any of claims 61-62, wherein the positively charged amino acid residues are selected from K, R, or a combination thereof.

64. The composition of any one of claims 1-63, wherein the knotted peptide is selected from a potassium channel agonist, a potassium channel antagonist, a sodium channel agonist, a sodium channel antagonist, a calcium channel agonist, a calcium channel antagonist, a hadrucalcin, a theraphotoxin, a huwentoxin, a kaliotoxin, a cobatoxin, a lectin, a GABA agonist, a GABA antagonist, a NR2A/NR2B agonist, a NR2A/NR2B antagonist, a nicotinic receptor agonist, a nicotinic receptor antagonist, a TRP agonist, or a TRP antagonist.

65. The composition of any one of claims 1-64, wherein at least one residue of the knotted peptide comprises a chemical modification.

66. The composition of claim 65, wherein the chemical modification is blocking the N-terminus of the knotted peptide.

67. The composition of claim 66, wherein the chemical modification is methylation, acetylation, or acylation.

68. The composition of claim 67, wherein the chemical modification is:
methylation of one or more lysine residues or analogue thereof;
methylation of the N-terminus; or
methylation of one or more lysine residue or analogue thereof and methylation of the N-terminus.

69. The composition of any one of claims 1-68, wherein the knotted peptide is linked to an acyl adduct.

70. The composition of any one of claims 1-69, wherein the knotted peptide is linked to an active agent.

71. The composition of claim 70, wherein the active agent is fused with the knotted peptide at an N-terminus or a C-terminus of the knotted peptide.

72. The composition of any one of claims 69-71, wherein the knotted peptide is linked to the active agent via a cleavable linker or a non-cleavable linker.

73. The composition of any one of claims 70-72, wherein the active agent is a detectable agent.

74. The composition of claim 73, wherein the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator.

75. A method of modulating an ion channel activity, the method comprising: administering to a subject a composition according to any one of claims 1-74.

76. A method of treating an ion channel-associated disorder in a subject in need thereof, the method comprising: administering to the subject in need thereof a composition according to any one of claims 1-74.

77. The method of claim 75 or 76, wherein the ion channel-associated disorder is selected from the group consisting of Bartter's syndrome, Andersen's syndrome, congenital hyperinsulinism, dilated cardiomyopathy, episodic ataxia type 1 or type 2, long QT syndrome, short QT syndrome, benign neonatal febrile convulsions, nonsyndromic deafness, polycystic kidney disease, familial episodic pain syndrome, focal segmental glomerulosclerosis, Retinitis pigmentosa, Severe myoclonic epilepsy, cerebellar ataxia, erythromelalgia, paroxysmal extreme pain disorder, congenital indifference to pain, benign familial neonatal seizures, Timothy syndrome, GI motility disorders, constipation, irritable bowel syndrome, Crohn's disease, diarrhea, inflammatory bowel disease, GI pain, rheumatoid arthritis, anklysosis spondylitis, multiple sclerosis, autoimmune disease, psoriasis, Hashimoto's thyroiditis, Sjorgen's syndrome, autoimmune glomerulonephritis, lupus, type-1 diabetes, pain, neuropathic pain, seizures, epilepsy, hypertension, renal hypertension, cancer, Parkinson's disease, neuromuscular disorders, cystic fibrosis, and dry eye.

78. The method of any one of claims 75-77, wherein the peptide binds to the ion channel.

79. The method of any one of claims 75-78, wherein the ion channel is a voltage-gated channel, a ligand-gated channel, or a ligand-activated channel, an inward rectifier channel, or a mechanosensitive ion channel.

80. The method of any one of claims 75-79, wherein the ion channel is a potassium channel, sodium channel, calcium channel, TRP channel, GABA receptor, NMDA receptor, ionotrophic glutamate receptor channel, acetylcholine receptor, nicotinic receptor, 5-HT3 receptor, or chloride channel.

81. The method of any one of claims 75-80, wherein the ion channel is selected from: 5-HT3a, alpha-4 beta-2 nicotinic receptor, alpha-3-beta-4 nicotinic receptor, Cav2.1, Cav2.2, GABA, hERG, Kir2.1, Kv1.1, Kv1.2, Kv1.3, Kv2.1, Nav1.5 (TP1), Nav1.5 (TP2), Nav1.7 (TP1), Nav1.7 (TP2), NR2A, NR2B, TRPA1, and TRPV1.

82. The method of claim 81, wherein the ion channel is selected from: 5HT3a, alpha-4 beta-2 nicotinic receptor, alpha-3-beta-4 nicotinic receptor, Cav2.2, Kv1.2, Kv2.1, NR2A, NR2B, and TRPV1.

83. The method of any one of claims 75-82, wherein the peptide inhibits or activates the ion channel.

84. The method of any one of claims 75-83, wherein the ion channel comprises a gain of function mutation or a loss of function mutation.

85. The method of any one of claims 75-84, wherein the ion channel is overexpressed or under-expressed in a target tissue or cell type.

86. The method of any one of claims 75-85, wherein ion channel activation or deactivation is associated with a disease state.

87. The method of any one of claims 75-86, wherein the peptide binds to the ion channel to induce a conformational change in the ion channel.

88. The method of any one of claims 75-87, wherein the peptide binds to the ion channel to block ion movement through the channel.

89. The method of any one of claims 75-88, wherein the peptide binds to the ion channel to enhance ion movement through the channel.

90. The method of any one of claims 75-88, wherein the peptide binds to the ion channel to block the ion channel from ligand interaction.

91. The method of any one of claims 75-90, wherein peptide affinity to an ion channel is in the range of 0.01 nM to 1000 nM.

92. The method of any one of claims 75-91, wherein the peptide inhibits ion channel activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% as measured by a ScreenPatch or ChanTest assay.

93. The method of any one of claims 75-92, wherein the peptide increases ion channel activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 100% as measured by a ScreenPatch or ChanTest assay.

94. The method of any one of claims 75-93, wherein peptide binding to the ion channel results in less off-target or toxicity effects as compared to a reference compound: mibefradil dihydrochloride for calcium ion channel; E-4031 for hERG; 4-aminopyridine for potassium ion channel; Verapamil for Kv2.1; lidocaine for sodium in channel; memantine for NR2A; capsazepine for TRP channel; picrotoxin for GABA channel; ondansetron for 5HT3a channel; mecamylamine for α3β4 or α4β2; or BaCl2 for Kir2.1.

95. The method of any one of claims 75-94, wherein the ion channel-related disease is a neurological disorder, cancer, autoimmune disease, GI motility disorder, irritable bowel syndrome, inflammatory bowel disease, constipation, dyspepsia, acquired neuromyotonia, renal disorder, ocular disorder, retinal disease, epilepsy, migraine, ataxia, polycystic kidney disease, seizure, long or short QT syndrome, paralysis, pain, neuropathic pain, severe pain, need for local anesthesia, migraine, cystic fibrosis, Bartter syndrome, endocrine disorder, rheumatoid arthritis, type 1 diabetes mellitus, multiple sclerosis, psoriasis, lupus, asthma, obesity, insulin resistance, hypertension, stroke, Alzheimer's disease, arrhythmia, neurodegenerative disease, or bone disease.

96. The method of claim 95, wherein cancer comprises breast cancer, cervical cancer, hepatocellular carcinoma, prostate cancer, colon cancer, squamous cell lung cancer, endometrial cancer, mammary gland cancer; adenocarcinoma, leukemia, chronic lymphocytic leukemia (CLL), acute myeloid leukemia (AML), glioma, glioblastoma, and neuroblastoma, or metastases.

97. The method of any one of claims 75-96, wherein the peptide targets a tissue or cell type comprising cardiac cells; renal cells; retinal cells; cancerous cells; gastrointestinal cells; epithelial cells; neurons, such as motor neuron, Purkinje cells, GABAergic neurons, excitatory neurons, sensory neurons, and interneurons; cartilage cells; immune cells, such as T and B lymphocytes; smooth muscle cells, and skeletal muscle cells.

98. The method of any one of claims 75-97, wherein administering comprises oral administration, rectal suppository, inhalation, intranasal administration, topical administration, intravenous administration, subcutaneous administration, intra-articular administration, intramuscular administration, intraperitoneal administration, intra-synovial administration, vaginal administration, rectal administration, pulmonary administration, ocular administration, buccal administration, sublingual administration, intrathecal administration, or any combination thereof.

99. The method of any one of claims 75-98, wherein the subject is a human.

100. The method of any one of claims 75-98, wherein the subject is a non-human animal.
